SEQUENCE LISTING (52 sequences)

SEQ ID NO: 1 - protein, 352 aa, synthetic construct
MDSYLLMWGLLTFIMVPGCQADVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDVAVAGCVFLLISVLLLSGLTWQRRQRKSRRTI

SEQ ID NO: 2 - protein, 636 aa, synthetic construct
MAAPALSWRLPLLILLLPLATSWASADVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 428 aa, synthetic construct
MLKPSLPFTSLLFLQLPLLGVGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 4 - protein, 539 aa, synthetic construct
MTILGTTFGMVFSLLQVVSGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 5 - protein, 391 aa, synthetic construct
MAPRRARGCRTLGLPALLLLLLLRPPATRGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDVAISTSTVLLCGLSAVSLLACYLKSRQTPPLASVEMEAMEALPVTWGTSSRDEDLENCSHHL

SEQ ID NO: 6 - protein, 624 aa, synthetic construct
MPRGWAAPLLLLLLQGGWGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 7 - protein, 1444 aa, synthetic construct
MAAPALSWRLPLLILLLPLATSWASADVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVTGSGATNFSLLKQAGDVEENPGPAMLKPSLPFTSLLFLQLPLLGVGDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGEGRGSLLTCGDVEENPGPWEMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 8 - DNA, 1062 bp, synthetic construct
  source 1..1062: mol_type = other DNA
atggacagctacctgctgatgtggggcctgctgaccttcatcatggtgcctggctgtcaggccgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggacttcgcctgtgatgtggccgtggccggatgtgtgtttctgctgatctctgtgctgctgctgagcggcctgacttggcaaagacggcagagaaagagccggcggaccatctgataa

SEQ ID NO: 9 - DNA, 1914 bp, synthetic construct
  source 1..1914: mol_type = other DNA
atggctgctccagctctgtcttggagactgcccctgctgattctgctgctgcctctggctacatcttgggcctctgccgacgtggtcatgacacagacaccactgagcctgcctgtgtctctgggagatcaggccagcatcagctgcagatccagccagtctctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacatcccctggctgggacatctgcttgttggactgtctggcgccttcggcttcatcatcctggtgtatctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagcccaggtggactggcccctgagattagccctctggaagtgctggaacgggacaaagtgacccagctgctcctccagcaggataaggtgccagaacctgccagcctgtccagcaatcacagcctgaccagctgctttaccaaccagggctacttcttcttccatctgcctgacgctctggaaatcgaggcctgccaggtgtacttcacctacgatccctacagcgaagaggaccccgatgaaggtgttgctggcgcccctacaggatcttctccacagcctctgcaacctctgagcggcgaggatgatgcctactgcacctttccaagcagggacgacctgctcctgttcagcccatctctgctcggaggaccatctcctccatctacagctccaggcggatctggcgctggcgaggaaagaatgccacctagcctgcaagagcgggtgcccagagattgggatcctcaacctctcggccctccaacacctggcgtgccagatctcgtggactttcagcctcctccagagctggtgctgagagaagctggcgaagaagtgccagacgctggccctagagagggcgttagctttccttggagcagacctcctggacagggcgagtttagggccctgaatgcaagactgcctctgaacaccgacgcctacctgtctctgcaagaactgcagggacaagaccccacacacctggtgtaatga

SEQ ID NO: 10 - DNA, 1290 bp, synthetic construct
  source 1..1290: mol_type = other DNA
atgctgaagcccagcctgccttttaccagcctgctgttcctgcagctgcctctgcttggcgtgggagatgtggtcatgacacagaccccactgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggacttcgcctgcgacgtggtcatctctgtgggctctatgggcctgatcatctccctgctgtgtgtgtacttttggctggaacggaccatgcctcggatccccacactgaagaacctggaagatctggtcaccgagtaccacggcaacttcagtgcttggagcggcgtgtcaaaaggactggccgaaagcctgcagcctgactactccgagagactgtgcctggtgtctgagatccctcctaaaggcggcgctctcggagaaggacctggtgcctctccatgcaatcagcacagcccttattgggcccctccttgctacaccctgaaacctgagacatgatga

SEQ ID NO: 11 - DNA, 3174 bp, synthetic construct
  source 1..3174: mol_type = other DNA
atgacaatcctgggcaccaccttcggcatggtgttcagtctgctgcaggtcgtgtctggcgacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgaccagcctgggcagcaatcaagaagaggcctacgtcaccatgagcagcttctaccagaaccagtgatgatgggaatggaacctaccagacctgggtggccagctcactgagccatgcgctatgtccgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttcttaggctcgaggtcgacggtatcgataagcttgatatccgcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacgcgtaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 12 - DNA, 1179 bp, synthetic construct
  source 1..1179: mol_type = other DNA
atggctcctcggagagccagaggctgtagaacacttggactgcccgctctgctgctgctcctgcttcttagacctcctgccacaagaggcgacgtggtcatgacacagacccctctgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggacttcgcctgtgacgtggccatcagcacaagcaccgttctgctgtgtggcctgtcagccgttagcctgctggcttgctacctgaagtccagacagacacctcctctggccagcgtggaaatggaagccatggaagctctgccagtgacctggggcacctccagcagagatgaggatctggaaaactgcagccaccacctgtgatga

SEQ ID NO: 13 - DNA, 1878 bp, synthetic construct
  source 1..1878: mol_type = other DNA
atgccaagaggatgggccgctcctcttctcctgttgctgcttcaaggcggctggggcgacgttgtgatgacacagacaccactgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggactttgcctgcgacggctggaatcctcatctgttgttgttgctcctcctggtcatcgtgttcatccccgccttttggagcctgaaaacacaccctctgtggcggctgtggaagaaaatctgggccgtgccatctcctgagcggttcttcatgcctctgtacaagggctgcagcggcgacttcaagaaatgggtcggagccccttttaccggcagctctctggaacttggaccttggagccctgaagtgcccagcacactggaagtgtacagctgtcaccctcctagaagccccgccaagagactgcagctcacagagctgcaagagcctgccgagctggtggaatctgatggcgtgcccaagcctagcttctggcccaccgctcaaaattctggcggcagcgcctacagcgaggaaagagatagaccttacggcctggtgtccatcgacaccgtgacagtgctggatgccgagggaccttgtacctggccttgtagctgcgaggacgatggctaccctgctctggatctggacgcaggactggaaccttctccaggcctcgaagatcctctgctggacgccggaacaacagtgctgtcttgtggctgtgtgtccgccggatctcctggacttggaggacctctgggaagcctgctggacagactgaaacctcctctggccgatggcgaagattgggctggtggacttccttggggcggaagatctccaggcggagtgtctgaatctgaggccggttctccactggccggcctggatatggataccttcgatagcggcttcgtgggcagcgattgcagcagccctgtggaatgcgacttcacatctcctggcgacgagggcccacctagaagctatctcagacagtgggtcgtgatccctccacctctgtctagtcctggaccacaggccagctgatga

SEQ ID NO: 14 - DNA, 4338 bp, synthetic construct
  source 1..4338: mol_type = other DNA
atggctgctccagctctgtcttggagactgcccctgctgattctgctgctgcctctggctacatcttgggcctctgccgacgtggtcatgacacagacaccactgagcctgcctgtgtctctgggagatcaggccagcatcagctgcagatccagccagtctctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacatcccctggctgggacatctgcttgttggactgtctggcgccttcggcttcatcatcctggtgtatctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagcccaggtggactggcccctgagattagccctctggaagtgctggaacgggacaaagtgacccagctgctcctccagcaggataaggtgccagaacctgccagcctgtccagcaatcacagcctgaccagctgctttaccaaccagggctacttcttcttccatctgcctgacgctctggaaatcgaggcctgccaggtgtacttcacctacgatccctacagcgaagaggaccccgatgaaggtgttgctggcgcccctacaggatcttctccacagcctctgcaacctctgagcggcgaggatgatgcctactgcacctttccaagcagggacgacctgctcctgttcagcccatctctgctcggaggaccatctcctccatctacagctccaggcggatctggcgctggcgaggaaagaatgccacctagcctgcaagagcgggtgcccagagattgggatcctcaacctctcggccctccaacacctggcgtgccagatctcgtggactttcagcctcctccagagctggtgctgagagaagctggcgaagaagtgccagacgctggccctagagagggcgttagctttccttggagcagacctcctggacagggcgagtttagggccctgaatgcaagactgcctctgaacaccgacgcctacctgtctctgcaagaactgcagggacaagaccccacacacctggtgactggatctggagcaacaaacttctcactactcaaacaagcaggtgacgtggaggagaatcccgggcctgccatgctgaagcccagcctgccttttaccagcctgctgttcctgcagctgcctctgcttggcgtgggagatgtggtcatgacacagaccccactgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggacttcgcctgcgacgtggtcatctctgtgggctctatgggcctgatcatctccctgctgtgtgtgtacttttggctggaacggaccatgcctcggatccccacactgaagaacctggaagatctggtcaccgagtaccacggcaacttcagtgcttggagcggcgtgtcaaaaggactggccgaaagcctgcagcctgactactccgagagactgtgcctggtgtctgagatccctcctaaaggcggcgctctcggagaaggacctggtgcctctccatgcaatcagcacagcccttattgggcccctccttgctacaccctgaaacctgagacaggctccggcgagggcaggggaagtcttctaacatgcggggacgtggaggaaaatcccggcccatgggaaatgcctcctcctcggctgctgttcttcctgctgtttctgacccctatggaagtgcggcccgaggaacctctggtggtcaaagttgaagagggcgacaacgccgtgctgcagtgtctgaagggcacatctgatggccccacacagcagctgacctggtctagagagagccctctgaagcccttcctgaagctgtctctgggactgcctggactgggcatccatatgaggcctctggccatctggctgttcatcttcaacgtgtcccagcagatgggcggcttctacctgtgtcaacctggacctccaagcgagaaggcttggcagcctggctggaccgtgaatgtggaaggatccggcgagctgttccggtggaatgtgtctgatctcggcggcctcggatgcggcctgaagaatagatctagcgagggccctagcagccccagcggaaaactgatgagccccaagctgtacgtgtgggccaaagacagacccgagatttgggagggcgagcctccttgtctgcctcctagagacagcctgaaccagagcctgagccaggacctgacaatggcccctggatctacactgtggctgagctgtggcgtgccacctgacagtgtgtctagaggccctctgtcttggacccacgtgcaccctaagggccctaagtctctgctgagcctggaactgaaggacgacaggcccgccagagatatgtgggtcatggaaacaggcctgctgctgcctagagccacagcacaggatgccggcaagtactactgccacagaggcaacctgaccatgagcttccacctggaaatcaccgccagacctgtcctgtggcactggctgcttagaaccggcggctggaaagtgtctgccgtgactctggcctacctgatcttctgcctgtgtagcctcgtgggcatcctgcatctgcagagagcactggtcctgcggcggaagcggaagagaatgaccgatcctaccagacggttctgatga

SEQ ID NO: 15 - protein, 21 aa, synthetic construct
MDSYLLMWGLLTFIMVPGCQA

SEQ ID NO: 16 - protein, 26 aa, synthetic construct
MAAPALSWRLPLLILLLPLATSWASA

SEQ ID NO: 17 - protein, 22 aa, synthetic construct
MLKPSLPFTSLLFLQLPLLGVG

SEQ ID NO: 18 - protein, 20 aa, synthetic construct
MTILGTTFGMVFSLLQVVSG

SEQ ID NO: 19 - protein, 30 aa, synthetic construct
MAPRRARGCRTLGLPALLLLLLLRPPATRG

SEQ ID NO: 20 - protein, 19 aa, synthetic construct
MPRGWAAPLLLLLLQGGWG

SEQ ID NO: 21 - protein, 254 aa, synthetic construct
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 22 - protein, 45 aa, synthetic construct
TTTPAPRPPTPAPTIASQPLSLRPEACRPAAGGAVHTRGLDFACD

SEQ ID NO: 23 - protein, 19 aa, synthetic construct
VAVAGCVFLLISVLLLSGL

SEQ ID NO: 24 - protein, 25 aa, synthetic construct
IPWLGHLLVGLSGAFGFIILVYLLI

SEQ ID NO: 25 - protein, 21 aa, synthetic construct
VVISVGSMGLIISLLCVYFWL

SEQ ID NO: 26 - protein, 25 aa, synthetic construct
PILLTISILSFFSVALLVILACVLW

SEQ ID NO: 27 - protein, 23 aa, synthetic construct
VAISTSTVLLCGLSAVSLLACYL

SEQ ID NO: 28 - protein, 21 aa, synthetic construct
GWNPHLLLLLLLVIVFIPAFW

SEQ ID NO: 29 - protein, 13 aa, synthetic construct
TWQRRQRKSRRTI

SEQ ID NO: 30 - protein, 286 aa, synthetic construct
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 31 - protein, 86 aa, synthetic construct
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 32 - protein, 195 aa, synthetic construct
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 33 - protein, 39 aa, synthetic construct
KSRQTPPLASVEMEAMEALPVTWGTSSRDEDLENCSHHL

SEQ ID NO: 34 - protein, 285 aa, synthetic construct
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 35 - protein, 22 aa, synthetic construct
GSGATNFSLLKQAGDVEENPGP

SEQ ID NO: 36 - protein, 21 aa, synthetic construct
GSGEGRGSLLTCGDVEENPGP

SEQ ID NO: 37 - protein, 23 aa, synthetic construct
GSGQCTNYALLKLAGDVESNPGP

SEQ ID NO: 38 - protein, 25 aa, synthetic construct
GSGVKQTLNFDLLKLAGDVESNPGP

SEQ ID NO: 39 - protein, 333 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 40 - DNA, 2550 bp, synthetic construct
  source 1..2550: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatctggcgccacaaacttctcactgctgaaacaggccggcgacgtcgaagagaatcctgggcctatgcctcctcctcggctgctgttcttcctgctgtttctgacccctatggaagtgcggcccgaggaacctctggtcgtgaaagttgaagagggcgacaacgccgtgctgcagtgtctgaagggcacatctgatggccccacacagcagctgacctggtctagagagagccctctgaagcccttcctgaagctcagtctgggactgcctggactgggcatccatatgaggccactggccatctggctgttcatctttaacgtgtcccagcagatgggaggcttctacctgtgtcagcctggacctcctagcgagaaagcttggcagcctggctggaccgtgaatgtggaaggatccggcgagctgttccggtggaatgtctctgatctcggcggcctcggatgcggcctgaagaatagatctagcgagggccctagcagcccctccggaaaactgatgagccccaagctgtacgtgtgggccaaagacagacccgagatttgggagggcgagcctccttgtctgccacctagagacagcctgaatcagagcctgagccaggacctgacaatggcccctggatctacactgtggctgagctgcggagtgcctcctgacagtgtgtctagaggcccactgagctggacacacgtgcaccctaagggccctaagagcctgctgagtctggaactgaaggacgacaggcccgccagagatatgtgggtcatggaaaccggactgctgctccctagagccactgctcaagatgccggcaagtactattgccaccggggcaacctgaccatgagcttccacctggaaattaccgccagaccagtgctgtggcattggctgcttagaaccggcggatggaaggtgtcagccgtgactctggcctacctgatcttttgtctgtgcagcctcgtgggcatcctgcatctgcaaagagccctggtcctgcggcggaagcggaagagaatgaccgatcctaccagacggttctgatga

SEQ ID NO: 41 - DNA, 1500 bp, synthetic construct
  source 1..1500: mol_type = other DNA
atggctctgcctgtcaccgctctgctgctgcctctggctctgctgctgcacgctgctcgccctcaggtcaaactggaagaaagtgggggaggcctggtgcaggcaggacggagcctgcgcctgagctgcgcagcatccgagcacaccttcagctcccacgtgatgggctggtttcggcaggccccaggcaaggagagagagtccgtggccgtgatcggctggagggacatctccacatcttacgccgattctgtgaagggccggttcaccatcagcagagacaacgccaagaagacactgtatctgcagatgaatagcctgaagcccgaggacaccgccgtgtactattgcgcagcaaggagaatcgacgcagcagactttgattcctggggccagggcacccaggtgacagtgtctagcggaggaggaggatctggaggaggaggaagcggaggaggaggatccgaggtgcagctggtggagtctggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagtccgtgaagggcaggtttaccatctctcgcgataacgccaagaatacagtggtgctgcagatgaacagcctgaaacctgaggacacagccctgtactattgtgccgccgatcggaagagcgtgatgagcattagacccgattattggggacagggcacacaggtgacagtgagtagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 42 - DNA, 1473 bp, synthetic construct
  source 1..1473: mol_type = other DNA
atggccctgccagtaactgctcttctcctgccactcgctcttctgctgcacgccgcacggcctcaaattcaacttgtgcagagtggccccgagctgaaaaagcccggcgaaacagtaaagatctcttgtaaggcgtctggatatatctttaccaattacggtatgaactgggtcaaacaggcacccggtaaaagctttaaatggatgggctggataaatacttataccggagaaagcacgtatagcgccgacttcaagggtagatttgcgttcagcctggaaacgtccgccagcactgcatacttgcacatcaatgacttgaaaaatgaggataccgcgacctacttttgtgctagaagtggtggatacgatcccatggattactggggacaggggacctctgtcacagtcagttctgggggcggcggttccggaggtggcggctcaggtggaggtgggtcagatatcgtgttgacacaaagccctgcttccctggccgtcagcttgggacagagagctactatctcctgtagagcaagtgagtctgtcgacaattacggcaacacctttatgcactggtaccagcagaagccaggccagccaccgaagctgttgatttaccgggcctcaaacctcgaaagcgggattccagctagattttctggcagcgggagtcgaaccgacttcacactgactattaacccagtggaagcagatgacgtagccacatattactgtcagcagagcaacgaagaccccccgacgtttggtgctggaaccaaactggagctgaaaacgacaacccccgctccacgacctcctactcccgctcccacaatcgcatctcagccactctccttgaggcctgaggcctgccgaccagctgcaggcggcgcagtacacaccagaggcctcgactttgcttgcgatatctacatatgggcgcccctggccgggacgtgtggggtattgctcctgagccttgtgatcactctttactgcaaaagaggtcggaaaaagcttctttacatctttaaacagccgttcatgcgcccggtgcaaacaacacaggaggaggatggatgcagctgcaggtttccggaggaggaggaaggcgggtgtgagctccgggtaaaattctccagaagcgcagacgcgccagcctatcagcaggggcagaatcagctctataacgaactcaatcttgggcgacgggaagaatacgatgtacttgataaacgccgaggtcgcgaccccgaaatgggaggcaagcctcagagacgcaagaatccccaagaaggcctgtataatgaactccagaaggataaaatggcggaggcctacagcgaaattggtatgaagggagaaagacgacgcggtaaaggccacgacggcctttaccagggcctgtcaacagctaccaaagatacgtatgatgcacttcacatgcaagccctgccgccccggtgatga

SEQ ID NO: 43 - DNA, 1473 bp, synthetic construct
  source 1..1473: mol_type = other DNA
atggctctgcctgtcaccgctctgctgctgcctctggctctgctgctgcacgctgctcgccctcaggtgcagctgcaggagtctggcccaggcctggtgaagcccagccagaccctgagcatcacctgcaccgtgagcggcttcagcctggccagctacaacatccactgggtgcggcagcccccaggcaagggcctggagtggctgggcgtgatctgggctggcggcagcaccaactacaacagcgccctgatgagccggctgaccatcagcaaggacaacagcaagaaccaggtgttcctgaagatgagcagcctgacagccgccgacaccgccgtgtactactgcgccaagcggagcgacgactacagctggttcgcctactggggccagggcaccctggtgaccgtgagctctggcggaggcggctctggcggaggcggctctggcggaggcggcagcgagaaccagatgacccagagccccagcagcttgagcgccagcgtgggcgaccgggtgaccatgacctgcagagccagcagcagcgtgagcagcagctacctgcactggtaccagcagaagagcggcaaggccccaaaggtgtggatctacagcaccagcaacctggccagcggcgtgaccagccggttcagcggcagcggcagcggcaccgactacaccctgaccatcagcagcctgcagcccgaggacttcgccacctactactgccagcagtacagcggctaccccatcaccttcggccagggcaccaaggtggagatcaagcggtcggatcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 44 - DNA, 1503 bp, synthetic construct
  source 1..1503: mol_type = other DNA
atggctctgccagtgacagctctgctgcttcctctggctctcctgctgcatgccgctagacctgagcagaaactcatctctgaagaggatctgcacgtgaaactgcaagagtctggccccggactggtgcagccttctcagtctctctctctgacctgtaccgtgtccggcttcagcctgaccgattatggcgtgcactgggttcgacaatccccaggcaaaggactcgagtggctgggagtgatttggagcggcggaggcacagcctataacacagccctgatcagcagactgaacatctaccgggacaactccaagaaccaggttttcctggaaatgaactccctgcaggcagaggacaccgccatgtactactgcgccagaagaggcagctacccctacaattacttcgacgcctggggctgtggcacaaccgtgacagtttctagtggcggaggcggatctggtggtggtggtagcggtggcggaggatctcaggccgtggttattcaagaaagcgccctgacaacccctcctggcgagacagtgacactgacatgtggcagctctacaggcgccgtgaccgccagcaattacgccaattgggtgcaagagaagcccgaccactgcttcacaggcctgatcggcggccacaacaatagacctccaggcgtgccagctagattcagcggatccctgatcggagacaaggccgctctgacaatcgccggcacacagacagaggacgaggccatctacttttgcgccctgtggtacagcgaccactgggttatcggcggaggaaccagactgacagtgctgggcacaacaacacccgcacctagaccaccaactccagcaccaacaatcgcctctcaacccctgagtctgaggccagaggcatgcagaccagccgctggcggtgcagttcacactagaggactggactttgcctgtgacatctacatctgggcccctctggctggaacatgtggtgtcctgctgctgtccctggtcatcaccctgtactgcaagcggggcagaaagaaactgctgtacatcttcaagcagcccttcatgcggcccgtgcagaccacacaagaggaagatggctgctcctgcagattccccgaggaagaagaaggcggctgcgagctgagagtgaagttctccagatctgccgacgctcccgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggaaagggacacgacggactgtaccagggcctgagcacagccaccaaggatacctatgacgccctgcacatgcaggccctgcctccaagatgatga

SEQ ID NO: 45 - DNA, 1512 bp, synthetic construct
  source 1..1512: mol_type = other DNA
atggctctgcctgtgacagctctgctgctgcctctggctctgcttctgcatgccgccagacctgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacttctgggtgctcgtggttgttggcggagtgctggcttgttactccctgctggttaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgccacctagatgatga

SEQ ID NO: 46 - DNA, 4866 bp, synthetic construct
  source 1..4866: mol_type = other DNA
atggctgctcctgctctgtcttggagactgcccctgctgattctgctgctgcctctggctacatcttgggcctctgccgattacaaggatgacgacgataaggacgtggtcatgacacagacaccactgagcctgcctgtgtctctgggagatcaggccagcatcagctgcagatccagccagtctctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacatcccctggctgggacatctgcttgttggactgtctggcgccttcggcttcatcatcctggtgtatctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagcccaggtggactggcccctgagattagccctctggaagtgctggaacgggacaaagtgacccagctgctcctccagcaggataaggtgccagaacctgccagcctgtccagcaatcacagcctgaccagctgctttaccaaccagggctacttcttcttccatctgcctgacgctctggaaatcgaggcctgccaggtgtacttcacctacgatccctacagcgaagaggaccccgatgaaggtgttgctggcgcccctacaggatcttctccacagcctctgcaacctctgagcggcgaggatgatgcctactgcacctttccaagcagggacgacctgctcctgttcagcccatctctgctcggaggaccatctcctccatctacagctccaggcggatctggcgctggcgaggaaagaatgccacctagcctgcaagagcgggtgcccagagattgggatcctcaacctctcggccctccaacacctggcgtgccagatctcgtggactttcagcctcctccagagctggtgctgagagaagctggcgaagaagtgccagacgctggccctagagagggcgttagctttccttggagcagacctcctggacagggcgagtttagggccctgaatgctagactgcctctgaacaccgacgcctacctgtctctgcaagaactgcagggacaagaccccacacacctcgttggaagcggagtgaagcagaccctgaacttcgacctgctgaaactggccggcgacgtggaaagcaaccctggacctatgctgaagcccagcctgccttttaccagcctgctgttcctgcagctcccactgcttggcgtgggatacccatacgatgttccagattacgctgatgtcgtgatgacccagactccactctctctgcctgtcagcctgggcgatcaagcctccatctcctgtagaagcagccagagcctggtccactccaatggcaatacctatctcagatggtatctccaaaagcctgggcagagcccaaaagtcctcatctacaaagtctctaatcgcgtcagcggagtgcccgacaggtttagcggttctggaagcggcacagatttcacgctcaagattaaccgcgtcgaggccgaagatctgggagtctatttctgcagtcagagcacacatgtgccctggacattcggcggaggcaccaagctcgagatcaagtctagcgccgatgatgctaagaaagatgcagctaaaaaggatgatgccaagaaggacgacgccaagaaagatggcggagtcaaactcgatgagactggcggaggcctggtgcaacccggtggtgctatgaagctgtcttgcgtgacctccggctttacatttgggcattattggatgaattgggttcgccaatctccagagaagggcctcgagtgggttgcacagtttcggaacaaaccgtacaattacgagacatattactccgactccgtgaaaggccggttcacaatctcccgcgacgactccaagtcctctgtctatcttcaaatgaacaatctgagagtcgaggacacggggatctactattgcacaggcgcctcttatggaatggaataccttggacagggaacctctgtgaccgtcagcaccacaacacccgctcctagacctccaactcctgctccaacaatcgcctctcaacccctcagcctcagacctgaggcatgtagaccagcagctggcggtgcagttcacaccagaggcctggactttgcctgtgacgtcgtgatcagcgtgggcagcatgggcctgatcatctccctgctgtgtgtgtacttttggctcgagcggaccatgcctcggatccccacactgaagaacctcgaggatctggtcaccgagtaccacggcaacttctctgcttggagcggcgtgtcaaaaggactggccgaaagcctgcagcctgactactccgagagactgtgcctggtgtctgagatccctcctaaaggcggcgctctcggagaaggacctggtgcctctccatgcaatcagcacagcccttattgggcccctccttgctacaccctgaaacctgagacaggcagcggcgaaggcagaggctctcttcttacatgtggcgacgtcgaagagaatcccggaccaatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctgatga

SEQ ID NO: 47 - DNA, 4254 bp, synthetic construct
  source 1..4254: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgcggatctggcgccacaaacttctcactgctgaaacaggccggcgacgtcgaagagaatcctgggcctatgcctcctcctcggctgctgttcttcctgctgtttctgacccctatggaagtgcggcccgaggaacctctggtcgtgaaagttgaagagggcgacaacgccgtgctgcagtgtctgaagggcacatctgatggccccacacagcagctgacctggtctagagagagccctctgaagcccttcctgaagctcagtctgggactgcctggactgggcatccatatgaggccactggccatctggctgttcatctttaacgtgtcccagcagatgggaggcttctacctgtgtcagcctggacctcctagcgagaaagcttggcagcctggctggaccgtgaatgtggaaggatccggcgagctgttccggtggaatgtctctgatctcggcggcctcggatgcggcctgaagaatagatctagcgagggccctagcagcccctccggaaaactgatgagccccaagctgtacgtgtgggccaaagacagacccgagatttgggagggcgagcctccttgtctgccacctagagacagcctgaatcagagcctgagccaggacctgacaatggcccctggatctacactgtggctgagctgcggagtgcctcctgacagtgtgtctagaggcccactgagctggacacacgtgcaccctaagggccctaagagcctgctgagtctggaactgaaggacgacaggcccgccagagatatgtgggtcatggaaaccggactgctgctccctagagccactgctcaagatgccggcaagtactattgccaccggggcaacctgaccatgagcttccacctggaaattaccgccagaccagtgctgtggcattggctgcttagaaccggcggatggaaggtgtcagccgtgactctggcctacctgatcttttgtctgtgcagcctcgtgggcatcctgcatctgcaaagagccctggtcctgcggcggaagcggaagagaatgaccgatcctaccagacggttctgatga

SEQ ID NO: 48 - DNA, 3204 bp, synthetic construct
  source 1..3204: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgcctgtcaccgctctgctgctgcctctggctctgctgctgcacgctgctcgccctcaggtcaaactggaagaaagtgggggaggcctggtgcaggcaggacggagcctgcgcctgagctgcgcagcatccgagcacaccttcagctcccacgtgatgggctggtttcggcaggccccaggcaaggagagagagtccgtggccgtgatcggctggagggacatctccacatcttacgccgattctgtgaagggccggttcaccatcagcagagacaacgccaagaagacactgtatctgcagatgaatagcctgaagcccgaggacaccgccgtgtactattgcgcagcaaggagaatcgacgcagcagactttgattcctggggccagggcacccaggtgacagtgtctagcggaggaggaggatctggaggaggaggaagcggaggaggaggatccgaggtgcagctggtggagtctggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagtccgtgaagggcaggtttaccatctctcgcgataacgccaagaatacagtggtgctgcagatgaacagcctgaaacctgaggacacagccctgtactattgtgccgccgatcggaagagcgtgatgagcattagacccgattattggggacagggcacacaggtgacagtgagtagcactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 49 - DNA, 3177 bp, synthetic construct
  source 1..3177: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggccctgccagtaactgctcttctcctgccactcgctcttctgctgcacgccgcacggcctcaaattcaacttgtgcagagtggccccgagctgaaaaagcccggcgaaacagtaaagatctcttgtaaggcgtctggatatatctttaccaattacggtatgaactgggtcaaacaggcacccggtaaaagctttaaatggatgggctggataaatacttataccggagaaagcacgtatagcgccgacttcaagggtagatttgcgttcagcctggaaacgtccgccagcactgcatacttgcacatcaatgacttgaaaaatgaggataccgcgacctacttttgtgctagaagtggtggatacgatcccatggattactggggacaggggacctctgtcacagtcagttctgggggcggcggttccggaggtggcggctcaggtggaggtgggtcagatatcgtgttgacacaaagccctgcttccctggccgtcagcttgggacagagagctactatctcctgtagagcaagtgagtctgtcgacaattacggcaacacctttatgcactggtaccagcagaagccaggccagccaccgaagctgttgatttaccgggcctcaaacctcgaaagcgggattccagctagattttctggcagcgggagtcgaaccgacttcacactgactattaacccagtggaagcagatgacgtagccacatattactgtcagcagagcaacgaagaccccccgacgtttggtgctggaaccaaactggagctgaaaacgacaacccccgctccacgacctcctactcccgctcccacaatcgcatctcagccactctccttgaggcctgaggcctgccgaccagctgcaggcggcgcagtacacaccagaggcctcgactttgcttgcgatatctacatatgggcgcccctggccgggacgtgtggggtattgctcctgagccttgtgatcactctttactgcaaaagaggtcggaaaaagcttctttacatctttaaacagccgttcatgcgcccggtgcaaacaacacaggaggaggatggatgcagctgcaggtttccggaggaggaggaaggcgggtgtgagctccgggtaaaattctccagaagcgcagacgcgccagcctatcagcaggggcagaatcagctctataacgaactcaatcttgggcgacgggaagaatacgatgtacttgataaacgccgaggtcgcgaccccgaaatgggaggcaagcctcagagacgcaagaatccccaagaaggcctgtataatgaactccagaaggataaaatggcggaggcctacagcgaaattggtatgaagggagaaagacgacgcggtaaaggccacgacggcctttaccagggcctgtcaacagctaccaaagatacgtatgatgcacttcacatgcaagccctgccgccccggtgatga

SEQ ID NO: 50 - DNA, 3177 bp, synthetic construct
  source 1..3177: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgcctgtcaccgctctgctgctgcctctggctctgctgctgcacgctgctcgccctcaggtgcagctgcaggagtctggcccaggcctggtgaagcccagccagaccctgagcatcacctgcaccgtgagcggcttcagcctggccagctacaacatccactgggtgcggcagcccccaggcaagggcctggagtggctgggcgtgatctgggctggcggcagcaccaactacaacagcgccctgatgagccggctgaccatcagcaaggacaacagcaagaaccaggtgttcctgaagatgagcagcctgacagccgccgacaccgccgtgtactactgcgccaagcggagcgacgactacagctggttcgcctactggggccagggcaccctggtgaccgtgagctctggcggaggcggctctggcggaggcggctctggcggaggcggcagcgagaaccagatgacccagagccccagcagcttgagcgccagcgtgggcgaccgggtgaccatgacctgcagagccagcagcagcgtgagcagcagctacctgcactggtaccagcagaagagcggcaaggccccaaaggtgtggatctacagcaccagcaacctggccagcggcgtgaccagccggttcagcggcagcggcagcggcaccgactacaccctgaccatcagcagcctgcagcccgaggacttcgccacctactactgccagcagtacagcggctaccccatcaccttcggccagggcaccaaggtggagatcaagcggtcggatcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatga

SEQ ID NO: 51 - DNA, 3207 bp, synthetic construct
  source 1..3207: mol_type = other DNA
atgaccatcctgggcaccaccttcggcatggtgtttagcctgctgcaggtcgtgtctggcgattacaaggatgacgacgataaggacgtggtcatgacacagaccccactgtctctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgcaaagaaagacgatgccaaaaaggatggcggcgtgaagctggacgaaacaggcggaggacttgttcagcctggcggagccatgaagctgagctgtgtgaccagcggcttcacctttggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgccagtgacagctctgctgcttcctctggctctcctgctgcatgccgctagacctgagcagaaactcatctctgaagaggatctgcacgtgaaactgcaagagtctggccccggactggtgcagccttctcagtctctctctctgacctgtaccgtgtccggcttcagcctgaccgattatggcgtgcactgggttcgacaatccccaggcaaaggactcgagtggctgggagtgatttggagcggcggaggcacagcctataacacagccctgatcagcagactgaacatctaccgggacaactccaagaaccaggttttcctggaaatgaactccctgcaggcagaggacaccgccatgtactactgcgccagaagaggcagctacccctacaattacttcgacgcctggggctgtggcacaaccgtgacagtttctagtggcggaggcggatctggtggtggtggtagcggtggcggaggatctcaggccgtggttattcaagaaagcgccctgacaacccctcctggcgagacagtgacactgacatgtggcagctctacaggcgccgtgaccgccagcaattacgccaattgggtgcaagagaagcccgaccactgcttcacaggcctgatcggcggccacaacaatagacctccaggcgtgccagctagattcagcggatccctgatcggagacaaggccgctctgacaatcgccggcacacagacagaggacgaggccatctacttttgcgccctgtggtacagcgaccactgggttatcggcggaggaaccagactgacagtgctgggcacaacaacacccgcacctagaccaccaactccagcaccaacaatcgcctctcaacccctgagtctgaggccagaggcatgcagaccagccgctggcggtgcagttcacactagaggactggactttgcctgtgacatctacatctgggcccctctggctggaacatgtggtgtcctgctgctgtccctggtcatcaccctgtactgcaagcggggcagaaagaaactgctgtacatcttcaagcagcccttcatgcggcccgtgcagaccacacaagaggaagatggctgctcctgcagattccccgaggaagaagaaggcggctgcgagctgagagtgaagttctccagatctgccgacgctcccgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggaaagggacacgacggactgtaccagggcctgagcacagccaccaaggatacctatgacgccctgcacatgcaggccctgcctccaagatgatga

SEQ ID NO: 52 - DNA, 3186 bp, synthetic construct
  source 1..3186: mol_type = other DNA
atgaccatcctgggcaccaccttcggcatggtgtttagcctgctgcaggtcgtgtctggcgattacaaggatgacgacgataagcacgtgaaactgcaagagtctggccccggactggtgcagccttctcagtctctctctctgacctgtaccgtgtccggcttcagcctgaccgattatggcgtgcactgggttcgacaatccccaggcaaaggactcgagtggctgggagtgatttggagcggcggaggcacagcctataacacagccctgatcagcagactgaacatctaccgggacaactccaagaaccaggttttcctggaaatgaactccctgcaggcagaggacaccgccatgtactactgcgccagaagaggcagctacccctacaattacttcgacgcctggggctgtggcacaaccgtgacagtttctagtggcggaggcggatctggtggtggtggtagcggtggcggaggatctcaggccgtggttattcaagaaagcgccctgacaacccctcctggcgagacagtgacactgacatgtggcagctctacaggcgccgtgaccgccagcaattacgccaattgggtgcaagagaagcccgaccactgcttcacaggcctgatcggcggccacaacaatagacctccaggcgtgccagctagattcagcggatccctgatcggagacaaggccgctctgacaatcgccggcacacagacagaggacgaggccatctacttttgcgccctgtggtacagcgaccactgggttatcggcggaggaaccagactgacagtgctgggcacaacaacccctgctcctcggcctcctacaccagctcctacaattgccagccagcctctgtctctgaggcccgaagcttgtagacctgctgccggcggagctgtgcatacaagaggcctggatttcgcctgcgatcccatcctgctgacaatcagcatcctgagctttttcagcgtggccctgctggtcatcctggcctgtgtgctgtggaagaagcggatcaagcccatcgtgtggcccagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctggaagagagcgagaagcagagactcggaggcgacgtgcagagccctaattgcccttctgaggacgtcgtgatcacccctgagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgagcctgggaaccaccaatagcacactgcctccaccattcagcctgcagagcggcatcctgacactgaaccctgttgctcagggccagccaatcctgacaagcctgggcagcaatcaagaagaggcctacgtcaccatgagcagcttctaccagaatcaaggctccggcgaaggcagaggctctctgcttacatgcggagatgtggaagagaaccccggacctatggctctgcctgtgacagctctgctgctgcctctggctctgcttctgcatgccgccagacctgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacttctgggtgctcgtggttgttggcggagtgctggcttgttactccctgctggttaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgccacctagatgatga

(19) United States
(12) Patent Application Publication
(10) Pub. No.: US 2025/0297220 A1
(43) Pub. Date: Sep. 25, 2025

(54) IMMUNE-ACTIVATING COMPLEXES

(71) Applicant: Dynamic Cell Therapies, Inc., Watertown, MA (US)

(72) Inventors: Jun Ren, Brookline, MA (US); Alberto Nobili, Boston, MA (US); Carl D. Novina, Newton, MA (US); Craig Stewart McKay, Medford, MA (US); John R. Newcomb, Woburn, MA (US); Fred H. Mermelstein, West Newton, MA (US)

(21) Appl. No.: 19/229,839

(22) Filed: Jun. 5, 2025

Related U.S. Application Data
(63) Continuation of application No. PCT/US2023/082604, filed on Dec. 5, 2023.
(60) Provisional application No. 63/386,299, filed on Dec. 6, 2022.
(60) Provisional application No. 63/386,453, filed on Dec. 7, 2022.

(51) Int. Cl. C12N 5/0783; C12N 15/86

(52) U.S. Cl. CPC C12N 5/0636; C12N 15/86; C12N 2510/00; C12N 2533/50; C12N 2740/15043

(57) ABSTRACT

Disclosed herein are immune cells expressing engineered cytokine receptor switches which, upon activation, can direct differentiation of the immune cells. The present disclosure further provides compositions and methods for transfecting, expanding, and activating these immune cells, including substrates which activate the engineered cytokine receptor switches disclosed herein.

### CROSS-REFERENCE TO RELATED APPLICATION(S)

The present application is a continuation of International Application No. PCT/US2023/082604, filed Dec. 5, 2023, which claims the benefit of priority to U.S. Provisional Application No. 63/386,299, filed Dec. 6, 2022, and U.S. Provisional Application No. 63/386,453, filed Dec. 7, 2022, each of which is incorporated by reference herein in its entirety.

### INCORPORATION BY REFERENCE OF SEQUENCE LISTING

The present application contains an electronic Sequence Listing in XML file format named “DCT_003WO_SL,” created on Nov. 30, 2023, and having a size of 99.3 kilobytes, the contents of which are incorporated by reference herein in their entirety.

### TECHNICAL FIELD

The present technology relates to immunotherapy and, in particular, immune-activating complexes.

### BACKGROUND

Immune cell engineering often yields immune cell populations with high potencies but short in vivo lifespans, rendering them ineffective for managing many long-term, recurrent, and chronic conditions. Engineered lymphoid cells (e.g., T- and B-cells) challenged with antigens during activation and expansion often develop primarily into terminal effector cells, which can be incapable of division and can exhibit low persistence in the absence of high target antigen concentrations. While such cells can be effective for reducing disease (e.g., tumor) burden in acute illnesses, they typically do not persist after the disease is pushed into a dormant or remissive state and can thus be incapable of achieving complete disease clearance and preventing recurrence and relapse.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A provides depictions of multiple sizes of functionalized beads consistent with the present disclosure.

FIG. 1B provides a depiction of a functionalized polymer matrix substrate consistent with the present disclosure.

FIG. 2A depicts carrier complexes having different linker lengths consistent with the present disclosure.

FIG. 2B depicts carrier complexes with oligomeric or polymeric constructs consistent with the present disclosure.

FIG. 2C depicts carrier structures with branched structures consistent with the present disclosure.

FIG. 3 depicts a method for coupling and uncoupling a carrier complex from a substrate consistent with the present disclosure.

FIG. 4 outlines a method for conjugating a small molecule activator to a carboxylate functionalized bead and controlling the linker length by using different bifunctional amines.

FIG. 5 provides fluorescein activator loading for 1.0 μm fluorescein-functionalized beads containing variable length polyethylene glycol (PEG) linkers.

FIG. 6 provides fluorescein activator loading for 2.8 μm fluorescein-functionalized beads containing a short PEG linker.

FIG. 7 provides fluorescein activator loading for 2.8 μm beads functionalized with fluorescein attached by variable length PEG linkers.

FIG. 8 outlines a scheme for functionalizing tosyl-activated beads with an activator.

FIG. 9 summarizes activator loading per mg of bead for of 4.5 μm fluorescein-functionalized beads containing variable length PEG linkers.

FIG. 10A schematically illustrates experiments used to probe the influence of beads on the fluorescence of fluorescein activator, including a titration in which beads are sequentially added to a fluorescein solution (top), and a titration in which fluorescein is sequentially added to a solution containing a constant mass of beads (bottom).

FIG. 10B provides fluorescence intensities from a fluorescein titration in a bead-free solution.

FIG. 10C provides fluorescence intensities from a fluorescein titration in a solution containing 0.1 mg of 1.0 μm diameter hydrophilic beads.

FIG. 10D provides fluorescence intensities from a fluorescein titration in a solution containing 0.1 mg of 1.0 μm diameter hydrophobic beads.

FIG. 10E provides fluorescence intensities from a fluorescein titration in a solution containing 0.1 mg of 2.8 μm diameter hydrophilic beads.

FIG. 10F provides fluorescence intensities from a fluorescein titration in a solution containing 0.1 mg of 2.8 μm diameter hydrophobic beads.

FIG. 11A provides fluorescence intensities of 100 nM fluorescein solutions in the presence of multiple concentrations of 1.0 μm beads.

FIG. 11B provides fluorescence intensities of 100 nM fluorescein solutions in the presence of multiple concentrations of 2.8 μm beads.

FIG. 12 provides a schematic depiction of a bead settling fluorescence assay consistent with the present disclosure.

FIG. 13 provides results of fluorescein titrations with settled and suspended beads of different sizes: 1.0 μm (left panels), 2.8 μm beads (middle panels) and 4.5 μm beads (right panels).

FIG. 14 depicts systems with a first chemical handle on a first carrier complex configured to bind to a substrate and to a second carrier complex.

FIG. 15A provides a scheme for labeling carboxylate-functionalized beads with an activator-labeled polysaccharide carrier complex.

FIG. 15B provides a scheme for labeling tosylate-functionalized beads with an activator-labeled polysaccharide carrier complex.

FIG. 16 provides fluorescein activator loading for 1.0 μm carboxylate surface-functionalized beads containing aminodextran (AD) polymers having different lengths. AD10×40 and AD40×60 refer to the polymer molecular weight (kDa) x amines/polymer.

FIG. 17 provides fluorescein activator loading for 2.8 μm hydrophilic, carboxylate functionalized beads.

FIG. 18 provides fluorescein activator loading for 4.5 μm hydrophobic, tosyl-activated beads.

FIG. 19 provides a scheme for functionalizing a streptavidin-functionalized bead with biotinylated carrier complexes containing small molecule activator of multiple lengths.

FIG. 20A provides fluorescein activator loading for 1.0 μm hydrophilic streptavidin beads conjugated to biotin-PEG-fluorescein derivatives containing PEG linkers with different lengths.

FIG. 20B provides fluorescein activator loading for 1.0 μm hydrophobic streptavidin beads conjugated to biotin-PEG-fluorescein derivatives containing PEG linkers with different lengths.

FIG. 20C provides fluorescein activator loading for 2.8 μm hydrophilic streptavidin beads functionalized with biotin-PEG-fluorescein derivatives containing PEG linkers with different lengths.

FIG. 20D provides fluorescein activator loading for 2.8 μm hydrophobic streptavidin beads functionalized with biotin-PEG-fluorescein derivatives containing PEG linkers with different lengths.

FIG. 21A provides fluorescein activator loading for 1.0 μm hydrophilic streptavidin beads functionalized with multiple concentrations of fluorescein-PEG2-biotin carrier complex.

FIG. 21B provides fluorescein activator loading for 1.0 μm hydrophilic streptavidin beads functionalized with multiple concentrations of fluorescein-PEG1k-biotin carrier complex.

FIG. 21C provides fluorescein activator loading for 1.0 μm hydrophilic streptavidin beads functionalized with multiple concentrations of fluorescein-PEG2k-biotin carrier complex.

FIG. 21D provides fluorescein activator loading for 1.0 μm hydrophilic streptavidin beads functionalized with multiple concentrations of fluorescein-PEG5k-biotin carrier complex.

FIG. 22A provides fluorescein activator loading for 1.0 μm hydrophobic streptavidin beads functionalized with multiple concentrations of fluorescein-PEG2-biotin carrier complex.

FIG. 22B provides fluorescein activator loading for 1.0 μm hydrophobic streptavidin beads functionalized with multiple concentrations of fluorescein-PEG1k-biotin carrier complex.

FIG. 22C provides fluorescein activator loading for 1.0 μm hydrophobic streptavidin beads functionalized with multiple concentrations of fluorescein-PEG2k-biotin carrier complex.

FIG. 22D provides fluorescein activator loading for 1.0 μm hydrophobic streptavidin beads functionalized with multiple concentrations of fluorescein-PEG5k-biotin carrier complex.

FIG. 23A provides fluorescein activator loading for 2.8 μm hydrophilic streptavidin beads functionalized with multiple concentrations of fluorescein-PEG2-biotin carrier complex.

FIG. 23B provides fluorescein activator loading for 2.8 μm hydrophilic streptavidin beads functionalized with multiple concentrations of fluorescein-PEG1k-biotin carrier complex.

FIG. 23C provides fluorescein activator loading for 2.8 μm hydrophilic streptavidin beads functionalized with multiple concentrations of fluorescein-PEG2k-biotin carrier complex.

FIG. 23D provides fluorescein activator loading for 2.8 μm hydrophilic streptavidin beads functionalized with multiple concentrations of fluorescein-PEG5k-biotin carrier complex.

FIG. 24A provides fluorescein activator loading for 2.8 μm hydrophobic streptavidin beads functionalized with multiple concentrations of fluorescein-PEG2-biotin carrier complex.

FIG. 24B provides fluorescein activator loading for 2.8 μm hydrophobic streptavidin beads functionalized with multiple concentrations of fluorescein-PEG1k-biotin carrier complex.

FIG. 24C provides fluorescein activator loading for 2.8 μm hydrophobic streptavidin beads functionalized with multiple concentrations of fluorescein-PEG2k-biotin carrier complex.

FIG. 24D provides fluorescein activator loading for 2.8 μm hydrophobic streptavidin beads functionalized with multiple concentrations of fluorescein-PEG5k-biotin carrier complex.

FIG. 25 provides a graphic depiction of carrier complex functionalization of a streptavidin-coated hydrogel substrate.

FIG. 26A provides fluorescence intensities from a fluorescein titration performed in the absence of hydrogel microspheres.

FIG. 26B provides fluorescence intensities from a fluorescein titration performed in the presence of hydrogel microspheres.

FIG. 27A provides fluorescein activator loading for hydrogel microspheres reacted with 10 μM of various carrier complexes containing different PEG linker lengths.

FIG. 27B provides fluorescein activator loading for hydrogel microspheres reacted with 100 μM of various carrier complexes containing different PEG linker lengths.

FIG. 27C provides fluorescein activator loading for hydrogel microspheres reacted with 1 mM of various carrier complexes containing different PEG linker lengths.

FIG. 27D provides fluorescein activator loading for hydrogel microspheres reacted with 10 mM of various carrier complexes containing different PEG linker lengths.

FIG. 28A provides a timeline for transfection, activation, differentiation, and expansion of engineered immune cells consistent with the present disclosure.

FIG. 28B provides a schematic depiction of engineered cytokine receptor switch activation by a carrier complex-functionalized substrate.

FIG. 28C provides effector memory, central memory, stem-cell memory, and terminally differentiated effector memory cell populations of engineered immune cells contacted to various activator-functionalized substrates or not contacted to an activator.

FIG. 29A provides percentages of various memory cell phenotypes among engineered immune cells either contacted or not contacted with an activator.

FIG. 29B provides counts of cells of various memory cell phenotypes among engineered immune cells either contacted or not contacted with an activator.

FIG. 30A depicts expression profiles of various T-cell phenotypes.

FIG. 30B provides T-cell phenotype ratios for engineered immune cells activated with various activator-functionalized substrates.

FIG. 30C provides CCR7 and CD45RA expression profiles of populations of engineered immune cells activated with various activator-functionalized substrates.

FIG. 31 provides an illustrative comparison of engineered T-cells cultured with activated engineered cytokine receptor switches (left) and T-cells which lack the engineered cytokine receptor switch (right).

FIG. 32 illustrates differentiation for engineered immune cells with activated engineered cytokine receptor switches.

FIG. 33A provides an image of a container with an unfunctionalized well, and which contains immune cells engineered to express an engineered cytokine receptor switch configured to bind fluorescein.

FIG. 33B provides an image of a container with a fluorescein-functionalized well, and which contains immune cells engineered to express an engineered cytokine receptor switch configured to bind fluorescein.

FIG. 34 provides cell counts and purity for immune cells cultured in an unfunctionalized well plate, adherent to a surface in an activator-functionalized well plate, and suspended in solution within an activator-functionalized well plate.

FIG. 35 depicts multiple schemes for contacting various fluorescein-labeled substrates to immune cells engineered to express engineered cytokine receptor switches with fluorescein binding affinity.

FIG. 36 provides profiling data for immune cells expressing engineered cytokine receptor switches with fluorescein binding affinity and contacted to various concentrations of activator-functionalized dextran.

FIG. 37A provides engineered immune cell CCR7 and CD45RA expression profiles following activation with various formulations of hydrophobic beads.

FIG. 37B provides engineered immune cell CCR7 and CD45RA expression profiles following activation with various formulations of hydrophilic beads.

FIG. 38A provides the percentages of multiple T-cell phenotypes among immune cells activated with hydrophobic beads functionalized with activators coupled through various length linkers at a ratio of 1 cell per 2 beads.

FIG. 38B provides the percentages of multiple T-cell phenotypes among immune cells activated with hydrophobic beads functionalized with activators coupled through various length linkers at a ratio of 1 cell per 5 beads.

FIG. 38C provides the percentages of multiple T-cell phenotypes among immune cells activated with hydrophobic beads functionalized with activators coupled through various length linkers at a ratio of 1 cell per 10 beads.

FIG. 39A provides the percentages of multiple T-cell phenotypes among immune cells activated with hydrophilic beads functionalized with activators coupled through various length linkers at a ratio of 1 cell per 2 beads.

FIG. 39B provides the percentages of multiple T-cell phenotypes among immune cells activated with hydrophilic beads functionalized with activators coupled through various length linkers at a ratio of 1 cell per 5 beads.

FIG. 39C provides the percentages of multiple T-cell phenotypes among immune cells activated with hydrophilic beads functionalized with activators coupled through various length linkers at a ratio of 1 cell per 10 beads.

FIG. 40 provides T-cell phenotypes among engineered immune cells contacted or not contacted to activator-functionalized dextran.

FIG. 41A provides a timeline for transfection, activation, differentiation, and expansion of engineered immune cells consistent with the present disclosure.

FIG. 41B provides CCR7 and CD45RA expression profiles of engineered immune cells contacted to various activator-functionalized substrates.

FIG. 42A outlines a method for functionalizing protein-derived thiols consistent with the present disclosure.

FIG. 42B details a method for reducing and functionalizing protein disulfide bonds with a conjugating group consistent with the present disclosure.

FIG. 43A overviews a lysine-selective functionalization method consistent with the present disclosure.

FIG. 43B outlines a method for functionalizing protein-derived lysine residues consistent with the present disclosure.

FIG. 44 is a flow diagram outlining an example method for stochastic cysteine conjugation consistent with the present disclosure.

FIG. 45 is a flow diagram outlining an example method for antibody purification via buffer exchange consistent with the present disclosure.

FIG. 46 is a flow diagram outlining an example method for antibody functionalization via lysine conjugation consistent with the present disclosure.

FIG. 47 provides UV-visible absorbance (UV-vis) spectra of “naked” antibody, unconjugated 5-maleimide fluorescein activator, and antibody-drug conjugates (ADCs) with different drug-antibody ratio (DAR). The differential absorbances of the antibody at 280 nm and fluorescein at 493 nm allows for determination of the concentrations of the conjugated antibody, conjugated fluorescein, and the DAR.

FIG. 48 provides UV-vis spectral overlay of multiple ADCs consistent with the present disclosure.

FIG. 49A outlines a time-resolved fluorescence (TRF) assay for determining the average concentration of metal-chelating conjugating groups coupled to an antibody.

FIG. 49B details the TRF method for determining the number of metal-chelating groups coupled to an antibody.

FIG. 50 provides an example of mass spectrometric ADC data. FIG. 50 Panel A provides a liquid chromatogram from liquid chromatography-mass spectrometry analysis of the ADC. FIG. 50 Panel B provides the apparent mass of multiply charged ADC species. FIG. 50 Panel C provides a deconvoluted mass spectrum of light and heavy chains of the ADC. FIG. 50 Panels D and E provide enlarged views of the mass spectra of the ADC light and heavy chains shown in Panel C.

FIG. 51 provides a schematic of ADC product distributions for different conjugation chemistries. FIG. 51 Panel A illustrates an antibody functionalized by stochastic cysteine conjugation and FIG. 51 Panel B provides DARs achieved through stochastic cysteine conjugation. FIG. 51 Panel C illustrates an antibody functionalized by lysine conjugation and FIG. 51 Panel D provides DARs achieved through lysine conjugation. FIG. 51 Panel E illustrates an antibody functionalized by site-specific conjugation and FIG. 51 Panel F provides DARs achieved through site-specific conjugation.

FIG. 52 provides a plot of DARs for stochastic cysteine ADCs prepared with varying amounts of reductant.

FIG. 53A provides the frequency of various DARs for an ADC preparation consistent with the present disclosure.

FIG. 53B depicts positional isomers of a stochastic cysteine ADC.

FIGS. 54A-54D provide size-exclusion chromatograms for multiple stochastic cysteine ADCs containing varying DARs. In each figure, the upper and lower panels provide the absorbances at the 280 nm and 493 nm channels, respectively. FIG. 54A provides data for an exemplary ADC preparation, while FIG. 54B, FIG. 54C, and FIG. 54D correspond to exemplary ADCs with DARs of 3.5, 5.4, and 7.8, respectively.

FIG. 55A illustrates the results of TCEP titration used for determining the number of TCEP equivalents required to generate an ADC with target DAR.

FIG. 55B provides antibody % monomeric, % low-molecular weight (LMW), and % high-molecular weight (HMW) content for multiple ADC preparations consistent with the present disclosure.

FIG. 56A provides an SEC chromatogram of an unfunctionalized antibody consistent with the present disclosure.

FIGS. 56B-56D provide SEC chromatograms collected at 280 nm (panel A) and 493 nm (panel B) showing area % of monomer, HMW and LMW impurities of an ADC consistent with the present disclosure.

FIG. 57 provides UV-vis spectra of multiple ADCs with different DARs.

FIG. 58A provides a correlation plot of DAR as a function of conjugating group equivalents used for lysine functionalization.

FIG. 58B provides relative frequencies of different DARs among a preparation of lysine-functionalized antibodies.

FIG. 58C provides UV-vis spectra of multiple lysine-functionalized antibodies with varying DARs.

FIG. 59A provides a correlation plot of antibody % monomer content as a function of DAR for multiple lysine-functionalized ADCs consistent with the present disclosure.

FIG. 59B provides a correlation plot of antibody % HMW and % LMW content as a function of DAR for multiple ADCs consistent with the present disclosure.

FIGS. 60A-60D provide SEC chromatograms for DAR 1.4 (FIG. 60A), DAR 2.9 (FIG. 60B), DAR 5.2 (FIG. 60C), and DAR 9.5 (FIG. 60D) ADCs consistent with the present disclosure.

FIG. 61A provides UV-vis spectra of multiple ADCs generated in mg-scale syntheses, as well as a UV-vis spectrum of an unfunctionalized antibody.

FIG. 61B provides UV-vis spectra of multiple ADCs generated in microgram-scale syntheses.

FIG. 61C provides a plot of DARs as a function of conjugating group equivalents used during synthesis for multiple ADCs generated from mg-scale syntheses.

FIG. 61D provides a plot of DARs as a function of conjugating group equivalents used during synthesis for multiple ADCs generated from microgram-scale syntheses.

FIGS. 61E-61H provide SEC chromatograms for multiple ADCs consistent with the present disclosure. FIG. 61E provides SEC chromatograms for DAR 3.9 ADCs generated from a mg-scale synthesis. FIG. 61F provides SEC chromatograms for DAR 7.8 ADCs generated from a mg-scale synthesis. FIG. 61G provides SEC chromatograms for DAR 4.1 ADCs generated from a microgram-scale synthesis. FIG. 61H provides SEC chromatograms for DAR 8.6 ADCs generated from a microgram-scale synthesis.

FIGS. 62A-62E provide SEC chromatograms for DAR 2.9 (FIG. 62A), 5.5 (FIG. 62B), 7.1 (FIG. 62C), 7.4 (FIG. 62D), and 7.5 (FIG. 62E) ADCs consistent with the present disclosure.

FIG. 63A provides an SEC chromatogram of an unfunctionalized antibody consistent with the present disclosure.

FIG. 63B provides SEC chromatograms of a cysteine-functionalized ADC consistent with the present disclosure.

FIG. 64A provides UV-vis spectra of ADCs generated with mg- and μg-scale syntheses.

FIG. 64B provides the UV-vis spectra of FIG. 64A normalized at 280 nm.

FIG. 65A and FIG. 65B provide overlaid 280 nm and 493 nm SEC chromatograms of a crude reaction mixture collected during antibody thiol-functionalization (FIG. 65A) and of the ADC following purification (FIG. 65B).

FIG. 66 provides SEC chromatograms of an ADC consistent with the present disclosure.

FIG. 67A and FIG. 67B provide raw (FIG. 67A) and normalized (FIG. 67B) UV-visible absorbance data for two ADCs prepared with different antibody titers.

FIG. 68A and FIG. 68B provide SEC chromatograms of a crude lysine ADC conjugation mixture (FIG. 68A) and of the purified ADC product (FIG. 68B).

FIG. 69A and FIG. 69B provide SEC chromatograms of a crude conjugation mixture (FIG. 69A) used to generate an ADC, and of the purified ADC product (FIG. 69B).

FIGS. 70A-70C provide SEC chromatograms of an unfunctionalized antibody (FIG. 70A) and of ADCs from generated with a 30 μg-scale synthesis (FIG. 70B) and ADCs generated with a 100 μg-synthesis (FIG. 70C).

FIG. 71 provides UV-vis spectra of two ADCs prepared from syntheses of varying scales.

FIGS. 72A-72C provide SEC chromatograms of crude (pre-purification) reaction mixtures of ADCs prepared with 5 equivalents (FIG. 72A), 10 equivalents (FIG. 72B), and 20 equivalents (FIG. 72C) of lysine conjugating groups.

FIG. 73A and FIG. 73B provide SEC chromatograms of multiple metal chelator functionalized ADCs consistent with the present disclosure.

FIG. 74A provides a plot of Eu(III) fluorescence signal intensity as a function of free or antibody-bound metal chelator concentration.

FIG. 74B provides a plot of fluorescence signal intensities as a function of Eu(III) concentration.

FIG. 75 plots DARs as a function of reducing equivalents used during syntheses for multiple preparations of ADCs containing DOTA.

FIG. 76 depicts a method for separating antibody chains with dithiothreitol.

FIG. 77A provides a plot of monomer content as a function of DAR for multiple lysine ADCs consistent with the present disclosure.

FIG. 77B provides a plot of HMW antibody content as a function of DAR for multiple lysine ADCs consistent with the present disclosure.

FIG. 77C provides a plot of LMW antibody content as a function of DAR for multiple lysine ADCs consistent with the present disclosure.

FIGS. 78A-78D provide schematics of various cleavable carrier-small molecule activator conjugates of the present disclosure for CAR-T cell release during ex vivo manufacturing. FIG. 78A provides a schematic representation of disulfide cleavage of a carrier from a small molecule under reducing conditions. FIG. 78B provides a schematic representation of photocleavage of a carrier from a small molecule after exposure to 365 nm light. FIG. 78C provides a schematic representation of protease-mediated cleavage of a carrier from a small molecule. FIG. 78D provides a schematic representation of a Staudinger reduction of an azide to an amine with concomitant release of a carrier from a small molecule.

FIGS. 79A-79C provide schematics of various methods of preparing non-cleavable and disulfide cleavable dextran-fluorescein conjugates of the present disclosure. FIG. 79A provides a schematic representation of a non-cleavable dextran-fluorescein conjugate prepared by direct acylation. FIG. 79B provides a schematic representation of a non-cleavable dextran-fluorescein conjugate prepared by sequential acylation and strain-promoted azide-alkyne cycloaddition (SPAAC). FIG. 79C provides a schematic representation of a disulfide-cleavable dextran-fluorescein conjugate prepared by sequential acylation and SPAAC.

FIG. 80A provides a graph showing the UV absorbance spectra of AD40×10-FL non-cleavable dextran-fluorescein conjugates at 0.75 mg/mL in PBS.

FIG. 80B provides a graph showing the UV absorbance spectra of AD40×10-FL non-cleavable dextran-fluorescein conjugates at 10 mg/mL in PBS.

FIGS. 81A-81C provide a series of graphs showing UV absorbance data for AD40×60-FL non-cleavable dextran-fluorescein conjugates obtained at different molar equivalents of FL NHS ester. Varying molar excess of FL NHS ester enables changing the relative loading of fluorescein per dextran.

FIG. 81A provides a graph showing the UV absorbance spectra of various AD40×60-FL conjugates containing different FL loadings at 0.375 mg/mL in PBS.

FIG. 81B provides a graph showing absorbance at 493 nm of the various AD40×60-FL conjugates showing the linear relationship between fluorescein loading and molar excess of FL NHS ester.

FIG. 81C provides a graph showing UV absorbance for 10 mg/mL solutions of AD40×60-FL containing low, medium, and high FL loading per dextran.

FIGS. 82A-82D provide a series of graphs showing UV absorbance of non-cleavable and disulfide-cleavable 40 kDa dextran-fluorescein conjugates containing different fluorescein loading per dextran. Absorbance spectra were obtained prior to immobilization on M-450 amine modified Dynabeads. FIG. 82A provides a graph showing UV absorbance of non-cleavable AD40×10-FL conjugates. FIG. 82B provides a graph showing UV absorbance of disulfide-cleavable AD40×10-SS-FAM conjugates. FIG. 82C provides a graph showing UV absorbance of non-cleavable AD40×60-FAM conjugates. FIG. 82D provides a graph showing UV absorbance of disulfide-cleavable AD40×60-SS-FAM conjugates.

FIG. 83 provides a schematic showing end group covalent immobilization of non-cleavable and disulfide-cleavable dextran-fluorescein conjugates to M-450 tosylactivated Dynabeads by reductive amination.

FIG. 84 provides a graph showing the relative fluorescence of non-cleavable and disulfide-cleavable 4.5 μm M-450 Dynabead-dextran-fluorescein conjugates.

FIG. 85 provides a schematic showing the preparation of non-cleavable and photocleavable 4.5 μm M-450 Dynabead-fluorescein conjugates by direct isothiocyanate/amine coupling or click chemistry.

FIG. 86A provides a graph showing the relative fluorescence and approximate concentrations of conjugated fluorescein for non-cleavable M-450-PEG-FITC Dynabeads.

FIG. 86B provides a graph showing the relative fluorescence and approximate concentrations of conjugated fluorescein for non-cleavable M-450-PEG-FAM Dynabeads.

FIG. 86C provides a graph showing the relative fluorescence and approximate concentrations of conjugated fluorescein for photocleavable M-450-PEG-PC-FAM Dynabeads.

FIG. 87A provides a schematic representation and graph showing FITC fluorescence in the absence of M-450 tosylactivated Dynabeads.

FIG. 87B provides a schematic representation and graph showing FITC fluorescence in the presence of suspended M-450 tosylactivated Dynabeads.

FIG. 87C provides a schematic representation and graph showing FITC fluorescence in the presence of settled M-450 tosylactivated Dynabeads.

FIG. 88 provides a timeline for an experiment design to assess IL7Rα SMAR activation by various conjugates of the present disclosure.

FIGS. 89A-89D provide a series of graphs showing that FITC-conjugated Dynabeads activate the IL7Rα SMAR, resulting in changes in T_{scm}, Tₑₘ, Tₑₘ, and Tₑₘᵣₐ memory populations within the CD8+ SMAR+ T cell population. The data shown is from Day 7 in the experiment timeline of FIG. 88, using a 5:1 bead: cell ratio. FIG. 89A provides a graph showing percentages of Tₑₘ phenotypes within the SMAR+CD8+ T-cell population. FIG. 89B provides a graph showing percentages of T_{scm} phenotypes within the SMAR+CD8+ T-cell population. FIG. 89C provides a graph showing percentages of Tₑₘᵣₐ phenotypes within the SMAR+CD8+ T-cell population. FIG. 89D provides a graph showing percentages of Tₑₘ phenotypes within the SMAR+CD8+ T-cell population.

FIGS. 90A-90D provide a series of graphs showing that FITC-conjugated Dynabeads activate the IL7Rα SMAR, resulting in changes in T_{scm}, Tₑₘ, Tₑₘ, and Tₑₘᵣₐ memory populations within the CD4+ SMAR+ T cell population. The data shown is from Day 7 in the experiment timeline of FIG. 88, using a 5:1 bead: cell ratio. FIG. 90A provides a graph showing percentages of T_{cm} phenotypes within the SMAR+CD4+ T-cell population. FIG. 90B provides a graph showing percentages of Tₛₑₘ phenotypes within the SMAR+CD4+ T-cell population. FIG. 90C provides a graph showing percentages of Tₑₘᵣₐ phenotypes within the SMAR+CD4+ T-cell population. FIG. 90D provides a graph showing percentages of Tₑₘ phenotypes within the SMAR+CD4+ T-cell population.

FIGS. 91A-91D provide a series of graphs showing that FITC-conjugated Dynabeads activate the IL7Rα SMAR, resulting in changes in T_{scm}, T_{cm}, Tₑₘ, and Tₑₘᵣₐ memory populations within the CD8+ SMAR+ T cell population. The data shown is from Day 7 in the experiment timeline of FIG. 88, using a 1:1 bead: cell ratio. FIG. 91A provides a graph showing percentages of T_{cm} phenotypes within the SMAR+CD8+ T-cell population. FIG. 91B provides a graph showing percentages of T_{scm} phenotypes within the SMAR+CD8+ T-cell population. FIG. 91C provides a graph showing percentages of Tₑₘᵣₐ phenotypes within the SMAR+CD8+ T-cell population. FIG. 91D provides a graph showing percentages of Tₑₘ phenotypes within the SMAR+CD8+ T-cell population.

FIGS. 92A-92D provide a series of graphs showing that FITC-conjugated Dynabeads activate the IL7Rα SMAR, resulting in changes in T_{scm}, T_{cm}, Tₑₘ, and Tₑₘᵣₐ memory populations within the CD4+ SMAR+ T cell population. The data shown is from Day 7 in the experiment timeline of FIG. 88, using a 1:1 bead: cell ratio. FIG. 92A provides a graph showing percentages of T_{cm} phenotypes within the SMAR+CD4+ T-cell population. FIG. 92B provides a graph showing percentages of T_{scm} phenotypes within the SMAR+CD4+ T-cell population. FIG. 92C provides a graph showing percentages of Tₑₘᵣₐ phenotypes within the SMAR+CD4+ T-cell population. FIG. 92D provides a graph showing percentages of Tₑₘ phenotypes within the SMAR+CD4+ T-cell population.

### DETAILED DESCRIPTION

Described herein are engineered immune cells, compositions for activating the engineered immune cells, associated methods for using the compositions to activate the engineered immune cells, and methods of treating subjects using the engineered immune cells. The immune cells can express engineered cytokine receptor switches, which can be configured to transduce a signal upon activation, often promoting differentiation to specific cell phenotypes. The compositions can include a species which binds to an activator binding domain of an engineered cytokine receptor switch, hereinafter referred to as an “activator,” which can activate the engineered cytokine receptor switch to direct differentiation towards particular phenotypes. The concentration, activator type, exposure time, and/or physical characteristics of the composition can be varied to generate desired phenotypes and phenotype ratios in the immune cells. In some cases, the immune cells are contacted with additional ligands, such as agonists of natively expressed receptors (e.g., CD3, CD28, and/or IL2). Collectively, these techniques can facilitate high degrees of control over immune cell activation, expansion, and/or differentiation. The compositions may direct differentiation of the engineered immune cells towards particular memory phenotypes while diminishing the prevalence of effector phenotypes.

An embodiment of the compositions disclosed herein includes a substrate coupled to an activator. The substrate can be a nano or microscale species, such as a bead or a protein (e.g., albumin or an antibody), or a surface, such as a wall of a tube or microwell. The substrate can be directly or indirectly coupled to the activator. For many of the substrates disclosed herein, the activator is covalently or noncovalently coupled through a linker. In some cases, the activator is coupled to a carrier complex which is coupled to the substrate. For example, the activator can be coupled to a dextran-based carrier complex coupled to substrate-bound streptavidin. Such designs can facilitate modularity in activator density and substrate properties, thereby providing high degrees of control over immune cell activation.

In some embodiments, the present disclosure provides compositions and methods for controlling differentiation during immune cell activation and expansion, yielding engineered immune cells with controlled memory and effector phenotype ratios. While effector T- and B-cells tend to have higher potencies (e.g., against target cancer cell types) than memory T- and B-cells, memory T- and B-cells tend to exhibit longer lifespans and greater ability to divide, leading to longer persistence in vivo. Accordingly, predominantly memory phenotype and mixed memory and effector phenotype engineered immune cell populations can be optimal for treating recalcitrant and recurrent diseases, including many forms of cancers, which require engineered immune cell persistence and longer treatment timelines.

The immune cells of the present disclosure often express an engineered cytokine receptor switch, which may be engineered to activate an intracellular response (e.g., a cytokine pathway) upon binding of an exogenous activator to an extracellular domain. For many of the immune cell activation and expansion methods disclosed herein, an engineered cytokine receptor switch of an engineered immune cell is activated to promote differentiation to a particular phenotype. In many cases, the engineered cytokine receptor switch comprises an activator binding domain, a transmembrane domain, and an intracellular signaling domain. For many of the engineered cytokine receptor switches disclosed herein, the activator binding domain binds an activator (e.g., a small molecule, a peptide, or an oligonucleotide) to activate the intracellular signaling domain.

### I. Engineered Immune Cells

Aspects of the present disclosure provide immune cells engineered to express controllable activation motifs and/or therapeutic activities. The engineered immune cells can express an engineered cytokine receptor switch which provides a handle for activation and activity, and can facilitate control over activity and phenotype. The cytokine receptor switch may comprise an activator binding domain which transduces an intracellular signal upon binding to an activator species. For many of the engineered cells disclosed herein, the activator domain is disposed in an extracellular space (e.g., is an ectodomain) and is operably coupled to an intracellular signaling domain, facilitating controlled activation of the engineered immune cells with exogenous activators (e.g., small molecules, peptides, or oligonucleotides configured to bind to the activator domain).

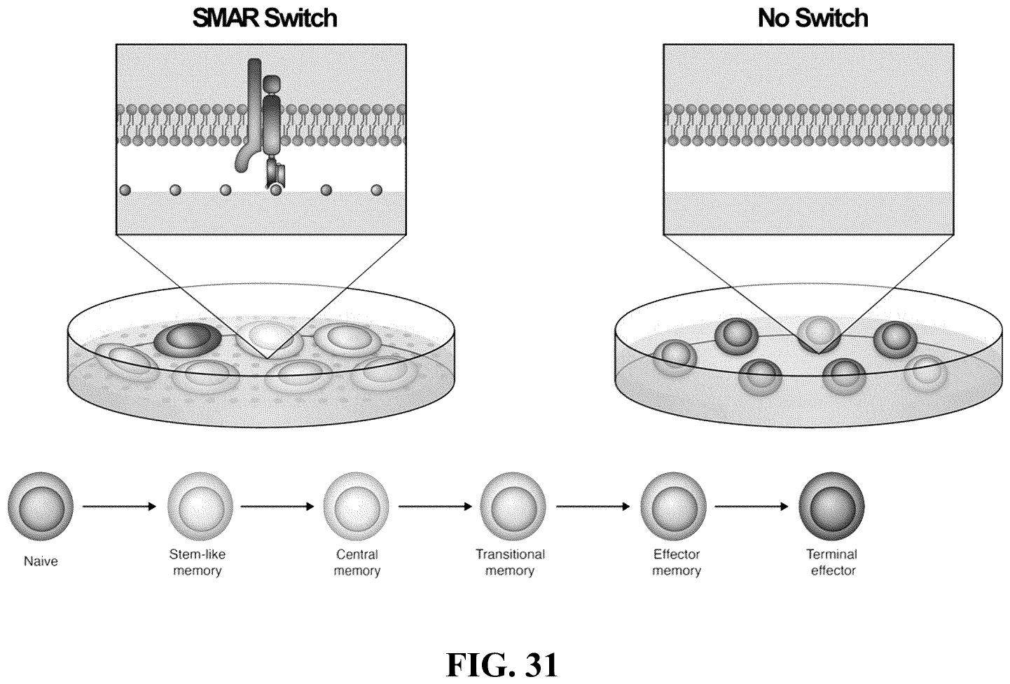

Activation of the cytokine receptor switch can modify the phenotype of the engineered immune cell, thereby altering its therapeutic activity. For many of the engineered immune cells disclosed herein, the cytokine receptor switch affects differentiation towards memory T-cell phenotypes with enhanced persistence in vivo, and diminishes the proportion of T-cells which differentiate into terminal effector cells. This concept is illustrated in FIG. 31, which provides an illustrative comparison of engineered T-cells cultured with activated engineered cytokine receptor switches (left) and T-cells which lack the engineered cytokine receptor switch (right). In this illustrative example, in the presence of small molecule activators of the engineered cytokine receptor switches, the engineered T-cell population primarily differentiate into stem-like memory and central memory T-cells, while the T-cell population lacking the engineered cytokine receptor switch primarily differentiate into terminal effector cells.

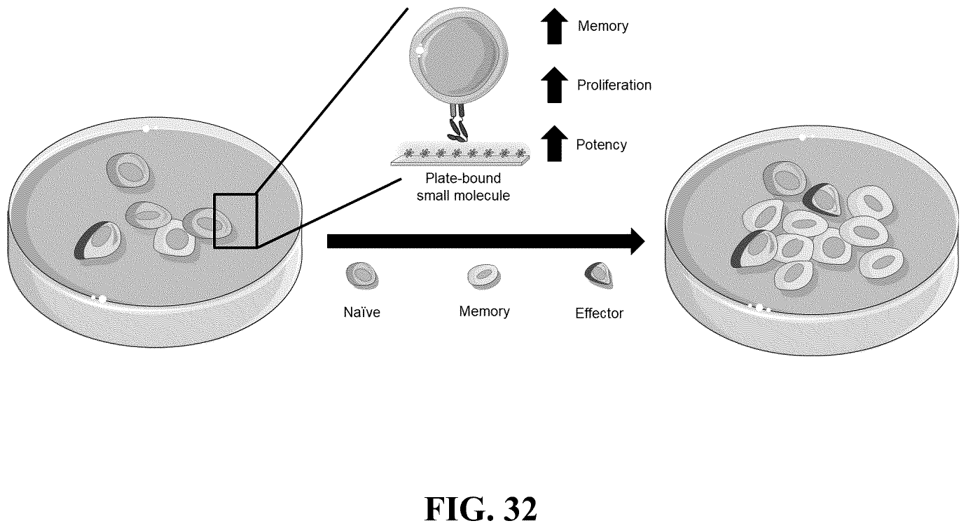

FIG. 32 illustrates an embodiment of activator-controlled differentiation of engineered immune cells with activated engineered cytokine receptor switches. In this illustrative example, an initial population of engineered immune cells collected from a donor and engineered to express engineered cytokine receptor switches is primarily comprised of naïve T-cells along with minor amounts of memory and effector cells. The cells are expanded in a container (e.g., a well, plate, tube, flask, or bioreactor). A surface of the container is coated with a small molecule activator of the engineered cytokine receptor switch (e.g., coupled to small molecule activator-functionalized dextran), such that the engineered cytokine receptor switches of the T-cells are activated. Activation of the engineered cytokine receptor switch can increase T-cell potency, increase T-cell proliferation, and promote differentiation into specific cell types, such as memory T-cells. In many cases, engineered cytokine receptor switch activation promotes differentiation into memory cell phenotypes and diminishes the proportion of cells which become terminal effector cells. In many cases, the engineered cytokine receptor switch induces differentiation towards specific memory cell phenotypes.

Activation of the cytokine receptor switch may alternatively or additionally promote homing of the engineered immune cell to lymphoid organs (e.g., lymph nodes, spleen, thymus, and/or bone marrow), where the lymphoid environment may facilitate activation, expansion, and/or conversion to memory cell phenotypes. For example, activation may upregulate expression of cell surface markers that enable homing of the immune cell to lymphoid organs (e.g., CD62L, CCR7), such as homing to the lymph nodes, spleen, thymus, and/or bone marrow.

The engineered immune cells can comprise additional exogenous, overexpressed, or engineered features configured for therapeutic activity. In some embodiments, the immune cell may be further engineered to express a chimeric antigen receptor (CAR). The CAR can be configured to activate the engineered immune cells in the presence of a particular antigen, such as a tumor antigen.

The engineered immune cells can comprise a single cell type, or can include a heterogenous population of cells. As non-limiting examples, the immune cell engineered to express a cytokine receptor switch may be a T-cell, a regulatory T-cell, a B-cell, a natural killer cell (NK cell), a FcεRIγ deficient NK cell (g-NK cell), a neutrophil, an eosinophil, a macrophage, a monocyte, a basophil, a γδ T-cell, or other immune cell type. In some cases, the vector causes activation, differentiation, and/or polarization of the immune cell. Often, the engineered immune cells are comprised primarily of lymphocytes. In some such cases, the engineered immune cells comprise at least 70%, at least 80%, at least 90%, at least 95%, at least 98%, or at least 99% lymphocytes. In some cases, the engineered immune cells are comprised primarily of T-cells. In some such cases, the engineered immune cells comprise at least 70%, at least 80%, at least 90%, at least 95%, at least 98%, or at least 99% T-cells. In some cases, the T-cells are at least 10%, at least 20%, at least 30%, at least 40%, at least 50%, at least 60%, at least 70%, or at least 80% memory T-cells.

### II. Engineered Cytokine Receptor Switches

An engineered cytokine receptor switch can comprise an activator binding domain, a transmembrane domain, and an intracellular signaling domain. The activator binding domain may bind an activator (e.g., a small molecule, a peptide, an oligonucleotide, or a protein) to activate the intracellular signaling domain. In some embodiments, the activator binding domain is a small molecule binding domain that binds a small molecule (e.g., fluorescein or a fluorescein derivative (e.g., fluorescein isothiocyanate (FITC)), tetraxetan (DOTA), biotin or linker-specific biotin, or 4-[(6-methylpyrazin-2-yl)oxy]benzoate (MPOB)). The activation signal may be communicated through the transmembrane domain to convert an extracellular stimulus (e.g., binding of the activator) to an intracellular effect (e.g., activation of a cytokine signaling pathway).

An engineered cytokine receptor switch of the present disclosure may comprise an activator binding domain. The activator binding domain may be positioned in an extracellular region of the engineered cytokine receptor switch and may be designed to bind an activator to activate intracellular signaling through the intracellular signaling domain. In some embodiments, the activator may be an exogenous activator (e.g., an exogenous small molecule, an exogenous peptide, or an exogenous oligonucleotide) to prevent activation of the engineered cytokine receptor switch in the absence of an external stimulus (e.g., administration of the activator), prevent cross-reactivity of the activator with other biological components, and to enable dynamic control of receptor signaling. Optionally, the engineered cytokine receptor switch may exhibit some activity independent of binding of the activator to the activator binding domain, as discussed further below.

In some embodiments, the engineered cytokine receptor switch may further comprise a hinge connecting the activator binding domain to the transmembrane domain. A hinge may increase flexibility of the engineered cytokine receptor switch, which may reduce spatial constraints between the activator binding domain and the activator (e.g., a small molecule activator adhered to a surface). The engineered cytokine receptor switch may further comprise a signal peptide to direct expression of the engineered cytokine receptor switch to the endoplasmic reticulum (ER). In some embodiments, a signal peptide present at the N-terminus of the protein may direct the protein to be synthesized in the ER membrane and subsequently trafficked to the plasma membrane as a transmembrane protein.

An engineered cytokine receptor switch of the present disclosure may comprise a domain (e.g., an intracellular signaling domain, a transmembrane domain, a hinge, a signal peptide, or combinations thereof) derived from an endogenous cytokine receptor. In some embodiments, an engineered cytokine receptor switch may comprise a domain derived from an interleukin 2 receptor subunit a (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, a CD8, a CD3, a CD4, a CD28, a 4-1BB, a CD28, an OX40, an inducible T-cell costimulatory (ICOS), a CD27, or combinations thereof.

As described herein, a cytokine receptor switch, also referred to as a small molecule activated receptor (SMAR) switch, may be engineered to activate an intracellular response (e.g., a cytokine pathway) upon binding of an activator to an extracellular domain. In some embodiments, an engineered cytokine receptor switch may comprise an activator binding domain, a transmembrane domain, and an intracellular signaling domain. The activator binding domain may bind an activator (e.g., a small molecule, a peptide, an oligonucleotide, or a protein) to activate the intracellular signaling domain. In some embodiments, the activator binding domain is a small molecule binding domain that binds a small molecule (e.g., a fluorescein, fluorescein derivative, DOTA, biotin, or MPOB). The activation signal may be communicated through the transmembrane domain to convert an extracellular stimulus (e.g., binding of the activator) to an intracellular effect (e.g., activation of a cytokine signaling pathway).

In some embodiments, the engineered cytokine receptor switch may further comprise a hinge connecting the activator binding domain to the transmembrane domain. A hinge may increase flexibility of the engineered cytokine receptor switch, which may reduce spatial constraints between the activator binding domain and the activator (e.g., a small molecule activator adhered to a surface). The engineered cytokine receptor switch may further comprise a signal peptide to direct expression of the engineered cytokine receptor switch to the endoplasmic reticulum (ER). In some embodiments, a signal peptide present at the N-terminus of the protein may direct the protein to be synthesized in the ER membrane and subsequently trafficked to the plasma membrane as a transmembrane protein.

An engineered cytokine receptor switch of the present disclosure may comprise a domain (e.g., an intracellular signaling domain, a transmembrane domain, a hinge, a signal peptide, or combinations thereof) derived from an endogenous cytokine receptor. In some embodiments, an engineered cytokine receptor switch may comprise a domain derived from an interleukin 2 receptor subunit α (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, a CD8, a CD3, a CD4, a CD28, a 4-1BB, a CD28, an OX40, an inducible T-cell costimulatory (ICOS), a CD27, or combinations thereof.

Examples of engineered cytokine receptor switches and their associated polynucleotide sequences are provided in Table 1.

TABLE 1
Representative Examples of Engineered Cytokine Receptor Switches

| | SEQ ID | |
|---|---|---|
| SMAR | NO | Sequence |
| IL2Rα | SEQ ID | MDSYLLMWGLLTFIMVPGCQADVVMTQTPLSLPVSLGDQASISCRSSQSLVHSNG |
| SMAR | NO: 1 | NTYLRWYLQKPGQSPKVLIYKVSNRVSGVPDRFSGSGSGTDFTLKINRVEAEDLG |
| | | VYFCSQSTHVPWTFGGGTKLEIKSSADDAKKDAAKKDDAKKDDAKKDGGVKLD |
| | | ETGGGLVQPGGAMKLSCVTSGFTFGHYWMNWVRQSPEKGLEWVAQFRNKPYN |
| | | YETYYSDSVKGRFTISRDDSKSSVYLQMNNLRVEDTGIYYCTGASYGMEYLGQG |
| | | TSVTVSTTTPAPRPPTPAPTIASQPLSLRPEACRPAAGGAVHTRGLDFACD |
| | | VAVAGCVFLLISVLLLSGLTWQRRQRKSRRTI |
| IL2Rβ | SEQ ID | MAAPALSWRLPLLILLLPLATSWASADVVMTQTPLSLPVSLGDQASISCRSSQSLV |
| SMAR | NO: 2 | HSNGNTYLRWYLQKPGQSPKVLIYKVSNRVSGVPDRFSGSGSGTDFTLKINRVEA |
| | | EDLGVYFCSQSTHVPWTFGGGTKLEIKSSADDAKKDAAKKDDAKKDDAKKDGG |
| | | VKLDETGGGLVQPGGAMKLSCVTSGFTFGHYWMNWVRQSPEKGLEWVAQFRN |
| | | KPYNYETYYSDSVKGRFTISRDDSKSSVYLQMNNLRVEDTGIYYCTGASYGMEYL |
| | | GQGTSVTVSTTTPAPRPPTPAPTIASQPLSLRPEACRPAAGGAVHTRGLDFACDIPW |
| | | LGHLLVGLSGAFGFIILVYLLINCRNTGPWLKKVLKCNTPDPSKFFSQLSSEHGGD |
| | | VQKWLSSPFPSSSFSPGGLAPEISPLEVLERDKVTQLLLQQDKVPEPASLSSNHSLT |
| | | SCFTNQGYFFFHLPDALEIEACQVYFTYDPYSEEDPDEGVAGAPTGSSPQPLQPLS |
| | | GEDDAYCTFPSRDDLLLFSPSLLGGPSPPSTAPGGSGAGEERMPPSLQERVPRDWD |
| | | PQPLGPPTPGVPDLVDFQPPPELVLREAGEEVPDAGPREGVSFPWSRPPGQGEFRA |
| | | LNARLPLNTDAYLSLQELQGQDPTHLV |
| IL2Rγ | SEQ ID | MLKPSLPFTSLLFLQLPLLGVGDVVMTQTPLSLPVSLGDQASISCRSSQSLVHSNG |
| SMAR | NO: 3 | NTYLRWYLQKPGQSPKVLIYKVSNRVSGVPDRFSGSGSGTDFTLKINRVEAEDLG |
| | | VYFCSQSTHVPWTFGGGTKLEIKSSADDAKKDAAKKDDAKKDDAKKDGGVKLD |
| | | ETGGGLVQPGGAMKLSCVTSGFTFGHYWMNWVRQSPEKGLEWVAQFRNKPYN |
| | | YETYYSDSVKGRFTISRDDSKSSVYLQMNNLRVEDTGIYYCTGASYGMEYLGQG |
| | | TSVTVSTTTPAPRPPTPAPTIASQPLSLRPEACRPAAGGAVHTRGLDFACDVVISVG |
| | | SMGLIISLLCVYFWLERTMPRIPTLKNLEDLVTEYHGNFSAWSGVSKGLAESLQPD |
| | | YSERLCLVSEIPPKGGALGEGPGASPCNQHSPYWAPPCYTLKPET |
| IL7Rα | SEQ ID | MTILGTTFGMVFSLLQVVSGDVVMTQTPLSLPVSLGDQASISCRSSQSLVHSNGNT |
| SMAR | NO: 4 | YLRWYLQKPGQSPKVLIYKVSNRVSGVPDRFSGSGSGTDFTLKINRVEAEDLGVY |
| | | FCSQSTHVPWTFGGGTKLEIKSSADDAKKDAAKKDDAKKDDAKKDGGVKLDET |
| | | GGGLVQPGGAMKLSCVTSGFTFGHYWMNWVRQSPEKGLEWVAQFRNKPYNYE |
| | | TYYSDSVKGRFTISRDDSKSSVYLQMNNLRVEDTGIYYCTGASYGMEYLGQGTSV |
| | | TVSTTTPAPRPPTPAPTIASQPLSLRPEACRPAAGGAVHTRGLDFACDPILLTISILSF |
| | | FSVALLVILACVLWKKRIKPIVWPSLPDHKKTLEHLCKKPRKNLNVSFNPESFLDC |
| | | QIHRVDDIQARDEVEGFLQDTFPQQLEESEKQRLGGDVQSPNCPSEDVVITPESFG |
| | | RDSSLTCLAGNVSACDAPILSSSRSLDCRESGKNGPHVYQDLLLSLGTTNSTLPPPF |
| | | SLQSGILTLNPVAQGQPILTSLGSNQEEAYVTMSSFYQNQ |
| IL15Rα | SEQ ID | MAPRRARGCRTLGLPALLLLLLLRPPATRGDVVMTQTPLSLPVSLGDQASISCRSS |
| SMAR | NO: 5 | QSLVHSNGNTYLRWYLQKPGQSPKVLIYKVSNRVSGVPDRFSGSGSGTDFTLKIN |
| | | RVEAEDLGVYFCSQSTHVPWTFGGGTKLEIKSSADDAKKDAAKKDDAKKDDAK |
| | | KDGGVKLDETGGGLVQPGGAMKLSCVTSGFTFGHYWMNWVRQSPEKGLEWVA |
| | | QFRNKPYNYETYYSDSVKGRFTISRDDSKSSVYLQMNNLRVEDTGIYYCTGASYG |
| | | MEYLGQGTSVTVSTTTPAPRPPTPAPTIASQPLSLRPEACRPAAGGAVHTRGLDFA |
| | | CDVAISTSTVLLCGLSAVSLLACYLKSRQTPPLASVEMEAMEALPVTWGTSSRDE |
| | | DLENCSHHL |
| IL21Rα | SEQ ID | MPRGWAAPLLLLLLQGGWGDVVMTQTPLSLPVSLGDQASISCRSSQSLVHSNGN |
| SMAR | NO: 6 | TYLRWYLQKPGQSPKVLIYKVSNRVSGVPDRFSGSGSGTDFTLKINRVEAEDLGV |
| | | YFCSQSTHVPWTFGGGTKLEIKSSADDAKKDAAKKDDAKKDDAKKDGGVKLDE |
| | | TGGGLVQPGGAMKLSCVTSGFTFGHYWMNWVRQSPEKGLEWVAQFRNKPYNY |
| | | ETYYSDSVKGRFTISRDDSKSSVYLQMNNLRVEDTGIYYCTGASYGMEYLGQGTS |
| | | VTVSTTTPAPRPPTPAPTIASQPLSLRPEACRPAAGGAVHTRGLDFACDGWNPHLL |
| | | LLLLLVIVFIPAFWSLKTHPLWRLWKKIWAVPSPERFFMPLYKGCSGDFKKWVGA |
| | | PFTGSSLELGPWSPEVPSTLEVYSCHPPRSPAKRLQLTELQEPAELVESDGVPKPSF |
| | | WPTAQNSGGSAYSEERDRPYGLVSIDTVTVLDAEGPCTWPCSCEDDGYPALDLD |
| | | AGLEPSPGLEDPLLDAGTTVLSCGCVSAGSPGLGGPLGSLLDRLKPPLADGEDWA |
| | | GGLPWGGRSPGGVSESEAGSPLAGLDMDTFDSGFVGSDCSSPVECDFTSPGDEGP |
| | | PRSYLRQWVVIPPPLSSPGPQAS |
| IL2Rβ/γ | SEQ ID | MAAPALSWRLPLLILLLPLATSWASADVVMTQTPLSLPVSLGDQASISCRSSQSLV |
| SMAR | NO: 7 | HSNGNTYLRWYLQKPGQSPKVLIYKVSNRVSGVPDRFSGSGSGTDFTLKINRVEA |
| | | EDLGVYFCSQSTHVPWTFGGGTKLEIKSSADDAKKDAAKKDDAKKDDAKKDGG |
| | | VKLDETGGGLVQPGGAMKLSCVTSGFTFGHYWMNWVRQSPEKGLEWVAQFRN |
| | | KPYNYETYYSDSVKGRFTISRDDSKSSVYLQMNNLRVEDTGIYYCTGASYGMEYL |
| | | GQGTSVTVSTTTPAPRPPTPAPTIASQPLSLRPEACRPAAGGAVHTRGLDFACDIPW |
| | | LGHLLVGLSGAFGFIILVYLLINCRNTGPWLKKVLKCNTPDPSKFFSQLSSEHGGD |
| | | VQKWLSSPFPSSSFSPGGLAPEISPLEVLERDKVTQLLLQQDKVPEPASLSSNHSLT |
| | | SCFTNQGYFFFHLPDALEIEACQVYFTYDPYSEEDPDEGVAGAPTGSSPQPLQPLS |
| | | GEDDAYCTFPSRDDLLLFSPSLLGGPSPPSTAPGGSGAGEERMPPSLQERVPRDWD |
| | | PQPLGPPTPGVPDLVDFQPPPELVLREAGEEVPDAGPREGVSFPWSRPPGQGEFRA |
| | | LNARLPLNTDAYLSLQELQGQDPTHLVTGSGATNFSLLKQAGDVEENPGPAMLKP |
| | | SLPFTSLLFLQLPLLGVGDVVMTQTPLSLPVSLGDQASISCRSSQSLVHSNGNTYLR |
| | | WYLQKPGQSPKVLIYKVSNRVSGVPDRFSGSGSGTDFTLKINRVEAEDLGVYFCS |
| | | QSTHVPWTFGGGTKLEIKSSADDAKKDAAKKDDAKKDDAKKDGGVKLDETGGG |
| | | LVQPGGAMKLSCVTSGFTFGHYWMNWVRQSPEKGLEWVAQFRNKPYNYETYYS |
| | | DSVKGRFTISRDDSKSSVYLQMNNLRVEDTGIYYCTGASYGMEYLGQGTSVTVST |
| | | TTPAPRPPTPAPTIASQPLSLRPEACRPAAGGAVHTRGLDFACDVVISVGSMGLIISL |
| | | LCVYFWLERTMPRIPTLKNLEDLVTEYHGNFSAWSGVSKGLAESLQPDYSERLCL |
| | | VSEIPPKGGALGEGPGASPCNQHSPYWAPPCYTLKPETGSGEGRGSLLTCGDVEEN |
| | | PGPWEMPPPRLLFFLLFLTPMEVRPEEPLVVKVEEGDNAVLQCLKGTSDGPTQQL |
| | | TWSRESPLKPFLKLSLGLPGLGIHMRPLAIWLFIFNVSQQMGGFYLCQPGPPSEKA |
| | | WQPGWTVNVEGSGELFRWNVSDLGGLGCGLKNRSSEGPSSPSGKLMSPKLYVW |
| | | AKDRPEIWEGEPPCLPPRDSLNQSLSQDLTMAPGSTLWLSCGVPPDSVSRGPLSWT |
| | | HVHPKGPKSLLSLELKDDRPARDMWVMETGLLLPRATAQDAGKYYCHRGNLTM |
| | | SFHLEITARPVLWHWLLRTGGWKVSAVTLAYLIFCLCSLVGILHLQRALVLRRKR |
| | | KRMTDPTRRF |
| IL2Rα | SEQ ID | ATGGACAGCTACCTGCTGATGTGGGGCCTGCTGACCTTCATCATGGTGCCTGG |
| SMAR | NO: 8 | CTGTCAGGCCGACGTGGTCATGACACAGACACCTCTGAGCCTGCCTGTGTCTC |
| | | TGGGAGATCAGGCCAGCATCAGCTGCAGATCTAGCCAGAGCCTGGTGCACAG |
| | | CAACGGCAACACCTACCTGCGGTGGTATCTGCAGAAGCCCGGCCAGTCTCCTA |
| | | AGGTGCTGATCTACAAGGTGTCCAACAGAGTGTCCGGCGTGCCCGATAGATTT |
| | | TCTGGCAGCGGCTCTGGCACCGACTTCACCCTGAAGATCAATAGAGTGGAAGC |
| | | CGAGGACCTGGGCGTGTACTTCTGTAGCCAGTCTACCCACGTGCCATGGACCT |
| | | TTGGCGGCGGAACAAAGCTGGAAATCAAGAGCAGCGCCGACGACGCCAAGAA |
| | | GGACGCCGCTAAGAAGGATGACGCCAAAAAAGACGATGCCAAAAAGGATGG |
| | | CGGCGTGAAGCTGGACGAAACAGGCGGAGGACTTGTTCAGCCTGGCGGAGCC |
| | | ATGAAGCTGAGCTGTGTGACCAGCGGCTTCACCTTCGGCCACTACTGGATGAA |
| | | CTGGGTCCGACAGAGCCCTGAGAAAGGCCTGGAATGGGTCGCCCAGTTCAGA |
| | | AACAAGCCCTACAACTACGAAACCTACTACAGCGACAGCGTGAAGGGCAGAT |
| | | TCACCATCAGCCGGGACGACAGCAAGTCCAGCGTGTACCTGCAGATGAACAA |
| | | CCTGCGCGTGGAAGATACCGGCATCTACTACTGTACCGGCGCCAGCTACGGCA |
| | | TGGAATATCTCGGCCAGGGCACCAGCGTGACCGTGTCTACAACAACCCCTGCT |
| | | CCTCGGCCTCCTACACCAGCTCCTACAATTGCCAGCCAGCCACTGTCTCTGAG |
| | | GCCCGAAGCTTGTAGACCTGCTGCAGGCGGAGCCGTGCATACAAGAGGACTG |
| | | GACTTCGCCTGTGATGTGGCCGTGGCCGGATGTGTGTTTCTGCTGATCTCTGTG |
| | | CTGCTGCTGAGCGGCCTGACTTGGCAAAGACGGCAGAGAAAGAGCCGGCGGA |
| | | CCATCTGATAA |
| IL2Rβ | SEQ ID | ATGGCTGCTCCAGCTCTGTCTTGGAGACTGCCCCTGCTGATTCTGCTGCTGCCT |
| SMAR | NO: 9 | CTGGCTACATCTTGGGCCTCTGCCGACGTGGTCATGACACAGACACCACTGAG |
| | | CCTGCCTGTGTCTCTGGGAGATCAGGCCAGCATCAGCTGCAGATCCAGCCAGT |
| | | CTCTGGTGCACAGCAACGGCAACACCTACCTGCGGTGGTATCTGCAGAAGCCC |
| | | GGCCAGTCTCCTAAGGTGCTGATCTACAAGGTGTCCAACAGAGTGTCCGGCGT |
| | | GCCCGATAGATTTTCTGGCAGCGGCTCTGGCACCGACTTCACCCTGAAGATCA |
| | | ATAGAGTGGAAGCCGAGGACCTGGGCGTGTACTTCTGTAGCCAGTCTACCCAC |
| | | GTGCCATGGACCTTTGGCGGCGGAACAAAGCTGGAAATCAAGAGCAGCGCCG |
| | | ACGACGCCAAGAAGGACGCCGCTAAGAAGGATGACGCCAAAAAAGACGATG |
| | | CCAAAAAGGATGGCGGCGTGAAGCTGGACGAAACAGGCGGAGGACTTGTTCA |
| | | GCCTGGCGGAGCCATGAAGCTGAGCTGTGTGACCAGCGGCTTCACCTTCGGCC |
| | | ACTACTGGATGAACTGGGTCCGACAGAGCCCTGAGAAAGGCCTGGAATGGGT |
| | | CGCCCAGTTCAGAAACAAGCCCTACAACTACGAAACCTACTACAGCGACAGC |
| | | GTGAAGGGCAGATTCACCATCAGCCGGGACGACAGCAAGTCCAGCGTGTACC |
| | | TGCAGATGAACAACCTGCGCGTGGAAGATACCGGCATCTACTACTGTACCGGC |
| | | GCCAGCTACGGCATGGAATATCTCGGCCAGGGCACCAGCGTGACCGTGTCTAC |
| | | AACAACCCCTGCTCCTCGGCCTCCTACACCAGCTCCTACAATTGCCAGCCAGC |
| | | CACTGTCTCTGAGGCCCGAAGCTTGTAGACCTGCTGCAGGCGGAGCCGTGCAT |
| | | ACAAGAGGACTGGATTTCGCCTGCGACATCCCCTGGCTGGGACATCTGCTTGT |
| | | TGGACTGTCTGGCGCCTTCGGCTTCATCATCCTGGTGTATCTGCTGATCAACTG |
| | | CCGGAACACAGGCCCTTGGCTGAAGAAAGTGCTGAAGTGCAACACCCCTGAT |
| | | CCGAGCAAGTTCTTTAGCCAGCTGAGCAGCGAGCATGGCGGCGACGTTCAGA |
| | | AATGGCTGTCTAGCCCATTTCCTAGCAGCAGCTTCAGCCCAGGTGGACTGGCC |
| | | CCTGAGATTAGCCCTCTGGAAGTGCTGGAACGGGACAAAGTGACCCAGCTGCT |
| | | CCTCCAGCAGGATAAGGTGCCAGAACCTGCCAGCCTGTCCAGCAATCACAGCC |
| | | TGACCAGCTGCTTTACCAACCAGGGCTACTTCTTCTTCCATCTGCCTGACGCTC |
| | | TGGAAATCGAGGCCTGCCAGGTGTACTTCACCTACGATCCCTACAGCGAAGAG |
| | | GACCCCGATGAAGGTGTTGCTGGCGCCCCTACAGGATCTTCTCCACAGCCTCT |
| | | GCAACCTCTGAGCGGCGAGGATGATGCCTACTGCACCTTTCCAAGCAGGGACG |
| | | ACCTGCTCCTGTTCAGCCCATCTCTGCTCGGAGGACCATCTCCTCCATCTACAG |
| | | CTCCAGGCGGATCTGGCGCTGGCGAGGAAAGAATGCCACCTAGCCTGCAAGA |
| | | GCGGGTGCCCAGAGATTGGGATCCTCAACCTCTCGGCCCTCCAACACCTGGCG |
| | | TGCCAGATCTCGTGGACTTTCAGCCTCCTCCAGAGCTGGTGCTGAGAGAAGCT |
| | | GGCGAAGAAGTGCCAGACGCTGGCCCTAGAGAGGGCGTTAGCTTTCCTTGGA |
| | | GCAGACCTCCTGGACAGGGCGAGTTTAGGGCCCTGAATGCAAGACTGCCTCTG |
| | | AACACCGACGCCTACCTGTCTCTGCAAGAACTGCAGGGACAAGACCCCACAC |
| | | ACCTGGTGTAATGA |
| IL2Rγ | SEQ ID | ATGCTGAAGCCCAGCCTGCCTTTTACCAGCCTGCTGTTCCTGCAGCTGCCTCTG |
| SMAR | NO: 10 | CTTGGCGTGGGAGATGTGGTCATGACACAGACCCCACTGAGCCTGCCTGTGTC |
| | | TCTGGGAGATCAGGCCAGCATCAGCTGCAGATCTAGCCAGAGCCTGGTGCACA |
| | | GCAACGGCAACACCTACCTGCGGTGGTATCTGCAGAAGCCCGGCCAGTCTCCT |
| | | AAGGTGCTGATCTACAAGGTGTCCAACAGAGTGTCCGGCGTGCCCGATAGATT |
| | | TTCTGGCAGCGGCTCTGGCACCGACTTCACCCTGAAGATCAATAGAGTGGAAG |
| | | CCGAGGACCTGGGCGTGTACTTCTGTAGCCAGTCTACCCACGTGCCATGGACC |
| | | TTTGGCGGCGGAACAAAGCTGGAAATCAAGAGCAGCGCCGACGACGCCAAGA |
| | | AGGACGCCGCTAAGAAGGATGACGCCAAAAAAGACGATGCCAAAAAGGATG |
| | | GCGGCGTGAAGCTGGACGAAACAGGCGGAGGACTTGTTCAGCCTGGCGGAGC |
| | | CATGAAGCTGAGCTGTGTGACCAGCGGCTTCACCTTCGGCCACTACTGGATGA |
| | | ACTGGGTCCGACAGAGCCCTGAGAAAGGCCTGGAATGGGTCGCCCAGTTCAG |
| | | AAACAAGCCCTACAACTACGAAACCTACTACAGCGACAGCGTGAAGGGCAGA |
| | | TTCACCATCAGCCGGGACGACAGCAAGTCCAGCGTGTACCTGCAGATGAACA |
| | | ACCTGCGCGTGGAAGATACCGGCATCTACTACTGTACCGGCGCCAGCTACGGC |
| | | ATGGAATATCTCGGCCAGGGCACCAGCGTGACCGTGTCTACAACAACCCCTGC |
| | | TCCTCGGCCTCCTACACCAGCTCCTACAATTGCCAGCCAGCCACTGTCTCTGAG |
| | | GCCCGAAGCTTGTAGACCTGCTGCAGGCGGAGCCGTGCATACAAGAGGACTG |
| | | GACTTCGCCTGCGACGTGGTCATCTCTGTGGGCTCTATGGGCCTGATCATCTCC |
| | | CTGCTGTGTGTGTACTTTTGGCTGGAACGGACCATGCCTCGGATCCCCACACTG |
| | | AAGAACCTGGAAGATCTGGTCACCGAGTACCACGGCAACTTCAGTGCTTGGAG |
| | | CGGCGTGTCAAAAGGACTGGCCGAAAGCCTGCAGCCTGACTACTCCGAGAGA |
| | | CTGTGCCTGGTGTCTGAGATCCCTCCTAAAGGCGGCGCTCTCGGAGAAGGACC |
| | | TGGTGCCTCTCCATGCAATCAGCACAGCCCTTATTGGGCCCCTCCTTGCTACAC |
| | | CCTGAAACCTGAGACATGATGA |
| IL7Rα | SEQ ID | ATGACAATCCTGGGCACCACCTTCGGCATGGTGTTCAGTCTGCTGCAGGTCGT |
| SMAR | NO: 11 | GTCTGGCGACGTGGTCATGACACAGACCCCACTGTCTCTGCCTGTGTCTCTGG |
| | | GAGATCAGGCCAGCATCAGCTGCAGATCTAGCCAGAGCCTGGTGCACAGCAA |
| | | CGGCAACACCTACCTGCGGTGGTATCTGCAGAAGCCCGGCCAGTCTCCTAAGG |
| | | TGCTGATCTACAAGGTGTCCAACAGAGTGTCCGGCGTGCCCGATAGATTTTCT |
| | | GGCAGCGGCTCTGGCACCGACTTCACCCTGAAGATCAATAGAGTGGAAGCCG |
| | | AGGACCTGGGCGTGTACTTCTGTAGCCAGTCTACCCACGTGCCATGGACCTTT |
| | | GGCGGCGGAACAAAGCTGGAAATCAAGAGCAGCGCCGACGACGCCAAGAAG |
| | | GACGCCGCTAAGAAGGATGACGCCAAAAAAGACGATGCCAAAAAGGATGGC |
| | | GGCGTGAAGCTGGACGAAACAGGCGGAGGACTTGTTCAGCCTGGCGGAGCCA |
| | | TGAAGCTGAGCTGTGTGACCAGCGGCTTCACCTTTGGCCACTACTGGATGAAC |
| | | TGGGTCCGACAGAGCCCTGAGAAAGGCCTGGAATGGGTCGCCCAGTTCAGAA |
| | | ACAAGCCCTACAACTACGAAACCTACTACAGCGACAGCGTGAAGGGCAGATT |
| | | CACCATCAGCCGGGACGACAGCAAGTCCAGCGTGTACCTGCAGATGAACAAC |
| | | CTGCGCGTGGAAGATACCGGCATCTACTACTGTACCGGCGCCAGCTACGGCAT |
| | | GGAATATCTCGGCCAGGGCACCAGCGTGACCGTGTCTACAACAACCCCTGCTC |
| | | CTCGGCCTCCTACACCAGCTCCTACAATTGCCAGCCAGCCTCTGTCTCTGAGGC |
| | | CCGAAGCTTGTAGACCTGCTGCCGGCGGAGCTGTGCATACAAGAGGCCTGGAT |
| | | TTCGCCTGCGATCCCATCCTGCTGACAATCAGCATCCTGAGCTTTTTCAGCGTG |
| | | GCCCTGCTGGTCATCCTGGCCTGTGTGCTGTGGAAGAAGCGGATCAAGCCCAT |
| | | CGTGTGGCCCAGCCTGCCTGACCACAAGAAAACCCTGGAACACCTGTGCAAG |
| | | AAGCCCCGGAAGAACCTGAACGTGTCCTTCAATCCCGAGAGCTTCCTGGACTG |
| | | CCAGATCCACAGAGTGGACGACATCCAGGCCAGGGACGAAGTGGAAGGCTTT |
| | | CTGCAGGACACATTCCCTCAGCAGCTGGAAGAGAGCGAGAAGCAGAGACTCG |
| | | GAGGCGACGTGCAGAGCCCTAATTGCCCTTCTGAGGACGTCGTGATCACCCCT |
| | | GAGAGCTTCGGCAGAGATAGCAGCCTGACATGTCTGGCCGGCAATGTGTCCGC |
| | | CTGTGATGCCCCTATCCTGAGCAGCAGCAGAAGCCTGGATTGCAGAGAGAGC |
| | | GGCAAGAACGGCCCTCACGTGTACCAGGATCTGCTCCTGAGCCTGGGAACCAC |
| | | CAATAGCACACTGCCTCCACCATTCAGCCTGCAGAGCGGCATCCTGACACTGA |
| | | ACCCTGTTGCTCAGGGCCAGCCAATCCTGACCAGCCTGGGCAGCAATCAAGAA |
| | | GAGGCCTACGTCACCATGAGCAGCTTCTACCAGAACCAGTGATGATGGGAATG |
| | | GAACCTACCAGACCTGGGTGGCCAGCTCACTGAGCCATGCGCTATGTCCGGGA |
| | | ATGGAACCTACCAGACCTGGGTGGCCACCAGGATTTGCCAAGGAGAGGAGCA |
| | | GAGGTTCACCTGCTACATGGAACACAGCGGGAATCACAGCACTCACCCTGTGC |
| | | CCTCTGGGAAAGTGCTGGTGCTTCAGAGTCATTGGCAGACATTCCATGTTTCTT |
| | | AGGCTCGAGGTCGACGGTATCGATAAGCTTGATATCCGCCCCCCCCCCTAACG |
| | | TTACTGGCCGAAGCCGCTTGGAATAAGGCCGGTGTGCGTTTGTCTATATGTTAT |
| | | TTTCCACCATATTGCCGTCTTTTGGCAATGTGAGGGCCCGGAAACCTGGCCCTG |
| | | TCTTCTTGACGAGCATTCCTAGGGGTCTTTCCCCTCTCGCCAAAGGAATGCAAG |
| | | GTCTGTTGAATGTCGTGAAGGAAGCAGTTCCTCTGGAAGCTTCTTGAAGACAA |
| | | ACAACGTCTGTAGCGACCCTTTGCAGGCAGCGGAACCCCCCACCTGGCGACAG |
| | | GTGCCTCTGCGGCCAAAAGCCACGTGTATAAGATACACCTGCAAAGGCGGCA |
| | | CAACCCCAGTGCCACGTTGTGAGTTGGATAGTTGTGGAAAGAGTCAAATGGCT |
| | | CTCCTCAAGCGTATTCAACAAGGGGCTGAAGGATGCCCAGAAGGTACCCCATT |
| | | GTATGGGATCTGATCTGGGGCCTCGGTGCACATGCTTTACATGTGTTTAGTCGA |
| | | GGTTAAAAAAACGTCTAGGCCCCCCGAACCACGGGGACGTGGTTTTCCTTTGA |
| | | AAAACACGATGATAATATGGCCACAACGCGTACCATGGTGAGCAAGGGCGAG |
| | | GAGGATAACATGGCCATCATCAAGGAGTTCATGCGCTTCAAGGTGCACATGGA |
| | | GGGCTCCGTGAACGGCCACGAGTTCGAGATCGAGGGCGAGGGCGAGGGCCGC |
| | | CCCTACGAGGGCACCCAGACCGCCAAGCTGAAGGTGACCAAGGGTGGCCCCC |
| | | TGCCCTTCGCCTGGGACATCCTGTCCCCTCAGTTCATGTACGGCTCCAAGGCCT |
| | | ACGTGAAGCACCCCGCCGACATCCCCGACTACTTGAAGCTGTCCTTCCCCGAG |
| | | GGCTTCAAGTGGGAGCGCGTGATGAACTTCGAGGACGGCGGCGTGGTGACCG |
| | | TGACCCAGGACTCCTCCCTGCAGGACGGCGAGTTCATCTACAAGGTGAAGCTG |
| | | CGCGGCACCAACTTCCCCTCCGACGGCCCCGTAATGCAGAAGAAGACCATGG |
| | | GCTGGGAGGCCTCCTCCGAGCGGATGTACCCCGAGGACGGCGCCCTGAAGGG |
| | | CGAGATCAAGCAGAGGCTGAAGCTGAAGGACGGCGGCCACTACGACGCTGAG |
| | | GTCAAGACCACCTACAAGGCCAAGAAGCCCGTGCAGCTGCCCGGCGCCTACA |
| | | ACGTCAACATCAAGTTGGACATCACCTCCCACAACGAGGACTACACCATCGTG |
| | | GAACAGTACGAACGCGCCGAGGGCCGCCACTCCACCGGCGGCATGGACGAGC |
| | | TGTACAAGTGA |
| IL15Rα | SEQ ID | ATGGCTCCTCGGAGAGCCAGAGGCTGTAGAACACTTGGACTGCCCGCTCTGCT |
| SMAR | NO: 12 | GCTGCTCCTGCTTCTTAGACCTCCTGCCACAAGAGGCGACGTGGTCATGACAC |
| | | AGACCCCTCTGTCTCTGCCTGTGTCTCTGGGAGATCAGGCCAGCATCAGCTGC |
| | | AGATCTAGCCAGAGCCTGGTGCACAGCAACGGCAACACCTACCTGCGGTGGT |
| | | ATCTGCAGAAGCCCGGCCAGTCTCCTAAGGTGCTGATCTACAAGGTGTCCAAC |
| | | AGAGTGTCCGGCGTGCCCGATAGATTTTCTGGCAGCGGCTCTGGCACCGACTT |
| | | CACCCTGAAGATCAATAGAGTGGAAGCCGAGGACCTGGGCGTGTACTTCTGTA |
| | | GCCAGTCTACCCACGTGCCATGGACCTTTGGCGGCGGAACAAAGCTGGAAATC |
| | | AAGAGCAGCGCCGACGACGCCAAGAAGGACGCCGCTAAGAAGGATGACGCC |
| | | AAAAAAGACGATGCCAAAAAGGATGGCGGCGTGAAGCTGGACGAAACAGGC |
| | | GGAGGACTTGTTCAGCCTGGCGGAGCCATGAAGCTGAGCTGTGTGACCAGCG |
| | | GCTTCACCTTCGGCCACTACTGGATGAACTGGGTCCGACAGAGCCCTGAGAAA |
| | | GGCCTGGAATGGGTCGCCCAGTTCAGAAACAAGCCCTACAACTACGAAACCT |
| | | ACTACAGCGACAGCGTGAAGGGCAGATTCACCATCAGCCGGGACGACAGCAA |
| | | GTCCAGCGTGTACCTGCAGATGAACAACCTGCGCGTGGAAGATACCGGCATCT |
| | | ACTACTGTACCGGCGCCAGCTACGGCATGGAATATCTCGGCCAGGGCACCAGC |
| | | GTGACCGTGTCTACAACAACCCCTGCTCCTCGGCCTCCTACACCAGCTCCTACA |
| | | ATTGCCAGCCAGCCACTGTCTCTGAGGCCCGAAGCTTGTAGACCTGCTGCAGG |
| | | CGGAGCCGTGCATACAAGAGGACTGGACTTCGCCTGTGACGTGGCCATCAGCA |
| | | CAAGCACCGTTCTGCTGTGTGGCCTGTCAGCCGTTAGCCTGCTGGCTTGCTACC |
| | | TGAAGTCCAGACAGACACCTCCTCTGGCCAGCGTGGAAATGGAAGCCATGGA |
| | | AGCTCTGCCAGTGACCTGGGGCACCTCCAGCAGAGATGAGGATCTGGAAAAC |
| | | TGCAGCCACCACCTGTGATGA |
| IL21Rα | SEQ ID | ATGCCAAGAGGATGGGCCGCTCCTCTTCTCCTGTTGCTGCTTCAAGGCGGCTG |
| SMAR | NO: 13 | GGGCGACGTTGTGATGACACAGACACCACTGAGCCTGCCTGTGTCTCTGGGAG |
| | | ATCAGGCCAGCATCAGCTGCAGATCTAGCCAGAGCCTGGTGCACAGCAACGG |
| | | CAACACCTACCTGCGGTGGTATCTGCAGAAGCCCGGCCAGTCTCCTAAGGTGC |
| | | TGATCTACAAGGTGTCCAACAGAGTGTCCGGCGTGCCCGATAGATTTTCTGGC |
| | | AGCGGCTCTGGCACCGACTTCACCCTGAAGATCAATAGAGTGGAAGCCGAGG |
| | | ACCTGGGCGTGTACTTCTGTAGCCAGTCTACCCACGTGCCATGGACCTTTGGC |
| | | GGCGGAACAAAGCTGGAAATCAAGAGCAGCGCCGACGACGCCAAGAAGGAC |
| | | GCCGCTAAGAAGGATGACGCCAAAAAAGACGATGCCAAAAAGGATGGCGGC |
| | | GTGAAGCTGGACGAAACAGGCGGAGGACTTGTTCAGCCTGGCGGAGCCATGA |
| | | AGCTGAGCTGTGTGACCAGCGGCTTCACCTTCGGCCACTACTGGATGAACTGG |
| | | GTCCGACAGAGCCCTGAGAAAGGCCTGGAATGGGTCGCCCAGTTCAGAAACA |
| | | AGCCCTACAACTACGAAACCTACTACAGCGACAGCGTGAAGGGCAGATTCAC |
| | | CATCAGCCGGGACGACAGCAAGTCCAGCGTGTACCTGCAGATGAACAACCTG |
| | | CGCGTGGAAGATACCGGCATCTACTACTGTACCGGCGCCAGCTACGGCATGGA |
| | | ATATCTCGGCCAGGGCACCAGCGTGACCGTGTCTACAACAACCCCTGCTCCTC |
| | | GGCCTCCTACACCAGCTCCTACAATTGCCAGCCAGCCACTGTCTCTGAGGCCC |
| | | GAAGCTTGTAGACCTGCTGCAGGCGGAGCCGTGCATACAAGAGGACTGGACT |
| | | TTGCCTGCGACGGCTGGAATCCTCATCTGTTGTTGTTGCTCCTCCTGGTCATCG |
| | | TGTTCATCCCCGCCTTTTGGAGCCTGAAAACACACCCTCTGTGGCGGCTGTGG |
| | | AAGAAAATCTGGGCCGTGCCATCTCCTGAGCGGTTCTTCATGCCTCTGTACAA |
| | | GGGCTGCAGCGGCGACTTCAAGAAATGGGTCGGAGCCCCTTTTACCGGCAGCT |
| | | CTCTGGAACTTGGACCTTGGAGCCCTGAAGTGCCCAGCACACTGGAAGTGTAC |
| | | AGCTGTCACCCTCCTAGAAGCCCCGCCAAGAGACTGCAGCTCACAGAGCTGCA |
| | | AGAGCCTGCCGAGCTGGTGGAATCTGATGGCGTGCCCAAGCCTAGCTTCTGGC |
| | | CCACCGCTCAAAATTCTGGCGGCAGCGCCTACAGCGAGGAAAGAGATAGACC |
| | | TTACGGCCTGGTGTCCATCGACACCGTGACAGTGCTGGATGCCGAGGGACCTT |
| | | GTACCTGGCCTTGTAGCTGCGAGGACGATGGCTACCCTGCTCTGGATCTGGAC |
| | | GCAGGACTGGAACCTTCTCCAGGCCTCGAAGATCCTCTGCTGGACGCCGGAAC |
| | | AACAGTGCTGTCTTGTGGCTGTGTGTCCGCCGGATCTCCTGGACTTGGAGGAC |
| | | CTCTGGGAAGCCTGCTGGACAGACTGAAACCTCCTCTGGCCGATGGCGAAGAT |
| | | TGGGCTGGTGGACTTCCTTGGGGCGGAAGATCTCCAGGCGGAGTGTCTGAATC |
| | | TGAGGCCGGTTCTCCACTGGCCGGCCTGGATATGGATACCTTCGATAGCGGCT |
| | | TCGTGGGCAGCGATTGCAGCAGCCCTGTGGAATGCGACTTCACATCTCCTGGC |
| | | GACGAGGGCCCACCTAGAAGCTATCTCAGACAGTGGGTCGTGATCCCTCCACC |
| | | TCTGTCTAGTCCTGGACCACAGGCCAGCTGATGA |
| IL2Rβ/γ | SEQ ID | ATGGCTGCTCCAGCTCTGTCTTGGAGACTGCCCCTGCTGATTCTGCTGCTGCCT |
| SMAR | NO: 14 | CTGGCTACATCTTGGGCCTCTGCCGACGTGGTCATGACACAGACACCACTGAG |
| | | CCTGCCTGTGTCTCTGGGAGATCAGGCCAGCATCAGCTGCAGATCCAGCCAGT |
| | | CTCTGGTGCACAGCAACGGCAACACCTACCTGCGGTGGTATCTGCAGAAGCCC |
| | | GGCCAGTCTCCTAAGGTGCTGATCTACAAGGTGTCCAACAGAGTGTCCGGCGT |
| | | GCCCGATAGATTTTCTGGCAGCGGCTCTGGCACCGACTTCACCCTGAAGATCA |
| | | ATAGAGTGGAAGCCGAGGACCTGGGCGTGTACTTCTGTAGCCAGTCTACCCAC |
| | | GTGCCATGGACCTTTGGCGGCGGAACAAAGCTGGAAATCAAGAGCAGCGCCG |
| | | ACGACGCCAAGAAGGACGCCGCTAAGAAGGATGACGCCAAAAAAGACGATG |
| | | CCAAAAAGGATGGCGGCGTGAAGCTGGACGAAACAGGCGGAGGACTTGTTCA |
| | | GCCTGGCGGAGCCATGAAGCTGAGCTGTGTGACCAGCGGCTTCACCTTCGGCC |
| | | ACTACTGGATGAACTGGGTCCGACAGAGCCCTGAGAAAGGCCTGGAATGGGT |
| | | CGCCCAGTTCAGAAACAAGCCCTACAACTACGAAACCTACTACAGCGACAGC |
| | | GTGAAGGGCAGATTCACCATCAGCCGGGACGACAGCAAGTCCAGCGTGTACC |
| | | TGCAGATGAACAACCTGCGCGTGGAAGATACCGGCATCTACTACTGTACCGGC |
| | | GCCAGCTACGGCATGGAATATCTCGGCCAGGGCACCAGCGTGACCGTGTCTAC |
| | | AACAACCCCTGCTCCTCGGCCTCCTACACCAGCTCCTACAATTGCCAGCCAGC |
| | | CACTGTCTCTGAGGCCCGAAGCTTGTAGACCTGCTGCAGGCGGAGCCGTGCAT |
| | | ACAAGAGGACTGGATTTCGCCTGCGACATCCCCTGGCTGGGACATCTGCTTGT |
| | | TGGACTGTCTGGCGCCTTCGGCTTCATCATCCTGGTGTATCTGCTGATCAACTG |
| | | CCGGAACACAGGCCCTTGGCTGAAGAAAGTGCTGAAGTGCAACACCCCTGAT |
| | | CCGAGCAAGTTCTTTAGCCAGCTGAGCAGCGAGCATGGCGGCGACGTTCAGA |
| | | AATGGCTGTCTAGCCCATTTCCTAGCAGCAGCTTCAGCCCAGGTGGACTGGCC |
| | | CCTGAGATTAGCCCTCTGGAAGTGCTGGAACGGGACAAAGTGACCCAGCTGCT |
| | | CCTCCAGCAGGATAAGGTGCCAGAACCTGCCAGCCTGTCCAGCAATCACAGCC |
| | | TGACCAGCTGCTTTACCAACCAGGGCTACTTCTTCTTCCATCTGCCTGACGCTC |
| | | TGGAAATCGAGGCCTGCCAGGTGTACTTCACCTACGATCCCTACAGCGAAGAG |
| | | GACCCCGATGAAGGTGTTGCTGGCGCCCCTACAGGATCTTCTCCACAGCCTCT |
| | | GCAACCTCTGAGCGGCGAGGATGATGCCTACTGCACCTTTCCAAGCAGGGACG |
| | | ACCTGCTCCTGTTCAGCCCATCTCTGCTCGGAGGACCATCTCCTCCATCTACAG |
| | | CTCCAGGCGGATCTGGCGCTGGCGAGGAAAGAATGCCACCTAGCCTGCAAGA |
| | | GCGGGTGCCCAGAGATTGGGATCCTCAACCTCTCGGCCCTCCAACACCTGGCG |
| | | TGCCAGATCTCGTGGACTTTCAGCCTCCTCCAGAGCTGGTGCTGAGAGAAGCT |
| | | GGCGAAGAAGTGCCAGACGCTGGCCCTAGAGAGGGCGTTAGCTTTCCTTGGA |
| | | GCAGACCTCCTGGACAGGGCGAGTTTAGGGCCCTGAATGCAAGACTGCCTCTG |
| | | AACACCGACGCCTACCTGTCTCTGCAAGAACTGCAGGGACAAGACCCCACAC |
| | | ACCTGGTGACTGGATCTGGAGCAACAAACTTCTCACTACTCAAACAAGCAGGT |
| | | GACGTGGAGGAGAATCCCGGGCCTGCCATGCTGAAGCCCAGCCTGCCTTTTAC |
| | | CAGCCTGCTGTTCCTGCAGCTGCCTCTGCTTGGCGTGGGAGATGTGGTCATGA |
| | | CACAGACCCCACTGAGCCTGCCTGTGTCTCTGGGAGATCAGGCCAGCATCAGC |
| | | TGCAGATCTAGCCAGAGCCTGGTGCACAGCAACGGCAACACCTACCTGCGGTG |
| | | GTATCTGCAGAAGCCCGGCCAGTCTCCTAAGGTGCTGATCTACAAGGTGTCCA |
| | | ACAGAGTGTCCGGCGTGCCCGATAGATTTTCTGGCAGCGGCTCTGGCACCGAC |
| | | TTCACCCTGAAGATCAATAGAGTGGAAGCCGAGGACCTGGGCGTGTACTTCTG |
| | | TAGCCAGTCTACCCACGTGCCATGGACCTTTGGCGGCGGAACAAAGCTGGAAA |
| | | TCAAGAGCAGCGCCGACGACGCCAAGAAGGACGCCGCTAAGAAGGATGACGC |
| | | CAAAAAAGACGATGCCAAAAAGGATGGCGGCGTGAAGCTGGACGAAACAGG |
| | | CGGAGGACTTGTTCAGCCTGGCGGAGCCATGAAGCTGAGCTGTGTGACCAGCG |
| | | GCTTCACCTTCGGCCACTACTGGATGAACTGGGTCCGACAGAGCCCTGAGAAA |
| | | GGCCTGGAATGGGTCGCCCAGTTCAGAAACAAGCCCTACAACTACGAAACCT |
| | | ACTACAGCGACAGCGTGAAGGGCAGATTCACCATCAGCCGGGACGACAGCAA |
| | | GTCCAGCGTGTACCTGCAGATGAACAACCTGCGCGTGGAAGATACCGGCATCT |
| | | ACTACTGTACCGGCGCCAGCTACGGCATGGAATATCTCGGCCAGGGCACCAGC |
| | | GTGACCGTGTCTACAACAACCCCTGCTCCTCGGCCTCCTACACCAGCTCCTACA |
| | | ATTGCCAGCCAGCCACTGTCTCTGAGGCCCGAAGCTTGTAGACCTGCTGCAGG |
| | | CGGAGCCGTGCATACAAGAGGACTGGACTTCGCCTGCGACGTGGTCATCTCTG |
| | | TGGGCTCTATGGGCCTGATCATCTCCCTGCTGTGTGTGTACTTTTGGCTGGAAC |
| | | GGACCATGCCTCGGATCCCCACACTGAAGAACCTGGAAGATCTGGTCACCGAG |
| | | TACCACGGCAACTTCAGTGCTTGGAGCGGCGTGTCAAAAGGACTGGCCGAAA |
| | | GCCTGCAGCCTGACTACTCCGAGAGACTGTGCCTGGTGTCTGAGATCCCTCCT |
| | | AAAGGCGGCGCTCTCGGAGAAGGACCTGGTGCCTCTCCATGCAATCAGCACA |
| | | GCCCTTATTGGGCCCCTCCTTGCTACACCCTGAAACCTGAGACAGGCTCCGGC |
| | | GAGGGCAGGGGAAGTCTTCTAACATGCGGGGACGTGGAGGAAAATCCCGGCC |
| | | CATGGGAAATGCCTCCTCCTCGGCTGCTGTTCTTCCTGCTGTTTCTGACCCCTA |
| | | TGGAAGTGCGGCCCGAGGAACCTCTGGTGGTCAAAGTTGAAGAGGGCGACAA |
| | | CGCCGTGCTGCAGTGTCTGAAGGGCACATCTGATGGCCCCACACAGCAGCTGA |
| | | CCTGGTCTAGAGAGAGCCCTCTGAAGCCCTTCCTGAAGCTGTCTCTGGGACTG |
| | | CCTGGACTGGGCATCCATATGAGGCCTCTGGCCATCTGGCTGTTCATCTTCAAC |
| | | GTGTCCCAGCAGATGGGCGGCTTCTACCTGTGTCAACCTGGACCTCCAAGCGA |
| | | GAAGGCTTGGCAGCCTGGCTGGACCGTGAATGTGGAAGGATCCGGCGAGCTG |
| | | TTCCGGTGGAATGTGTCTGATCTCGGCGGCCTCGGATGCGGCCTGAAGAATAG |
| | | ATCTAGCGAGGGCCCTAGCAGCCCCAGCGGAAAACTGATGAGCCCCAAGCTG |
| | | TACGTGTGGGCCAAAGACAGACCCGAGATTTGGGAGGGCGAGCCTCCTTGTCT |
| | | GCCTCCTAGAGACAGCCTGAACCAGAGCCTGAGCCAGGACCTGACAATGGCC |
| | | CCTGGATCTACACTGTGGCTGAGCTGTGGCGTGCCACCTGACAGTGTGTCTAG |
| | | AGGCCCTCTGTCTTGGACCCACGTGCACCCTAAGGGCCCTAAGTCTCTGCTGA |
| | | GCCTGGAACTGAAGGACGACAGGCCCGCCAGAGATATGTGGGTCATGGAAAC |
| | | AGGCCTGCTGCTGCCTAGAGCCACAGCACAGGATGCCGGCAAGTACTACTGCC |
| | | ACAGAGGCAACCTGACCATGAGCTTCCACCTGGAAATCACCGCCAGACCTGTC |
| | | CTGTGGCACTGGCTGCTTAGAACCGGCGGCTGGAAAGTGTCTGCCGTGACTCT |
| | | GGCCTACCTGATCTTCTGCCTGTGTAGCCTCGTGGGCATCCTGCATCTGCAGAG |
| | | AGCACTGGTCCTGCGGCGGAAGCGGAAGAGAATGACCGATCCTACCAGACGG |
| | | TTCTGATGA |

An engineered cytokine receptor switch may comprise a signal peptide, an activator binding domain, a hinge, a transmembrane domain, and an intracellular signaling domain. In some embodiments, an engineered cytokine receptor switch may comprise a signal peptide of any one of SEQ ID NO: 15-SEQ ID NO: 20, an activator binding domain of SEQ ID NO: 21, a transmembrane domain of any one of SEQ ID NO: 23-SEQ ID NO: 28, and an intracellular signaling domain of any one of SEQ ID NO: 29-SEQ ID NO: 34. In some embodiments, an engineered cytokine receptor switch may further comprise a hinge (e.g., SEQ ID NO: 22), a cleavage sequence (e.g., any one of SEQ ID NO: 35-SEQ ID NO: 38), a marker (e.g., SEQ ID NO: 39), or combinations thereof.

In some embodiments, an engineered cytokine receptor switch may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 1-SEQ ID NO: 7. In some embodiments, an engineered cytokine receptor switch is encoded by a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 8-SEQ ID NO: 14. In some embodiments, the engineered cytokine receptor switch may comprise a sequence of any one of SEQ ID NO: 1-SEQ ID NO: 7. In some embodiments, the engineered cytokine receptor switch is encoded by a sequence of any one of SEQ ID NO: 8-SEQ ID NO: 14.

In some embodiments, an engineered cytokine receptor switch may be a single-chain cytokine receptor switch, such as any one of SEQ ID NO: 1-SEQ ID NO: 6 (based on IL2Rα, IL2Rß, IL2Rγ, IL7Rα, IL15Rα, and IL21Rα, respectively). A single-chain cytokine receptor switch can be derived from a single cytokine receptor chain (e.g., an α, β, or γ chain). The cytokine receptor chain may be a wild-type cytokine receptor chain, or may be a chimeric or mutant cytokine receptor chain. In some embodiments, the single cytokine receptor chain is capable of initiating signaling via dimerization with an endogenous cytokine receptor chain. For example, IL7 signaling occurs through the IL7R receptor, which is composed of the IL7Rα and IL2Rγ chains. The IL2Rγ chain (also known as the common gamma chain (γc)) is shared by other members of the in the common gamma chain receptor family. Accordingly, a single-chain cytokine receptor switch (e.g., derived from IL7Rα) may heterodimerize with an endogenous cytokine receptor (e.g., IL2Rγ) and bind to an activator to initiate intracellular signaling. Some cytokine receptor chains are capable of initiating signaling via homodimerization (e.g., IL7Rα can form homodimers and initiate IL7 signaling without IL2Rγ). Thus, in some embodiments, a pair of single-chain cytokine receptor switches (e.g., derived from IL7Rα) may bind to respective activators and homodimerize with each other to initiate intracellular signaling. Single-chain receptor switches may be used to initiate novel signaling pathways by dimerization with endogenous cytokine receptor chains, depending on how the dimerization occurs and which receptor chains are dimerized. Additionally, in some embodiments, production of viral vectors and engineered immune cells may be easier for single-chain cytokine receptor switches.

In some embodiments, an engineered cytokine receptor switch may be a dual-chain cytokine receptor switch. For example, a dual-chain cytokine receptor switch may include a first cytokine receptor chain of SEQ ID NO: 2 (based on IL2Rß) and a second cytokine receptor chain of SEQ ID NO: 3 (based on IL2Rγ). As another example, a dual-chain cytokine receptor switch may include a first cytokine receptor chain of SEQ ID NO: 4 (based on IL7Rα) and a second cytokine receptor chain of SEQ ID NO: 3 (based on IL2Rγ). In a further example, a dual-chain cytokine receptor switch may include a first cytokine receptor chain of SEQ ID NO: 6 (based on IL21Rα) and a second cytokine receptor chain of SEQ ID NO:3 (based on IL2Rγ). In some embodiments, each chain of a dual-chain cytokine receptor switch may bind to a respective activator and heterodimerize with each other to activate intracellular signaling. A dual-chain cytokine receptor switch can be derived from two cytokine receptor chains (e.g., a combination of α, β, or γ chains), each of which can be independently selected from any of the cytokine receptor chains described herein. For example, a dual-chain cytokine receptor switch including a first cytokine receptor chain derived from IL2Rß and a second cytokine receptor chain derived from IL2Rγ can mimic the IL2-IL2R signaling pathway. In some embodiments, each chain of a dual-chain cytokine receptor switch may bind to a respective activator and heterodimerize with each other to activate intracellular signaling. Optionally, a dual-chain cytokine receptor switch can be expressed as a single protein including both cytokine receptor chains. The single protein can be subsequently cleaved (e.g., via the inclusion of a 2A peptide or other cleavage sequence) to produce the two separate cytokine receptor chains. SEQ ID NO: 7 provides an example of a dual-chain cytokine receptor switch that is initially expressed as a single protein.

In some embodiments, an engineered cytokine receptor switch includes one or more cytokine receptor chains with one or more chimeric, tandem, and/or mutant intracellular domains. For example, a cytokine receptor switch can include a chimeric cytokine receptor chain including a first intracellular domain derived from IL2Rß and a second intracellular domain derived from IL2Rγ. As another example, a cytokine receptor switch can include a chimeric cytokine receptor chain including a first intracellular domain derived from IL7Rα and a second intracellular domain derived from IL2Rγ. In another example, a cytokine receptor switch can include a chimeric cytokine receptor chain including a first intracellular domain derived from IL21Rα and a second intracellular domain derived from IL2Rγ. In a further example, a cytokine receptor switch can include a tandem cytokine receptor chain including first and second intracellular domains derived from IL2Rß. As yet another example, a cytokine receptor switch can include a tandem cytokine receptor chain including first and second intracellular domains derived from IL7Rα. As another example, a cytokine receptor switch can include a mutant cytokine receptor chain including a mutant intracellular domain derived from IL2Rß. In another example, a cytokine receptor switch can include a mutant cytokine receptor chain including a mutant intracellular domain derived from IL7Rα. A mutant intracellular domain can include one or more mutations relative to the wild-type intracellular domain, such as point mutations, truncations, etc.

Optionally, the engineered cytokine receptor switch may exhibit activity (e.g., a cytokine signaling activity) without binding of an activator to the activator binding domain, referred to herein as “activator-independent activity” or “ligand-independent activity.” Without wishing to be bound by theory, it is hypothesized that activator-independent activity may be due to dimerization of a cytokine receptor chain of an engineered cytokine receptor switch with another cytokine receptor chain (e.g., of the engineered cytokine receptor switch or an endogenous cytokine receptor) that occurs even in the absence of the activator. Dimerization may occur between the extracellular and/or transmembrane domains of the cytokine receptor chains. Activator-independent activity may also occur due to interactions of the engineered cytokine receptor switch with other co-expressed receptors, such as a CAR. Such interactions may comprise physical interactions (e.g., dimerization) as well as interactions in downstream signaling pathways. The strength of activator-independent activity can be increased or decreased by changing the extracellular domain of the engineered cytokine receptor switch, and/or by increasing or decreasing the length of the hinge between domains of the cytokine receptor switch.

In some embodiments, activator-independent activity provides similar effects as activation of the engineered cytokine receptor switch (e.g., enhancement of memory phenotypes and/or lymphoid homing), but with reduced magnitude and/or shorter duration. In some embodiments, activator-independent activity primes the immune cell for subsequent activation, e.g., the magnitude and/or duration of the effects following administration of the activator is greater if the immune cell has previously exhibited activator-independent activity, versus an immune cell that does not exhibit activator-independent activity.

In some embodiments, the level of activator-independent activity exhibited by an engineered cytokine receptor switch depends at least partially on the structure of the engineered cytokine receptor switch. For instance, a shorter hinge may be associated with higher levels of activator-independent activity, e.g., due to enhanced dimerization facilitated by the reduced flexibility of the extracellular and/or transmembrane domains of the engineered cytokine receptor switch. The shorter hinge can be no more than 30 amino acids, 25 amino acids, 20 amino acids, 15 amino acids, 10 amino acids, 9 amino acids, 8 amino acids, 7 amino acids, 6 amino acids, 5 amino acids, 4 amino acids, 3 amino acids, 2 amino acids, or 1 amino acid in length. Conversely, a longer hinge may be associated with lower levels of activator-independent activity, e.g., due to reduced dimerization attributable to the increased flexibility of the extracellular and/or transmembrane domains of the engineered cytokine receptor switch. The longer hinge can be at least 20 amino acids, 25 amino acids, 30 amino acids, 35 amino acids, 40 amino acids, 45 amino acids, or 50 amino acids in length. Other structural features that may influence activator-independent activity include the size of the extracellular domain, the size of the transmembrane domain, and/or the size of the intracellular domain.

The structure of the engineered cytokine receptor switch (e.g., length of the hinge) can be selected to produce a desired level of activator-independent activity. Activator-independent activity can be beneficial, for example, to provide constitutive enhancement of memory phenotypes and/or lymphoid homing (e.g., in embodiments the engineered cytokine receptor switch is co-expressed with a direct CAR). Conversely, lower levels of activator-independent activity may be advantageous in situations where switchable control over immune cell activity is desired (e.g., in embodiments the engineered cytokine receptor switch is co-expressed with an indirect CAR).

Additional details of engineered cytokine receptor switches and methods of use thereof are provided in International Application No. WO 2024/123835, filed concurrently with the present application, which is incorporated herein by reference in its entirety.

### A. Activator Binding Domain

An engineered cytokine receptor switch of the present disclosure may comprise an activator binding domain. The activator binding domain may be positioned in an extracellular region of the engineered cytokine receptor switch and may be designed to bind an activator (e.g., a small molecule, a peptide, an oligonucleotide, a protein) to activate intracellular signaling through the intracellular signaling domain. In some embodiments, an activator may be selected to have low toxicity, low immunogenicity, low cross-reactivity, or combinations thereof to reduce unfavorable side effects when administered to a subject (e.g., a human subject). For example, the activator may be an exogenous activator (e.g., an exogenous small molecule, an exogenous peptide, an exogenous oligonucleotide, or an exogenous protein) that is not naturally present in a target environment (e.g., a human subject) to prevent activation of the engineered cytokine receptor switch in the absence of an external stimulus (e.g., administration of the activator), prevent cross-reactivity of the activator with other biological components, and to enable dynamic control of receptor signaling. Additional examples of activators are provided in Section VIII.D below.

The activator binding domain can be any protein, protein fragment, or peptide capable of selectively binding the activator. In some embodiments, for example, the activator binding domain may comprise an antibody (e.g., a monoclonal antibody), an antibody fragment, a single chain variable fragment (scFv), a nanobody, or a peptide. In some embodiments, an activator binding domain may comprise a fragment of an antibody (e.g., a variable fragment) that binds to a selected activator. Antibodies, antibody fragments, scFvs, and nanobodies may be produced using various methods known in the art to target a specific activator. In some embodiments, the activator binding domain is an scFv, a heavy chain variable domain (V_{H}), or a light chain variable domain (V_{L}) of an antibody, or a VHH antibody that recognizes any of the activators described herein, e.g., in Section VIII.D below. For example, the activator binding domain can be an scFv, a V_{H}, or a V_{L} of an anti-FITC antibody (e.g., a 4M5.3 anti-FITC antibody). As another example, the activator binding domain can be an scFv, a V_{H}, or a V_{L} of an anti-DOTA antibody (e.g., a C8.2.5 anti-DOTA antibody). In a further example, the activator binding domain can be an scFv, a V_{H}, or a V_{L} of an anti-MPOB antibody.

In some embodiments, the activator binding domain may be synthetic (e.g., engineered de novo to bind a specific small molecule activator or other activator type). In some embodiments, a small molecule binding domain may be humanized to reduce immunogenicity and prevent an immune reaction to the engineered cytokine receptor switch when administered to a subject (e.g., a human subject). Commercially available small molecule binding domains may be suitable for use as an activator binding domain in an engineered cytokine receptor switch.

In some embodiments, an activator binding domain may be suitable for use in an engineered cytokine receptor switch of the present disclosure if the activator binding domain does not target a small molecule produced in humans. An activator binding domain may be suitable for use in an engineered cytokine receptor switch of the present disclosure if the activator binding domain binds to a molecule that is non-toxic to humans, included in an Inactive Ingredients Database, or both.

The activator binding domain may have a molecular weight (e.g., number average or weight average molecular weight) of from about 1 kDa to about 150 kDa, from about 1 kDa to about 100 kDa, from about 1 kDa to about 90 kDa, from about 1 kDa to about 80 kDa, from about 1 kDa to about 70 kDa, from about 1 kDa to about 60 kDa, from about 1 kDa to about 50 kDa, from about 1 kDa to about 40 kDa, from about 1 kDa to about 35 kDa, from about 1 kDa to about 30 kDa, from about 1 kDa to about 25 kDa, from about 1 kDa to about 10 kDa, from about 5 kDa to about 150 kDa, from about 5 kDa to about 100 kDa, from about 5 kDa to about 90 kDa, from about 5 kDa to about 80 kDa, from about 5 kDa to about 70 kDa, from about 5 kDa to about 60 kDa, from about 5 kDa to about 50 kDa, from about 5 kDa to about 40 kDa, from about 5 kDa to about 35 kDa, from about 5 kDa to about 30 kDa, from about 5 kDa to about 25 kDa, from about 5 kDa to about 10 kDa, from about 10 kDa to about 150 kDa, from about 10 kDa to about 100 kDa, from about 10 kDa to about 90 kDa, from about 10 kDa to about 80 kDa, from about 10 kDa to about 70 kDa, from about 10 kDa to about 60 kDa, from about 10 kDa to about 50 kDa, from about 10 kDa to about 40 kDa, from about 10 kDa to about 35 kDa, from about 10 kDa to about 30 kDa, from about 10 kDa to about 25 kDa, from about 20 kDa to about 150 kDa, from about 20 kDa to about 100 kDa, from about 20 kDa to about 90 kDa, from about 20 kDa to about 80 kDa, from about 20 kDa to about 70 kDa, from about 20 kDa to about 60 kDa, from about 20 kDa to about 50 kDa, from about 20 kDa to about 40 kDa, from about 20 kDa to about 35 kDa, or from about 20 kDa to about 30 kDa. For example, the activator binding domain may comprise an scFv having a molecular weight of about 20 kDa to about 35 kDa. The activator binding domain may comprise a peptide having a molecular weight of about 1 kDa to about 10 kDa.

Examples of activator binding domains that may be used in an engineered cytokine receptor switch and corresponding activators are provided in Table 2.

TABLE 2
Representative Examples of Activator Binding Domains

| Activator | | | |
|---|---|---|---|
| Binding | | | |
| Domain | SEQ ID NO | Sequence | Activator |
| anti- | SEQ ID NO: 21 | DVVMTQTPLSLPVSLGDQASISCRSSQSLVHSNGNTYLR | Fluorescein |
| fluorescein | | WYLQKPGQSPKVLIYKVSNRVSGVPDRFSGSGSGTDFTL | or |
| scFv | | KINRVEAEDLGVYFCSQSTHVPWTFGGGTKLEIKSSADD | fluorescein |
| | | AKKDAAKKDDAKKDDAKKDGGVKLDETGGGLVQPGG | derivatives |
| | | AMKLSCVTSGFTFGHYWMNWVRQSPEKGLEWVAQFRN | (e.g., FITC) |
| | | KPYNYETYYSDSVKGRFTISRDDSKSSVYLQMNNLRVED | |
| | | TGIYYCTGASYGMEYLGQGTSVTVS | |

In some embodiments, an engineered cytokine receptor switch may comprise an activator binding domain comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to SEQ ID NO: 21. In some embodiments, the engineered cytokine receptor switch may comprise an activator binding domain of SEQ ID NO: 21.

### B. Intracellular Domain

An engineered cytokine receptor switch of the present disclosure may comprise an intracellular domain (also referred to herein as an “intracellular signaling domain”). The intracellular signaling domain may be positioned in an intracellular region of the engineered cytokine receptor switch and may be designed to activate intracellular signaling upon binding of an activator to an extracellular activator binding domain. Optionally, the intracellular signaling domain may exhibit activity independent of binding of the activator to the activator binding domain (activator-independent activity), as described elsewhere herein. The intracellular signaling domain may activate a cytokine signaling pathway, such as a Jak-STAT pathway. In some embodiments, activation of the cytokine signaling pathway may promote conversion of an immune cell expressing the engineered cytokine receptor switch to a memory phenotype (e.g., a central memory phenotype, a stem cell memory phenotype, an effector memory phenotype, or an effector memory re-expressing CD45RA phenotype). Alternatively or in combination, activation of the cytokine signaling pathway may upregulate expression of cell surface markers that enable homing of the immune cell to lymphoid organs (e.g., CD62L, CCR7), such as homing to the lymph nodes, spleen, thymus, and/or bone marrow.

An intracellular signaling domain may be derived from an endogenous cytokine receptor. For example, an intracellular signaling domain may be derived from an interleukin 2 receptor subunit α (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, or a GM-CSF.

In some embodiments, the intracellular signaling domain may comprise an intracellular domain, a fragment of an intracellular domain, or a variant of an intracellular domain of an endogenous cytokine receptor. For example, the intracellular signaling domain may comprise an intracellular domain, a fragment of an intracellular domain, or a variant of an intracellular domain of an interleukin 2 receptor subunit α (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, or an GM-CSF. The intracellular domain, fragment of the intracellular domain, or variant of the intracellular domain may be capable of activating the cytokine signaling pathway activated by the endogenous cytokine receptor from which it was derived.

In some embodiments, an engineered cytokine receptor switch includes a single intracellular signaling domain. Alternatively, an engineered cytokine receptor switch can include a plurality of intracellular signaling domains in tandem (e.g., two, three, four, five, or more intracellular domains in tandem). In such embodiments, some or all of the intracellular signaling domains may be the same intracellular signaling domain, or some or all of the intracellular signaling domains may be different intracellular signaling domains.

Examples of intracellular signaling domains that may be used in an engineered cytokine receptor switch are provided in Table 3.

TABLE 3
Representative Examples of Intracellular Signaling Domains

| Receptor | SEQ ID NO | Sequence |
|---|---|---|
| IL2Rα | SEQ ID NO: 29 | TWQRRQRKSRRTI |
| IL2Rβ | SEQ ID NO: 30 | NCRNTGPWLKKVLKCNTPDPSKFFSQLSSEHGGDVQKWLSSPFPSSSFSP |
| | | GGLAPEISPLEVLERDKVTQLLLQQDKVPEPASLSSNHSLTSCFTNQGYF |
| | | FFHLPDALEIEACQVYFTYDPYSEEDPDEGVAGAPTGSSPQPLQPLSGED |
| | | DAYCTFPSRDDLLLFSPSLLGGPSPPSTAPGGSGAGEERMPPSLQERVPR |
| | | DWDPQPLGPPTPGVPDLVDFQPPPELVLREAGEEVPDAGPREGVSFPWS |
| | | RPPGQGEFRALNARLPLNTDAYLSLQELQGQDPTHLV |
| IL2Rγ | SEQ ID NO: 31 | ERTMPRIPTLKNLEDLVTEYHGNFSAWSGVSKGLAESLQPDYSERLCLV |
| | | SEIPPKGGALGEGPGASPCNQHSPYWAPPCYTLKPET |
| IL7Rα | SEQ ID NO: 32 | KKRIKPIVWPSLPDHKKTLEHLCKKPRKNLNVSFNPESFLDCQIHRVDDI |
| | | QARDEVEGFLQDTFPQQLEESEKQRLGGDVQSPNCPSEDVVITPESFGRD |
| | | SSLTCLAGNVSACDAPILSSSRSLDCRESGKNGPHVYQDLLLSLGTTNST |
| | | LPPPFSLQSGILTLNPVAQGQPILTSLGSNQEEAYVTMSSFYQNQ |
| IL15Rα | SEQ ID NO: 33 | KSRQTPPLASVEMEAMEALPVTWGTSSRDEDLENCSHHL |
| IL21Rα | SEQ ID NO: 34 | SLKTHPLWRLWKKIWAVPSPERFFMPLYKGCSGDFKKWVGAPFTGSSL |
| | | ELGPWSPEVPSTLEVYSCHPPRSPAKRLQLTELQEPAELVESDGVPKPSF |
| | | WPTAQNSGGSAYSEERDRPYGLVSIDTVTVLDAEGPCTWPCSCEDDGYP |
| | | ALDLDAGLEPSPGLEDPLLDAGTTVLSCGCVSAGSPGLGGPLGSLLDRL |
| | | KPPLADGEDWAGGLPWGGRSPGGVSESEAGSPLAGLDMDTFDSGFVGS |
| | | DCSSPVECDFTSPGDEGPPRSYLRQWVVIPPPLSSPGPQAS |

In some embodiments, an engineered cytokine receptor switch may comprise an intracellular signaling domain comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 29-SEQ ID NO: 34. In some embodiments, the engineered cytokine receptor switch may comprise an intracellular signaling domain of any one of SEQ ID NO: 29-SEQ ID NO: 34.

In some embodiments, an intracellular signaling domain may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to an intracellular domain of an endogenous cytokine receptor (e.g., an interleukin 2 receptor subunit α (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, or a GM-CSF).

### C. Transmembrane Domain

An engineered cytokine receptor switch of the present disclosure may comprise a transmembrane domain. The transmembrane domain may connect an intracellular portion and an extracellular portion of the engineered cytokine receptor and may be designed to span a cell membrane and transduce a signal from an activator binding domain to an intracellular signaling domain upon binding of an activator to the activator binding domain.

A transmembrane domain may be derived from an endogenous cytokine receptor or from another type of receptor. For example, a transmembrane domain may be derived from an interleukin 2 receptor subunit α (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, an immunoglobulin (e.g., an IgG1, an IgG2, an IgG3, an IgG4, an IgM, an IgA, an IgD, an IgE), a CD8, a CD28, a GM-CSF, or an erythropoietin receptor (EpoR).

In some embodiments, the transmembrane domain may comprise a transmembrane domain or a variant of a transmembrane domain of an endogenous cytokine receptor or another type of receptor. For example, the transmembrane domain may comprise a transmembrane domain or a variant of a transmembrane domain of an interleukin 2 receptor subunit α (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, an immunoglobulin, a CD8, a CD28, a GM-CSF, or an EpoR. The transmembrane domain, fragment of the transmembrane domain, or variant of the transmembrane domain may be capable of activating the cytokine signaling pathway activated by the endogenous cytokine receptor from which it was derived.

In some embodiments, the transmembrane domain may comprise a transmembrane domain derived from any transmembrane protein. In some embodiments, the transmembrane domain may be a synthetic transmembrane domain. For example, the transmembrane domain may comprise a synthetic transmembrane α-helix, helical bundle, or ß-barrel.

Examples of transmembrane domains that may be used in an engineered cytokine receptor switch are provided in Table 4.

TABLE 4
Representative Examples of Transmembrane Domains

| Receptor | SEQ ID NO | Sequence |
|---|---|---|
| IL2Rα | SEQ ID NO: 23 | VAVAGCVFLLISVLLLSGL |
| IL2Rβ | SEQ ID NO: 24 | IPWLGHLLVGLSGAFGFIILVYLLI |
| IL2Rγ | SEQ ID NO: 25 | VVISVGSMGLIISLLCVYFWL |
| IL7Rα | SEQ ID NO: 26 | PILLTISILSFFSVALLVILACVLW |
| IL15Rα | SEQ ID NO: 27 | VAISTSTVLLCGLSAVSLLACYL |
| IL21Rα | SEQ ID NO: 28 | GWNPHLLLLLLLVIVFIPAFW |

In some embodiments, an engineered cytokine receptor switch may comprise a transmembrane domain comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 23-SEQ ID NO: 28. In some embodiments, the engineered cytokine receptor switch may comprise a transmembrane domain of any one of SEQ ID NO: 23-SEQ ID NO: 28.

In some embodiments, the transmembrane domain may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to a transmembrane domain of an endogenous cytokine receptor or another receptor (e.g., an interleukin 2 receptor subunit α (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, an immunoglobulin, a CD8, a CD28, a GM-CSF, or an EpoR).

### D. Signal Peptide

An engineered cytokine receptor switch of the present disclosure may comprise a signal peptide. The signal peptide may be positioned at the N-terminus of the engineered cytokine receptor and may be designed to direct expression of the engineered cytokine receptor switch to the endoplasmic reticulum (ER). The engineered cytokine receptor may be synthesized in the ER membrane and may be trafficked to the plasma membrane as a transmembrane protein.

A signal peptide may be derived from an endogenous cytokine receptor or another type of receptor. For example, signal peptide may be derived from an interleukin 2 receptor subunit a (IL2Rα), an interleukin 2 receptor subunit ß (IL2Rß), an interleukin 2 receptor subunit γ (IL2R), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, an immunoglobulin (e.g., an IgG1, an IgG2, an IgG3, an IgG4, an IgM, an IgA, an IgD, an IgE), a CD8, a CD28, or a GM-CSF.

In some embodiments, a signal peptide may comprise the signal peptide portion of an endogenous cytokine receptor or another receptor. For example, the signal peptide may comprise the signal peptide portion of an interleukin 2 receptor subunit α (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), a CD130, an immunoglobulin, a CD8, a CD28, or a GM-CSF.

In some embodiments, the signal peptide may be a signal peptide from any transmembrane or membrane-bound protein. The signal peptide may be sufficient to direct expression of the engineered cytokine receptor switch to the ER.

Examples of signal peptides that may be used in an engineered cytokine receptor switch are provided in Table 5.

TABLE 5
Representative Examples of Signal Peptides

| Receptor | SEQ ID NO | Sequence |
|---|---|---|
| IL2Rα | SEQ ID NO: 15 | MDSYLLMWGLLTFIMVPGCQA |
| IL2Rβ | SEQ ID NO: 16 | MAAPALSWRLPLLILLLPLATSWASA |
| IL2Rγ | SEQ ID NO: 17 | MLKPSLPFTSLLFLQLPLLGVG |
| IL7Rα | SEQ ID NO: 18 | MTILGTTFGMVFSLLQVVSG |
| IL15Rα | SEQ ID NO: 19 | MAPRRARGCRTLGLPALLLLLLLRPPATRG |
| IL21Rα | SEQ ID NO: 20 | MPRGWAAPLLLLLLQGGWG |

In some embodiments, an engineered cytokine receptor switch may comprise a signal peptide comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 15-SEQ ID NO: 20. In some embodiments, the engineered cytokine receptor switch may comprise a signal peptide of any one of SEQ ID NO: 15-SEQ ID NO: 20.

In some embodiments, the signal peptide may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to a signal peptide of an endogenous cytokine receptor or another receptor (e.g., an interleukin 2 receptor subunit α (IL2Rα), an interleukin 2 receptor subunit ß (IL2Rß), an interleukin 2 receptor subunit γ (IL2Rγ), an interleukin 4 receptor subunit α (IL4Rα), an interleukin 7 receptor subunit α (IL7Rα), an interleukin 15 receptor subunit α (IL15Rα), an interleukin 21 receptor subunit α (IL21Rα), an interleukin 1 receptor (ILIR), a CD123, a CD124, an interleukin 5 receptor subunit α (IL5Rα), an interleukin 5 receptor subunit ß (IL5Rß), a CD126, a CD132, a CD129, an interleukin 11 receptor subunit α (IL11Rα), an interleukin 12 receptor subunit ß1 (IL12Rß1), an interleukin 12 receptor subunit ß2 (IL12Rß2), interleukin 13 receptor subunit α1 (IL13Rα1), a CD122, an interleukin 18 receptor (IL18R), an interleukin 23 receptor (IL23R), an interleukin 27 receptor subunit α (IL27Rα), an immunoglobulin, a CD8, a CD28, or a CD130).

### E. Hinge

An engineered cytokine receptor switch of the present disclosure may comprise a hinge (also referred to herein as a “hinge domain”). The hinge may be positioned between the activator binding domain and the transmembrane domain and may be designed to increase the flexibility of the engineered cytokine receptor switch. Increased flexibility may reduce spatial constraints between the activator binding domain and the activator (e.g., a small molecule activator adhered to a surface), facilitating access to the activator. In some embodiments, the hinge may be engineered to provide a desired distance between the plasma membrane of a cell expressing the engineered cytokine receptor switch and an activator bound to the engineered cytokine receptor switch.

In some embodiments, the hinge may be a synthetic peptide designed to provide a desired length, flexibility, or both. In some embodiments, the hinge may be derived from an endogenous transmembrane protein. For example, the hinge may be derived from a CD8 (e.g., a CD8a), a CD3, a CD4, a CD28, a 4-1BB, a CD28, an OX40, an inducible T cell costimulatory (ICOS), a CD27, an immunoglobulin (e.g., an IgG1, an IgG2, an IgG3, an IgG4, an IgM, an IgA, an IgD, an IgE), or an EpoR. In some embodiments, the hinge may comprise a hinge of an endogenous transmembrane protein. For example, the hinge may be derived from a hinge of a CD8 (e.g., a CD8a), a CD3, a CD4, a CD28, a 4-1BB, a CD28, an OX40, an ICOS, a CD27, an immunoglobulin, or an EpoR.

Examples of hinges that may be used in an engineered cytokine receptor switch are provided in Table 6.

TABLE 6
Representative Examples of Hinges

| Hinge | SEQ ID NO | Sequence |
|---|---|---|
| CD8a Hinge | SEQ ID NO: 22 | TTTPAPRPPTPAPTIASQPLSLRPEACRPAAGGAVHTRGLDFACD |

In some embodiments, an engineered cytokine receptor switch may comprise a hinge comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to SEQ ID NO: 22. In some embodiments, the engineered cytokine receptor switch may comprise a hinge of SEQ ID NO: 22.

In some embodiments, the signal peptide may comprise a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to a hinge of an endogenous transmembrane protein (e.g., a CD8 (e.g., a CD8a), a CD3, a CD4, a CD28, a 4-1BB, a CD28, an OX40, an ICOS, a CD27, an immunoglobulin, or an EpoR).

As described herein, the length of the hinge may affect whether the engineered cytokine receptor switch exhibits activator-independent activity. For example, the hinge can be no more than 50 amino acids, 45 amino acids, 40 amino acids, 30 amino acids, 25 amino acids, 20 amino acids, 15 amino acids, 10 amino acids, 9 amino acids, 8 amino acids, 7 amino acids, 6 amino acids, 5 amino acids, 4 amino acids, 3 amino acids, 2 amino acids, or 1 amino acid in length. Alternatively or in combination, the hinge can be at least 1 amino acid, 2 amino acids, 3 amino acids, 4 amino acids, 5 amino acids, 6 amino acids, 7 amino acids, 8 amino acids, 9 amino acids, 10 amino acids, 15 amino acids, 20 amino acids, 25 amino acids, 30 amino acids, 35 amino acids, 40 amino acids, 45 amino acids, or 50 amino acids in length.

### F. Additional Components

In some embodiments, an engineered cytokine receptor switch of the present disclosure may comprise or be co-expressed with one or more additional components. Additional components that may be included in or co-expressed with an engineered cytokine receptor switch are provided in Table 7.

TABLE 7
Additional Components

| Component | SEQ ID NO | Sequence |
|---|---|---|
| P2A peptide | SEQ ID NO: 35 | GSGATNFSLLKQAGDVEENPGP |
| T2A peptide | SEQ ID NO: 36 | GSGEGRGSLLTCGDVEENPGP |
| E2A peptide | SEQ ID NO: 37 | GSGQCTNYALLKLAGDVESNPGP |
| F2A peptide | SEQ ID NO: 38 | GSGVKQTLNFDLLKLAGDVESNPGP |
| Truncated | SEQ ID NO: 39 | MPPPRLLFFLLFLTPMEVRPEEPLVVKVEEGDNAVLQCLKGTSD |
| CD19 | | GPTQQLTWSRESPLKPFLKLSLGLPGLGIHMRPLAIWLFIFNVSQ |
| | | QMGGFYLCQPGPPSEKAWQPGWTVNVEGSGELFRWNVSDLGG |
| | | LGCGLKNRSSEGPSSPSGKLMSPKLYVWAKDRPEIWEGEPPCLP |
| | | PRDSLNQSLSQDLTMAPGSTLWLSCGVPPDSVSRGPLSWTHVH |
| | | PKGPKSLLSLELKDDRPARDMWVMETGLLLPRATAQDAGKYY |
| | | CHRGNLTMSFHLEITARPVLWHWLLRTGGWKVSAVTLAYLIFC |
| | | LCSLVGILHLQRALVLRRKRKRMTDPTRRF |

In some embodiments, an engineered cytokine receptor switch may comprise or be co-expressed with a marker domain. The marker domain may be co-expressed with the cytokine receptor chain(s) of the engineered cytokine receptor switch for purposes of identifying immune cells that are expressing the engineered cytokine receptor switch (“positive cells”), enriching and purifying positive cells, acting as a conditional suicide switch for positive cells, and/or other relevant functions. The marker domain can be truncated (e.g., in the intracellular domain) such that the expressed truncated marker does not have the biological function of the native marker. The marker domain can be any cell surface molecule that is not present on natural T cells. For example, an engineered cytokine receptor switch may comprise a CD19 domain (e.g., a truncated CD19 domain of SEQ ID NO: 39), a CD20 domain (e.g., a truncated CD20 domain), a CD22 domain (e.g., a truncated CD22 domain), a CD34 domain (e.g., a truncated CD34 domain), or an EGFR domain (e.g., a truncated EGFR domain). In some embodiments, an engineered cytokine receptor switch may comprise a marker domain comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to SEQ ID NO: 39. In some embodiments, the engineered cytokine receptor switch may comprise a marker domain of SEQ ID NO: 39.

Alternatively or in combination, the marker domain can be a detection marker, such as a fluorescent protein. For example, an engineered cytokine receptor switch may comprise EBFP, Sapphire, T-Sapphire, ECFP, mCFP, Cerulean, CyPet, AmCyan1, Midori-Ishi Cyan, mTFP1, GFP, EGFP, AcGFP, TurboGFP, Emerald, Azami Green, ZsGreen, EYFP, Topaz, Venus, mCitrine, yPet, PhiYFP, ZsYellow1, mBanana, Kusabira Orange, mOrange, dTomato, tdTomato, DsRed, DsRed2, DsRed-Express, DsRed-Monomer, mTangerine, mStrawberry, AsRed2, mRFP1, JREd, mCherry, HcRed1, mRaspberry, HcRed-Tandem, mPlum, or AQ143.

In some embodiments, a construct for expression of an engineered cytokine receptor switch may comprise a cleavage sequence, such as a 2A self-cleaving peptide sequence (e.g., a P2A peptide of SEQ ID NO: 35, a T2A peptide of SEQ ID NO: 36, a E2A peptide of SEQ ID NO: 37, or a F2A peptide of SEQ ID NO: 38). A 2A self-cleaving peptide sequence (also known as a “2A peptide”) may be included to link an engineered cytokine receptor switch to one or more additional engineered cytokine receptor switches for co-expression. In some embodiments, a 2A peptide may link a first engineered cytokine receptor switch to a second engineered cytokine receptor switch. For example, a 2A peptide (e.g., of any one of SEQ ID NO: 35-SEQ ID NO: 38) may link a first cytokine receptor chain (e.g., an IL2Rß cytokine receptor chain or an IL2RY cytokine receptor chain) to a second cytokine receptor chain (e.g., an IL2Rß cytokine receptor chain or an IL2Rγ cytokine receptor chain) to form a single protein that encompasses both chains of an engineered dual-chain cytokine receptor switch (e.g., a dual-chain cytokine receptor switch of SEQ ID NO: 7). After expression, the protein can be cleaved at the cleavage sequence to produce the separate two cytokine receptor chains of the dual-chain cytokine receptor switch. As another example, a 2A peptide (e.g., of any one of SEQ ID NO: 35-SEQ ID NO: 38) may be included to link a cytokine receptor chain to a marker domain (e.g., the marker domain of SEQ ID NO: 39) to form a single protein that encompasses the cytokine receptor chain and marker domain. After expression, the protein can be cleaved at the cleavage sequence to separate the cytokine receptor chain from the marker domain.

In some embodiments, a construct for expression of an engineered cytokine receptor switch may comprise a cleavage sequence comprising at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to SEQ ID NO: 35-SEQ ID NO: 38. In some embodiments, the construct for expression of the engineered cytokine receptor switch may comprise a cleavage sequence of SEQ ID NO: 35-SEQ ID NO: 38.

Alternatively or in combination, a construct for expression of an engineered cytokine receptor switch may comprise an internal ribosome entry site (IRES) to allow for co-expression of an engineered cytokine receptor switch to an additional engineered cytokine receptor switch and/or to a marker domain. In some embodiments, a IRES may link a first engineered cytokine receptor switch to a second engineered cytokine receptor switch in an expression construct, such that the first engineered cytokine receptor switch and the second engineered cytokine receptor switch are translated as separate proteins. In some embodiments, an IRES may link an engineered cytokine receptor switch to a marker domain, such that the engineered cytokine receptor switch and the marker domain are translated as separate proteins.

### III. Chimeric Antigen Receptors

Provided herein are immune cells comprising an engineered cytokine receptor switch of the present disclosure and a chimeric antigen receptor (CAR). An engineered cytokine receptor switch of the present disclosure may be used in conjunction with a CAR to produce a therapeutic effect (e.g., an anti-cancer effect). In some embodiments, an engineered cytokine receptor switch may be expressed in an immune cell along with a CAR. For example, a CAR T-cell of the present disclosure may be engineered to co-express an engineered cytokine receptor switch and a CAR that binds a tumor antigen. The CAR may bind to an antigen (e.g., a tumor antigen) on a target cell to recruit the immune cell to the target cell. For example, a CAR may be engineered to bind to a surface antigen on a tumor cell via an antigen binding domain and activate intracellular signaling through an intracellular signaling domain to produce a targeted immune response against the tumor cell. The CAR may recruit the immune cell to a target cell after the immune cell has been activated. As described herein, activation of an immune cell may occur ex vivo (e.g., via binding of an activator to the engineered cytokine receptor switch during manufacturing), in vivo (e.g., via binding of an activator to the engineered cytokine receptor switch and/or activation of the immune cell in a lymphoid organ), or a combination thereof. Optionally, the CAR may recruit the immune cell to a target cell without activation of the immune cell (e.g., no activator is provided to the immune cell either ex vivo or in vivo but the engineered cytokine receptor switch may still exhibit activator-independent activity).

A CAR may comprise an extracellular domain, a transmembrane domain, and an intracellular domain. The extracellular domain of the CAR may comprise an antigen binding domain that binds specifically to an antigen. The antigen binding domain can be any protein, protein fragment, or peptide capable of selectively binding the antigen. In some embodiments, for example, the antigen binding domain may comprise an antibody (e.g., a monoclonal antibody), an antibody fragment, an scFv, a nanobody, or a peptide. In some embodiments, an antigen binding domain may comprise a fragment of an antibody (e.g., a variable fragment) that binds to a selected antigen. Antibodies, antibody fragments, scFvs, and nanobodies may be produced using various methods known in the art to target a specific antigen. In some embodiments, the antigen binding domain may comprise a VHH antibody, an scFv, a V_{H}, a V_{L}, or a ligand specific for a target antigen. The antigen binding domain of the CAR may bind to a specific epitope of the target antigen.

In some embodiments, the antigen may be a tumor antigen, such as a tumor cell surface marker, a tumor-specific antigen, or a tumor-associated antigen. For example, the antigen may be CD19, CD20, CD22, CD123, CD33, CD3, CD4, CD8, CD38, SLAMF7, BCMA, GD2, GPRC5D, MUC16, HER2, EGFR, EGFRVIII, CLL-1, CD44v6, folate receptor-α, mesothelin, CD20, CD37, ROR1, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-1, IGF-II, IGF-1 receptor, IL13Rα2, B7-H3 (CD276), EPHA2, GRP78, NKG2D, or CD70. In some embodiments, the CAR may be selected to target a tumor cell antigen associated with a cancer of interest. A CAR having an antigen binding domain that recognizes an antigen expressed by a target cell (e.g., a tumor antigen expressed by a cancer cell) may be referred to herein as a “direct CAR.” A direct CAR may be targeted to a target cell via direct binding of the antigen binding domain to the antigen expressed by the target cell.

In some embodiments, the antigen binding domain is a VHH antibody, an scFv, a V_{H}, or a V_{L} of an antibody, or a ligand that recognizes any of the tumor antigens described herein. For example, the antigen binding domain can be a VHH antibody, an scFv, a V_{H}, or a V_{L} of an anti-BCMA antibody (e.g., as described in U.S. Patent Publication Nos. 2020/0261501 and 2022/0127371, the disclosures of which are incorporated herein by reference in their entirety). As another example, the antigen binding domain can be a VHH antibody, an scFv, a V_{H}, or a V_{L} of an anti-CD123 antibody (e.g., as described in U.S. Patent Publication No. 2020/0254023, the disclosure of which is incorporated by reference herein in its entirety). In a further example, the antigen binding domain can be a VHH antibody, an scFv, a V_{H}, or a V_{L} of an anti-GD2 antibody.

In some embodiments, the antigen binding domain of the CAR may recognize an antigen that is not expressed by the target cell of the CAR. For instance, the antigen may be a “synthetic antigen” that is not expressed by normal cells or cancer cells of the subject. A CAR that binds to a synthetic antigen may be referred to herein as an “indirect CAR.” An indirect CAR may be targeted to a target cell via a bispecific agent including (1) the synthetic antigen and (2) a targeting moiety that binds to an antigen expressed by the target cell (e.g., a tumor antigen expressed by a tumor cell). The indirect CAR may bind indirectly to the target cell by binding of the antigen binding domain to the synthetic antigen of the bispecific agent, and by binding of the targeting moiety of the bispecific agent to the antigen expressed by the target cell. The targeting moiety and tumor antigen can be any of the embodiments described herein.

The synthetic antigen may be a small molecule, a peptide, an oligonucleotide, or a protein. In some embodiments, a synthetic antigen may be selected to have low toxicity, low immunogenicity, low cross-reactivity, or combinations thereof to reduce unfavorable side effects when administered to a subject (e.g., a human subject). For instance, the synthetic antigen can be a molecule that is non-toxic to humans, included in an Inactive Ingredients Database, or both. In some embodiments, the synthetic antigen may be an exogenous antigen (e.g., an exogenous small molecule, an exogenous peptide, an exogenous oligonucleotide, or an exogenous protein) that is not naturally present in a target environment (e.g., a human subject) to prevent activation of the indirect CAR in the absence of an external stimulus (e.g., administration of the bispecific agent), prevent cross-reactivity of the synthetic antigen with other biological components, and to enable dynamic control of CAR activity.

Examples of exogenous small molecules (e.g., haptens) that may be recognized by an antigen binding domain of an indirect CAR include fluorophores (e.g., fluorescein, fluorescein derivatives, indocyanines, indocyanine derivatives, cyanines, cyanine derivatives), chelators (e.g., DOTA), or other small molecules. For example, the fluorescein derivative may be fluorescein isothiocyanate (FITC), fluorescein 5-maleimide, fluorescein-5-carboxamide, fluorescein-6-carboxamide, or 6-FAM phosphoramidite. Additional examples of small molecules that may bind to an antigen binding domain of an indirect CAR include Topiramate hemisuccinate, Creatine, Acetaminophen, Ketamine, Propofol, Lidocaine, Ractopamine, Salicylate, Salicylic Acid, Sulfasalazine, Dapsone, Albendazole, Ivermectin, Levamisole, Permethrin, Pyrantel, Thiabendazole, Procainamide, Sulfamethazine, Amikacin, Amoxicillin, Ampicillin, Cefazolin, Cefuroxime, Cephalexin, Chloramphenicol, Chloramphenicol, Ciprofloxacin, Clenbuterol, Cloxacillin, Colistin A, Dicloxacillin, Enrofloxacin, Furaltadone, Gentamicin, Gentamicin, Kanamycin, Kanamycin, Kincomycin, Lincomycin, Metronidazole, Nafcillin, Nalidixic Acid, Neomycin, Neomycin, Nitrofurazone, Norfloxacin, Ofloxacin, Oxacillin, Spectinomycin, Streptomycin, Streptomycin, Sulfabenzamide, Sulfacetamide, Sulfadiazine, Sulfadimidine, Sulfametoxydiazine, Sulfanilamide, Trimethoprim, Carbamazepine, Ethosuximide, Lamotrigine, Primidone, Cetirizine, Chlorpheniramine, Diphenhydramine, Doxylamine, Promethazine, Sulfadimethoxine, Benzothiazinone, Butylated Hydroxytoluene, Tripelennamine, Chlorpromazine, Clozapine, Haloperidol, Olanzapine, Paliperidone, Quetiapine, Ribavirin, Meprobamate, Acebutolol, Atenolol, Penbutolol, Warfarin, Salmeterol, Aflatoxin B1, Tetraxetan

(DOTA), MPOB, Biotin, Melamine, Methotrexate, Amphetamine, Diethylpropion, Dextromethorphan, Pseudoephedrine, Dihydrochlorothiazide, Hydrochlorothiazide, Clonazepam, Diazepam, Nitrazepam, Rhodamine B, Fluorescent Brightener Ksn, Zearalenone, Sudan Red1, Acetominophen, Acrylamide, Benzoic Acid, Benzophenone, Benzothiazine,

Mercaptobenzothiazole, Erythrosine, Sudan, Tartrazine, Erythromycin, Sirolimus, Atropine, Ethyl glucuronide, Aflatoxin M1, Methocarbamol, Fentanyl, Hydromorphone, Morphine, Remifentanil, Tapentadol, Tramadol, Pregabalin, Gabapentin, Amitriptyline, Desipramine, Imipramine, Nortriptyline, Venlafaxine, Dinitrophenyl (DNP), His-Tag, PEG methoxy group, Etodolac, Ibuprofen, Ketoprofen, Meclofenamic Acid, Phenylbutazone, Acetyl Salicylic Acid, Acetamiprid, Acetochlor, Carbadazim, Carbaryl, Chlorothalonil, Chlorpyrifos, Fenpropathrin, Imazalil, Imidacloprid, Parathion, Abscisic acid, Dibutyl Phthalate, Clonazepam, Lorazepam, Oxazepam, Phenobarbital, Secobarbital, Zaleplon, Zolpidem, Trazodone, Fluoxetine, Fluvoxamine, Cortisone, Dexamethasone, Dihydrotestosterone, Fluocinolone, Methylprednisolone, Prednisolone, Stanozolol, Triamcinolone, Mazindol, Methamphetamine, Methylphenidate, Modafinil, Chrysoidine, Deoxynivalenol, Fumonisin, Microcystin Lr, Ochratoxin, Sterigmatocystin, T-2 toxin, Sildenafil, Tadalafil, Scopolamine, Florfenicol, Pirlimycin, or Sulfaquinoxaline.

In some embodiments, the antigen binding domain is a VHH antibody, an scFv, a V_{H}, or a V_{L} of an antibody that recognizes any of the exogenous antigens described herein. For example, the antigen binding domain can be a VHH antibody, an scFv, a V_{H}, or a V_{L} of an anti-FITC antibody (e.g., a 4M5.3 anti-FITC antibody). As another example, the antigen binding domain can be a VHH antibody, an scFv, a V_{H}, or a V_{L} of an anti-DOTA antibody (e.g., a C8.2.5 anti-DOTA antibody). In a further example, the antigen binding domain can be a VHH antibody, an scFv, a V_{H}, or a V_{L} of an anti-MPOB antibody.

In some embodiments, the antigen recognized by the indirect CAR is the same as the activator recognized by the engineered cytokine receptor switch (e.g., the antigen binding domain of the CAR can be the same as the activator binding domain of the engineered cytokine receptor switch). In such embodiments, the indirect CAR may recognize the same epitope on the activator as the engineered cytokine receptor switch, or may recognize a different epitope on the activator than the engineered cytokine receptor switch. Alternatively, the antigen recognized by the indirect CAR can be different from the activator recognized by the engineered cytokine, receptor switch (e.g., the antigen binding domain of the CAR can be different than the activator binding domain of the engineered cytokine receptor switch). In some embodiments, the antigen binding domain of the indirect CAR binds to a first small molecule, and the activator binding domain of the engineered cytokine receptor switch binds to a second small molecule, where the first small molecule may be the same as or different than the second small molecule. In some embodiments, the antigen binding domain of the indirect CAR binds to a first epitope on a small molecule, and the activator binding domain of the engineered cytokine receptor switch binds to a second epitope on the small molecule, where the first epitope may be the same as or different than the second epitope.

The transmembrane domain of the CAR (e.g., a direct CAR or an indirect CAR) may link the extracellular domain to the intracellular domain. In some embodiments, the transmembrane domain may be a transmembrane domain derived from any transmembrane protein. The transmembrane domain may comprise a transmembrane region of alpha, beta, or zeta chain of the T-cell receptor a chain, T-cell receptor β chain, T-cell receptor ζ chain, CD28, CD3 epsilon, CD45, CD4, CD5, CD8, CD9, CD16, CD22, CD33, CD37, CD64, CD80, CD86, CD134, CD137, or CD154. For example, the transmembrane domain may comprise a CD8 transmembrane domain, including a CD8a hinge domain and/or a CD8a transmembrane domain. In some embodiments, the transmembrane domain may be synthetic. For example, a synthetic transmembrane domain may comprise mostly hydrophobic residues (e.g., glycine, leucine, isoleucine, alanine, valine, proline, methionine, phenylalanine, and tryptophan). In some embodiments, a first peptide linker (e.g., comprising glycine, serine, or combinations thereof) may connect the transmembrane domain to the extracellular domain. In some embodiments, a second peptide linker (e.g., comprising glycine, serine, or combinations thereof) may connect the transmembrane domain to the intracellular domain.

The intracellular domain of the CAR (e.g., a direct CAR or an indirect CAR), also referred to as the cytoplasmic domain, may be capable of activating a specialized immune cell function (e.g., an immune response). For example, the specialized immune cell function of a T-cell may comprise cytolytic activity, cytokine secretion, or both. In some embodiments, the intracellular domain of the CAR may comprise the intracellular domain of TCR zeta, FcR gamma, FcR beta, CD3 zeta, CD3 gamma, CD3 delta, CD3 epsilon, CD5, CD22, CD79a, CD79b, or CD66d. In some embodiments, the intracellular domain of the CAR may comprise a portion of the 53-intracellular domain of TCR zeta, FcR gamma, FcR beta, CD3 zeta, CD3 gamma, CD3 delta, CD3 epsilon, CD5, CD22, CD79a, CD79b, or CD66d sufficient to activate the specialized immune cell function. In some embodiments, the intracellular domain of the CAR may comprise a CD137 (4-1BB) signaling domain, a CD28 signaling domain, and/or a CD3 zeta signal domain.

Binding of the antigen to the antigen binding domain of the CAR may initiate signal transduction through the transmembrane domain to the cytoplasmic domain to activate the specialized immune cell function. For example, binding of the antigen binding domain to a tumor antigen may activate the intracellular domain of the CAR to trigger cytokine release. In some embodiments, the CAR may facilitate antigen-specific cancer cell killing by binding directly or indirectly to a tumor cell surface antigen present on the cancer cell via the antigen binding domain, transducing a signal through the transmembrane domain to the intracellular domain, and activating the specialized immune cell function (e.g., cytokine release) via activation of the intracellular domain. The specialized immune cell function may kill the cancer cell or may facilitate killing of the cancer cell.

Examples of polynucleotide sequences for CARs are provided in Table 8.

TABLE 8
Representative Examples of CARs

| CAR | SEQ ID NO | Sequence |
|---|---|---|
| bb2121 (anti- | SEQ ID NO: 40 | ATGGCACTCCCCGTCACCGCCCTTCTCTTGCCCCTCGCCCTGC |
| BCMA CAR)- | | TGCTGCATGCTGCCAGGCCCGACATTGTGCTCACTCAGTCAC |
| P2A-truncated | | CTCCCAGCCTGGCCATGAGCCTGGGAAAAAGGGCCACCATC |
| CD19 | | TCCTGTAGAGCCAGTGAGTCCGTCACAATCTTGGGGAGCCAT |
| | | CTTATTCACTGGTATCAGCAGAAGCCCGGGCAGCCTCCAACC |
| | | CTTCTTATTCAGCTCGCGTCAAACGTCCAGACGGGTGTACCT |
| | | GCCAGATTTTCTGGTAGCGGGTCCCGCACTGATTTTACACTG |
| | | ACCATAGATCCAGTGGAAGAAGACGATGTGGCCGTGTATTA |
| | | TTGTCTGCAGAGCAGAACGATTCCTCGCACATTTGGTGGGGG |
| | | TACTAAGCTGGAGATTAAGGGAAGCACGTCCGGCTCAGGGA |
| | | AGCCGGGCTCCGGCGAGGGAAGCACGAAGGGGCAAATTCAG |
| | | CTGGTCCAGAGCGGACCTGAGCTGAAAAAACCCGGCGAGAC |
| | | TGTTAAGATCAGTTGTAAAGCATCTGGCTATACCTTCACCGA |
| | | CTACAGCATAAATTGGGTGAAACGGGCCCCTGGAAAGGGCC |
| | | TCAAATGGATGGGTTGGATCAATACCGAAACTAGGGAGCCT |
| | | GCTTATGCATATGACTTCCGCGGGAGATTCGCCTTTTCACTC |
| | | GAGACATCTGCCTCTACTGCTTACCTCCAAATAAACAACCTC |
| | | AAGTATGAAGATACAGCCACTTACTTTTGCGCCCTCGACTAT |
| | | AGTTACGCCATGGACTACTGGGGACAGGGAACCTCCGTTACC |
| | | GTCAGTTCCGCGGCCGCAACCACAACACCTGCTCCAAGGCCC |
| | | CCCACACCCGCTCCAACTATAGCCAGCCAACCATTGAGCCTC |
| | | AGACCTGAAGCTTGCAGGCCCGCAGCAGGAGGCGCCGTCCA |
| | | TACGCGAGGCCTGGACTTCGCGTGTGATATTTATATTTGGGC |
| | | CCCTTTGGCCGGAACATGTGGGGTGTTGCTTCTCTCCCTTGTG |
| | | ATCACTCTGTATTGTAAGCGCGGGAGAAAGAAGCTCCTGTAC |
| | | ATCTTCAAGCAGCCTTTTATGCGACCTGTGCAAACCACTCAG |
| | | GAAGAAGATGGGTGTTCATGCCGCTTCCCCGAGGAGGAAGA |
| | | AGGAGGGTGTGAACTGAGGGTGAAATTTTCTAGAAGCGCCG |
| | | ATGCTCCCGCATATCAGCAGGGTCAGAATCAGCTCTACAATG |
| | | AATTGAATCTCGGCAGGCGAGAAGAGTACGATGTTCTGGAC |
| | | AAGAGACGGGGCAGGGATCCCGAGATGGGGGGAAAGCCCC |
| | | GGAGAAAAAATCCTCAGGAGGGGTTGTACAATGAGCTGCAG |
| | | AAGGACAAGATGGCTGAAGCCTATAGCGAGATCGGAATGAA |
| | | AGGCGAAAGACGCAGAGGCAAGGGGCATGACGGTCTGTACC |
| | | AGGGTCTCTCTACAGCCACCAAGGACACTTATGATGCGTTGC |
| | | ATATGCAAGCCTTGCCACCCCGCGGATCTGGCGCCACAAACT |
| | | TCTCACTGCTGAAACAGGCCGGCGACGTCGAAGAGAATCCT |
| | | GGGCCTATGCCTCCTCCTCGGCTGCTGTTCTTCCTGCTGTTTC |
| | | TGACCCCTATGGAAGTGCGGCCCGAGGAACCTCTGGTCGTGA |
| | | AAGTTGAAGAGGGCGACAACGCCGTGCTGCAGTGTCTGAAG |
| | | GGCACATCTGATGGCCCCACACAGCAGCTGACCTGGTCTAGA |
| | | GAGAGCCCTCTGAAGCCCTTCCTGAAGCTCAGTCTGGGACTG |
| | | CCTGGACTGGGCATCCATATGAGGCCACTGGCCATCTGGCTG |
| | | TTCATCTTTAACGTGTCCCAGCAGATGGGAGGCTTCTACCTG |
| | | TGTCAGCCTGGACCTCCTAGCGAGAAAGCTTGGCAGCCTGGC |
| | | TGGACCGTGAATGTGGAAGGATCCGGCGAGCTGTTCCGGTG |
| | | GAATGTCTCTGATCTCGGCGGCCTCGGATGCGGCCTGAAGAA |
| | | TAGATCTAGCGAGGGCCCTAGCAGCCCCTCCGGAAAACTGA |
| | | TGAGCCCCAAGCTGTACGTGTGGGCCAAAGACAGACCCGAG |
| | | ATTTGGGAGGGCGAGCCTCCTTGTCTGCCACCTAGAGACAGC |
| | | CTGAATCAGAGCCTGAGCCAGGACCTGACAATGGCCCCTGG |
| | | ATCTACACTGTGGCTGAGCTGCGGAGTGCCTCCTGACAGTGT |
| | | GTCTAGAGGCCCACTGAGCTGGACACACGTGCACCCTAAGG |
| | | GCCCTAAGAGCCTGCTGAGTCTGGAACTGAAGGACGACAGG |
| | | CCCGCCAGAGATATGTGGGTCATGGAAACCGGACTGCTGCTC |
| | | CCTAGAGCCACTGCTCAAGATGCCGGCAAGTACTATTGCCAC |
| | | CGGGGCAACCTGACCATGAGCTTCCACCTGGAAATTACCGCC |
| | | AGACCAGTGCTGTGGCATTGGCTGCTTAGAACCGGCGGATG |
| | | GAAGGTGTCAGCCGTGACTCTGGCCTACCTGATCTTTTGTCT |
| | | GTGCAGCCTCGTGGGCATCCTGCATCTGCAAAGAGCCCTGGT |
| | | CCTGCGGCGGAAGCGGAAGAGAATGACCGATCCTACCAGAC |
| | | GGTTCTGATGA |
| Carvykti (anti- | SEQ ID NO: 41 | ATGGCTCTGCCTGTCACCGCTCTGCTGCTGCCTCTGGCTCTGC |
| BCMA CAR) | | TGCTGCACGCTGCTCGCCCTCAGGTCAAACTGGAAGAAAGTG |
| | | GGGGAGGCCTGGTGCAGGCAGGACGGAGCCTGCGCCTGAGC |
| | | TGCGCAGCATCCGAGCACACCTTCAGCTCCCACGTGATGGGC |
| | | TGGTTTCGGCAGGCCCCAGGCAAGGAGAGAGAGTCCGTGGC |
| | | CGTGATCGGCTGGAGGGACATCTCCACATCTTACGCCGATTC |
| | | TGTGAAGGGCCGGTTCACCATCAGCAGAGACAACGCCAAGA |
| | | AGACACTGTATCTGCAGATGAATAGCCTGAAGCCCGAGGAC |
| | | ACCGCCGTGTACTATTGCGCAGCAAGGAGAATCGACGCAGC |
| | | AGACTTTGATTCCTGGGGCCAGGGCACCCAGGTGACAGTGTC |
| | | TAGCGGAGGAGGAGGATCTGGAGGAGGAGGAAGCGGAGGA |
| | | GGAGGATCCGAGGTGCAGCTGGTGGAGTCTGGAGGCGGCCT |
| | | GGTGCAGGCCGGAGGCTCTCTGAGGCTGAGCTGTGCAGCAT |
| | | CCGGAAGAACCTTCACAATGGGCTGGTTTAGGCAGGCACCA |
| | | GGAAAGGAGAGGGAGTTCGTGGCAGCAATCAGCCTGTCCCC |
| | | TACCCTGGCCTACTATGCCGAGTCCGTGAAGGGCAGGTTTAC |
| | | CATCTCTCGCGATAACGCCAAGAATACAGTGGTGCTGCAGAT |
| | | GAACAGCCTGAAACCTGAGGACACAGCCCTGTACTATTGTGC |
| | | CGCCGATCGGAAGAGCGTGATGAGCATTAGACCCGATTATT |
| | | GGGGACAGGGCACACAGGTGACAGTGAGTAGCACTAGTACC |
| | | ACGACGCCAGCGCCGCGACCACCAACACCGGCGCCCACCAT |
| | | CGCGTCGCAGCCCCTGTCCCTGCGCCCAGAGGCGTGCCGGCC |
| | | AGCGGCGGGGGGCGCAGTGCACACGAGGGGGCTGGACTTCG |
| | | CCTGTGATATCTACATCTGGGCGCCCTTGGCCGGGACTTGTG |
| | | GGGTCCTTCTCCTGTCACTGGTTATCACCCTTTACTGCAAACG |
| | | GGGCAGAAAGAAACTCCTGTATATATTCAAACAACCATTTAT |
| | | GAGACCAGTACAAACTACTCAAGAGGAAGATGGCTGTAGCT |
| | | GCCGATTTCCAGAAGAAGAAGAAGGAGGATGTGAACTGAGA |
| | | GTGAAGTTCAGCAGGAGCGCAGACGCCCCCGCGTACCAGCA |
| | | GGGCCAGAACCAGCTCTATAACGAGCTCAATCTAGGACGAA |
| | | GAGAGGAGTACGATGTTTTGGACAAGAGACGTGGCCGGGAC |
| | | CCTGAGATGGGGGGAAAGCCGAGAAGGAAGAACCCTCAGG |
| | | AAGGCCTGTACAATGAACTGCAGAAAGATAAGATGGCGGAG |
| | | GCCTACAGTGAGATTGGGATGAAAGGCGAGCGCCGGAGGGG |
| | | CAAGGGGCACGATGGCCTTTACCAGGGTCTCAGTACAGCCA |
| | | CCAAGGACACCTACGACGCCCTTCACATGCAGGCCCTGCCCC |
| | | CTCGCTAATGA |
| anti-CD123 | SEQ ID NO: 42 | ATGGCCCTGCCAGTAACTGCTCTTCTCCTGCCACTCGCTCTTC |
| CAR | | TGCTGCACGCCGCACGGCCTCAAATTCAACTTGTGCAGAGTG |
| | | GCCCCGAGCTGAAAAAGCCCGGCGAAACAGTAAAGATCTCT |
| | | TGTAAGGCGTCTGGATATATCTTTACCAATTACGGTATGAAC |
| | | TGGGTCAAACAGGCACCCGGTAAAAGCTTTAAATGGATGGG |
| | | CTGGATAAATACTTATACCGGAGAAAGCACGTATAGCGCCG |
| | | ACTTCAAGGGTAGATTTGCGTTCAGCCTGGAAACGTCCGCCA |
| | | GCACTGCATACTTGCACATCAATGACTTGAAAAATGAGGATA |
| | | CCGCGACCTACTTTTGTGCTAGAAGTGGTGGATACGATCCCA |
| | | TGGATTACTGGGGACAGGGGACCTCTGTCACAGTCAGTTCTG |
| | | GGGGCGGCGGTTCCGGAGGTGGCGGCTCAGGTGGAGGTGGG |
| | | TCAGATATCGTGTTGACACAAAGCCCTGCTTCCCTGGCCGTC |
| | | AGCTTGGGACAGAGAGCTACTATCTCCTGTAGAGCAAGTGA |
| | | GTCTGTCGACAATTACGGCAACACCTTTATGCACTGGTACCA |
| | | GCAGAAGCCAGGCCAGCCACCGAAGCTGTTGATTTACCGGG |
| | | CCTCAAACCTCGAAAGCGGGATTCCAGCTAGATTTTCTGGCA |
| | | GCGGGAGTCGAACCGACTTCACACTGACTATTAACCCAGTGG |
| | | AAGCAGATGACGTAGCCACATATTACTGTCAGCAGAGCAAC |
| | | GAAGACCCCCCGACGTTTGGTGCTGGAACCAAACTGGAGCT |
| | | GAAAACGACAACCCCCGCTCCACGACCTCCTACTCCCGCTCC |
| | | CACAATCGCATCTCAGCCACTCTCCTTGAGGCCTGAGGCCTG |
| | | CCGACCAGCTGCAGGCGGCGCAGTACACACCAGAGGCCTCG |
| | | ACTTTGCTTGCGATATCTACATATGGGCGCCCCTGGCCGGGA |
| | | CGTGTGGGGTATTGCTCCTGAGCCTTGTGATCACTCTTTACTG |
| | | CAAAAGAGGTCGGAAAAAGCTTCTTTACATCTTTAAACAGCC |
| | | GTTCATGCGCCCGGTGCAAACAACACAGGAGGAGGATGGAT |
| | | GCAGCTGCAGGTTTCCGGAGGAGGAGGAAGGCGGGTGTGAG |
| | | CTCCGGGTAAAATTCTCCAGAAGCGCAGACGCGCCAGCCTAT |
| | | CAGCAGGGGCAGAATCAGCTCTATAACGAACTCAATCTTGG |
| | | GCGACGGGAAGAATACGATGTACTTGATAAACGCCGAGGTC |
| | | GCGACCCCGAAATGGGAGGCAAGCCTCAGAGACGCAAGAAT |
| | | CCCCAAGAAGGCCTGTATAATGAACTCCAGAAGGATAAAAT |
| | | GGCGGAGGCCTACAGCGAAATTGGTATGAAGGGAGAAAGAC |
| | | GACGCGGTAAAGGCCACGACGGCCTTTACCAGGGCCTGTCA |
| | | ACAGCTACCAAAGATACGTATGATGCACTTCACATGCAAGCC |
| | | CTGCCGCCCCGGTGATGA |
| anti-GD2 CAR | SEQ ID NO: 43 | ATGGCTCTGCCTGTCACCGCTCTGCTGCTGCCTCTGGCTCTGC |
| | | TGCTGCACGCTGCTCGCCCTCAGGTGCAGCTGCAGGAGTCTG |
| | | GCCCAGGCCTGGTGAAGCCCAGCCAGACCCTGAGCATCACC |
| | | TGCACCGTGAGCGGCTTCAGCCTGGCCAGCTACAACATCCAC |
| | | TGGGTGCGGCAGCCCCCAGGCAAGGGCCTGGAGTGGCTGGG |
| | | CGTGATCTGGGCTGGCGGCAGCACCAACTACAACAGCGCCC |
| | | TGATGAGCCGGCTGACCATCAGCAAGGACAACAGCAAGAAC |
| | | CAGGTGTTCCTGAAGATGAGCAGCCTGACAGCCGCCGACAC |
| | | CGCCGTGTACTACTGCGCCAAGCGGAGCGACGACTACAGCT |
| | | GGTTCGCCTACTGGGGCCAGGGCACCCTGGTGACCGTGAGCT |
| | | CTGGCGGAGGCGGCTCTGGCGGAGGCGGCTCTGGCGGAGGC |
| | | GGCAGCGAGAACCAGATGACCCAGAGCCCCAGCAGCTTGAG |
| | | CGCCAGCGTGGGCGACCGGGTGACCATGACCTGCAGAGCCA |
| | | GCAGCAGCGTGAGCAGCAGCTACCTGCACTGGTACCAGCAG |
| | | AAGAGCGGCAAGGCCCCAAAGGTGTGGATCTACAGCACCAG |
| | | CAACCTGGCCAGCGGCGTGACCAGCCGGTTCAGCGGCAGCG |
| | | GCAGCGGCACCGACTACACCCTGACCATCAGCAGCCTGCAG |
| | | CCCGAGGACTTCGCCACCTACTACTGCCAGCAGTACAGCGGC |
| | | TACCCCATCACCTTCGGCCAGGGCACCAAGGTGGAGATCAA |
| | | GCGGTCGGATCCCACCACGACGCCAGCGCCGCGACCACCAA |
| | | CACCGGCGCCCACCATCGCGTCGCAGCCCCTGTCCCTGCGCC |
| | | CAGAGGCGTGCCGGCCAGCGGCGGGGGGCGCAGTGCACACG |
| | | AGGGGGCTGGACTTCGCCTGTGATATCTACATCTGGGCGCCC |
| | | TTGGCCGGGACTTGTGGGGTCCTTCTCCTGTCACTGGTTATCA |
| | | CCCTTTACTGCAAACGGGGCAGAAAGAAACTCCTGTATATAT |
| | | TCAAACAACCATTTATGAGACCAGTACAAACTACTCAAGAG |
| | | GAAGATGGCTGTAGCTGCCGATTTCCAGAAGAAGAAGAAGG |
| | | AGGATGTGAACTGAGAGTGAAGTTCAGCAGGAGCGCAGACG |
| | | CCCCCGCGTACCAGCAGGGCCAGAACCAGCTCTATAACGAG |
| | | CTCAATCTAGGACGAAGAGAGGAGTACGATGTTTTGGACAA |
| | | GAGACGTGGCCGGGACCCTGAGATGGGGGGAAAGCCGAGA |
| | | AGGAAGAACCCTCAGGAAGGCCTGTACAATGAACTGCAGAA |
| | | AGATAAGATGGCGGAGGCCTACAGTGAGATTGGGATGAAAG |
| | | GCGAGCGCCGGAGGGGCAAGGGGCACGATGGCCTTTACCAG |
| | | GGTCTCAGTACAGCCACCAAGGACACCTACGACGCCCTTCAC |
| | | ATGCAGGCCCTGCCCCCTCGCTAATGA |
| anti-DOTA | SEQ ID NO: 44 | ATGGCTCTGCCAGTGACAGCTCTGCTGCTTCCTCTGGCTCTCC |
| CAR | | TGCTGCATGCCGCTAGACCTGAGCAGAAACTCATCTCTGAAG |
| | | AGGATCTGCACGTGAAACTGCAAGAGTCTGGCCCCGGACTG |
| | | GTGCAGCCTTCTCAGTCTCTCTCTCTGACCTGTACCGTGTCCG |
| | | GCTTCAGCCTGACCGATTATGGCGTGCACTGGGTTCGACAAT |
| | | CCCCAGGCAAAGGACTCGAGTGGCTGGGAGTGATTTGGAGC |
| | | GGCGGAGGCACAGCCTATAACACAGCCCTGATCAGCAGACT |
| | | GAACATCTACCGGGACAACTCCAAGAACCAGGTTTTCCTGGA |
| | | AATGAACTCCCTGCAGGCAGAGGACACCGCCATGTACTACT |
| | | GCGCCAGAAGAGGCAGCTACCCCTACAATTACTTCGACGCCT |
| | | GGGGCTGTGGCACAACCGTGACAGTTTCTAGTGGCGGAGGC |
| | | GGATCTGGTGGTGGTGGTAGCGGTGGCGGAGGATCTCAGGC |
| | | CGTGGTTATTCAAGAAAGCGCCCTGACAACCCCTCCTGGCGA |
| | | GACAGTGACACTGACATGTGGCAGCTCTACAGGCGCCGTGA |
| | | CCGCCAGCAATTACGCCAATTGGGTGCAAGAGAAGCCCGAC |
| | | CACTGCTTCACAGGCCTGATCGGCGGCCACAACAATAGACCT |
| | | CCAGGCGTGCCAGCTAGATTCAGCGGATCCCTGATCGGAGA |
| | | CAAGGCCGCTCTGACAATCGCCGGCACACAGACAGAGGACG |
| | | AGGCCATCTACTTTTGCGCCCTGTGGTACAGCGACCACTGGG |
| | | TTATCGGCGGAGGAACCAGACTGACAGTGCTGGGCACAACA |
| | | ACACCCGCACCTAGACCACCAACTCCAGCACCAACAATCGC |
| | | CTCTCAACCCCTGAGTCTGAGGCCAGAGGCATGCAGACCAG |
| | | CCGCTGGCGGTGCAGTTCACACTAGAGGACTGGACTTTGCCT |
| | | GTGACATCTACATCTGGGCCCCTCTGGCTGGAACATGTGGTG |
| | | TCCTGCTGCTGTCCCTGGTCATCACCCTGTACTGCAAGCGGG |
| | | GCAGAAAGAAACTGCTGTACATCTTCAAGCAGCCCTTCATGC |
| | | GGCCCGTGCAGACCACACAAGAGGAAGATGGCTGCTCCTGC |
| | | AGATTCCCCGAGGAAGAAGAAGGCGGCTGCGAGCTGAGAGT |
| | | GAAGTTCTCCAGATCTGCCGACGCTCCCGCCTATCAGCAGGG |
| | | ACAGAACCAGCTGTACAACGAGCTGAACCTGGGGAGAAGAG |
| | | AAGAGTACGACGTGCTGGACAAGCGGAGAGGCAGAGATCCT |
| | | GAGATGGGCGGAAAGCCCCAGCGGAGAAAGAATCCTCAAGA |
| | | GGGCCTGTATAATGAGCTGCAAAAGGACAAGATGGCCGAGG |
| | | CCTACAGCGAGATCGGAATGAAGGGCGAGCGCAGAAGAGG |
| | | AAAGGGACACGACGGACTGTACCAGGGCCTGAGCACAGCCA |
| | | CCAAGGATACCTATGACGCCCTGCACATGCAGGCCCTGCCTC |
| | | CAAGATGATGA |
| anti-FITC | SEQ ID NO: 45 | ATGGCTCTGCCTGTGACAGCTCTGCTGCTGCCTCTGGCTCTGC |
| CAR | | TTCTGCATGCCGCCAGACCTGACGTGGTCATGACACAGACAC |
| | | CTCTGAGCCTGCCTGTGTCTCTGGGAGATCAGGCCAGCATCA |
| | | GCTGCAGATCTAGCCAGAGCCTGGTGCACAGCAACGGCAAC |
| | | ACCTACCTGCGGTGGTATCTGCAGAAGCCCGGCCAGTCTCCT |
| | | AAGGTGCTGATCTACAAGGTGTCCAACAGAGTGTCCGGCGT |
| | | GCCCGATAGATTTTCTGGCAGCGGCTCTGGCACCGACTTCAC |
| | | CCTGAAGATCAATAGAGTGGAAGCCGAGGACCTGGGCGTGT |
| | | ACTTCTGTAGCCAGTCTACCCACGTGCCATGGACCTTTGGCG |
| | | GCGGAACAAAGCTGGAAATCAAGAGCAGCGCCGACGACGCC |
| | | AAGAAGGACGCCGCTAAGAAGGATGACGCCAAAAAAGACG |
| | | ATGCCAAAAAGGATGGCGGCGTGAAGCTGGACGAAACAGGC |
| | | GGAGGACTTGTTCAGCCTGGCGGAGCCATGAAGCTGAGCTG |
| | | TGTGACCAGCGGCTTCACCTTCGGCCACTACTGGATGAACTG |
| | | GGTCCGACAGAGCCCTGAGAAAGGCCTGGAATGGGTCGCCC |
| | | AGTTCAGAAACAAGCCCTACAACTACGAAACCTACTACAGC |
| | | GACAGCGTGAAGGGCAGATTCACCATCAGCCGGGACGACAG |
| | | CAAGTCCAGCGTGTACCTGCAGATGAACAACCTGCGCGTGG |
| | | AAGATACCGGCATCTACTACTGTACCGGCGCCAGCTACGGCA |
| | | TGGAATATCTCGGCCAGGGCACCAGCGTGACCGTGTCTACAA |
| | | CAACCCCTGCTCCTCGGCCTCCTACACCAGCTCCTACAATTG |
| | | CCAGCCAGCCACTGTCTCTGAGGCCCGAAGCTTGTAGACCTG |
| | | CTGCAGGCGGAGCCGTGCATACAAGAGGACTGGATTTCGCC |
| | | TGCGACTTCTGGGTGCTCGTGGTTGTTGGCGGAGTGCTGGCT |
| | | TGTTACTCCCTGCTGGTTACCGTGGCCTTCATCATCTTTTGGG |
| | | TCAAGCGGGGCAGAAAGAAGCTGCTGTACATCTTCAAGCAG |
| | | CCCTTCATGCGGCCCGTGCAGACCACACAAGAGGAAGATGG |
| | | CTGCTCCTGCAGATTCCCCGAGGAAGAAGAAGGCGGCTGCG |
| | | AGCTGAGAGTGAAGTTCAGCAGATCCGCCGACGCTCCTGCCT |
| | | ATCAGCAGGGACAGAACCAGCTGTACAACGAGCTGAACCTG |
| | | GGGAGAAGAGAAGAGTACGACGTGCTGGACAAGCGGAGAG |
| | | GCAGAGATCCTGAGATGGGCGGAAAGCCCCAGCGGAGAAAG |
| | | AATCCTCAAGAGGGCCTGTATAATGAGCTGCAGAAAGACAA |
| | | GATGGCCGAGGCCTACAGCGAGATCGGAATGAAGGGCGAGC |
| | | GCAGAAGAGGCAAGGGACACGATGGACTGTACCAGGGCCTG |
| | | AGCACCGCCACCAAGGATACCTATGATGCCCTGCACATGCA |
| | | GGCCCTGCCACCTAGATGATGA |

In some embodiments, a CAR is encoded by a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 40-SEQ ID NO: 45. In some embodiments, the CAR is encoded by a sequence of any one of SEQ ID NO: 40-SEQ ID NO: 45.

### IV. Methods of Making Engineered Immune Cells

A polynucleotide expression cassette encoding the engineered cytokine receptor switch may be delivered to the immune cell using a vector, as described herein, thereby engineering the immune cell to express the engineered cytokine receptor switch. In some embodiments, the immune cell may be further engineered to express an additional component, such as a CAR or a second engineered cytokine receptor switch. The immune cell engineered to express a cytokine receptor switch may be a T-cell, a regulatory T-cell, a B-cell, a natural killer (NK) cell, a FcεRIγ deficient NK cell (g-NK cell), a neutrophil, an eosinophil, a macrophage, a γδ T-cell, or other immune cell type.

### A. Collection

Precursors to engineered immune cells of the present disclosure can be obtained from a donor. The precursor cells may be obtained from blood (e.g., peripheral blood mononuclear cells), bone marrow, lymph node tissue, cord blood, thymus tissue, tissue from a site of infection, ascites, pleural effusion, spleen tissue, tumors, or combinations thereof collected from the subject. Precursor cells may be collected from a subject using any technique known in the art (e.g., Ficoll separation). In some cases, the precursor cells are collected from blood, for example through apheresis, leukapheresis, or buffy coat preparation.

In some embodiments, the precursor cells (e.g., cells to be engineered to express a cytokine receptor switch, a CAR, or both) are immune cells collected from the donor. The immune cells can be of a single type, or a heterogeneous collection of cell types, and can include T-cells, B-cells, natural killer cells (NK cells), FεERIγ deficient NK cells (g-NK cells), macrophages, monocytes, basophils, eosinophils, neutrophils, megakaryocytes, thrombocytes, or combinations thereof. In some cases, the immune cells are CD4⁺ or CD8⁺ T-cells. In some cases, the immune cells are naïve T and/or naïve B-cells.

The immune cells can be enriched for specific cell types. For many of the methods disclosed herein, blood-derived immune cells are separated from other whole blood components, for example through monocyte depletion, centrifugation, filtration, or clotting. The immune cells can also be subjected to positive or negative selection for certain cell types. In many cases, immune cells collected from a subject are separated from other peripheral blood mononuclear cells (PBMCs) through negative selection for surface markers expressed by non-target cells, such as CD25, CD45, CD103, or FOXP3. In specific cases, the immune cells are depleted of memory T and/or B-cells (e.g., central memory T-cells, effector memory T-cells, virtual memory T-cells, memory B-cells, etc.). In some cases, the immune cells are enriched for a particular type of T or B-cell, such as a γδ T-cell, a T_{H}1 cell, a T_{H}2 cell, a T_{H}17 cell, a T_{H}22 cell, a T helper cell, a T regulatory cell, or a combination thereof. As a nonlimiting example, immune cell enrichment can include selectively binding one or more cell type-specific surface markers on a magnetically separatable bead or column. In some cases, the immune cells are of a single cell type.

The precursor cells can also include a totipotent, pluripotent, multipotent, or oligopotent cell. In many such cases, the precursor cells include immune precursor cells, such as common myeloid progenitor cells, granulocyte progenitor cells, myeloblasts, monocytes, common lymphoid progenitor cells, lymphoid progenitor cells, progenitor B-cells, or combinations thereof. In some cases, the precursor cells include a stem cell, such as a tetraploid reprogrammed cell, an induced pluripotent stem cell, an embryoblast, a lymphoid stem cell, or a myeloid stem cell.

### B. Transfection

A precursor cell can be transfected with a nucleic acid to generate an engineered cell of the present disclosure. The nucleic acid can encode an engineered protein such as a CAR and/or an engineered cytokine receptor switch. The nucleic acid can also encode elements for gene editing, nucleases, reverse transcriptases, integrases, recombinases, and combinations thereof. As non-limiting examples, a nuclease encoded by the nucleic acid can include a zinc finger nuclease, a transcription activator-like effector nuclease (TALEN), a Cas3 nuclease, a Cas9 nuclease, a CRISPR/Cas12 nuclease, a CRISPR/Cas14 nuclease, a Fok1 nuclease, or a combination thereof. The nucleic acid can also encode additional transcripts and proteins which further affect cell phenotype, such as small interfering RNA (siRNA), microRNA (miRNA), short hairpin RNA (shRNA), ribozymes, transcription factors, or immunomodulatory elements.

The immune cells can be transfected with a polynucleotide expression cassette encoding the engineered cytokine receptor switch and/or CAR, thereby engineering the immune cell to express the cytokine receptor switch and/or CAR. The polynucleotide can be a plasmid, a cosmid, a viral vector, or a combination thereof. The polynucleotide can be naked or delivered via a nonviral vector (e.g., liposomes or lipid nanoparticles). The expression cassette can have one or more control sequences which affect expression of the engineered cytokine receptor switch. The expression cassette can also include one or more selection markers.

The polynucleotide expression cassette may comprise an RNA sequence or a DNA sequence encoding an engineered cytokine receptor switch and/or CAR, or an RNA sequence or a DNA sequence reverse complementary to a sequence encoding an engineered cytokine receptor switch and/or CAR. In some embodiments, the polynucleotide encoding the engineered cytokine receptor switch and/or CAR may be part of a polynucleotide expression cassette capable of expressing the engineered cytokine receptor switch and/or CAR in a cell (e.g., an immune cell). The polynucleotide expression cassette may comprise a promoter, an open reading frame (e.g., encoding the engineered cytokine receptor switch and/or CAR), a 3′ untranslated region, or combinations thereof. In some embodiments, the polynucleotide expression cassette may comprise two or more open reading frames. For example, the polynucleotide expression cassette may comprise a first open reading frame encoding the engineered cytokine receptor switch and a second open reading frame encoding a CAR. The expression cassette may further comprise an origin of replication, a restriction endonuclease site, a selectable marker, or combinations thereof. The expression cassette may be capable of expressing both the engineered cytokine receptor switch and the CAR in a cell (e.g., an immune cell). In some embodiments, the cell may be a mammalian cell (e.g., a human cell). For example, the cell may be a human T-cell. A polynucleotide or polynucleotide expression cassette may be obtained using recombinant methods known in the art. Alternatively or in addition, the polynucleotide or polynucleotide expression cassette may be generated synthetically.

Also provided herein are vectors comprising the polynucleotide expression cassette. The vector may be capable of delivering the polynucleotide expression cassette to a target cell (e.g., an immune cell). Upon delivery, a protein encoded by the polynucleotide expression cassette (e.g., an engineered cytokine receptor switch, a CAR, or combinations thereof) may be expressed in the cell. In some embodiments, the vector may be a viral vector. In some cases, the viral vector comprises a lentiviral vector, an adeno-associated viral vector, a vaccinia viral vector, a poxvirus viral vector, a herpes viral vector, an alphavirus viral vector, gamma retrovirus, a polyoma viral vector, or a combination thereof. In some cases, the viral vector is a lentiviral vector. In some cases, the viral vector has a titer of between about 106 and about 109 virions per ml. In some cases, transfection includes delivery of nonviral vectors (e.g., in a lipid or chitosan nanoparticle or with a colloidal dispersion system), for example mRNA encoding an engineered cytokine receptor switch, a CAR, a transcription factor, or a nuclease (e.g., a zing finger protein, a TAL-effector domain protein, or a CRISPR/Cas nuclease); DNA (e.g., DNA encoding an engineered cytokine receptor switch) for targeted recombination; a nuclease, signaling molecule, or transcription factor; siRNA; miRNA; or a combination thereof. In some cases, between about 10% and about 30%, between about 10% and about 50%, between about 20% and about 60%, between about 40% and about 90%, or between about 60% and about 95% of naïve T cells and B cells from among the immune cell population are transfected.

A vector encoding an engineered cytokine receptor switch may be generated and delivered to an immune cell using standard cloning and gene delivery protocols, for example as described in Sambrook et al. (2001, Molecular Cloning: A Laboratory Manual, Cold Spring Harbor Laboratory, New York), which is herein incorporated by reference. A vector may encode a selectable marker, a reporter gene, or both to facilitate selection of cells (e.g., immune cells) successfully transfected and expressing a protein encoded by the expression cassette (e.g., the engineered cytokine receptor switch, the CAR, or both).

Alternatively, or in addition thereto, a polynucleotide expression cassette may be introduced into a target cell using physical or chemical means. In some embodiments, a polynucleotide expression cassette may be introduced into a target cell using calcium phosphate precipitation, lipofection, particle bombardment, microinjection, electroporation, and the like. In some embodiments, the polynucleotide expression cassette may be introduced using colloidal dispersion systems (e.g., macromolecule complexes), nanocapsules, microspheres, beads, lipid-based systems (e.g., oil-in-water emulsions, micelles, mixed micelles, liposomes), and the like. Additional transfection or infection methods are described in Sambrook et al. (2001, Molecular Cloning: A Laboratory Manual, Cold Spring Harbor Laboratory, New York).

Examples of polynucleotide constructs encoding an engineered cytokine switch (SMAR) and a CAR are provided in Table 9.

TABLE 9
Representative Examples of SMAR-CAR Constructs

| SMAR-CAR | | |
|---|---|---|
| Construct | SEQ ID NO | Sequence |
| anti-FITC | SEQ ID NO: 46 | ATGGCTGCTCCTGCTCTGTCTTGGAGACTGCCCCTGCTGATTC |
| IL2Rβ SMAR- | | TGCTGCTGCCTCTGGCTACATCTTGGGCCTCTGCCGATTACA |
| F2A-anti- | | AGGATGACGACGATAAGGACGTGGTCATGACACAGACACCA |
| FITC IL2Rγ | | CTGAGCCTGCCTGTGTCTCTGGGAGATCAGGCCAGCATCAGC |
| SMAR-T2A- | | TGCAGATCCAGCCAGTCTCTGGTGCACAGCAACGGCAACAC |
| bb2121 | | CTACCTGCGGTGGTATCTGCAGAAGCCCGGCCAGTCTCCTAA |
| | | GGTGCTGATCTACAAGGTGTCCAACAGAGTGTCCGGCGTGCC |
| | | CGATAGATTTTCTGGCAGCGGCTCTGGCACCGACTTCACCCT |
| | | GAAGATCAATAGAGTGGAAGCCGAGGACCTGGGCGTGTACT |
| | | TCTGTAGCCAGTCTACCCACGTGCCATGGACCTTTGGCGGCG |
| | | GAACAAAGCTGGAAATCAAGAGCAGCGCCGACGACGCCAAG |
| | | AAGGACGCCGCTAAGAAGGATGACGCAAAGAAAGACGATG |
| | | CCAAAAAGGATGGCGGCGTGAAGCTGGACGAAACAGGCGG |
| | | AGGACTTGTTCAGCCTGGCGGAGCCATGAAGCTGAGCTGTGT |
| | | GACCAGCGGCTTCACCTTCGGCCACTACTGGATGAACTGGGT |
| | | CCGACAGAGCCCTGAGAAAGGCCTGGAATGGGTCGCCCAGT |
| | | TCAGAAACAAGCCCTACAACTACGAAACCTACTACAGCGAC |
| | | AGCGTGAAGGGCAGATTCACCATCAGCCGGGACGACAGCAA |
| | | GTCCAGCGTGTACCTGCAGATGAACAACCTGCGCGTGGAAG |
| | | ATACCGGCATCTACTACTGTACCGGCGCCAGCTACGGCATGG |
| | | AATATCTCGGCCAGGGCACCAGCGTGACCGTGTCTACAACA |
| | | ACCCCTGCTCCTCGGCCTCCTACACCAGCTCCTACAATTGCC |
| | | AGCCAGCCACTGTCTCTGAGGCCCGAAGCTTGTAGACCTGCT |
| | | GCAGGCGGAGCCGTGCATACAAGAGGACTGGATTTCGCCTG |
| | | CGACATCCCCTGGCTGGGACATCTGCTTGTTGGACTGTCTGG |
| | | CGCCTTCGGCTTCATCATCCTGGTGTATCTGCTGATCAACTGC |
| | | CGGAACACAGGCCCTTGGCTGAAGAAAGTGCTGAAGTGCAA |
| | | CACCCCTGATCCGAGCAAGTTCTTTAGCCAGCTGAGCAGCGA |
| | | GCATGGCGGCGACGTTCAGAAATGGCTGTCTAGCCCATTTCC |
| | | TAGCAGCAGCTTCAGCCCAGGTGGACTGGCCCCTGAGATTAG |
| | | CCCTCTGGAAGTGCTGGAACGGGACAAAGTGACCCAGCTGC |
| | | TCCTCCAGCAGGATAAGGTGCCAGAACCTGCCAGCCTGTCCA |
| | | GCAATCACAGCCTGACCAGCTGCTTTACCAACCAGGGCTACT |
| | | TCTTCTTCCATCTGCCTGACGCTCTGGAAATCGAGGCCTGCC |
| | | AGGTGTACTTCACCTACGATCCCTACAGCGAAGAGGACCCCG |
| | | ATGAAGGTGTTGCTGGCGCCCCTACAGGATCTTCTCCACAGC |
| | | CTCTGCAACCTCTGAGCGGCGAGGATGATGCCTACTGCACCT |
| | | TTCCAAGCAGGGACGACCTGCTCCTGTTCAGCCCATCTCTGC |
| | | TCGGAGGACCATCTCCTCCATCTACAGCTCCAGGCGGATCTG |
| | | GCGCTGGCGAGGAAAGAATGCCACCTAGCCTGCAAGAGCGG |
| | | GTGCCCAGAGATTGGGATCCTCAACCTCTCGGCCCTCCAACA |
| | | CCTGGCGTGCCAGATCTCGTGGACTTTCAGCCTCCTCCAGAG |
| | | CTGGTGCTGAGAGAAGCTGGCGAAGAAGTGCCAGACGCTGG |
| | | CCCTAGAGAGGGCGTTAGCTTTCCTTGGAGCAGACCTCCTGG |
| | | ACAGGGCGAGTTTAGGGCCCTGAATGCTAGACTGCCTCTGAA |
| | | CACCGACGCCTACCTGTCTCTGCAAGAACTGCAGGGACAAG |
| | | ACCCCACACACCTCGTTGGAAGCGGAGTGAAGCAGACCCTG |
| | | AACTTCGACCTGCTGAAACTGGCCGGCGACGTGGAAAGCAA |
| | | CCCTGGACCTATGCTGAAGCCCAGCCTGCCTTTTACCAGCCT |
| | | GCTGTTCCTGCAGCTCCCACTGCTTGGCGTGGGATACCCATA |
| | | CGATGTTCCAGATTACGCTGATGTCGTGATGACCCAGACTCC |
| | | ACTCTCTCTGCCTGTCAGCCTGGGCGATCAAGCCTCCATCTC |
| | | CTGTAGAAGCAGCCAGAGCCTGGTCCACTCCAATGGCAATA |
| | | CCTATCTCAGATGGTATCTCCAAAAGCCTGGGCAGAGCCCAA |
| | | AAGTCCTCATCTACAAAGTCTCTAATCGCGTCAGCGGAGTGC |
| | | CCGACAGGTTTAGCGGTTCTGGAAGCGGCACAGATTTCACGC |
| | | TCAAGATTAACCGCGTCGAGGCCGAAGATCTGGGAGTCTATT |
| | | TCTGCAGTCAGAGCACACATGTGCCCTGGACATTCGGCGGAG |
| | | GCACCAAGCTCGAGATCAAGTCTAGCGCCGATGATGCTAAG |
| | | AAAGATGCAGCTAAAAAGGATGATGCCAAGAAGGACGACGC |
| | | CAAGAAAGATGGCGGAGTCAAACTCGATGAGACTGGCGGAG |
| | | GCCTGGTGCAACCCGGTGGTGCTATGAAGCTGTCTTGCGTGA |
| | | CCTCCGGCTTTACATTTGGGCATTATTGGATGAATTGGGTTC |
| | | GCCAATCTCCAGAGAAGGGCCTCGAGTGGGTTGCACAGTTTC |
| | | GGAACAAACCGTACAATTACGAGACATATTACTCCGACTCCG |
| | | TGAAAGGCCGGTTCACAATCTCCCGCGACGACTCCAAGTCCT |
| | | CTGTCTATCTTCAAATGAACAATCTGAGAGTCGAGGACACGG |
| | | GGATCTACTATTGCACAGGCGCCTCTTATGGAATGGAATACC |
| | | TTGGACAGGGAACCTCTGTGACCGTCAGCACCACAACACCC |
| | | GCTCCTAGACCTCCAACTCCTGCTCCAACAATCGCCTCTCAA |
| | | CCCCTCAGCCTCAGACCTGAGGCATGTAGACCAGCAGCTGGC |
| | | GGTGCAGTTCACACCAGAGGCCTGGACTTTGCCTGTGACGTC |
| | | GTGATCAGCGTGGGCAGCATGGGCCTGATCATCTCCCTGCTG |
| | | TGTGTGTACTTTTGGCTCGAGCGGACCATGCCTCGGATCCCC |
| | | ACACTGAAGAACCTCGAGGATCTGGTCACCGAGTACCACGG |
| | | CAACTTCTCTGCTTGGAGCGGCGTGTCAAAAGGACTGGCCGA |
| | | AAGCCTGCAGCCTGACTACTCCGAGAGACTGTGCCTGGTGTC |
| | | TGAGATCCCTCCTAAAGGCGGCGCTCTCGGAGAAGGACCTG |
| | | GTGCCTCTCCATGCAATCAGCACAGCCCTTATTGGGCCCCTC |
| | | CTTGCTACACCCTGAAACCTGAGACAGGCAGCGGCGAAGGC |
| | | AGAGGCTCTCTTCTTACATGTGGCGACGTCGAAGAGAATCCC |
| | | GGACCAATGGCACTCCCCGTCACCGCCCTTCTCTTGCCCCTC |
| | | GCCCTGCTGCTGCATGCTGCCAGGCCCGACATTGTGCTCACT |
| | | CAGTCACCTCCCAGCCTGGCCATGAGCCTGGGAAAAAGGGC |
| | | CACCATCTCCTGTAGAGCCAGTGAGTCCGTCACAATCTTGGG |
| | | GAGCCATCTTATTCACTGGTATCAGCAGAAGCCCGGGCAGCC |
| | | TCCAACCCTTCTTATTCAGCTCGCGTCAAACGTCCAGACGGG |
| | | TGTACCTGCCAGATTTTCTGGTAGCGGGTCCCGCACTGATTTT |
| | | ACACTGACCATAGATCCAGTGGAAGAAGACGATGTGGCCGT |
| | | GTATTATTGTCTGCAGAGCAGAACGATTCCTCGCACATTTGG |
| | | TGGGGGTACTAAGCTGGAGATTAAGGGAAGCACGTCCGGCT |
| | | CAGGGAAGCCGGGCTCCGGCGAGGGAAGCACGAAGGGGCA |
| | | AATTCAGCTGGTCCAGAGCGGACCTGAGCTGAAAAAACCCG |
| | | GCGAGACTGTTAAGATCAGTTGTAAAGCATCTGGCTATACCT |
| | | TCACCGACTACAGCATAAATTGGGTGAAACGGGCCCCTGGA |
| | | AAGGGCCTCAAATGGATGGGTTGGATCAATACCGAAACTAG |
| | | GGAGCCTGCTTATGCATATGACTTCCGCGGGAGATTCGCCTT |
| | | TTCACTCGAGACATCTGCCTCTACTGCTTACCTCCAAATAAA |
| | | CAACCTCAAGTATGAAGATACAGCCACTTACTTTTGCGCCCT |
| | | CGACTATAGTTACGCCATGGACTACTGGGGACAGGGAACCT |
| | | CCGTTACCGTCAGTTCCGCGGCCGCAACCACAACACCTGCTC |
| | | CAAGGCCCCCCACACCCGCTCCAACTATAGCCAGCCAACCAT |
| | | TGAGCCTCAGACCTGAAGCTTGCAGGCCCGCAGCAGGAGGC |
| | | GCCGTCCATACGCGAGGCCTGGACTTCGCGTGTGATATTTAT |
| | | ATTTGGGCCCCTTTGGCCGGAACATGTGGGGTGTTGCTTCTC |
| | | TCCCTTGTGATCACTCTGTATTGTAAGCGCGGGAGAAAGAAG |
| | | CTCCTGTACATCTTCAAGCAGCCTTTTATGCGACCTGTGCAA |
| | | ACCACTCAGGAAGAAGATGGGTGTTCATGCCGCTTCCCCGAG |
| | | GAGGAAGAAGGAGGGTGTGAACTGAGGGTGAAATTTTCTAG |
| | | AAGCGCCGATGCTCCCGCATATCAGCAGGGTCAGAATCAGC |
| | | TCTACAATGAATTGAATCTCGGCAGGCGAGAAGAGTACGAT |
| | | GTTCTGGACAAGAGACGGGGCAGGGATCCCGAGATGGGGGG |
| | | AAAGCCCCGGAGAAAAAATCCTCAGGAGGGGTTGTACAATG |
| | | AGCTGCAGAAGGACAAGATGGCTGAAGCCTATAGCGAGATC |
| | | GGAATGAAAGGCGAAAGACGCAGAGGCAAGGGGCATGACG |
| | | GTCTGTACCAGGGTCTCTCTACAGCCACCAAGGACACTTATG |
| | | ATGCGTTGCATATGCAAGCCTTGCCACCCCGCTGATGA |
| anti-FITC | SEQ ID NO: 47 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-bb2121- | | GGACGTGGTCATGACACAGACCCCACTGTCTCTGCCTGTGTC |
| P2A- | | TCTGGGAGATCAGGCCAGCATCAGCTGCAGATCTAGCCAGA |
| Truncated | | GCCTGGTGCACAGCAACGGCAACACCTACCTGCGGTGGTATC |
| CD19 | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCACTCCCCGTCACCGCCCTTCTCTTGCCCCTCG |
| | | CCCTGCTGCTGCATGCTGCCAGGCCCGACATTGTGCTCACTC |
| | | AGTCACCTCCCAGCCTGGCCATGAGCCTGGGAAAAAGGGCC |
| | | ACCATCTCCTGTAGAGCCAGTGAGTCCGTCACAATCTTGGGG |
| | | AGCCATCTTATTCACTGGTATCAGCAGAAGCCCGGGCAGCCT |
| | | CCAACCCTTCTTATTCAGCTCGCGTCAAACGTCCAGACGGGT |
| | | GTACCTGCCAGATTTTCTGGTAGCGGGTCCCGCACTGATTTT |
| | | ACACTGACCATAGATCCAGTGGAAGAAGACGATGTGGCCGT |
| | | GTATTATTGTCTGCAGAGCAGAACGATTCCTCGCACATTTGG |
| | | TGGGGGTACTAAGCTGGAGATTAAGGGAAGCACGTCCGGCT |
| | | CAGGGAAGCCGGGCTCCGGCGAGGGAAGCACGAAGGGGCA |
| | | AATTCAGCTGGTCCAGAGCGGACCTGAGCTGAAAAAACCCG |
| | | GCGAGACTGTTAAGATCAGTTGTAAAGCATCTGGCTATACCT |
| | | TCACCGACTACAGCATAAATTGGGTGAAACGGGCCCCTGGA |
| | | AAGGGCCTCAAATGGATGGGTTGGATCAATACCGAAACTAG |
| | | GGAGCCTGCTTATGCATATGACTTCCGCGGGAGATTCGCCTT |
| | | TTCACTCGAGACATCTGCCTCTACTGCTTACCTCCAAATAAA |
| | | CAACCTCAAGTATGAAGATACAGCCACTTACTTTTGCGCCCT |
| | | CGACTATAGTTACGCCATGGACTACTGGGGACAGGGAACCT |
| | | CCGTTACCGTCAGTTCCGCGGCCGCAACCACAACACCTGCTC |
| | | CAAGGCCCCCCACACCCGCTCCAACTATAGCCAGCCAACCAT |
| | | TGAGCCTCAGACCTGAAGCTTGCAGGCCCGCAGCAGGAGGC |
| | | GCCGTCCATACGCGAGGCCTGGACTTCGCGTGTGATATTTAT |
| | | ATTTGGGCCCCTTTGGCCGGAACATGTGGGGTGTTGCTTCTC |
| | | TCCCTTGTGATCACTCTGTATTGTAAGCGCGGGAGAAAGAAG |
| | | CTCCTGTACATCTTCAAGCAGCCTTTTATGCGACCTGTGCAA |
| | | ACCACTCAGGAAGAAGATGGGTGTTCATGCCGCTTCCCCGAG |
| | | GAGGAAGAAGGAGGGTGTGAACTGAGGGTGAAATTTTCTAG |
| | | AAGCGCCGATGCTCCCGCATATCAGCAGGGTCAGAATCAGC |
| | | TCTACAATGAATTGAATCTCGGCAGGCGAGAAGAGTACGAT |
| | | GTTCTGGACAAGAGACGGGGCAGGGATCCCGAGATGGGGGG |
| | | AAAGCCCCGGAGAAAAAATCCTCAGGAGGGGTTGTACAATG |
| | | AGCTGCAGAAGGACAAGATGGCTGAAGCCTATAGCGAGATC |
| | | GGAATGAAAGGCGAAAGACGCAGAGGCAAGGGGCATGACG |
| | | GTCTGTACCAGGGTCTCTCTACAGCCACCAAGGACACTTATG |
| | | ATGCGTTGCATATGCAAGCCTTGCCACCCCGCGGATCTGGCG |
| | | CCACAAACTTCTCACTGCTGAAACAGGCCGGCGACGTCGAA |
| | | GAGAATCCTGGGCCTATGCCTCCTCCTCGGCTGCTGTTCTTCC |
| | | TGCTGTTTCTGACCCCTATGGAAGTGCGGCCCGAGGAACCTC |
| | | TGGTCGTGAAAGTTGAAGAGGGCGACAACGCCGTGCTGCAG |
| | | TGTCTGAAGGGCACATCTGATGGCCCCACACAGCAGCTGACC |
| | | TGGTCTAGAGAGAGCCCTCTGAAGCCCTTCCTGAAGCTCAGT |
| | | CTGGGACTGCCTGGACTGGGCATCCATATGAGGCCACTGGCC |
| | | ATCTGGCTGTTCATCTTTAACGTGTCCCAGCAGATGGGAGGC |
| | | TTCTACCTGTGTCAGCCTGGACCTCCTAGCGAGAAAGCTTGG |
| | | CAGCCTGGCTGGACCGTGAATGTGGAAGGATCCGGCGAGCT |
| | | GTTCCGGTGGAATGTCTCTGATCTCGGCGGCCTCGGATGCGG |
| | | CCTGAAGAATAGATCTAGCGAGGGCCCTAGCAGCCCCTCCG |
| | | GAAAACTGATGAGCCCCAAGCTGTACGTGTGGGCCAAAGAC |
| | | AGACCCGAGATTTGGGAGGGCGAGCCTCCTTGTCTGCCACCT |
| | | AGAGACAGCCTGAATCAGAGCCTGAGCCAGGACCTGACAAT |
| | | GGCCCCTGGATCTACACTGTGGCTGAGCTGCGGAGTGCCTCC |
| | | TGACAGTGTGTCTAGAGGCCCACTGAGCTGGACACACGTGC |
| | | ACCCTAAGGGCCCTAAGAGCCTGCTGAGTCTGGAACTGAAG |
| | | GACGACAGGCCCGCCAGAGATATGTGGGTCATGGAAACCGG |
| | | ACTGCTGCTCCCTAGAGCCACTGCTCAAGATGCCGGCAAGTA |
| | | CTATTGCCACCGGGGCAACCTGACCATGAGCTTCCACCTGGA |
| | | AATTACCGCCAGACCAGTGCTGTGGCATTGGCTGCTTAGAAC |
| | | CGGCGGATGGAAGGTGTCAGCCGTGACTCTGGCCTACCTGAT |
| | | CTTTTGTCTGTGCAGCCTCGTGGGCATCCTGCATCTGCAAAG |
| | | AGCCCTGGTCCTGCGGCGGAAGCGGAAGAGAATGACCGATC |
| | | CTACCAGACGGTTCTGATGA |
| anti-FITC | SEQ ID NO: 48 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-Carvykti | | GGACGTGGTCATGACACAGACCCCACTGTCTCTGCCTGTGTC |
| | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCTCTGCCTGTCACCGCTCTGCTGCTGCCTCTG |
| | | GCTCTGCTGCTGCACGCTGCTCGCCCTCAGGTCAAACTGGAA |
| | | GAAAGTGGGGGAGGCCTGGTGCAGGCAGGACGGAGCCTGCG |
| | | CCTGAGCTGCGCAGCATCCGAGCACACCTTCAGCTCCCACGT |
| | | GATGGGCTGGTTTCGGCAGGCCCCAGGCAAGGAGAGAGAGT |
| | | CCGTGGCCGTGATCGGCTGGAGGGACATCTCCACATCTTACG |
| | | CCGATTCTGTGAAGGGCCGGTTCACCATCAGCAGAGACAAC |
| | | GCCAAGAAGACACTGTATCTGCAGATGAATAGCCTGAAGCC |
| | | CGAGGACACCGCCGTGTACTATTGCGCAGCAAGGAGAATCG |
| | | ACGCAGCAGACTTTGATTCCTGGGGCCAGGGCACCCAGGTG |
| | | ACAGTGTCTAGCGGAGGAGGAGGATCTGGAGGAGGAGGAA |
| | | GCGGAGGAGGAGGATCCGAGGTGCAGCTGGTGGAGTCTGGA |
| | | GGCGGCCTGGTGCAGGCCGGAGGCTCTCTGAGGCTGAGCTG |
| | | TGCAGCATCCGGAAGAACCTTCACAATGGGCTGGTTTAGGCA |
| | | GGCACCAGGAAAGGAGAGGGAGTTCGTGGCAGCAATCAGCC |
| | | TGTCCCCTACCCTGGCCTACTATGCCGAGTCCGTGAAGGGCA |
| | | GGTTTACCATCTCTCGCGATAACGCCAAGAATACAGTGGTGC |
| | | TGCAGATGAACAGCCTGAAACCTGAGGACACAGCCCTGTAC |
| | | TATTGTGCCGCCGATCGGAAGAGCGTGATGAGCATTAGACCC |
| | | GATTATTGGGGACAGGGCACACAGGTGACAGTGAGTAGCAC |
| | | TAGTACCACGACGCCAGCGCCGCGACCACCAACACCGGCGC |
| | | CCACCATCGCGTCGCAGCCCCTGTCCCTGCGCCCAGAGGCGT |
| | | GCCGGCCAGCGGGGGGGGCGCAGTGCACACGAGGGGGCTG |
| | | GACTTCGCCTGTGATATCTACATCTGGGCGCCCTTGGCCGGG |
| | | ACTTGTGGGGTCCTTCTCCTGTCACTGGTTATCACCCTTTACT |
| | | GCAAACGGGGCAGAAAGAAACTCCTGTATATATTCAAACAA |
| | | CCATTTATGAGACCAGTACAAACTACTCAAGAGGAAGATGG |
| | | CTGTAGCTGCCGATTTCCAGAAGAAGAAGAAGGAGGATGTG |
| | | AACTGAGAGTGAAGTTCAGCAGGAGCGCAGACGCCCCCGCG |
| | | TACCAGCAGGGCCAGAACCAGCTCTATAACGAGCTCAATCT |
| | | AGGACGAAGAGAGGAGTACGATGTTTTGGACAAGAGACGTG |
| | | GCCGGGACCCTGAGATGGGGGGAAAGCCGAGAAGGAAGAA |
| | | CCCTCAGGAAGGCCTGTACAATGAACTGCAGAAAGATAAGA |
| | | TGGCGGAGGCCTACAGTGAGATTGGGATGAAAGGCGAGCGC |
| | | CGGAGGGGCAAGGGGCACGATGGCCTTTACCAGGGTCTCAG |
| | | TACAGCCACCAAGGACACCTACGACGCCCTTCACATGCAGG |
| | | CCCTGCCCCCTCGCTAATGA |
| anti-FITC | SEQ ID NO: 49 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-anti- | | GGACGTGGTCATGACACAGACCCCACTGTCTCTGCCTGTGTC |
| CD123 CAR | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCCCTGCCAGTAACTGCTCTTCTCCTGCCACTC |
| | | GCTCTTCTGCTGCACGCCGCACGGCCTCAAATTCAACTTGTG |
| | | CAGAGTGGCCCCGAGCTGAAAAAGCCCGGCGAAACAGTAAA |
| | | GATCTCTTGTAAGGCGTCTGGATATATCTTTACCAATTACGG |
| | | TATGAACTGGGTCAAACAGGCACCCGGTAAAAGCTTTAAAT |
| | | GGATGGGCTGGATAAATACTTATACCGGAGAAAGCACGTAT |
| | | AGCGCCGACTTCAAGGGTAGATTTGCGTTCAGCCTGGAAACG |
| | | TCCGCCAGCACTGCATACTTGCACATCAATGACTTGAAAAAT |
| | | GAGGATACCGCGACCTACTTTTGTGCTAGAAGTGGTGGATAC |
| | | GATCCCATGGATTACTGGGGACAGGGGACCTCTGTCACAGTC |
| | | AGTTCTGGGGGCGGCGGTTCCGGAGGTGGCGGCTCAGGTGG |
| | | AGGTGGGTCAGATATCGTGTTGACACAAAGCCCTGCTTCCCT |
| | | GGCCGTCAGCTTGGGACAGAGAGCTACTATCTCCTGTAGAGC |
| | | AAGTGAGTCTGTCGACAATTACGGCAACACCTTTATGCACTG |
| | | GTACCAGCAGAAGCCAGGCCAGCCACCGAAGCTGTTGATTT |
| | | ACCGGGCCTCAAACCTCGAAAGCGGGATTCCAGCTAGATTTT |
| | | CTGGCAGCGGGAGTCGAACCGACTTCACACTGACTATTAACC |
| | | CAGTGGAAGCAGATGACGTAGCCACATATTACTGTCAGCAG |
| | | AGCAACGAAGACCCCCCGACGTTTGGTGCTGGAACCAAACT |
| | | GGAGCTGAAAACGACAACCCCCGCTCCACGACCTCCTACTCC |
| | | CGCTCCCACAATCGCATCTCAGCCACTCTCCTTGAGGCCTGA |
| | | GGCCTGCCGACCAGCTGCAGGCGGCGCAGTACACACCAGAG |
| | | GCCTCGACTTTGCTTGCGATATCTACATATGGGCGCCCCTGG |
| | | CCGGGACGTGTGGGGTATTGCTCCTGAGCCTTGTGATCACTC |
| | | TTTACTGCAAAAGAGGTCGGAAAAAGCTTCTTTACATCTTTA |
| | | AACAGCCGTTCATGCGCCCGGTGCAAACAACACAGGAGGAG |
| | | GATGGATGCAGCTGCAGGTTTCCGGAGGAGGAGGAAGGCGG |
| | | GTGTGAGCTCCGGGTAAAATTCTCCAGAAGCGCAGACGCGC |
| | | CAGCCTATCAGCAGGGGCAGAATCAGCTCTATAACGAACTC |
| | | AATCTTGGGCGACGGGAAGAATACGATGTACTTGATAAACG |
| | | CCGAGGTCGCGACCCCGAAATGGGAGGCAAGCCTCAGAGAC |
| | | GCAAGAATCCCCAAGAAGGCCTGTATAATGAACTCCAGAAG |
| | | GATAAAATGGCGGAGGCCTACAGCGAAATTGGTATGAAGGG |
| | | AGAAAGACGACGCGGTAAAGGCCACGACGGCCTTTACCAGG |
| | | GCCTGTCAACAGCTACCAAAGATACGTATGATGCACTTCACA |
| | | TGCAAGCCCTGCCGCCCCGGTGATGA |
| anti-FITC | SEQ ID NO: 50 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-anti-GD2 | | GGACGTGGTCATGACACAGACCCCACTGTCTCTGCCTGTGTC |
| CAR | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCTCTGCCTGTCACCGCTCTGCTGCTGCCTCTG |
| | | GCTCTGCTGCTGCACGCTGCTCGCCCTCAGGTGCAGCTGCAG |
| | | GAGTCTGGCCCAGGCCTGGTGAAGCCCAGCCAGACCCTGAG |
| | | CATCACCTGCACCGTGAGCGGCTTCAGCCTGGCCAGCTACAA |
| | | CATCCACTGGGTGCGGCAGCCCCCAGGCAAGGGCCTGGAGT |
| | | GGCTGGGCGTGATCTGGGCTGGCGGCAGCACCAACTACAAC |
| | | AGCGCCCTGATGAGCCGGCTGACCATCAGCAAGGACAACAG |
| | | CAAGAACCAGGTGTTCCTGAAGATGAGCAGCCTGACAGCCG |
| | | CCGACACCGCCGTGTACTACTGCGCCAAGCGGAGCGACGAC |
| | | TACAGCTGGTTCGCCTACTGGGGCCAGGGCACCCTGGTGACC |
| | | GTGAGCTCTGGCGGAGGCGGCTCTGGCGGAGGCGGCTCTGG |
| | | CGGAGGCGGCAGCGAGAACCAGATGACCCAGAGCCCCAGCA |
| | | GCTTGAGCGCCAGCGTGGGCGACCGGGTGACCATGACCTGC |
| | | AGAGCCAGCAGCAGCGTGAGCAGCAGCTACCTGCACTGGTA |
| | | CCAGCAGAAGAGCGGCAAGGCCCCAAAGGTGTGGATCTACA |
| | | GCACCAGCAACCTGGCCAGCGGCGTGACCAGCCGGTTCAGC |
| | | GGCAGCGGCAGCGGCACCGACTACACCCTGACCATCAGCAG |
| | | CCTGCAGCCCGAGGACTTCGCCACCTACTACTGCCAGCAGTA |
| | | CAGCGGCTACCCCATCACCTTCGGCCAGGGCACCAAGGTGG |
| | | AGATCAAGCGGTCGGATCCCACCACGACGCCAGCGCCGCGA |
| | | CCACCAACACCGGCGCCCACCATCGCGTCGCAGCCCCTGTCC |
| | | CTGCGCCCAGAGGCGTGCCGGCCAGCGGCGGGGGGCGCAGT |
| | | GCACACGAGGGGGCTGGACTTCGCCTGTGATATCTACATCTG |
| | | GGCGCCCTTGGCCGGGACTTGTGGGGTCCTTCTCCTGTCACT |
| | | GGTTATCACCCTTTACTGCAAACGGGGCAGAAAGAAACTCCT |
| | | GTATATATTCAAACAACCATTTATGAGACCAGTACAAACTAC |
| | | TCAAGAGGAAGATGGCTGTAGCTGCCGATTTCCAGAAGAAG |
| | | AAGAAGGAGGATGTGAACTGAGAGTGAAGTTCAGCAGGAGC |
| | | GCAGACGCCCCCGCGTACCAGCAGGGCCAGAACCAGCTCTA |
| | | TAACGAGCTCAATCTAGGACGAAGAGAGGAGTACGATGTTT |
| | | TGGACAAGAGACGTGGCCGGGACCCTGAGATGGGGGGAAAG |
| | | CCGAGAAGGAAGAACCCTCAGGAAGGCCTGTACAATGAACT |
| | | GCAGAAAGATAAGATGGCGGAGGCCTACAGTGAGATTGGGA |
| | | TGAAAGGCGAGCGCCGGAGGGGCAAGGGGCACGATGGCCTT |
| | | TACCAGGGTCTCAGTACAGCCACCAAGGACACCTACGACGC |
| | | CCTTCACATGCAGGCCCTGCCCCCTCGCTAATGA |
| anti-FITC | SEQ ID NO: 51 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-anti- | | GGACGTGGTCATGACACAGACCCCACTGTCTCTGCCTGTGTC |
| DOTA CAR | | TCTGGGAGATCAGGCCAGCATCAGCTGCAGATCTAGCCAGA |
| | | GCCTGGTGCACAGCAACGGCAACACCTACCTGCGGTGGTATC |
| | | TGCAGAAGCCCGGCCAGTCTCCTAAGGTGCTGATCTACAAGG |
| | | TGTCCAACAGAGTGTCCGGCGTGCCCGATAGATTTTCTGGCA |
| | | GCGGCTCTGGCACCGACTTCACCCTGAAGATCAATAGAGTGG |
| | | AAGCCGAGGACCTGGGCGTGTACTTCTGTAGCCAGTCTACCC |
| | | ACGTGCCATGGACCTTTGGCGGCGGAACAAAGCTGGAAATC |
| | | AAGAGCAGCGCCGACGACGCCAAGAAGGACGCCGCTAAGA |
| | | AGGATGACGCAAAGAAAGACGATGCCAAAAAGGATGGCGG |
| | | CGTGAAGCTGGACGAAACAGGCGGAGGACTTGTTCAGCCTG |
| | | GCGGAGCCATGAAGCTGAGCTGTGTGACCAGCGGCTTCACCT |
| | | TTGGCCACTACTGGATGAACTGGGTCCGACAGAGCCCTGAG |
| | | AAAGGCCTGGAATGGGTCGCCCAGTTCAGAAACAAGCCCTA |
| | | CAACTACGAAACCTACTACAGCGACAGCGTGAAGGGCAGAT |
| | | TCACCATCAGCCGGGACGACAGCAAGTCCAGCGTGTACCTG |
| | | CAGATGAACAACCTGCGCGTGGAAGATACCGGCATCTACTA |
| | | CTGTACCGGCGCCAGCTACGGCATGGAATATCTCGGCCAGG |
| | | GCACCAGCGTGACCGTGTCTACAACAACCCCTGCTCCTCGGC |
| | | CTCCTACACCAGCTCCTACAATTGCCAGCCAGCCTCTGTCTCT |
| | | GAGGCCCGAAGCTTGTAGACCTGCTGCCGGCGGAGCTGTGC |
| | | ATACAAGAGGCCTGGATTTCGCCTGCGATCCCATCCTGCTGA |
| | | CAATCAGCATCCTGAGCTTTTTCAGCGTGGCCCTGCTGGTCA |
| | | TCCTGGCCTGTGTGCTGTGGAAGAAGCGGATCAAGCCCATCG |
| | | TGTGGCCCAGCCTGCCTGACCACAAGAAAACCCTGGAACAC |
| | | CTGTGCAAGAAGCCCCGGAAGAACCTGAACGTGTCCTTCAAT |
| | | CCCGAGAGCTTCCTGGACTGCCAGATCCACAGAGTGGACGA |
| | | CATCCAGGCCAGGGACGAAGTGGAAGGCTTTCTGCAGGACA |
| | | CATTCCCTCAGCAGCTGGAAGAGAGCGAGAAGCAGAGACTC |
| | | GGAGGCGACGTGCAGAGCCCTAATTGCCCTTCTGAGGACGTC |
| | | GTGATCACCCCTGAGAGCTTCGGCAGAGATAGCAGCCTGAC |
| | | ATGTCTGGCCGGCAATGTGTCCGCCTGTGATGCCCCTATCCT |
| | | GAGCAGCAGCAGAAGCCTGGATTGCAGAGAGAGCGGCAAG |
| | | AACGGCCCTCACGTGTACCAGGATCTGCTCCTGAGCCTGGGA |
| | | ACCACCAATAGCACACTGCCTCCACCATTCAGCCTGCAGAGC |
| | | GGCATCCTGACACTGAACCCTGTTGCTCAGGGCCAGCCAATC |
| | | CTGACAAGCCTGGGCAGCAATCAAGAAGAGGCCTACGTCAC |
| | | CATGAGCAGCTTCTACCAGAATCAAGGCTCCGGCGAAGGCA |
| | | GAGGCTCTCTGCTTACATGCGGAGATGTGGAAGAGAACCCC |
| | | GGACCTATGGCTCTGCCAGTGACAGCTCTGCTGCTTCCTCTG |
| | | GCTCTCCTGCTGCATGCCGCTAGACCTGAGCAGAAACTCATC |
| | | TCTGAAGAGGATCTGCACGTGAAACTGCAAGAGTCTGGCCC |
| | | CGGACTGGTGCAGCCTTCTCAGTCTCTCTCTCTGACCTGTACC |
| | | GTGTCCGGCTTCAGCCTGACCGATTATGGCGTGCACTGGGTT |
| | | CGACAATCCCCAGGCAAAGGACTCGAGTGGCTGGGAGTGAT |
| | | TTGGAGCGGCGGAGGCACAGCCTATAACACAGCCCTGATCA |
| | | GCAGACTGAACATCTACCGGGACAACTCCAAGAACCAGGTT |
| | | TTCCTGGAAATGAACTCCCTGCAGGCAGAGGACACCGCCAT |
| | | GTACTACTGCGCCAGAAGAGGCAGCTACCCCTACAATTACTT |
| | | CGACGCCTGGGGCTGTGGCACAACCGTGACAGTTTCTAGTGG |
| | | CGGAGGCGGATCTGGTGGTGGTGGTAGCGGTGGCGGAGGAT |
| | | CTCAGGCCGTGGTTATTCAAGAAAGCGCCCTGACAACCCCTC |
| | | CTGGCGAGACAGTGACACTGACATGTGGCAGCTCTACAGGC |
| | | GCCGTGACCGCCAGCAATTACGCCAATTGGGTGCAAGAGAA |
| | | GCCCGACCACTGCTTCACAGGCCTGATCGGCGGCCACAACA |
| | | ATAGACCTCCAGGCGTGCCAGCTAGATTCAGCGGATCCCTGA |
| | | TCGGAGACAAGGCCGCTCTGACAATCGCCGGCACACAGACA |
| | | GAGGACGAGGCCATCTACTTTTGCGCCCTGTGGTACAGCGAC |
| | | CACTGGGTTATCGGCGGAGGAACCAGACTGACAGTGCTGGG |
| | | CACAACAACACCCGCACCTAGACCACCAACTCCAGCACCAA |
| | | CAATCGCCTCTCAACCCCTGAGTCTGAGGCCAGAGGCATGCA |
| | | GACCAGCCGCTGGCGGTGCAGTTCACACTAGAGGACTGGAC |
| | | TTTGCCTGTGACATCTACATCTGGGCCCCTCTGGCTGGAACA |
| | | TGTGGTGTCCTGCTGCTGTCCCTGGTCATCACCCTGTACTGCA |
| | | AGCGGGGCAGAAAGAAACTGCTGTACATCTTCAAGCAGCCC |
| | | TTCATGCGGCCCGTGCAGACCACACAAGAGGAAGATGGCTG |
| | | CTCCTGCAGATTCCCCGAGGAAGAAGAAGGCGGCTGCGAGC |
| | | TGAGAGTGAAGTTCTCCAGATCTGCCGACGCTCCCGCCTATC |
| | | AGCAGGGACAGAACCAGCTGTACAACGAGCTGAACCTGGGG |
| | | AGAAGAGAAGAGTACGACGTGCTGGACAAGCGGAGAGGCA |
| | | GAGATCCTGAGATGGGCGGAAAGCCCCAGCGGAGAAAGAAT |
| | | CCTCAAGAGGGCCTGTATAATGAGCTGCAAAAGGACAAGAT |
| | | GGCCGAGGCCTACAGCGAGATCGGAATGAAGGGCGAGCGCA |
| | | GAAGAGGAAAGGGACACGACGGACTGTACCAGGGCCTGAGC |
| | | ACAGCCACCAAGGATACCTATGACGCCCTGCACATGCAGGC |
| | | CCTGCCTCCAAGATGATGA |
| anti-DOTA | SEQ ID NO: 52 | ATGACCATCCTGGGCACCACCTTCGGCATGGTGTTTAGCCTG |
| IL7Rα SMAR- | | CTGCAGGTCGTGTCTGGCGATTACAAGGATGACGACGATAA |
| T2A-anti- | | GCACGTGAAACTGCAAGAGTCTGGCCCCGGACTGGTGCAGC |
| FITC CAR | | CTTCTCAGTCTCTCTCTCTGACCTGTACCGTGTCCGGCTTCAG |
| | | CCTGACCGATTATGGCGTGCACTGGGTTCGACAATCCCCAGG |
| | | CAAAGGACTCGAGTGGCTGGGAGTGATTTGGAGCGGCGGAG |
| | | GCACAGCCTATAACACAGCCCTGATCAGCAGACTGAACATCT |
| | | ACCGGGACAACTCCAAGAACCAGGTTTTCCTGGAAATGAAC |
| | | TCCCTGCAGGCAGAGGACACCGCCATGTACTACTGCGCCAG |
| | | AAGAGGCAGCTACCCCTACAATTACTTCGACGCCTGGGGCTG |
| | | TGGCACAACCGTGACAGTTTCTAGTGGCGGAGGCGGATCTG |
| | | GTGGTGGTGGTAGCGGTGGCGGAGGATCTCAGGCCGTGGTT |
| | | ATTCAAGAAAGCGCCCTGACAACCCCTCCTGGCGAGACAGT |
| | | GACACTGACATGTGGCAGCTCTACAGGCGCCGTGACCGCCA |
| | | GCAATTACGCCAATTGGGTGCAAGAGAAGCCCGACCACTGC |
| | | TTCACAGGCCTGATCGGCGGCCACAACAATAGACCTCCAGG |
| | | CGTGCCAGCTAGATTCAGCGGATCCCTGATCGGAGACAAGG |
| | | CCGCTCTGACAATCGCCGGCACACAGACAGAGGACGAGGCC |
| | | ATCTACTTTTGCGCCCTGTGGTACAGCGACCACTGGGTTATC |
| | | GGCGGAGGAACCAGACTGACAGTGCTGGGCACAACAACCCC |
| | | TGCTCCTCGGCCTCCTACACCAGCTCCTACAATTGCCAGCCA |
| | | GCCTCTGTCTCTGAGGCCCGAAGCTTGTAGACCTGCTGCCGG |
| | | CGGAGCTGTGCATACAAGAGGCCTGGATTTCGCCTGCGATCC |
| | | CATCCTGCTGACAATCAGCATCCTGAGCTTTTTCAGCGTGGC |
| | | CCTGCTGGTCATCCTGGCCTGTGTGCTGTGGAAGAAGCGGAT |
| | | CAAGCCCATCGTGTGGCCCAGCCTGCCTGACCACAAGAAAA |
| | | CCCTGGAACACCTGTGCAAGAAGCCCCGGAAGAACCTGAAC |
| | | GTGTCCTTCAATCCCGAGAGCTTCCTGGACTGCCAGATCCAC |
| | | AGAGTGGACGACATCCAGGCCAGGGACGAAGTGGAAGGCTT |
| | | TCTGCAGGACACATTCCCTCAGCAGCTGGAAGAGAGCGAGA |
| | | AGCAGAGACTCGGAGGCGACGTGCAGAGCCCTAATTGCCCT |
| | | TCTGAGGACGTCGTGATCACCCCTGAGAGCTTCGGCAGAGAT |
| | | AGCAGCCTGACATGTCTGGCCGGCAATGTGTCCGCCTGTGAT |
| | | GCCCCTATCCTGAGCAGCAGCAGAAGCCTGGATTGCAGAGA |
| | | GAGCGGCAAGAACGGCCCTCACGTGTACCAGGATCTGCTCCT |
| | | GAGCCTGGGAACCACCAATAGCACACTGCCTCCACCATTCAG |
| | | CCTGCAGAGCGGCATCCTGACACTGAACCCTGTTGCTCAGGG |
| | | CCAGCCAATCCTGACAAGCCTGGGCAGCAATCAAGAAGAGG |
| | | CCTACGTCACCATGAGCAGCTTCTACCAGAATCAAGGCTCCG |
| | | GCGAAGGCAGAGGCTCTCTGCTTACATGCGGAGATGTGGAA |
| | | GAGAACCCCGGACCTATGGCTCTGCCTGTGACAGCTCTGCTG |
| | | CTGCCTCTGGCTCTGCTTCTGCATGCCGCCAGACCTGACGTG |
| | | GTCATGACACAGACACCTCTGAGCCTGCCTGTGTCTCTGGGA |
| | | GATCAGGCCAGCATCAGCTGCAGATCTAGCCAGAGCCTGGT |
| | | GCACAGCAACGGCAACACCTACCTGCGGTGGTATCTGCAGA |
| | | AGCCCGGCCAGTCTCCTAAGGTGCTGATCTACAAGGTGTCCA |
| | | ACAGAGTGTCCGGCGTGCCCGATAGATTTTCTGGCAGCGGCT |
| | | CTGGCACCGACTTCACCCTGAAGATCAATAGAGTGGAAGCC |
| | | GAGGACCTGGGCGTGTACTTCTGTAGCCAGTCTACCCACGTG |
| | | CCATGGACCTTTGGCGGCGGAACAAAGCTGGAAATCAAGAG |
| | | CAGCGCCGACGACGCCAAGAAGGACGCCGCTAAGAAGGATG |
| | | ACGCCAAAAAAGACGATGCCAAAAAGGATGGCGGCGTGAA |
| | | GCTGGACGAAACAGGCGGAGGACTTGTTCAGCCTGGCGGAG |
| | | CCATGAAGCTGAGCTGTGTGACCAGCGGCTTCACCTTCGGCC |
| | | ACTACTGGATGAACTGGGTCCGACAGAGCCCTGAGAAAGGC |
| | | CTGGAATGGGTCGCCCAGTTCAGAAACAAGCCCTACAACTA |
| | | CGAAACCTACTACAGCGACAGCGTGAAGGGCAGATTCACCA |
| | | TCAGCCGGGACGACAGCAAGTCCAGCGTGTACCTGCAGATG |
| | | AACAACCTGCGCGTGGAAGATACCGGCATCTACTACTGTACC |
| | | GGCGCCAGCTACGGCATGGAATATCTCGGCCAGGGCACCAG |
| | | CGTGACCGTGTCTACAACAACCCCTGCTCCTCGGCCTCCTAC |
| | | ACCAGCTCCTACAATTGCCAGCCAGCCACTGTCTCTGAGGCC |
| | | CGAAGCTTGTAGACCTGCTGCAGGCGGAGCCGTGCATACAA |
| | | GAGGACTGGATTTCGCCTGCGACTTCTGGGTGCTCGTGGTTG |
| | | TTGGCGGAGTGCTGGCTTGTTACTCCCTGCTGGTTACCGTGG |
| | | CCTTCATCATCTTTTGGGTCAAGCGGGGCAGAAAGAAGCTGC |
| | | TGTACATCTTCAAGCAGCCCTTCATGCGGCCCGTGCAGACCA |
| | | CACAAGAGGAAGATGGCTGCTCCTGCAGATTCCCCGAGGAA |
| | | GAAGAAGGCGGCTGCGAGCTGAGAGTGAAGTTCAGCAGATC |
| | | CGCCGACGCTCCTGCCTATCAGCAGGGACAGAACCAGCTGT |
| | | ACAACGAGCTGAACCTGGGGAGAAGAGAAGAGTACGACGTG |
| | | CTGGACAAGCGGAGAGGCAGAGATCCTGAGATGGGCGGAAA |
| | | GCCCCAGCGGAGAAAGAATCCTCAAGAGGGCCTGTATAATG |
| | | AGCTGCAGAAAGACAAGATGGCCGAGGCCTACAGCGAGATC |
| | | GGAATGAAGGGCGAGCGCAGAAGAGGCAAGGGACACGATG |
| | | GACTGTACCAGGGCCTGAGCACCGCCACCAAGGATACCTAT |
| | | GATGCCCTGCACATGCAGGCCCTGCCACCTAGATGATGA |

In some embodiments, a polynucleotide construct includes a sequence having at least about 70%, at least about 75%, at least about 80%, at least about 85%, at least about 87%, at least about 90%, at least about 93%, at least about 95%, at least about 96%, at least about 97%, at least about 98%, at least about 99%, at least about 99.5%, or about 100% sequence identity to any one of SEQ ID NO: 46-SEQ ID NO: 52. In some embodiments, the polynucleotide construct has a sequence of any one of SEQ ID NO: 46-SEQ ID NO: 52.

### V. Methods of Ex Vivo Immune Cell Activation

Aspects of the present disclosure provide compositions and methods for ex vivo engineered immune cell activation. The methods can comprise contacting an engineered immune cell to an activator of an engineered cytokine receptor switch, thereby activating the intracellular signaling domain of the engineered cytokine receptor switch to affect cell phenotype. For example, activation of an engineered cytokine receptor switch with an IL2Rα intracellular signaling domain can activate a range of intracellular signaling pathways by means of transduction through the JAK-STAT pathway, for example in some cases promoting longevity by inhibiting apoptosis. In this way, engineered cytokine receptor switch activation can serve as a handle for controlling and modifying engineered immune cell phenotype during preparation ex vivo, such that the engineered immune cells can have a desired phenotype or phenotype ratio (e.g., 50% terminal effector cells, 50% memory cells) upon administration to a subject.

A method of activating an immune cell population can comprise: providing an immune cell population expressing an engineered cytokine receptor switch comprising an activator binding domain configured to bind to an activator, a transmembrane domain, and an intracellular domain; contacting the immune cell population to a substrate comprising the activator, thereby activating the cytokine receptor switch; and converting at least a portion of the immune cell population to an effector phenotype, a memory phenotype, or a combination thereof, thereby activating the immune cell population. In some cases, the method is completed within 3 to 21 days. In some cases, the method is completed within 7 to 21 days. In some cases, the method is completed within about 5 to about 14 days. In some cases, the method is completed within about 5 to about 10 days.

The activator binding domain can comprise a peptide, such as a single-chain variable fragment, an antibody fragment antigen-binding (Fab) domain, a nanobody, a cytokine receptor extracellular domain, or a combination thereof. The activator binding domain can bind to the activator with a high degree of specificity. Activator binding domain binding affinity can be selected based on the desired degree of engineered cytokine receptor switch activation. In some cases, the activator binding domain has a relatively high affinity for its activator, for example between 1 nM and 1 fM, enabling high degrees of engineered cytokine receptor switch activation, even in the presence of relatively low concentrations of activator. The activator binding domain can alternatively have a relatively low binding affinity for its activator, for example between 100 μM and 5 μM, enabling correspondingly lower degrees of engineered cytokine receptor switch activation in the absence of high activator concentrations, for example following multifold immune cell expansion during some ex vivo activation methods. In some cases, the activator binding domain comprises a molecular weight of about 1 kDa to about 10 kDa. In some cases, the activator binding domain is humanized, for example to minimize immune responses against the activator binding domain. In some cases, the activator binding domain comprises a binding affinity of between about 10 UM and about 1 fM for the activator.

The intracellular domain can be configured to transduce a signal upon activation of the engineered cytokine receptor switch. In many cases, the intracellular domain activates a signal native to the immune cell engineered to express the engineered cytokine receptor switch, for example a JAK-STAT pathway, allowing the engineered cytokine receptor switch to utilize signaling machinery present in the immune cell, and circumventing the need to further modify its signaling repertoire. In some cases, the intracellular domain comprises a cytokine receptor intracellular domain. The intracellular domain can be derived from the endogenous cytokine receptor intracellular domain, for example comprising a sequence having at least about 80%, at least about 90%, at least about 95%, at least about 97%, or 100% sequence identity to the endogenous cytokine intracellular domain.

The transmembrane domain can connect the activator binding domain to the intracellular domain. In many cases, this connection facilitates communication of activator binding by the activator binding domain to the intracellular domain, activating the intracellular domain to transduce a signal (e.g., through STAT1 or STAT2 phosphorylation). In some cases, the activator binding domain, the intracellular domain, and the transmembrane domain are a single expression construct (e.g., comprised within a single peptide sequence). In some cases, the transmembrane domain is a cytokine receptor transmembrane domain. In some cases, the transmembrane domain is derived from an endogenous cytokine receptor transmembrane domain. In some cases, the endogenous cytokine receptor transmembrane domain is an IL2Rα transmembrane domain, an IL2Rß transmembrane domain, an IL2Rγ transmembrane domain, an IL4Rα transmembrane domain, an IL7Rα transmembrane domain, an IL9Rα transmembrane domain, an IL15Rα transmembrane domain, or an IL21Rα transmembrane domain. In some cases, the transmembrane domain comprises a sequence having at least 80%, at least 90%, at least 95%, at least 97%, or 100% sequence identity to the endogenous cytokine receptor transmembrane domain.

In some cases, the engineered cytokine receptor switch comprises a hinge domain. In some cases, the hinge domain is derived from an endogenous hinge domain. In some cases, the endogenous hinge domain is a CD8 hinge, a CD3 hinge, a CD4 hinge, or a CD28 hinge. In some cases, the hinge domain comprises a sequence having at least 80%, at least 90%, at least 95%, at least 97%, or 100% sequence identity to the endogenous hinge domain.

In some cases, the engineered cytokine receptor switch comprises a signal peptide. The signal peptide can promote subcellular localization of the engineered cytokine receptor switch, for example to the endoplasmic reticulum for cell surface export. In some cases, the signal peptide is a cytokine receptor signal peptide, such as an IL2Rα signal peptide, an IL2Rß signal peptide, an IL2Rγ signal peptide, an IL4Rα signal peptide, an IL7Rα signal peptide, an IL9Rα signal peptide, an IL15Rα signal peptide, or an IL21Rα signal peptide. In some cases, the cytokine receptor signal peptide is derived from an endogenous cytokine signal peptide. In some cases, the signal peptide comprises a sequence having at least 80%, at least 90%, at least 95%, at least 97%, or 100% sequence identity to the endogenous cytokine receptor signal peptide. In some cases, the signal peptide is an endoplasmic reticulum localization tag.

In some cases, the method comprises transfecting the immune cell population with a vector encoding the engineered cytokine receptor switch. The immune cells can be further transfected to express a CAR. For example, the vector encoding the engineered cytokine receptor switch can also encode the CAR, or the immune cells can be contacted with separate viral vectors encoding the engineered cytokine receptor switch and the CAR. In some cases, the vector is a viral vector. In some cases, the viral vector is a lentiviral vector, an adeno-associated viral vector, a vaccinia viral vector, a poxvirus viral vector, a herpes viral vector, an alphavirus viral vector, gamma retrovirus, a polyoma viral vector, or a combination thereof. In some cases, the viral vector is a gamma retrovirus, an adeno-associated viral vector or a lentiviral vector. In some cases, the viral vector is a lentiviral vector. In some cases, the viral vector has a titer of between about 106 and about 109 virions per ml. In some cases, between about 10% and about 30%, between about 10% and about 50%, between about 20% and about 60%, between about 40% and about 90%, or between about 60% and about 95% of naïve T-cells and B-cells from among the immune cell population are transfected.

The immune cell population to be transfected can be obtained as further described herein, for example from peripheral blood. In some cases, the immune cell population is obtained through leukapheresis, bone marrow biopsy, or a combination thereof. In some cases, the immune cell population comprises at least about 10%, at least about 20%, at least about 30%, at least about 40%, at least about 50%, at least about 60%, at least about 70%, or at least about 80% naïve T-cells and naïve B-cells as a percentage of total cell population prior to contacting the immune cells with the activator.

In many cases, the activator is a small molecule, a peptide, or an oligonucleotide, including any of the activators disclosed herein. In some cases, the activator is a small molecule. In some cases, the activator comprises fluorescein, tetraxetan, butylated hydroxytoluene, Rhodamine B, benzoic acid, erythrosine, tartrazine, PEG methoxy group, dinitrophenyl (DNP), and histidine tags (e.g., His6), a derivative thereof, or a combination thereof. In some cases, the activator comprises fluorescein, tetraxetan, a derivative thereof, or a combination thereof. Additional examples of activators are provided elsewhere herein.

In some cases, the activator is coupled to the substrate via a linker. The linker can be flexible and water soluble, or can be rigid. In some cases, the linker comprises a synthetic polymer or a biopolymer (e.g., a peptide). In some cases, the linker comprises polyethylene glycol (PEG). In some cases, the linker comprises a length of between about 5 nm and about 100 nm. In some cases, the linker is rigid, for example comprises a length which is shorter than its persistence length, comprises a length which is at most about 0.5-times its persistence length, or comprises a length which is at most about 0.1-times its persistence length. In some cases, the linker is flexible, for example comprises a length which is longer than its persistence length, comprises a length which is at least about 5-times longer than its persistence length, comprises a length which is at least about 10-times longer than its persistence length, or comprises a length which is at least about 25-times longer than its persistence length. In some cases, the activator is coupled to a carrier complex coupled to the substrate. In some cases, the activator is coupled to a carrier complex coupled to a binding protein coupled to the substrate. In some cases, the binding protein is streptavidin.

In some cases, the substrate comprises a peptide, an antibody, a minibody, a nanobody, a fragment antigen-binding, a nanoparticle, a microparticle, a polymer matrix, a surface, a surface functionalization (e.g., a dextran polymer functionalized with the activator), a carbon nanomaterial, a quantum dot, or a combination thereof. In some cases, at the time of contacting the substrate to the immune cell population, a ratio of the substrate to immune cells of the immune cell population is between about 100:1 and about 1:100. In some cases, at the time of contacting the substrate to the immune cell population, the substrate is in stoichiometric excess of immune cells of the immune cell population. In some cases, a ratio of the substrate to immune cells of the immune cell population is between about 1:1 and about 10:1, between about 1:1 and about 25:1, between about 1:1 and about 50:1, between about 1:1 and about 100:1, between about 2:1 and about 25:1, between about 5:1 and about 25:1, between about 5:1 and about 50:1, between about 10:1 and about 50:1, or between about 10:1 and about 100:1. In some cases, immune cells of the immune cell population are in stoichiometric excess of the substrate. In some cases, a ratio of the substrate to immune cells of the immune cell population is between about 1:1 and about 1:10, between about 1:1 and about 1:25, between about 1:1 and about 1:50, between about 1:1 and about 1:100, between about 1:2 and about 1:25, between about 1:5 and about 1:25, between about 1:5 and about 1:50, between about 1:10 and about 1:50, or between about 1:10 and about 1:100. In some cases, the immune cell population is contacted to the substrate for between about 1 day and about 5 days, between about 1 day and about 10 days, between about 2 days and about 7 days, between about 2 days and about 10 days, between about 3 days and about 10 days, between about 4 days and about 14 days, or between about 7 days and about 21 days.

In some cases, the activator has a density of between about 10³ and about 2×10⁶ molecules per μm² of a surface of the substrate. In some cases, the activator has a density of between about 5×10³ and about 5×10⁵, between about 5×10³ and about 10⁵, between about 104 and about 2×10⁵, between about 2×10⁴ and about 5×10⁵, between about 5×10⁴ and about 10⁶ per μm² of a surface of the substrate, or between about 5×10⁴ and about 2×10⁶ per μm² of a surface of the substrate.

In some cases, the immune cell population is expanded while in contact with the substrate. In some cases, the immune cell population is expanded by about 2-fold, about 5-fold, about 10-fold, about 25-fold, about 50-fold, or about 100-fold while in contact with the substrate. In some cases, the immune cell population is expanded by between about 2-fold and about 10-fold, by between about 2-fold and about 25-fold, by between about 5-fold and about 50-fold, or by between about 10-fold and about 100-fold while in contact with the substrate.

In some cases, the immune cell population is contacted to a receptor agonist concurrently with the contacting to the substrate. In some cases, the receptor agonist is selected from the group consisting of granulocyte macrophage-colony stimulating factor (GM-CSF), stem cell factor (SCF), interleukin-1 (IL1), interleukin-2 (IL2), interleukin-3 (IL3), a CD3 agonist, a CD4 agonist, a CD8 agonist, a CD16 agonist, a CD23 agonist, a CD28 agonist, a CD47 agonist, a CD80 agonist, a CD113 agonist, a CD131 agonist, a CD137 agonist, an HLA-E agonist, a 41BBL agonist, and a combination thereof. In some cases, the receptor agonist is selected from the group consisting of a CD3 agonist, a CD23 agonist, a CD28 agonist, an IL2 receptor agonist, and a combination thereof. In some cases, the CD3 agonist is an antigen. In some cases, the receptor agonist is coupled to a second substrate. In some cases, the second substrate comprises a peptide, an antibody, a minibody, a nanobody, a fragment antigen-binding, a nanoparticle, a microparticle, a polymer matrix, a surface, a surface functionalization (e.g., a dextran polymer functionalized with the activator), a carbon nanomaterial, a quantum dot, or a combination thereof. In some cases, the second substrate is contacted to the immune cell population at a ratio of between about 50:1 and about 1:1 (second substrate to immune cells).

In some cases, prior to transfecting the immune cell population and contacting the immune cell population to the substrate, the immune cell population is expanded for between about 0.25 days and about 10 days, between about 0.25 days and about 2 days, between about 0.5 days and about 3 days, between about 1 and about 5 days, between about 2 and about 7 days, or between about 2.5 and about 10 days. In some cases, the expansion is performed in the presence of a receptor agonist. In some cases, the receptor agonist is selected from the group consisting of a CD3 agonist, a CD4 agonist, a CD8 agonist, a CD16 agonist, a CD23 agonist, a CD28 agonist, a CD47 agonist, a CD80 agonist, a CD113 agonist, a CD131 agonist, a CD137 agonist, an IL2 receptor agonist, an HLA-E agonist, a 41BBL agonist, and a combination thereof. In some cases, the receptor agonist is selected from the group consisting of a CD3 agonist, a CD23 agonist, a CD28 agonist, an IL2 receptor agonist, and a combination thereof. In some cases, the receptor agonist is coupled to a second substrate. In some cases, the second substrate comprises a peptide, an antibody, a minibody, a nanobody, a fragment antigen-binding, a nanoparticle, a microparticle, a polymer matrix, a surface, a surface functionalization (e.g., a dextran polymer functionalized with the activator), a carbon nanomaterial, a quantum dot, or a combination thereof. In some cases, the immune cells are contacted with a CD3 agonist and a CD28 agonist prior to transfection. In some cases, the immune cells are contacted with IL2 prior to transfection. In some cases, the immune cells are contacted with IL2 during transfection. In some cases, the immune cell population is expanded by about 2-fold, about 5-fold, about 10-fold, about 25-fold, about 50-fold, or about 100-fold prior to transfection and the contacting to the substrate. In some cases, the immune cell population is expanded by between about 2-fold and about 10-fold, by between about 2-fold and about 25-fold, by between about 5-fold and about 50-fold, or by between about 10-fold and about 100-fold prior to transfection and the contacting to the substrate.

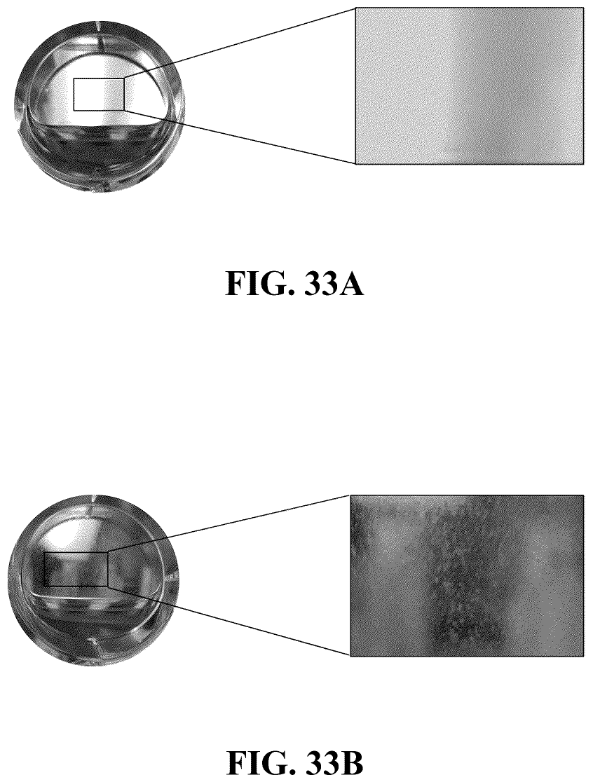

Following expansion, transfection, and/or activation, the engineered immune cell population can be fractionated on a substrate. In some cases, the engineered immune cells are collected on a substrate that is functionalized with an activator (e.g., a species which binds to an activator binding domain of an engineered cytokine receptor switch). As depicted in FIG. 33A and FIG. 33B, which shows an activator-functionalized surface in the presence of cells which do not express an engineered cytokine receptor switch and in the presence of cells which express an engineered cytokine receptor switch, respectively, activator functionalized substrates can selectively bind cells which express engineered cytokine receptor switches. Leveraging this discovery, a method can comprise separating engineered cytokine receptor switch expressing immune cells from immune cells which do not express the engineered cytokine receptor switch by binding the engineered cytokine receptor switch expressing immune cells to a substrate. Unbound cells can then be washed or removed, and the engineered cytokine receptor switch expressing immune cells can be collected from the substrate. Alternatively, the substrate (along with bound immune cells) can be separated from unbound immune cells, for example through gravimetric or magnetic separation.

In some cases, the engineered immune cell population is fractionated by cell phenotype. During such processes, subsets of the engineered immune cell population which express a particular cell marker (e.g., CCR7 or CD45RA) can be captured and separated from subsets of the engineered immune cell population which do not express the cell marker. For example, T-cells can be separated from non-T-cells of the immune cell population through CD3 affinity collection. In some cases, a method includes selecting memory T-cells from an immune cell population, for example by collecting CCR7+ and/or CCR7+/CD45RA+ cells from the immune cell population.

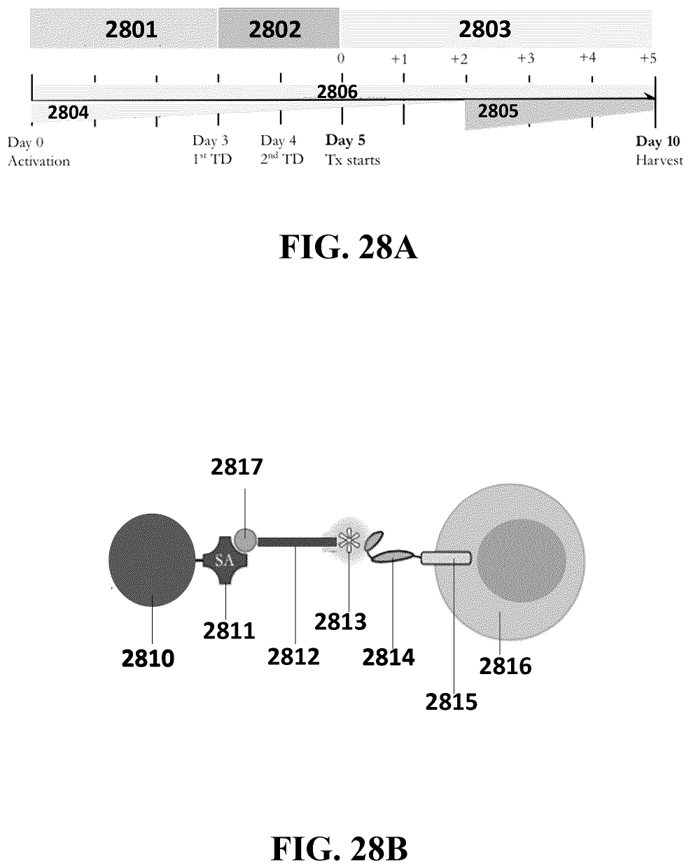

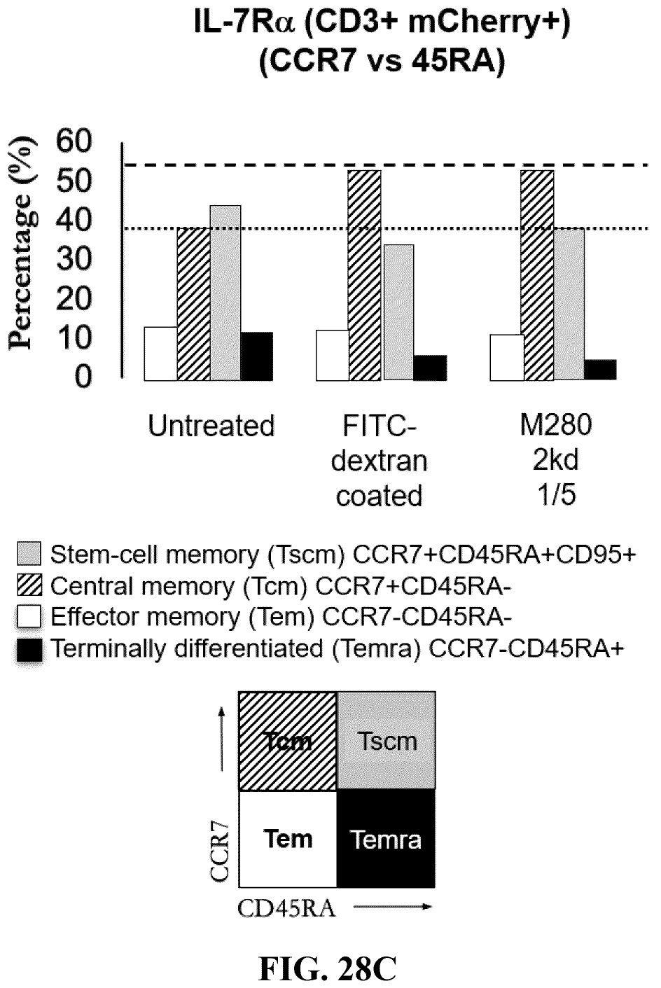

An example of a method for immune cell activation consistent with the present disclosure is outlined in FIG. 28A. Following immune cell collection (e.g., through leukapheresis), the immune cells can be expanded 2801 in the presence of an agonist or antagonist of a receptor native to the immune cells 2804, such as CD3, CD23, CD28, IL2R, or a combination thereof. Following multiple days of expansion, the cells can be transfected 2802 with a vector encoding an engineered cytokine receptor switch, and optionally further encoding an additional species, such as a CAR. The immune cells can be contacted to an activator of the engineered cytokine receptor switch 2803. During this time, the cells can optionally be contacted with a second dose of the agonist or antagonist of the receptor native to the immune cells 2805. Optionally, the immune cells can be contacted with a constant dose of an additional agonist or antagonist 2806 for the entirety of 2801-2803 (e.g., 20-100 units/ml IL2). While FIG. 28A depicts 5 days of activation in the presence of the engineered cytokine receptor switch activator, this period may last for anywhere between about 0.25 days to about 14 days, between about 0.25 days to about 3 days, about 0.5 to about 3 days, about 1 to about 10 days, about 1 to about 7 days, about 2 to about 7 days, or about 7 to about 14 days. Similarly, the initial expansion phase 2801 can last for between about 0.25 days to about 10 days, between about 0.25 days to about 2 days, about 0.5 to about 3 days, about 1 to about 7 days, about 1 to about 5 days, about 1.5 to about 5 days, or about 1 to about 4 days. The transfecting 2802 can last for between about 0.25 days to about 5 days, between about 0.25 days to about 2 days, about 0.5 to about 3 days, about 1 to about 5 days, about 1 to about 4 days, about 1 to about 2.5 days, or about 1 to about 2 days. The method can comprise a total time of about 3 to about 21 days, between about 3 and about 10 days, between about 3 and about 7 days, between about 2 and about 5 days, between about 4 and about 10 days, between about 4 and about 12 days, or between about 7 and about 21 days.

### A. Immune Cell Phenotypes

The method of activating can generate particular phenotypes within the immune cell population. When challenged with antigens, immune cells often differentiate towards terminal effector phenotypes, which typically have high potencies but relatively short lifespans in vivo in the absence of antigen. While such immune cells can have pronounced activities against acute diseases, rapid immune cell death following partial disease clearance can allow disease recurrence. Memory phenotypes typically comprise longer in vivo lifespans, as well as a greater ability to divide, facilitating greater persistence in the absence of their targets. Accordingly, controlling terminal effector and memory phenotype ratios can be important for tailoring a treatment to a subject.

For some methods disclosed herein, following activation, a ratio of effector and memory phenotypes in an immune cell population is between about 20:1 and about 1:20, between about 5:1 and about 1:5, or between about 2:1 and about 1:2. In some cases, following activation, a ratio of effector and memory phenotypes in an immune cell population is between about 100:1 and about 10:1, between about 50:1 and about 5:1, between about 25:1 and about 5:1, between about 15:1 and about 3:1, between about 10:1 and about 2:1, between about 5:1 and about 3:2, or between about 4:1 and about 3:2. In some cases, following activation, a ratio of effector and memory phenotypes in an immune cell population is between about 1:100 and about 1:10, between about 1:50 and about 1:5, between about 1:25 and about 1:5, between about 1:15 and about 1:3, between about 1:10 and about 1:2, between about 1:5 and about 2:3, or between about 1:4 and about 2:3. In some cases, the activation converts at least about 50%, at least about 60%, at least about 70%, at least about 80%, at least about 90%, at least about 95%, at least about 98%, or at least about 99% of the immune cell population to a memory phenotype.

In some cases, following activation, at least about 40% of T-cells of the immune cell population are central memory cells. In some cases, following activation, between about 40% and about 85% of T-cells of the immune cell population are central memory cells. In some cases, following activation, between about 40% and about 75% of T-cells of the immune cell population are central memory cells. In some cases, following activation, between about 45% and about 75% of T-cells of the immune cell population are central memory cells. In some cases, following activation, between about 50% and about 75% of T-cells of the immune cell population are central memory cells. In some cases, following activation, between about 60% and about 90% of T-cells of the immune cell population are central memory cells.

In some cases, following activation, between about 3% and about 30% of T-cells of the immune cell population are stem memory T-cells. In some cases, following activation, between about 4% and about 25% of T-cells of the immune cell population are stem memory T-cells. In some cases, following activation, between about 4% and about 20% of T-cells of the immune cell population are stem memory T-cells. In some cases, following activation, between about 3% and about 15% of T-cells of the immune cell population are stem memory T-cells.

In some cases, following activation, between about 5% and about 60% of T-cells of the immune cell population are effector memory T-cells. In some cases, following activation, between about 5% and about 50% of T-cells of the immune cell population are effector memory T-cells. In some cases, following activation, between about 8% and about 40% of T-cells of the immune cell population are effector memory T-cells. In some cases, following activation, between about 8% and about 35% of T-cells of the immune cell population are effector memory T-cells. In some cases, following activation, between about 5% and about 35% of T-cells of the immune cell population are effector memory T-cells. In some cases, following activation, between about 5% and about 25% of T-cells of the immune cell population are effector memory T-cells. In some cases, following activation, between about 5% and about 20% of T-cells of the immune cell population are effector memory T-cells.

In some cases, following activation, less than about 20% of T-cells of the immune cell population are terminally differentiated effector memory T-cells. In some cases, following activation, less than about 15% of T-cells of the immune cell population are terminally differentiated effector memory T-cells. In some cases, following activation, less than about 10% of T-cells of the immune cell population are terminally differentiated effector memory T-cells. In some cases, following activation, less than about 8% of T-cells of the immune cell population are terminally differentiated effector memory T-cells. In some cases, following activation, less than about 6% of T-cells of the immune cell population are terminally differentiated effector memory T-cells. In some cases, following activation, less than about 4% of T-cells of the immune cell population are terminally differentiated effector memory T-cells. In some cases, following activation, between about 0.5% and about 20% of T-cells of the immune cell population are terminally differentiated effector memory T-cells. In some cases, following activation, between about 0.5% and about 10% of T-cells of the immune cell population are terminally differentiated effector memory T-cells. In some cases, following activation, between about 0.5% and about 6% of T-cells of the immune cell population are terminally differentiated effector memory T-cells.

### VI. Compositions Comprising Engineered Immune Cells

Provided herein are engineered immune cells suitable for administration to a subject in need thereof. The engineered immune cells or precursor cells thereof may be cultured and formulated for delivery to a subject (e.g., a donor of precursors of the engineered immune cells).

In some cases, precursor cells are cultured prior to transfection with a polynucleotide encoding a cytokine receptor switch. In such cases, the precursor cells can be activated, differentiated, and or expanded. The cells can be further cultured following transfection, for example to further expand the cells or to affect terminal differentiation. In some cases, precursor cells are transfected with the cytokine receptor switch prior to culturing. The engineered immune cells can express an engineered cytokine receptor switch, and optionally can further express a CAR. In many cases, the engineered immune cells are activated and expanded ex vivo prior to administration to a subject.

In some embodiments, activation and expansion may be performed ex vivo. For example, activation may be performed on engineered immune cells prior to administration to a subject. The engineered immune cells or precursor cells can be cultured for 12 hours to 2 months, for example for 1 day to 1 week, 1 day to 2 weeks, 3 days to 1 week, 3 days to 2 weeks, 1 to 2 weeks, 1 week to one month, one week to 2 months, 2 weeks to 1 month, 2 weeks to 2 months, or 1 to 2 months. The engineered immune cells or precursor cells can be expanded to at least 5-fold, at least 10-fold, at least 25-fold, at least 50-fold, at least 100-fold, at least 200-fold, at least 500-fold, at least 1000-fold, at least 5000-fold, at least 10000-fold, at least 50000-fold, or at least 100000-fold using culturing and expansion methods disclosed herein.

An engineered immune cell composition can be formulated with a solution tolerated by the immune cells. The immune cells are formulated with a solution of biological origin, such as plasma; a synthetic solution, such as saline, dextrose solution, Ringer's solution, phosphate buffered saline; water; or a combination thereof. The formulation can also include a nonaqueous vehicle, such as ethyl oleate or a fatty acid triglyceride.

An engineered immune cell composition can include a pharmaceutically acceptable carrier, diluent, or excipient. In some cases, the composition comprises a viscosity-enhancing agent (e.g., sodium carboxymethylcellulose, dextran, or glycerol). In some cases, the composition comprises an isotonicity imparting agent, such as sodium chloride, potassium chloride, or monosodium phosphate. In some cases, the composition comprises a stabilizing agent, such as carboxymethyl cellulose, alginate, polyethylene glycol, or a polyol. In some cases, the composition comprises a preservative, such as thimerosal, m- or o-cresol, formalin or benzyl alcohol. In some cases, the composition comprises an adjuvant, such as aluminum hydroxide. In some cases, the composition comprises a buffer, such as bicarbonate, TRIS, HEPES, MOPS, CHES, CHAPS, or phosphate buffered saline.

Engineered immune cell compositions can either be liquid injectables or solids which can be taken up in a suitable liquid as a suspension or solution for injection. Thus, in a non-liquid formulation, the excipient can comprise, for example, dextrose, human serum albumin, and/or preservatives to which sterile water or saline can be added prior to administration.

The present disclosure provides compositions and methods for controlling immune cell phenotypes generated during activation. Engineered immune cells (e.g., engineered immune cells of a therapeutic composition for administration to a subject) can have a ratio of effector and memory phenotypes of between about 20:1 and about 1:20, between about 5:1 and about 1:5, or between about 2:1 and about 1:2. In some cases, the engineered immune cells have a ratio of effector and memory phenotypes of between about 100:1 and about 10:1, between about 50:1 and about 5:1, between about 25:1 and about 5:1, between about 15:1 and about 3:1, between about 10:1 and about 2:1, between about 5:1 and about 3:2, or between about 4:1 and about 3:2. In some cases, the engineered immune cells have a ratio of effector and memory phenotypes of between about 1:100 and about 1:10, between about 1:50 and about 1:5, between about 1:25 and about 1:5, between about 1:15 and about 1:3, between about 1:10 and about 1:2, between about 1:5 and about 2:3, or between about 1:4 and about 2:3. In some cases, the engineered immune cells comprise at least about 50%, at least about 60%, at least about 70%, at least about 80%, at least about 90%, at least about 95%, at least about 98%, or at least about 99% memory phenotype cells. In some cases, at least about 50%, at least about 60%, at least about 70%, at least about 80%, at least about 90%, at least about 95%, at least about 98%, or at least about 99% of T-cells of the engineered immune cells are memory T-cells. In some cases, at least about 50%, at least about 60%, at least about 70%, at least about 80%, at least about 90%, at least about 95%, at least about 98%, or at least about 99% of B-cells of the engineered immune cells are memory B-cells.

In some cases, at least about 40% of T-cells of the engineered immune cells are central memory cells. In some cases, between about 40% and about 85% of T-cells of the engineered immune cells are central memory cells. In some cases, between about 40% and about 75% of T-cells of the engineered immune cells are central memory cells. In some cases, between about 45% and about 75% of T-cells of the engineered immune cells are central memory cells. In some cases, between about 50% and about 75% of T-cells of the engineered immune cells are central memory cells. In some cases, between about 60% and about 90% of T-cells of the engineered immune cells are central memory cells.

In some cases, between about 3% and about 30% of T-cells of the engineered immune cells are stem memory T-cells. In some cases, between about 4% and about 25% of T-cells of the engineered immune cells are stem memory T-cells. In some cases, between about 4% and about 20% of T-cells of the engineered immune cells are stem memory T-cells. In some cases, between about 3% and about 15% of T-cells of the engineered immune cells are stem memory T-cells.

In some cases, between about 5% and about 60% of T-cells of the engineered immune cells are effector memory T-cells. In some cases, between about 5% and about 50% of T-cells of the engineered immune cells are effector memory T-cells. In some cases, between about 8% and about 40% of T-cells of the engineered immune cells are effector memory T-cells. In some cases, between about 8% and about 35% of T-cells of the engineered immune cells are effector memory T-cells. In some cases, between about 5% and about 25% of T-cells of the engineered immune cells are effector memory T-cells. In some cases, between about 5% and about 20% of T-cells of the engineered immune cells are effector memory T-cells.

### VII. Methods of Treatment

Compositions comprising engineered immune cell populations of the present disclosure (e.g., an engineered immune cell population generating according to a method disclosed herein) can be administered to a subject in need thereof. In some cases, the administering treats a cancer in the subject. In some cases, between about 10⁵ and about 5×10⁷ immune cells are administered to the subject. In some cases, between about 5×10⁴ and about 3×10⁷ memory immune cells are administered to the subject. In some cases, between about 5×10⁴ and about 3×10⁷ effector immune cells are administered to the subject. In some cases, a method of treatment comprises administering a first dose of engineered immune cells comprising at least 50% effector phenotype immune cells, and a second dose of engineered immune cells comprising at least 50% memory phenotype immune cells. In some cases, a method of treatment comprises administering a first dose of engineered immune cells comprising a greater number of effector phenotype immune cells than memory phenotype immune cells, and a second dose of engineered immune cells comprising a greater number of memory phenotype immune cells than effector phenotype immune cells. In some cases, the first dose of engineered immune cells and the second dose of engineered immune cells express an engineered cytokine receptor switch. In some cases, the first dose of engineered immune cells and the second dose of engineered immune cells are subjected to different activation conditions prior to administration. In some cases, the second dose of engineered immune cells is administered at least 1 day, at least 3 days, at least 7 days, at least 14 days, at least 28 days, or at least 60 days after the first dose of engineered immune cells.

In many cases, the engineered immune cells express a CAR. The CAR can recruit an engineered immune cell of the engineered immune cell population to a cancer cell by binding a CAR expressed by the engineered immune cell to a target antigen on the cancer cell, in some cases activating a response against the cancer cell and/or killing the cancer cell. In some cases, the cancer is acute myeloid leukemia, multiple myeloma, ovarian cancer, mesothelioma, non-Hodgkin lymphoma, acute lymphoblastic leukemia, mantle cell lymphoma, follicular lymphoma, glioma (e.g., diffuse midline glioma), pancreatic cancer, prostate cancer, or gastric cancer. In some cases, the target antigen comprises CD19, CD20, CD22, CD123, CD33, CD3, CD4, CD8, CD38, SLAMF7, BCMA, GD2, GPRC5D, MUC16, HER2, EGFR, EGFRVIII, CLL-1, CD44v6, folate receptor-α, B7-H3, EphA2, GRP78, NKG2D, CD70, or mesothelin. In some cases, treating the cancer comprises preventing recurrence of the cancer.

In some embodiments, an immune cell engineered to express an engineered cytokine receptor switch and a direct CAR that directly recognizes a tumor antigen may be used to treat a cancer. For example, an immune cell engineered to express a cytokine receptor switch and an anti-CD123 CAR or an anti-CD33 CAR may be used to treat acute myeloid leukemia. In another example, an immune cell engineered to express a cytokine receptor switch and an anti-BCMA CAR or an anti-GPRC5D CAR may be used to treat multiple myeloma. In another example, an immune cell engineered to express a cytokine receptor switch and an anti-MUC16 CAR, an anti-HER2 CAR, an anti-mesothelin CAR, or an anti-folate receptor-a CAR may be used to treat ovarian cancer. In another example, an immune cell engineered to express a cytokine receptor switch and an anti-mesothelin CAR may be used to treat mesothelioma. In a further example, an immune cell engineered to express a cytokine receptor switch and an anti-GD2 CAR may be used to treat glioma.

In some embodiments, an immune cell engineered to express an engineered cytokine receptor switch and an indirect CAR that recognizes a bispecific agent that binds to a tumor antigen may be used to treat a cancer. For example, an immune cell engineered to express a cytokine receptor switch and an indirect CAR that recognizes a bispecific agent including an anti-CD123 antibody or an anti-CD33 antibody may be used to treat acute myeloid leukemia. In another example, an immune cell engineered to express a cytokine receptor switch and an indirect CAR that recognizes a bispecific agent including an anti-BCMA antibody or an anti-GPRC5D antibody may be used to treat multiple myeloma. In another example, an immune cell engineered to express a cytokine receptor switch and an indirect CAR that recognizes a bispecific agent including an anti-MUC16 antibody, an anti-HER2 antibody, an anti-mesothelin antibody, or an anti-folate receptor-a antibody may be used to treat ovarian cancer. In another example, an immune cell engineered to express a cytokine receptor switch and an indirect CAR that recognizes a bispecific agent including an anti-mesothelin antibody may be used to treat mesothelioma. In a further example, an immune cell engineered to express a cytokine receptor switch and an indirect CAR that recognizes a bispecific agent including an anti-GD2 antibody may be used to treat glioma.

In some cases, the engineered immune cell persists in the subject for at least 1 week, at least 1 month, at least 1 year, or at least 10 years. In some cases, the engineered immune cell persists in the subject at least 2 times, at least 5 times, or at least 10 times as long as an immune cell that does not express the engineered cytokine receptor switch. In some cases, 2 weeks after the administering, the immune cell population comprises at least 5%, at least 10%, at least 15%, at least 20%, at least about 25%, at least about 30%, at least about 40%, or at least about 50% as many cells as the immune cell population at the time of administration. In some cases, 4 weeks after the administering, the immune cell population comprises at least 5%, at least 10%, at least 15%, at least 20%, at least about 25%, at least about 30%, at least about 40%, or at least about 50% as many cells as the immune cell population at the time of administration. In some cases, 6 weeks after the administering, the immune cell population comprises at least 5%, at least 10%, at least 15%, at least 20%, at least about 25%, at least about 30%, at least about 40%, or at least about 50% as many cells as the immune cell population at the time of administration.

In some embodiments, a method of treating a cancer in a subject is provided. The method can include administering to the subject an immune cell population including immune cells expressing an engineered cytokine receptor switch and a CAR (e.g., a direct CAR or an indirect CAR). The engineered cytokine receptor switch can include an activator binding domain that binds an activator (e.g., an exogenous small molecule), a signal peptide, a hinge domain, a transmembrane domain, and/or an intracellular domain.

In some embodiments, the immune cells are activated ex vivo with the activator before being administered to the subject, e.g., by exposing the immune cells to the activator on a substrate. The ex vivo activation can cause conversion of the immune cells to a memory phenotype, upregulation of lymphoid homing markers on the immune cell, or a combination thereof. The immune cells can be exposed to the activator for at least 1 hour, 2 hours, 3 hours, 4 hours, 5 hours, 6 hours, 7 hours, 8 hours, 9 hours, 10 hours, 11 hours, 12 hours, or 24 hours. Ex vivo activation may be performed at least 1 hour, 2 hours, 3 hours, 4 hours, 5 hours, 6 hours, 7 hours, 8 hours, 9 hours, 10 hours, 11 hours, 12 hours, or 24 hours before administration of the engineered immune cells to the subject. The concentration of the activator for ex vivo activation can be within a range from 1 nM to 1000 nM, 1 nM to 500 nM, 1 nM to 100 nM, 1 nM to 50 nM, 1 nM to 10 nM, 10 nM to 1000 n, 10 nM to 500 nM, 10 nM to 100 nM, 10 nM to 50 nM, 50 nM to 1000 nM, 50 nM to 500 nM, 50 nM to 100 nM, 100 nM to 1000 nM, 100 nM to 500 nM, or 500 nM to 1000 nM. In other embodiments, however, the immune cells are not activated ex vivo before being administered to the subject.

The method can further include activating the immune cells in vivo concurrently with or after the immune cells are administered to the subject, e.g., by administering the activator to the subject concurrently with or after the immune cells are administered to the subject. The in vivo activation can cause conversion of the immune cells to a memory phenotype, upregulation of lymphoid homing markers on the immune cell, or a combination thereof. In some embodiments, a bispecific agent including the activator is administered to the subject. The bispecific agent can include a targeting moiety conjugated to the activator. The targeting moiety can recognize a target for the immune cell, such as a cancer cell, a cell of a lymphoid organ, or another cell type. In some embodiments, the targeting moiety is a lymphoid-targeting protein that directs the immune cell to a lymphoid organ. In some embodiments, the targeting moiety binds a tumor antigen. Optionally, the activator can be administered multiple times, e.g., to re-activate the immune cells in vivo. In other embodiments, the immune cells are not activated in vivo.

The dosage of the activator (e.g., a bispecific agent including the activator) for in vivo activation can be within a range from 0.1 mg/kg to 10 mg/kg, 0.1 mg/kg to 5 mg/kg, 0.1 mg/kg to 2 mg/kg, 0.1 mg/kg to 1 mg/kg, 0.1 mg/kg to 0.5 mg/kg, 0.5 mg/kg to 10 mg/kg, 0.5 mg/kg to 5 mg/kg, 0.5 mg/kg to 2 mg/kg, 0.5 mg/kg to 1 mg/kg, 1 mg/kg to 10 mg/kg, 1 mg/kg to 5 mg/kg, 1 mg/kg to 2 mg/kg, 2 mg/kg to 10 mg/kg, 2 mg/kg to 5 mg/kg, or 5 mg/kg to 10 mg/kg. In some embodiments, the activator (e.g., a bispecific agent including the activator) is administered to the subject at least 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, or 48 hours before the subject receives the engineered immune cells. Alternatively or in combination, the activator (e.g., a bispecific agent including the activator) is administered to the subject concurrently with the engineered immune cells. Alternatively or in combination, the activator (e.g., a bispecific agent including the activator) is administered to the subject at least 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, or 48 hours after the subject receives the engineered immune cells.

In some embodiments, the method includes causing conversion of the immune cells to a memory phenotype, upregulation of lymphoid homing markers on the immune cell, or a combination thereof, without activating the immune cells with the activator. In some embodiments, the method includes causing conversion of the immune cells to a memory phenotype, upregulation of lymphoid homing markers on the immune cell, or a combination thereof, via activator-independent activity of the engineered cytokine receptor switch. The activator-independent activity can include dimerization of the engineered cytokine receptor switch without binding of the activator to the engineered cytokine receptor switch and/or interactions of the engineered cytokine receptor switch another receptor on the immune cell (e.g., the CAR).

In some embodiments, the activator (e.g., a bispecific agent including the activator) is administered to the subject a single time. Alternatively, the activator (e.g., a bispecific agent including the activator) may be administered to the subject multiple times, e.g., two, three, four, five, or more times. The administration frequency can be at any suitable time interval, such as daily, weekly, biweekly, monthly, yearly, etc. In such embodiments, the bispecific agent that is administered to the subject may be the same for some or all of the administrations, or may be different for some or all of the administrations. For instance, a first bispecific agent including the activator and a targeting moiety for a lymphoid organ may be provided to the subject at a first time point to recruit the engineered immune cells to a lymphoid organ; and a second bispecific agent including the activator and a targeting moiety for a tumor antigen may be provided to the subject at a second, later time point to recruit the engineered immune cells to a tumor cell.

In embodiments where the immune cell is engineered to express an indirect CAR, the method can further include administering a bispecific agent to the subject, where the bispecific agent includes a synthetic antigen recognized by the indirect CAR, and a targeting moiety that recognizes a tumor antigen on a cancer cell. The bispecific agent can be administered concurrently with or after the immune cells are administered to the subject. The dosage of the bispecific agent can be within a range from 0.1 mg/kg to 10 mg/kg, 0.1 mg/kg to 5 mg/kg, 0.1 mg/kg to 2 mg/kg, 0.1 mg/kg to 1 mg/kg, 0.1 mg/kg to 0.5 mg/kg, 0.5 mg/kg to 10 mg/kg, 0.5 mg/kg to 5 mg/kg, 0.5 mg/kg to 2 mg/kg, 0.5 mg/kg to 1 mg/kg, 1 mg/kg to 10 mg/kg, 1 mg/kg to 5 mg/kg, 1 mg/kg to 2 mg/kg, 2 mg/kg to 10 mg/kg, 2 mg/kg to 5 mg/kg, or 5 mg/kg to 10 mg/kg.

In some embodiments, the method includes administering a first bispecific agent to the subject, and administering a second bispecific agent to the subject. For example, the first bispecific agent can include an activator for an engineered cytokine receptor switch and a first targeting moiety; and the second bispecific agent can include a synthetic antigen for an indirect CAR and a second targeting moiety. The activator can be the same as the synthetic antigen, or the activator can be different than the synthetic antigen. The first targeting moiety can be the same as the second targeting moiety, or the first targeting moiety can be different than the second targeting moiety. The first targeting moiety can recognize the same epitope as the second targeting moiety, or can recognize a different epitope than the second targeting moiety. The dosage of the first targeting moiety can be the same as, greater than, or less than the dosage of the second targeting moiety.

As another example, the first bispecific agent can include an activator for an engineered cytokine receptor switch and a first targeting moiety; and the second bispecific agent can include the activator for the engineered cytokine receptor switch and a second targeting moiety. The first targeting moiety can be different than the second targeting moiety. For instance, the first targeting moiety can recognize a first cell type (e.g., a cell of a lymphoid organ) and the second targeting moiety can recognize a second cell type (e.g., a cancer cell). The dosage of the first targeting moiety can be the same as, greater than, or less than the dosage of the second targeting moiety.

The first bispecific agent may be administered before, concurrently with, and/or after the second bispecific agent. In some embodiments, the first bispecific agent is administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 16 days, at least about 18 days, at least about 20 days, at least about 22 days, at least about 24 days, at least about 26 days, at least about 28 days, at least about 35 days, at least about 42 days, at least about 49 days, at least about 56 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months before the second bispecific agent is administered.

In some embodiments, the second bispecific agent is administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 16 days, at least about 18 days, at least about 20 days, at least about 22 days, at least about 24 days, at least about 26 days, at least about 28 days, at least about 35 days, at least about 42 days, at least about 49 days, at least about 56 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months before the first bispecific agent is administered.

VIII. Substrates For Immune Cell Activation

An activator of a cytokine receptor switch can be coupled to a substrate. In addition to improving in vivo and in vitro activator stability, activator-substrate attachment can prevent phagocytic activator uptake and diminish promiscuous degradation by bystander macrophages in immune preparations. Furthermore, substrate attachment can facilitate efficient activator removal through magnetic, chromatographic, or gravimetric substrate separation. A substrate can comprise a solid, such as a ceramic, a gel, such as a lipid bilayer, a multiphasic material, such as a hydrogel, or a combination thereof. The substrate can be a discrete species, such as a nanoparticle, or an extended structure, such as a surface (e.g., of a microwell or tube). The substrate can be smaller than, of equivalent size to, or larger than a target cell (e.g., an engineered immune cell expressing a cytokine receptor switch). For example, an engineered T lymphocyte with a diameter of about 10 microns can be activated by a 1 micron diameter activator-functionalized bead, a 5-15 micron diameter activator-functionalized polymer matrix, or an activator-functionalized surface.

### A. Beads

A substrate can comprise a bead. As used herein, a bead can refer to a micrometer or nanometer dimensioned solid structure, and can be taken to encompass the terms “nanoparticle” and “microparticle.” While beads often comprise a spherical or oblong character, a bead can also be rough, for example, jagged fragments of larger glass or ceramic structures. A bead can comprise a microparticle, in which cases the bead can comprise a largest dimension (e.g., a diameter or length) of about 1 μm, about 2 μm, about 3 μm, about 4 μm, about 5 μm, about 6 μm, about 8 μm, about 10 μm, about 12 μm, about 15 μm, about 20 μm, about 25 μm, about 30 μm, about 40 μm, or about 50 μm. A bead can comprise a nanoparticle, in which cases the bead can comprise a largest dimension of about 1 nm, about 2 nm, about 3 nm, about 4 nm, about 5 nm, about 6 nm, about 8 nm, about 10 nm, about 12 nm, about 15 nm, about 20 nm, about 25 nm, about 30 nm, about 40 nm, about 50 nm, about 60 nm, about 80 nm, about 100 nm, about 125 nm, about 150 nm, about 200 nm, about 250 nm, about 300 nm, about 400 nm, about 500 nm, about 600 nm, or about 800 nm.

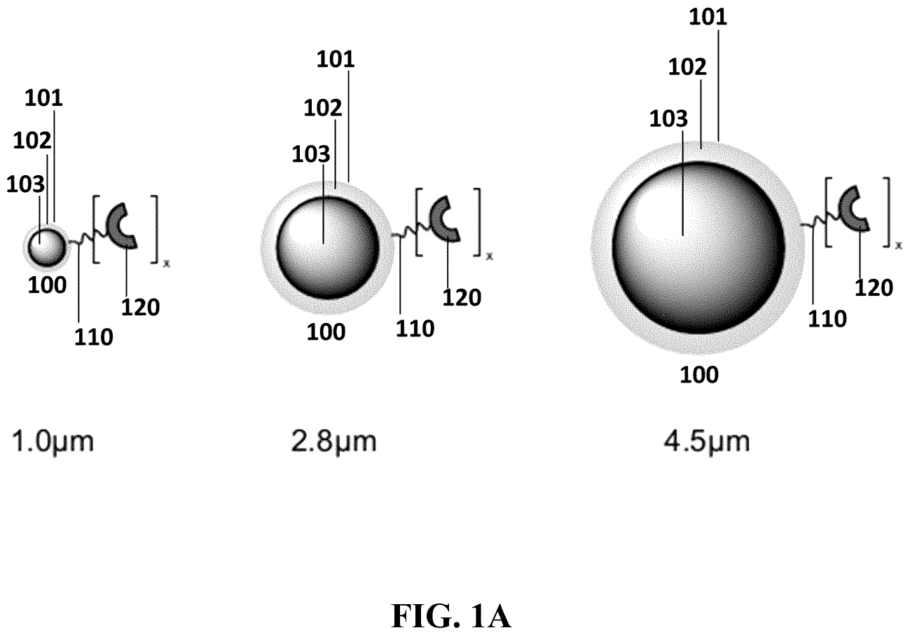

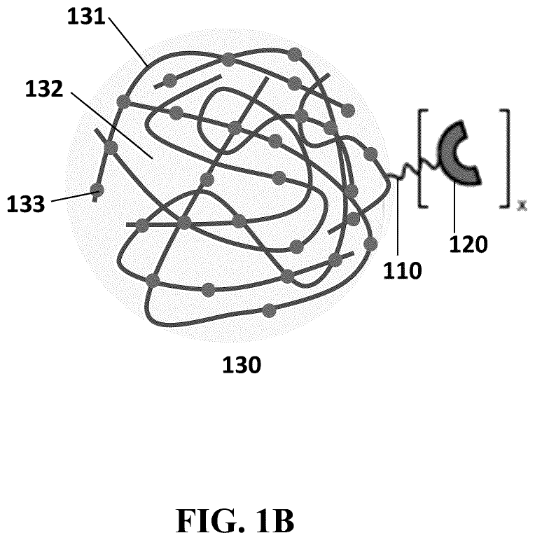

FIG. 1A provides examples of various size beads consistent with the present disclosure. Each activator complex comprises a bead 101. The left, middle, and right panels of the figure depict activator complexes with 1.0 μm, 2.8 μm, and 4.5 μm beads 101, respectively. The beads can be comprised of a single material, or can have separate regions (e.g., layers) with distinct materials. For example, a bead can have a first core material 103 and a second shell material 102 defining a surface 101 of the bead. Alternatively, or in addition thereto, two material constituents of the bead can be heterogeneously dispersed throughout the entirety of the bead or a region of the bead, such as superparamagnetic iron oxide inclusions interspersed within low density polystyrene. Each material (e.g., 102 and 103) can be solid or porous.

Each bead can comprise a chemical handle 120. As described further below, the chemical handle can be an activator of an engineered cytokine receptor switch, a ligand of a natively expressed immune cell receptor, or a combination thereof. The chemical handle can be configured to bind to a chemical handle of a carrier complex, be configured to generate a detectable signal (e.g., a fluorescence signal), or a combination thereof. The chemical handle can be directly coupled to the surface of the bead or coupled to the bead by a linker 110. The linker can be covalently or non-covalently coupled to the bead and chemical handle. For example, a linker consistent with the present disclosure could comprise a covalent succinimide attachment to the bead and a non-covalent attachment to an N-terminal tag on a streptavidin chemical handle.

Beads can be comprised of a single material or can be composites of multiple materials. In some cases, a bead comprises distinct core and shell materials. In some cases, a bead or a portion of a bead comprises solid inclusions, such as superparamagnetic iron oxide nanostructures. A bead can comprise a metal, a polymer, a ceramic, or a combination thereof. A bead can include a magnetic material, including ferromagnetic, ferrimagnetic, paramagnetic, and/or superparamagnetic micro or nanostructures. A bead can include a biomacromolecular structure, such as a polysaccharide, a nucleic acid, a peptide, a protein, or a polyphenol.

### B. Polymer Matrices

A substrate can comprise a polymer matrix. As used herein, a “polymer matrix” can refer to a polymeric or oligomeric material with interstitial phases and/or space. A polymer matrix may be composed of a single polymer or oligomer or a plurality of polymers and/or oligomers, and optionally can include non-oligomeric or polymeric species interspersed within the interstitial phases and/or spaces, such as proteins or nanobeads. In many cases, the polymer matrix comprises a hydrogel.

FIG. 1B provides a diagram of a polymer matrix substrate consistent with the present disclosure. The polymer matrix 130 can include a polymer or oligomer 131 which defines one or more interstitial spaces 132. The interstitial spaces can be empty or can be occupied by solvent, solute, gas, or a combination thereof. The polymer matrix can also include one or more intercalants 133, such as ions (e.g., Ca²⁺), proteins, polymeric particles, or metal oxide, nanostructures. The intercalants can be coupled (e.g., covalently coupled) to the polymer matrix, adsorbed to the polymer matrix, trapped within the polymer matrix, complexed by the polymer matrix, or a combination thereof. The polymer matrix can optionally be crosslinked. The polymer matrix can include linear polymers and/or oligomers, branched polymers and/or oligomers, cyclic polymers and/or oligomers, or a combination thereof. The polymer matrix 130 can be coupled to a chemical handle 120 to functionalize (e.g., surface modify) the polymer matrix 130. The chemical handle can be coupled to a polymer, oligomer, and/or macromolecular structure of the polymer matrix, either directly or through a linker 110.

### C. Surfaces

A substrate can include a functionalized surface. The surface can comprise a portion of a container for holding immune cells, such as a nanowell, a microwell, a milliliter-sized well, a tube (e.g., an Eppendorf tube), a channel (e.g., a micron- or nanometer-dimensioned channel in a microfluidic device), or a combination thereof. The surface can be coupled directly to a chemical handle (e.g., a small molecule activator), coupled to a chemical handle through a linker, coupled to a chemical handle through a carrier group, or a combination thereof. In certain cases, the surface is coupled to a saccharide carrier complex, such as a small molecule activator-functionalized aminodextran oligosaccharide or polysaccharide.

### D. Chemical Handles

Substrate functionalizations, hereinafter referred to as “chemical handles,” can have binding affinity for a molecular or macromolecular target species such as a protein (e.g., an engineered cytokine receptor switch of an engineered immune cell) or a separate chemical handle of a carrier complex. In many cases, the chemical handle is an activator of a receptor, such as an engineered cytokine receptor switch or a native receptor of an immune cell. Optionally the chemical handle can have a chemical, biological, and/or physical activity. The chemical handle can be detectable, for example capable of generating fluorescence signals.

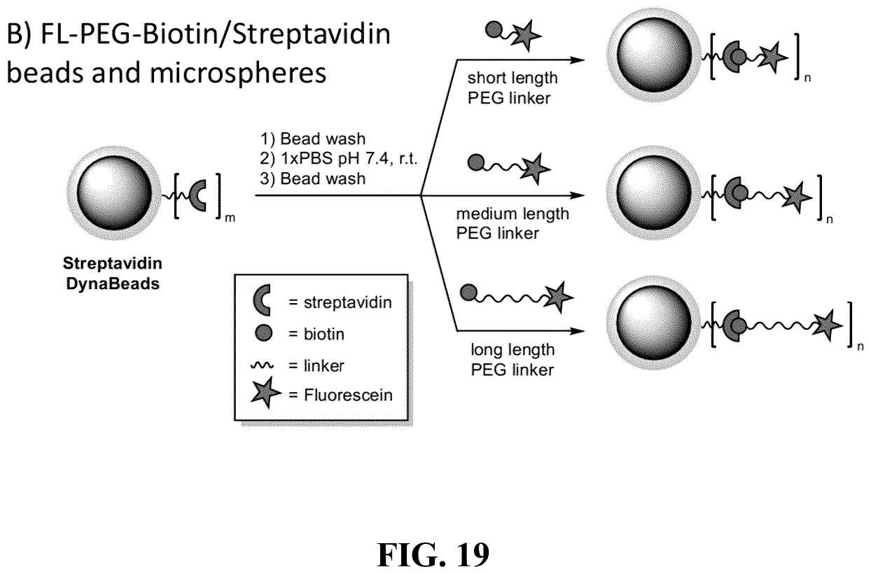

Alternatively, or in addition thereto, the chemical handle can be configured to bind to a target species. In some cases, the target species is another chemical handle (e.g., a chemical handle on a carrier complex). The chemical handle can be configured to covalently or non-covalently couple to a target species. For example, as outlined in FIG. 19, a bead can be functionalized with a streptavidin chemical handle configured to non-covalently couple to biotinylated carrier complexes containing small molecular activators and linkers of varying lengths. Alternatively or in combination, the bead can be functionalized with an azide chemical handle configured to covalently couple to an alkyne group of a carrier complex (not shown).

A chemical handle can comprise a species which binds to an activator binding domain of an engineered cytokine receptor switch. The activator binding domain can be configured to bind to (and in many cases be activated by) the chemical handle, in which cases the chemical handle can be referred to as an “activator.”

An activator may activate an immune cell of the present disclosure by binding to the activator binding domain of an engineered cytokine receptor switch and activating cytokine signaling via the intracellular signaling domain. The activator may be a small molecule, a peptide, an oligonucleotide, or a protein. In some embodiments, an activator may be selected to have low toxicity, low immunogenicity, low cross-reactivity, or combinations thereof to reduce unfavorable side effects when administered to a subject (e.g., a human subject). For instance, the activator can be a molecular that is non-toxic to humans, included in an Inactive Ingredients Database, or both. In some embodiments, the activator may be an exogenous activator (e.g., an exogenous small molecule, an exogenous peptide, an exogenous oligonucleotide, or an exogenous protein) that is not naturally present in a target environment (e.g., a human subject) to prevent activation of the engineered cytokine receptor switch in the absence of an external stimulus (e.g., administration of the activator), prevent cross-reactivity of the activator with other biological components, and to enable dynamic control of receptor signaling.

In many cases, the activator is a small molecule activator (e.g., comprises a small molecule which an activator binding domain of an engineered cytokine receptor switch is configured to bind). The small molecule activator may be an antioxidant, a chelator (e.g., a Ca²⁺ chelator), a dye, a food additive (e.g., a preservative such as benzoic acid or an animal feed additive such as ractopamine), an antibiotic, an antiparasitic agent, an antibacterial agent, an antimycobacterial agent, an antihistamine, an antiviral agent, a chemotherapeutic, a cough medicine (e.g., Randox), a diuretic, a fat, an inflammatory drug, a pesticide, a plant hormone, a plasticizer, or a combination thereof.

In some cases, the small molecule activator is selected from the group consisting of fluorescein, DOTA, butylated hydroxytoluene, Rhodamine B, benzoic acid, erythrosine, tartrazine, PEG methoxy group, derivatives thereof, and combinations thereof. In some cases, the small molecule activator is selected from the group consisting of fluorescein, DOTA, butylated hydroxytoluene, Rhodamine B, benzoic acid, erythrosine, tartrazine, and PEG methoxy group. In some cases, the small molecule activator is selected from the group consisting of fluorescein, DOTA, butylated hydroxytoluene, Rhodamine B, benzoic acid, erythrosine, tartrazine, PEG methoxy group, dinitrophenyl (DNP), and polyhistidine tags (e.g., His6), a derivative thereof, and a combination thereof. In some cases, the small molecule activator is selected from the group consisting of fluorescein, DOTA, butylated hydroxytoluene, Rhodamine B, benzoic acid, erythrosine, tartrazine, PEG methoxy group, dinitrophenyl (DNP), and polyhistidine tags (e.g., His6).

Examples of small molecule activators (e.g., haptens) that may be used to activate an engineered cytokine receptor switch include fluorophores (e.g., fluorescein, fluorescein derivatives, indocyanines, indocyanine derivatives, cyanines, or cyanine or cyanine derivatives), chelators (e.g., DOTA), or other small molecules. For example, the fluorescein derivative may be fluorescein isothiocyanate (FITC), fluorescein 5-maleimide, fluorescein-5-carboxamide, fluorescein-6-carboxamide, or 6-FAM phosphoramidite. Additional examples of small molecules that may bind to an activator binding domain of a cytokine receptor switch include Topiramate hemisuccinate, Creatine, Acetaminophen, Ketamine, Propofol, Lidocaine, Ractopamine, Salicylate, Salicylic Acid, Sulfasalazine, Dapsone, Albendazole, Ivermectin, Levamisole, Permethrin, Pyrantel, Thiabendazole, Procainamide, Sulfamethazine, Amikacin, Amoxicillin, Ampicillin, Cefazolin, Cefuroxime, Cephalexin, Chloramphenicol, Chloramphenicol, Ciprofloxacin, Clenbuterol, Cloxacillin, Colistin A, Dicloxacillin, Enrofloxacin, Furaltadone, Gentamicin, Gentamicin, Kanamycin, Kanamycin, Kincomycin, Lincomycin, Metronidazole, Nafcillin, Nalidixic Acid, Neomycin, Neomycin, Nitrofurazone, Norfloxacin, Ofloxacin, Oxacillin, Spectinomycin, Streptomycin, Streptomycin, Sulfabenzamide, Sulfacetamide, Sulfadiazine, Sulfadimidine, Sulfametoxydiazine, Sulfanilamide, Trimethoprim, Carbamazepine, Ethosuximide, Lamotrigine, Primidone, Cetirizine, Chlorpheniramine, Diphenhydramine, Doxylamine, Promethazine, Sulfadimethoxine, Benzothiazinone, Butylated Hydroxytoluene, Tripelennamine, Chlorpromazine, Clozapine, Haloperidol, Olanzapine, Paliperidone, Quetiapine, Ribavirin, Meprobamate, Acebutolol, Atenolol, Penbutolol, Warfarin, Salmeterol, Aflatoxin B1, Tetraxetan (DOTA), MPOB, Biotin, Melamine, Methotrexate, Amphetamine, Diethylpropion, Dextromethorphan, Pseudoephedrine, Dihydrochlorothiazide, Hydrochlorothiazide, Clonazepam, Diazepam, Nitrazepam, Rhodamine B, Fluorescent Brightener Ksn, Zearalenone, Sudan Red1, Acetominophen, Acrylamide, Benzoic Acid, Benzophenone, Benzothiazine, Mercaptobenzothiazole, Erythrosine, Sudan, Tartrazine, Erythromycin, Sirolimus, Atropine, Ethyl glucuronide, Aflatoxin M1, Methocarbamol, Fentanyl, Hydromorphone, Morphine, Remifentanil, Tapentadol, Tramadol, Pregabalin, Gabapentin, Amitriptyline, Desipramine, Imipramine, Nortriptyline, Venlafaxine, Dinitrophenyl (DNP), His-Tag, PEG methoxy group, Etodolac, Ibuprofen, Ketoprofen, Meclofenamic Acid, Phenylbutazone, Acetyl Salicylic Acid, Acetamiprid, Acetochlor, Carbadazim, Carbaryl, Chlorothalonil, Chlorpyrifos, Fenpropathrin, Imazalil, Imidacloprid, Parathion, Abscisic acid, Dibutyl Phthalate, Clonazepam, Lorazepam, Oxazepam, Phenobarbital, Secobarbital, Zaleplon, Zolpidem, Trazodone, Fluoxetine, Fluvoxamine, Cortisone, Dexamethasone, Dihydrotestosterone, Fluocinolone, Methylprednisolone, Prednisolone, Stanozolol, Triamcinolone, Mazindol, Methamphetamine, Methylphenidate, Modafinil, Chrysoidine, Deoxynivalenol, Fumonisin, Microcystin Lr, Ochratoxin, Sterigmatocystin, T-2 toxin, Sildenafil, Tadalafil, Scopolamine, Florfenicol, Pirlimycin, Sulfaquinoxaline, derivatives thereof, and combinations thereof. In some cases, the small molecule activator is selected from the group consisting of Topiramate hemisuccinate, Creatine, Acetaminophen, Ketamine, Propofol, Lidocaine, Ractopamine, Salicylate, Salicylic Acid, Sulfasalazine, Dapsone, Albendazole, Ivermectin, Levamisole, Permethrin, Pyrantel, Thiabendazole, Procainamide, Sulfamethazine, Amikacin, Amoxicillin, Ampicillin, Cefazolin, Cefuroxime, Cephalexin, Chloramphenicol, Chloramphenicol, Ciprofloxacin, Clenbuterol, Cloxacillin, Colistin A, Dicloxacillin, Enrofloxacin, Furaltadone, Gentamicin, Gentamicin, Kanamycin, Kanamycin, Kincomycin, Lincomycin, Metronidazole, Nafcillin, Nalidixic Acid, Neomycin, Neomycin, Nitrofurazone, Norfloxacin, Ofloxacin, Oxacillin, Spectinomycin, Streptomycin, Streptomycin, Sulfabenzamide, Sulfacetamide, Sulfadiazine, Sulfadimidine, Sulfametoxydiazine, Sulfanilamide, Trimethoprim, Carbamazepine, Ethosuximide, Lamotrigine, Primidone, Cetirizine, Chlorpheniramine, Diphenhydramine, Doxylamine, Promethazine, Sulfadimethoxine, Benzothiazinone, Butylated Hydroxytoluene, Tripelennamine, Chlorpromazine, Clozapine, Haloperidol, Olanzapine, Paliperidone, Quetiapine, Ribavirin, Meprobamate, Acebutolol, Atenolol, Penbutolol, Warfarin, Salmeterol, Aflatoxin B1, Tetraxetan (DOTA), Melamine, Methotrexate, Amphetamine, Diethylpropion, Dextromethorphan, Pseudoephedrine, Dihydrochlorothiazide, Hydrochlorothiazide, Clonazepam, Diazepam, Nitrazepam, Rhodamine B, Fluorescent Brightener Ksn, Zearalenone, Sudan Red1, Acetominophen, Acrylamide, Benzoic Acid, Benzophenone, Benzothiazine, Mercaptobenzothiazole, Erythrosine, Sudan, Tartrazine, Erythromycin, Sirolimus, Atropine, Ethyl glucuronide, Aflatoxin M1, Methocarbamol, Fentanyl, Hydromorphone, Morphine, Remifentanil, Tapentadol, Tramadol, Pregabalin, Gabapentin, Amitriptyline, Desipramine, Imipramine, Nortriptyline, Venlafaxine, Dinitrophenyl (DNP), His-Tag, PEG methoxy group, Etodolac, Ibuprofen, Ketoprofen, Meclofenamic Acid, Phenylbutazone, Acetyl Salicylic Acid, Acetamiprid, Acetochlor, Carbadazim, Carbaryl, Chlorothalonil, Chlorpyrifos, Fenpropathrin, Imazalil, Imidacloprid, Parathion, Abscisic acid, Dibutyl Phthalate, Clonazepam, Lorazepam, Oxazepam, Phenobarbital, Secobarbital, Zaleplon, Zolpidem, Trazodone, Fluoxetine, Fluvoxamine, Cortisone, Dexamethasone, Dihydrotestosterone, Fluocinolone, Methylprednisolone, Prednisolone, Stanozolol, Triamcinolone, Mazindol, Methamphetamine, Methylphenidate, Modafinil, Chrysoidine, Deoxynivalenol, Fumonisin, Microcystin Lr, Ochratoxin, Sterigmatocystin, T-2 toxin, Sildenafil, Tadalafil, Scopolamine, Florfenicol, Pirlimycin, and Sulfaquinoxaline.

In some embodiments, the activator is a surface antigen specific for the type of cancer to be treated, or is a portion of such a surface antigen. Examples of surface antigens that may be used to activate an engineered cytokine receptor switch include CD19, CD20, CD22, CD123, CD33, CD3, CD4, CD8, CD38, SLAMF7, BCMA, GD2, GPRC5D, MUC16, HER2, EGFR, EGFRVIII, CLL-1, CD44v6, folate receptor-α, B7-H3, EphA2, GRP78, NKG2D, CD70, or mesothelin, or a portion thereof.

In some embodiments, the activator is an immunosuppressive molecule, or is a portion of a such an immunosuppressive molecule. For example, the activator can be a molecule that induces inhibition of CAR T-cell activity, such as a ligand of an immune checkpoint family member. In some embodiments, the engineered cytokine receptor switch binds to any of the following or to a ligand of any of the following: CD2, CD95 (Fas), CTLA4 (CD152), CD172A (SIRPa), CD200R, CD223 (LAG3), CD279 (PD-1), CD272 (BTLA), CD300, CD366 (TIM3), A2aR, KIR, LPA5, TIGIT (e.g., CD155 (PVR), CD112 (PVRL2/nectin-2)), TGFß, CD58 (LFA3), CD178 (Fas-L), CD80 (B7-1), CD86 (B7-2), CD47, CD200, LAG-3 (e.g., MHCII, FGL-1, Gal-3, LSECtin, α-syn), CD273 (PD-L2), CD274 (PD-L1), CD258 (HVEM), CD300, CD94 (NKG2A), TIM3 (e.g., Galectin 9, PtdSer, HMGB1, CEACAM1), GPR92, IL6, IL10, or adenosine.

In some embodiments, an activator may be used to activate an immune cell expressing the engineered cytokine receptor switch. For example, the activator may be used to promote conversion of the immune cell to a memory phenotype (e.g., a stem cell memory phenotype, a central memory phenotype, an effector memory phenotype, or an effector memory re-expressing CD45RA phenotype). Activation may be performed ex vivo (e.g., during immune cell manufacturing) or in vivo (e.g., during immune cell therapy treatment). In some embodiments, activation is performed both ex vivo and in vivo. In some embodiments, activation is performed ex vivo but not in vivo. In some embodiments, activation is performed in vivo but not ex vivo.

For ex vivo activation, the activator may be conjugated (e.g., via covalent or non-covalent linkages) or otherwise attached (e.g., adsorbed, adhered) to a substrate, such as a surface (e.g., a plate surface), a bead (e.g., a polystyrene paramagnetic bead), a carrier protein (e.g., an antibody), a carrier polymer (e.g., a synthetic polymer, a biopolymer), a carrier nucleic acid (e.g., an oligonucleotide, a polynucleotide), or combinations thereof. For example, the activator may be conjugated to a carrier protein by classical stochastic cysteine and lysine conjugations, or through a site-specific conjugation technology. In some embodiments, the activator may be adhered to a surface, and immune cells expressing the engineered cytokine receptor may be added to the surface to activate the immune cells. In some embodiments, the activator may be adsorbed to beads, and the beads may be added to a suspension of immune cells to activate the immune cells. The beads may be removed prior to administration of the immune cells to a subject.

For in vivo activation, the activator may be part of a bispecific agent that is administered to the subject. A bispecific agent can include an activator that binds to an engineered immune cell and a targeting moiety that binds to a target for the immune cell (e.g., a cancer cell, a cell of a lymphoid organ, or another cell type). The targeting moiety can be a carrier protein, such as an antibody, an antibody fragment, a single chain variable fragment (scFv), a nanobody, or a peptide. In some embodiments, the targeting moiety (e.g., carrier protein) may be humanized to reduce immunogenicity. The activator can be conjugated (e.g., via covalent or non-covalent linkages) to the targeting moiety to form the bispecific agent. For example, the activator may be conjugated to the targeting moiety (e.g., carrier protein) by classical stochastic cysteine and lysine conjugations, or through a site-specific conjugation technology. The bispecific agent (e.g., activator-carrier protein conjugate) may be administered to a subject who has been or will be treated with immune cells expressing the engineered cytokine receptor switch.

In some embodiments, the targeting moiety may be an antibody, antibody fragment, scFv, nanobody, peptide, etc., that binds to a tumor antigen. For example, the antigen may be CD19, CD20, CD22, CD123, CD33, CD3, CD4, CD8, CD38, SLAMF7, BCMA, GD2, GPRC5D, MUC16, HER2, EGFR, EGFRVIII, CLL-1, CD44v6, folate receptor-α, mesothelin, CD20, CD37, ROR1, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-1, IGF-II, IGF-1 receptor, IL13Rα2, B7-H3 (CD276), EPHA2, GRP78, NKG2D, or CD70.

Binding of the bispecific agent to the tumor antigen and to an engineered cytokine receptor switch may recruit an immune cell expressing the engineered cytokine receptor switch to a tumor cell. In some embodiments, the immune cell is also engineered to express a CAR for a tumor antigen of the tumor cell, which may or may not be the same as the tumor antigen recognized by the bispecific agent. Accordingly, binding of the bispecific agent to its respective tumor antigen may facilitate and/or enhance binding of the CAR to its respective tumor antigen, which in turn may facilitate killing of the tumor cell.

In some embodiments, the targeting moiety is a lymphoid-targeting protein that directs the immune cells to a lymphoid organ, such as a lymph node (e.g., a tumor-draining lymph node), a spleen, a thymus, or bone marrow. The lymphoid-targeting protein may comprise an antibody, an antibody fragment, a single chain variable fragment (scFv), a miniprotein, a nanobody, or a peptide. The lymphoid-targeting protein may cause the immune cell to bind an antigen presenting cell, a T-cell, a B-cell, a lymphocyte, a lymphatic endothelial cell, a B cell, a macrophage, or a lymphoid organ stroma cell. The lymphoid-targeting protein may be engineered to bind a lymphoid marker. In some embodiments, the lymphoid marker is a surface marker (e.g., a cell surface protein) expressed in one or more lymphoid organs, such as the lymph nodes (e.g., tumor-draining lymph nodes, non-tumor-draining lymph nodes), spleen, thymus, bone marrow, or combinations thereof. For example, the lymphoid marker can be a surface-exposed epitope of a cell surface or transmembrane protein. The lymphoid marker can be expressed by a cell that is resident in or otherwise associated with a lymphoid organ, such as an antigen presenting cell (e.g., a dendritic cell), a T-cell, a B-cell, a lymphocyte, a lymphatic endothelial cell, a B cell, a macrophage, or a lymphoid organ stromal cell. The lymphoid marker can be a surface marker expressed by lymphocytes that reside in lymphoid tissue, such as CD3, CD45, CD4, CD2, CD5, CD8, γδ-T-cell receptor, T19, CD45, CD205, cell-surface immunoglobulin (sIg), and L-selectin. The lymphoid marker can be a marker of a stromal cell of a lymphoid organ (e.g., lymph node stromal cells), such as PNAd, VEGFR-3, LYVE-1, Prox-1, podoplanin, CD31, MadCAM1, CXCL13, RANKL, CXCL12, APRIL, BAFF, IL-7, CCL19, CCL21, and Spns2.

Binding of the bispecific agent to the lymphoid marker and to an engineered cytokine receptor switch may recruit an immune cell expressing the engineered cytokine receptor switch to a lymphoid organ. The bispecific agent may bind the immune cell to a cell resident in or otherwise associated with a lymphoid organ, such as an antigen presenting cell (e.g., a dendritic cell), a T-cell, a B-cell, a lymphocyte, a lymphatic endothelial cell, a B cell, a macrophage, or a lymphoid organ stromal cell. The bispecific agent may activate cytokine signaling in the immune cell via the engineered cytokine receptor switch. Recruitment to the lymphoid organ may promote activation and expansion of the immune cell in the lymphoid environment. In some embodiments, once in the lymphoid organ, costimulatory molecules and adhesion molecules present in the lymphoid organ may promote conversion the engineered immune cell to a memory cell phenotype (e.g., a stem-cell memory phenotype, a central memory phenotype, an effector memory phenotype, an effector memory re-expressing CD45RA phenotype). For instance, in embodiments where the immune cell is a T-cell, the T-cells may differentiate into memory T-cells in the lymph nodes and/or other lymphoid organs, and the memory T-cells may contribute to a long-term immune response against antigens recognized by the memory T-cells. The costimulatory and adhesion molecules may also promote activation and clonal expansion of the engineered immune cell. Conversion of the engineered immune cell to a memory cell phenotype may increase the persistence and prolong the efficacy of the engineered immune cell in a subject treated with both the engineered immune cell and the bispecific agent. The lymphoid organ may provide an environment conducive to immune cell activation, facilitating immune cell differentiation and expansion prior to delivery to an immune repressive tumor microenvironment.

In some embodiments, the bispecific agent may bind to the lymphoid marker (e.g., via the lymphoid-targeting protein) with an equilibrium dissociation constant (K_{D}) of no more than 1 μM, no more than 100 nM, no more than 10 nM, or no more than 1 nM. The bispecific agent may retain the engineered immune cell in the lymphoid organ for an amount of time sufficient to activate the immune cell. For example, the bispecific agent may retain the engineered immune cell in the lymphoid organ for 6 to 96 hours, 12 to 72 hours, or 24 to 48 hours.

In some embodiments, it may be important or even necessary for the activator to be attached to a surface to effectively activate immune cells expressing the engineering cytokine receptor switch. For example, the use of such “surface-bound activators” may be beneficial for producing tissue-specific activation of the engineered immune cells. The surface can be the surface of a container (e.g., a plate, tube, bag, bioreactor, chamber, cassette, column), the surface of a bead (e.g., a microparticle, a microsphere), the surface of a tissue (e.g., a lymphoid tissue), the surface of another cell (e.g., a tumor cell, a cell of a lymphoid organ), or any other surface having a high degree of rigidity compared to the immune cell. The surface can be an organic surface, an inorganic surface, or a combination thereof. The activator can be directly attached to the surface, or can be indirectly attached to the surface via a carrier (e.g., a carrier protein, a carrier polymer, a carrier oligonucleotide, or any other bifunctional molecule capable of attaching to both the activator and to the surface). In some embodiments, a plurality of activators are attached to the surface at a sufficiently high density such that binding of two cytokine receptor chains to two proximate activators causes dimerization of the two cytokine receptor chains (e.g., homodimerization of two single-chain cytokine receptor switches or heterodimerization of the two cytokine receptor chains of a dual-chain cytokine receptor switch).

In other embodiments, however, the engineered immune cells may be activated by an activator that is not bound to any surface. For example, the activator can be provided as part of a free-floating, soluble activator-carrier complex. In some embodiments, the activator-carrier complex includes at least two activators such that binding of two cytokine receptor chains to two proximate activators causes dimerization of the two cytokine receptor chains (e.g., homodimerization of two single-chain cytokine receptor switches or heterodimerization of the two cytokine receptor chains of a dual-chain cytokine receptor switch).

### E. Substrate Properties

A substrate can have a single chemical handle or a plurality of chemical handles. Multiple chemical handles can be coupled to a single linker (for example, a dendrimeric linker), or each chemical handle may be individually coupled to the substrate. A substrate can have one type of chemical handle, or multiple types of chemical handles. A substrate can have a density of less than about 1.0 g/cm³ (and therefore float or remain suspended in many aqueous solutions). A substrate can have a density of greater than about 1.0 g/cm³ (and therefore settle from most aqueous solutions). A substrate can be magnetic, for example, having a magnetic hematite core.

### F. Linkers

A substrate can be coupled to a chemical handle by a linker. The linker can comprise a linear or branched structure, and can be configured to spatially separate the substrate from the chemical handle. In many cases, the linker comprises a polymer. Nonlimiting examples of such polymers include polyethylene glycol (PEG), polypropylene glycol (PPG), polyethylene, polyacrylamide, polyacrylate, polyurea, polycarbonate, polycarbamate, polyester, polyvinyl alcohol, polyvinylchloride, polypeptides (e.g., polyalanine), an oligomeric portion thereof, or a combination thereof. The polymer can be water soluble, for example, PEG or polyvinyl alcohol. The polymer can be rigid (e.g., such as polyphenylene) or flexible (e.g., such as PEG).

The linker can have any suitable length. For instance, in embodiments where the linker includes a plurality of repeating units (e.g., ethylene oxide groups in a PEG linker), the linker can include at least 2 repeating units, 5 repeating units, 10 repeating units, 15 repeating units, 20 repeating units, 25 repeating units, 30 repeating units, 35 repeating units, 40 repeating units, 45 repeating units, 50 repeating units, 55 repeating units, 60 repeating units, 65 repeating units, 70 repeating units, 75 repeating units, 80 repeating units, 85 repeating units, 90 repeating units, 95 repeating units, 100 repeating units, 110 repeating units, 120 repeating units, 130 repeating units, 140 repeating units, 150 repeating units, 160 repeating units, 170 repeating units, 180 repeating units, 190 repeating units, 200 repeating units, 500 repeating units, 1000 repeating units, or 2000 repeating units. Alternatively or in combination, the linker can include no more than 2000 repeating units, 1000 repeating units, 500 repeating units, 200 repeating units, 190 repeating units, 180 repeating units, 170 repeating units, 160 repeating units, 150 repeating units, 140 repeating units, 130 repeating units, 120 repeating units, 110 repeating units, 100 repeating units, 95 repeating units, 90 repeating units, 85 repeating units, 80 repeating units, 75 repeating units, 70 repeating units, 65 repeating units, 60 repeating units, 55 repeating units, 50 repeating units, 45 repeating units, 40 repeating units, 35 repeating units, 30 repeating units, 25 repeating units, 20 repeating units, 15 repeating units, 10 repeating units, 5 repeating units, or 2 repeating units.

In some embodiments, the linker is a rigid linker. The rigidity of the linker can also be a tunable parameter to control activation. Linker rigidity may help to hold the activator in an orientation that is beneficial or necessary for activation. A rigid linker may be or include aromatic rings, highly substituted carbon centers, peptides, and/or other groups that restrict linker rotation. In other embodiments, however, the linker can be a flexible linker.

The linker length and/or rigidity may control accessibility of the chemical handle (e.g., activator) for binding to a receptor on an immune cell (e.g., to an engineered cytokine receptor switch or a CAR). In some embodiments, a short rigid linker may be desirable to hold an activator in a particular orientation that is beneficial or necessary to activate signaling. A shorter linker may also be desirable for providing a shorter immunological synapse for efficient T-cell signaling. However, in some instances, excessively short linkers may not provide good recognition by the immune cell, while excessively long linkers may provide suboptimal linkers. Accordingly, the length and/or rigidity of the linker can be selected to achieve optimal performance (e.g., binding and/or activation of signaling).

In some embodiments, the linker is a cleavable linker. For example, the cleavable linker can be a photocleavable linker that is cleaved upon exposure to light, such as ultraviolet light, visible light, near infrared light, infrared light, or suitable combinations thereof. The photocleavable linker can be cleaved by light having a wavelength within a range from 100 nm to 280 nm, 280 m to 315 nm, 315 nm to 400 nm, 380 nm to 450 nm, 450 nm to 485 nm, 485 nm to 500 nm, 500 nm to 565 nm, 565 nm to 590 nm, 590 nm to 625 nm, 625 nm to 750 nm, or 700 nm to 1 mm. As another example, the cleavable linker can be a chemically cleavable linker, such as a disulfide linker, a pH-sensitive linker (e.g., hydrazone), or a linker that is cleaved by a Staudinger reduction of an azide to an amine. In a further example, the cleavable linker can be an enzymatically cleavable linker, such as a linker that is cleaved by a protease (e.g., cathepsin B). Alternatively or in combination, enzymes that degrade the carrier itself may be used (e.g., dextranase, collagenase).

The use of a cleavable linker may be advantageous to facilitate separation of an immune cell that is bound to the chemical handle (e.g., activator) and the substrate to which the chemical handle is coupled. For example, during manufacturing, immune cells that express the engineered cytokine receptor switch may be separated from cells that do not express the engineered cytokine receptor switch by exposing the immune cells to a substrate that is coupled to the activator. For instance, the activator can be conjugated to a bead, surface, carrier protein, carrier polymer, etc. Immune cells expressing the engineered cytokine receptor switch can bind to the activator and thus become coupled to the substrate, while immune cells that do not express the engineered cytokine receptor switch may remain unbound. The binding between the engineered cytokine receptor switch and the activator can thus be used to separate and collect immune cells that express the engineered cytokine receptor switch, thereby yielding a purified population of immune cells that express the engineered cytokine receptor switch. Subsequently, the linker between the activator and the substrate can be cleaved to release the immune cells from the substrate, e.g., before the immune cells are administered to a subject.

In other embodiments, however, the linker can be a non-cleavable linker, such that a cleavage step is not performed during manufacturing of the immune cells. In such embodiments, the immune cell may be separated from the substrate via mechanical disruption, such as using a magnet (e.g., in embodiments where the substrate is magnetic), aspirating the solution with a pipette to release the cells, etc.

### G. Carrier Complexes

A substrate can be functionalized by a carrier complex, which hereinafter can refer to a species comprising one or more chemical handles and which is configured to bind to a substrate (e.g., configured to bind to a chemical handle of a substrate). The carrier complex can comprise a single chemical handle or a plurality of chemical handles, each of which may be identical to or distinct from the chemical handle of the substrate. In many cases, the carrier complex comprises a first chemical handle which is configured to couple to a chemical handle of the substrate, and a second chemical handle configured to bind to a target species or perform a chemical, physical, or biological function. Carrier complex-substrate binding can be reversible or irreversible, providing modularity and multifunctionality to the substrates of the present disclosure.

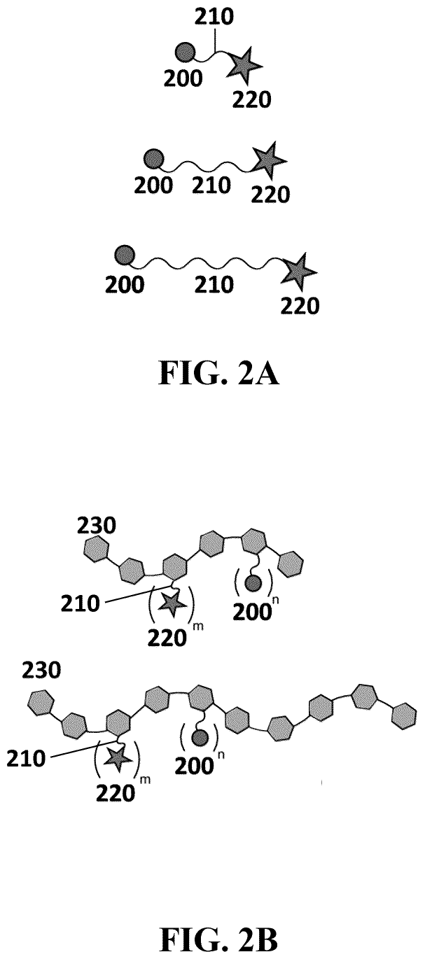

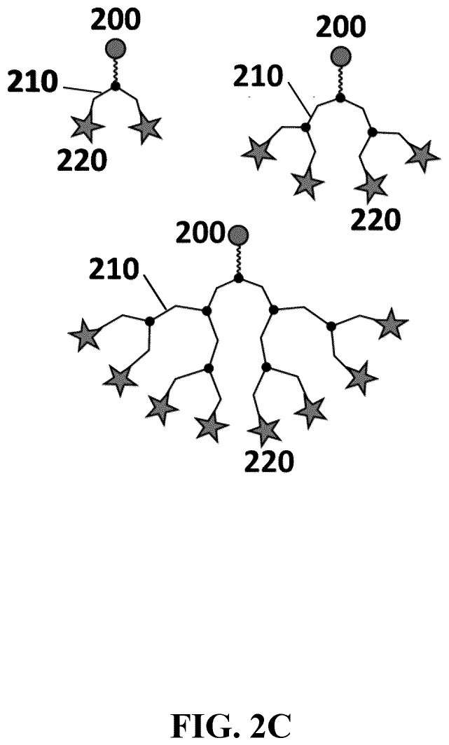

As depicted in FIG. 2A, a carrier complex can include a first chemical handle 200 configured to couple to a substrate (e.g., beads, a polymer matrix) or to a chemical handle of a substrate, a second chemical handle 220 with configured to bind to a target molecule, macromolecular species, or material, and optionally a linker 210. The linker 210 can have different lengths, as shown in FIG. 2A. In one possible construction, a carrier complex can comprise a single instance of the first chemical handle and a single instance of the second chemical handle optionally connected by a linker.

Alternatively, a carrier complex can include a plurality of first chemical handles and/or second. In certain cases, a carrier complex comprises a plurality of first chemical handles and a plurality of second chemical handles. Alternatively, a carrier complex can comprise a single instance of the first chemical handle and a plurality of second chemical handles or a plurality of first chemical handles and a single second chemical handle.

Examples of such carrier complexes are provided in FIG. 2B, which depict carrier oligomeric and polymeric constructs (e.g., semi-flexible synthetic polymers) comprised of monomer units 230. An oligomeric or polymeric carrier complex can comprise one type of monomer unit or multiple types of monomer units 230. For example, a carrier complex can be a homopolymeric or homooligomeric construct, a heteropolymeric or heterooligomeric construct, a block co-polymeric or co-oligomeric construct, a random co-polymeric or co-oligomeric construct, an ordered co-polymeric or co-oligomeric construct, or a combination thereof. The carrier complex can also be branched or linear. Each monomer unit can be coupled to one or more first chemical handles 200, one or more second chemical handles 220, or a combination thereof. Each coupling moiety and chemical handle can independently be coupled directly to a monomer unit or indirectly to the monomer unit through a linker 210. For some systems disclosed herein, a carrier complex with multiple first chemical handles can be configured to multiply bind to a substrate, or may be configured to bind to at most one chemical handle of the substrate.

Each monomer unit can have one or more first chemical handles or one or more second chemical handles. For example, each monomer unit can have at most one first chemical handle 200 and at most one second chemical handle 220. Alternatively, each monomer unit can have at most one chemical handle (e.g., coupled to one first chemical handle and no second chemical handles, coupled to one second chemical handle and no first chemical handles, or coupled to no chemical handles).

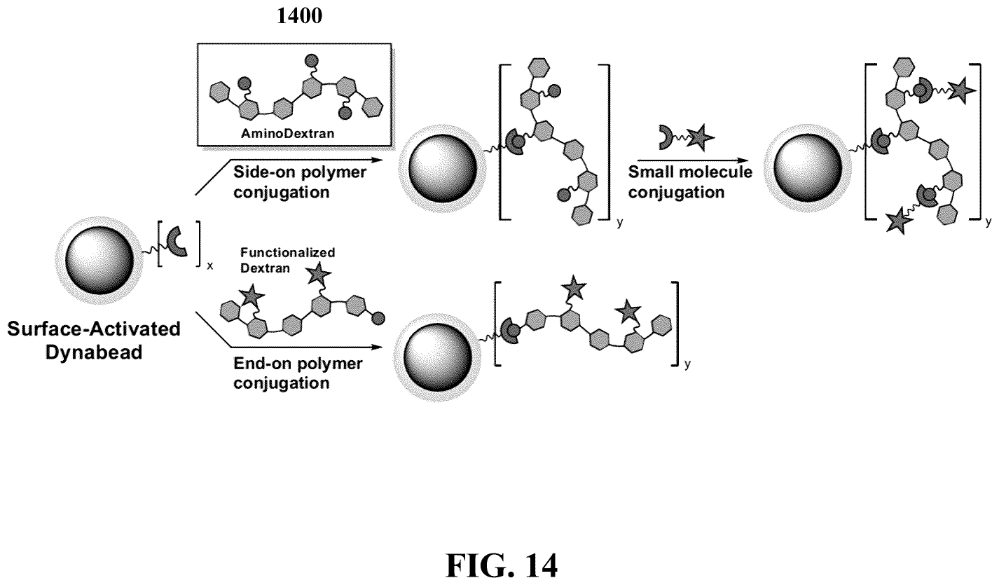

In some cases, a first chemical handle on a first carrier complex is configured to bind to a substrate and to a second carrier complex. FIG. 14 depicts a general strategy for producing such systems. Specifically, the top example provides a “side-on” carrier complex 1400 with multiple first chemical handles 200 (e.g., an aminodextran polymer) capable of coupling to a bead 100 with chemical handles 120 (e.g., a carboxylate-functionalized bead) and, subsequently, to first chemical handles of a second carrier complex (e.g., a carrier complex including a small molecular activator). Following binding to a chemical handle of a bead, the side-on carrier complex can have multiple remaining chemical handles available to couple to copies of the second carrier complex, allowing the side-on carrier complex to adopt multifold functionalization. The distribution of first chemical handles 200 along the side-on carrier complex can be ordered or random. As non-limiting examples, the carrier complex can be generated through a step-wise synthesis with controlled order and distribution of first and/or second chemical handle-functionalized monomer units, or through a random copolymerization process of chemical handle-functionalized and unfunctionalized monomer units, thereby yielding carrier groups with random spacings between chemical handles.

As depicted in the bottom example of FIG. 14, a carrier complex 1410 can also comprise a single first chemical handle at a terminal monomer unit (e.g., a polymer end group of an activator-modified polymer carrier), configuring the carrier complex to bind to a substrate (e.g., a carboxylate-functionalized bead) in an “end-on′” fashion. The carrier complex can comprise one or multiple second chemical handles, and can be branched, cyclic, or linear.

A carrier complex can also comprise a branched dendrimeric structure. Non-limiting examples of such carrier complexes are provided in FIG. 2C, in which each carrier complex comprises a single first chemical handle 200 and a plurality of second chemical handles 220 coupled through linkers 210 configured in a branching structure. The illustrated embodiments show dendronized synthetic polymers having 1, 2, and 3 generations, respectively. Such carrier complexes can be used to increase the functionalization density on a substrate. For example, a substrate with a chemical handle density of 25 nm² (e.g., 1 chemical handle per 25 nm² surface area, or 4×10⁴ chemical handles per μm² of a surface of the substrate) can be made to have 12.5 nm-2, 5 nm-2, or 3.125 nm-2 surface densities of second chemical handles through functionalization with a carrier complex which has 2-, 5-, or 8-second chemical handles, respectively.

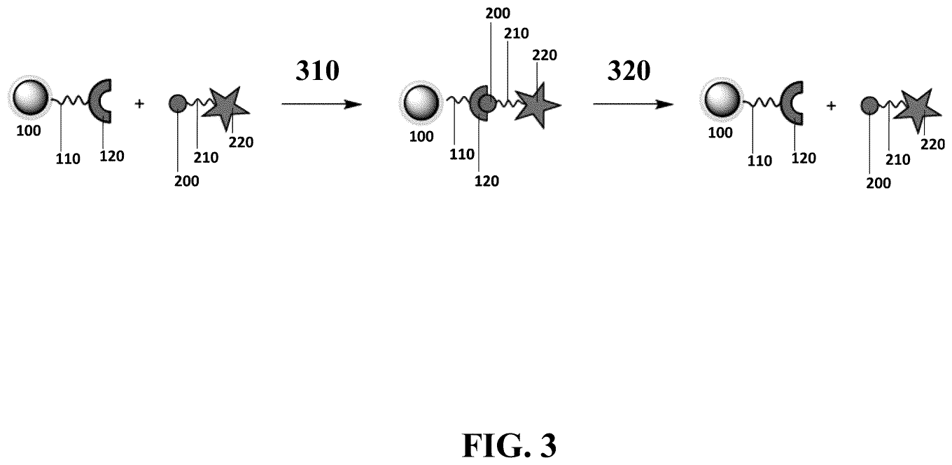

A method of the present disclosure can include coupling a carrier complex to a substrate (e.g., as depicted in FIG. 3). The carrier complex can be contacted to the substrate under conditions 310 which enable coupling between the chemical handle 120 and the coupling moiety 200. The coupling can be reversible or irreversible. For reversibly coupled substrate-carrier complex systems, the carrier complex can, in some cases, be decoupled from the chemical handle in a controlled manner. Such as step 320 can include subjecting a substrate-carrier complex adduct with a condition that releases the carrier complex, contacting the substrate-carrier complex adduct with a species which displaces the carrier complex from the substrate, heating the substrate-carrier complex adduct, irradiating the substrate-carrier complex adduct, or a combination thereof.

### H. Exemplary Substrates

In some cases, an immune-activating substrate comprises: (i) a substrate comprising a first chemical handle, and (ii) a carrier complex comprising a plurality of subunits, of which a first monomeric subunit comprises a second chemical handle and a second monomeric subunit is comprises a third chemical handle; wherein the first chemical handle is coupled to the second chemical handle. In some cases, the third chemical handle is an engineered cytokine receptor switch activator.

In some cases, the first chemical handle is noncovalently coupled to the second chemical handle. In some cases, the first chemical handle comprises a protein and the second chemical handle comprises a small molecule. In some cases, the first chemical handle comprises streptavidin and the second chemical handle comprises biotin. In some cases, the first chemical handle is covalently coupled to the second chemical handle.

In some cases, the first chemical handle is covalently coupled to the second chemical handle. In some cases, the first chemical handle is covalently coupled to the second chemical handle by an amide, thiolsuccinimide, alkylamino, ester, thioester, carbonate, thiocarbonate, carbamate, thiocarbamate, urea, thiourea, ether, thioether or triazole linkage.

In some cases, the second chemical handle is different than the third chemical handle. In some cases, the third chemical handle comprises a fluorophore or a metal chelator. In some cases, the third chemical handle comprises an activator as disclosed herein. In some cases, the third chemical handle comprises a primary amine. In some cases, the third chemical handle comprises an average distance of about 5.0 to about 100 μm from a surface of the substrate.

In some cases, the first monomeric subunit of the carrier complex and the second monomeric subunit of the carrier complex comprise identical structures. For example, each subunit of the carrier complex may be 3-Amino-d-glucose. In some cases, the plurality of subunits comprises a plurality of copies of the first monomeric subunit and a plurality of copies of the second monomeric subunit. In some cases, the plurality of subunits comprises a third monomeric subunit which is not coupled to a chemical handle. In some cases, the first, second, and third monomeric subunits have identical structures. In some cases, the plurality of subunits are randomly ordered within the carrier complex. In some cases, the plurality of subunits comprise an ordered distribution within the carrier complex. In some cases, the plurality of subunits comprises from 2 to 500 monomeric subunits. In some cases, the first monomeric subunit is at least about 50% of subunits of the plurality of subunits. In some cases, the second monomeric subunit is at least about 50% of subunits of the plurality of subunits. In some cases, the third monomeric subunit is at least about 50% of subunits of the plurality of subunits. In some cases, the first monomeric subunit is at least about 75% of subunits of the plurality of subunits. In some cases, the second monomeric subunit is at least about 75% of subunits of the plurality of subunits. In some cases, the third monomeric subunit is at least about 75% of subunits of the plurality of subunits. In some cases, the first monomeric subunit is at least about 90% of subunits of the plurality of subunits. In some cases, the second monomeric subunit is at least about 90% of subunits of the plurality of subunits. In some cases, the third monomeric subunit is at least about 90% of subunits of the plurality of subunits. In some cases, the carrier complex comprises a single copy of the first subunit and a single copy of the second subunit. In some cases, the carrier complex comprises a single copy of the first monomeric subunit, a single copy of the second monomeric subunit, and at least 5, at least 10, at least 20, at least 30, at least 50, at least 100, at least 200, or at least 400 copies of the third monomeric subunit. In some cases, the first monomeric subunit is disposed at an end of the carrier complex. In some cases, the second monomeric subunit is disposed at an end of the carrier complex. In some cases, the first monomeric subunit and the second monomeric subunit are disposed at opposite ends of the carrier complex. In some cases, the carrier complex is linear. In some cases, the carrier complex is branched. In some cases, the carrier complex is cyclic. In some cases, the first monomeric subunit, the second monomeric subunit, the third monomeric subunit, or a combination thereof comprises a saccharide. In some cases, the saccharide comprises dextran. In some cases, the first subunit comprises a C5-C9 saccharide functionalized with a primary amine, a secondary amine, a tertiary amine, a thiol, an azide, an alkyne, or a combination thereof. In some cases, the first and second subunits each comprise a C5-C9 saccharide. In some cases, the first and second subunits each comprise a hexose sugar. In some cases, the third subunit comprises a C5-C9 saccharide. In some cases, the third subunit comprises a hexose sugar.

In some cases, the substrate comprises a nanoparticle, a microparticle, a polymer matrix, a surface, a carbon nanomaterial, a quantum dot, or a combination thereof. In some cases, the substrate comprises a nanoparticle or a microparticle. In some cases, the substrate comprises a surface (e.g., the surface of a nanowell, a microwell, or an Eppendorf tube). In some cases, the substrate comprises a largest dimension of about 0.5 to about 25 microns (μm). In some cases, the substrate comprises a largest dimension of about 1 to about 5 microns (μm). In some cases, the substrate is magnetic. In some cases, the substrate is hydrophobic. In some cases, the substrate is hydrophilic.

In some cases, an immune-activating substrate comprises: a substrate and a dendrimeric carrier complex comprising a single point of attachment to the substrate and a plurality of branches terminating in a first chemical handle; wherein a density of the first chemical handle (defined as a ratio of the number of instances of the first chemical handle to surface area of the substrate) is between about 0.001 and about 100 per nm² of a surface of the substrate. In some cases, the density of the first chemical handle is between about 0.01 and about 10 per nm² of the surface of the substrate. In some cases, the density of the first chemical handle is between about 0.1 and about 10 per nm² of the surface of the substrate. In some cases, the density of the first chemical handle is between about 0.01 and about 1 per nm² of the surface of the substrate.

In some cases, the first chemical handle is an activator of an engineered cytokine receptor switch. In some cases, the first chemical handle comprises a fluorophore, a metal chelator, or a combination thereof. In some cases, changes in an intensity of the detectable signal are linear with respect to changes in the density of the first chemical handle on the surface of the substrate over at least a one order of magnitude density range (i.e., to at least one order of magnitude above or below the density of the first chemical handle on the surface of the substrate). For example, in some cases, changes in an intensity of the detectable signal are linear with respect to changes in density of the first chemical handle on the surface of the substrate over the density range of about 0.01 and 10 per nm² of the substrate.

In some cases, the single point of attachment of the carrier complex to the substrate comprises an amide. In some cases, the first chemical handle is homogeneously distributed over the surface of the substrate. In some cases, the dendrimeric complex comprises between about 2 and about 100 instances of the first chemical handle. In some cases, each instance of the first chemical handle is separated from the point of attachment to the substrate by an equivalent-length linker. In some cases, each instance of the first chemical handle is separated from the point of attachment to the substrate by between about 5 nm and about 100 nm (e.g., as measured by the shortest distance through the carrier complex to the point of attachment).

In some cases, the substrate comprises a nanoparticle, a microparticle, a polymer matrix, a surface, a carbon nanomaterial, a quantum dot, or a combination thereof. In some cases, the substrate comprises a nanoparticle or a microparticle. In some cases, the substrate comprises a largest dimension of about 0.5 to about 25 microns (μm). In some cases, the substrate comprises a largest dimension of about 1 to about 5 microns (μm). In some cases, the substrate is magnetic. In some cases, the substrate is hydrophobic. In some cases, the substrate is hydrophilic.

In some cases, an immune-activating substrate comprises a particle covalently coupled to a plurality of polymeric linkers, a first subset of which linkers terminate in a first chemical handle and a second subset of which linkers terminate in a second chemical handle. In some cases, the particle comprises a diameter of between 0.5 and 10 millimeters (mm). In some cases, the plurality of polymeric linkers share a single type of structure. In some cases, the plurality of polymeric linkers are homogeneously distributed over the surface of the particle. In some cases, a density of the first chemical handle on a surface of the substrate is between about 0.01 and about 10 per nm² of the surface of the substrate. In some cases, the particle is covalently coupled to the homogeneous population of polymeric linkers through amide bonds.

In some cases, the plurality of polymeric linkers comprise PEG backbones. In some cases, the first subset of the plurality of polymeric linkers comprise structures according to FORMULA (IA):

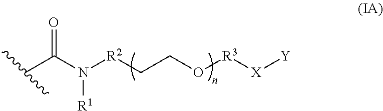

wherein
[symbol] is a point of covalent attachment to the particle;
R¹ is H or optionally substituted C₁₋₆ alkyl;
R² and R³ are each independently selected from the group consisting of a bond, optionally substituted C₁-C₆ alkyl, optionally substituted C₃-C₈ cycloalkyl, optionally substituted C₂-C₇ heterocycloalkyl, optionally substituted aryl, and optionally substituted heteroaryl;
X is the first chemical handle and is selected from the group consisting of —NR⁴C(═O)—, —NR⁴C(═O) NR⁴, —C(═O) NR⁴—, —O—C(═O)—, —C(═O)—O—, —O—C(═O)—O—, —S—C(═O)—, and —C(═O)—S—;
Y is the second chemical handle, and
each instance of R⁴ is independently selected from the group consisting of H and C₁₋₆ alkyl; and
subscript n is an integer of between 2 and 2000.

In some cases, the second subset of the plurality of polymeric linkers comprise structures according to FORMULA (IB):

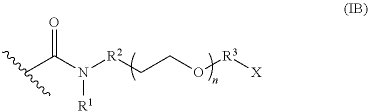

wherein
[symbol] is a point of covalent attachment to the particle;
R¹ is optionally substituted C₁₋₆ alkyl;
R² and R³ are each independently selected from the group consisting of a bond, optionally substituted C₁-C₆ alkyl, optionally substituted C₃-C₈ cycloalkyl, optionally substituted C₂-C₇ heterocycloalkyl, optionally substituted aryl, and optionally substituted heteroaryl;
X is the first chemical handle and is selected from the group consisting of —NHR⁴, —OR⁴, —C(═O)R⁵, —OR⁵, and —SR⁵;
each instance of R⁴ is independently selected from the group consisting of H and C₁₋₆ alkyl;
each instance of R⁵ is independently selected from the group consisting of H, halogen, and hydroxyl; and
subscript n is an integer of between 2 and 2000.

### I. Conjugating Groups

Aspects of the present disclosure provide chemical complexes configured to covalently modify target biomolecules. Such complexes, which hereinafter may be referred to as “conjugating groups,” typically comprise a coupling moiety configured to couple to a target biomolecule and a chemical handle configured to perform a chemical or physical function. In many cases, the chemical handle is an activator of an engineered cytokine receptor switch or a chimeric antigen receptor agonist. Covalent modification of the target biomolecule with one or more conjugating groups can thus impart the chemical functionality of the chemical handle to the target biomolecule.

In many cases, a conjugating group of the present disclosure targets a specific type of biomolecule or portion of a biomolecule for covalent coupling. The coupling moiety of the conjugating group can be configured to couple to multiple types of sites of the target biomolecule, and in this way may be capable of generically and stochastically coupling to a class or type of biomolecule, such as a protein, a nucleic acid, or a saccharide. As an example, a first functionality can comprise a Michael acceptor with sufficient electrophilicity for coupling to any nucleobase of a target nucleic acid, or may comprise an alkyl halide which is capable of coupling to multiple hydroxyls of a target polysaccharide. Alternatively, the first functionality of the conjugating group can be configured to couple to a single type of site or subset of sites of the target molecule, such as an NHS ester configured to selectively couple to lysine side chains of a target peptide, a maleimide configured to selectively couple to cysteine side chains of a target peptide, or an epoxide configured to selectively couple to histidine side chains of a target peptide.

The chemical handle of the conjugating group can perform a chemical or physical function. In some cases, the chemical handle generates a detectable signal, such as a spectroscopic, mass spectrometric, colorimetric, electrical, or chemical signal. For example, the chemical handle can comprise a fluorophore which generates absorbance and fluorescence signals or a ligand which modifies the spectroscopic signals of another species. In specific cases, the chemical handle comprises fluorescein or 2,2′,2″,2′″-(1,4,7,10-Tetraazacyclododecane-1,4,7,10-tetrayl)tetraacetic acid (DOTA). The chemical handle can also be configured to non-covalently bind to a molecular or biomolecular target, such as a monosaccharide or a protein. In some cases, the chemical handle can comprise a binding affinity of at most about 100 μM (e.g., the binding affinity can be 80 μM, 4 μM, 2 nM, etc.), at most about 50 μM, at most about 10 μM at most about 5 μM, at most about 1 μM, at most about 500 nM, at most about 100 nM, at most about 50 nM, at most about 10 nM, at most about 5 nM, or at most about 1 nM. In some cases, the chemical handle is an agonist or an antagonist of the biomolecular target. In many cases, the chemical handle comprises a plurality of chemical and/or physical functions, for example fluorescence and biomolecular antagonist activity.

The coupling moiety and chemical handle of the conjugating group can be coupled by a linker. Linker length and physical and chemical properties can affect the coupling dynamics (e.g., reactivity and stochasticity) of unbound conjugating groups, as well as the dynamics and accessibility of the chemical handle of a bound conjugating group. Alternatively, the first and chemical handle may be directly coupled (e.g., by a bond). For example, while a flexible hydrophilic linker may, in some cases, increase the activity of a chemical handle of a conjugating group coupled to an antibody, a rigid hydrophobic linker may, in some cases, decrease the accessibility and corresponding activity of the chemical handle when the conjugating group is coupled to the antibody.

Table 10 includes exemplary conjugating groups consistent with the present disclosure.

TABLE 10

| Compound | Structure |
|---|---|
| Compound 7 | 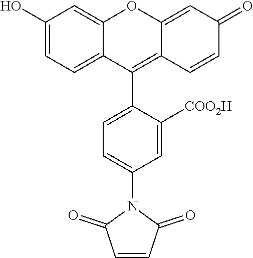 |
| Compound 8 | 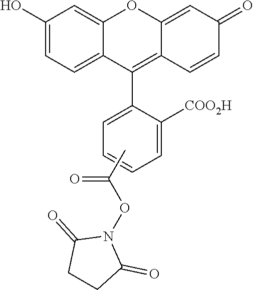 |
| Compound 9 | 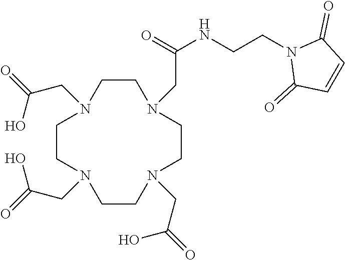 [SMILES: O=C(O)CN1CCN(CC(=O)O)CCN(CC(=O)NCCN2C(=O)C=CC2=O)CCN(CC(=O)O)CC1] |
| Compound 10 | 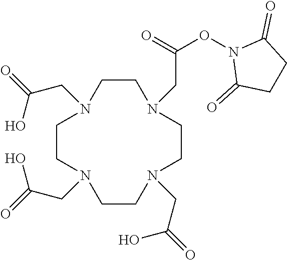 [SMILES: O=C(O)CN1CCN(CC(=O)O)CCN(CC(=O)ON2C(=O)CCC2=O)CCN(CC(=O)O)CC1] |

### J. Cysteine Functionalization

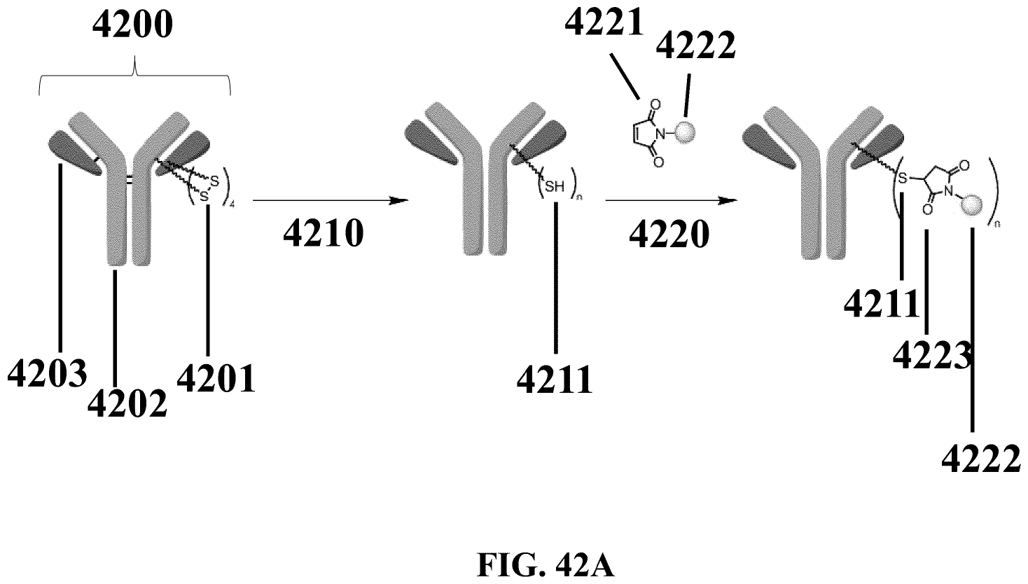

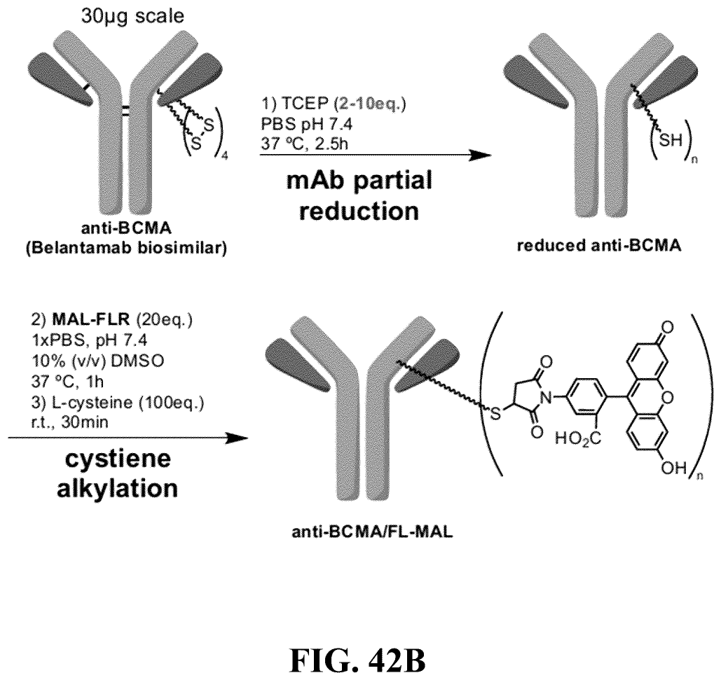

FIG. 42A outlines a method consistent with the present disclosure for functionalizing protein-derived cysteine residues. While this figure covers antibody functionalization, the process which it outlines is broadly applicable to thiol and disulfide-containing protein substrates.

The method can optionally include a disulfide reduction 4210 to liberate reactive thiols 4211 from less reactive disulfide linkages 4201. While thiols are amenable to a range of coupling chemistries, they are also often sequestered as low-reactivity disulfide dimers and/or buried, which can prevent coupling to chemical handles. As outlined in FIG. 42A, antibodies 4200 typically contain interchain disulfide linkages 4201 connecting heavy 4202 and light 4203 chain subunits and intrachain disulfide linkages connecting heavy chain subunits 4202, which are usually solvent exposed and cleave under mild reducing conditions using tris(1-carboxyethyl) phosphine (TCEP). Many antibodies further contain intrachain disulfides (e.g., comprised of two cysteines from the same heavy 4202 or light 4203 chain) which do not cleave under mild or moderate reducing conditions, but which can be cleaved in the presence of strong reducing agents. However, in some cases, an antibody (or non-antibody protein substrate) includes a solvent exposed intrachain or intradomain disulfide linkage which cleaves under moderate or mild reducing conditions. For example, an antibody can contain an engineered CH2 disulfide linkage to block Fcγ (III) receptor binding.

Disulfide reduction 4210 can cleave all solvent exposed disulfide linkages 4201, or can cleave only a subset of reductant-accessible disulfide linkages 4201. Often, disulfide reduction 4210 is performed with an excess of reductant and sufficient incubation times to affect complete interchain disulfide cleavage. For example, IgG1 disulfide reduction typically reduces all 4 IgG1 interchain disulfides, thereby generating eight 8 thiols available for chemical coupling. However, in other cases, sub-stoichiometric reductant or short reduction timelines can affect partial interchain disulfide 4201 cleavage. As solvent-exposed interchain disulfides 4201 typically vary minimally in terms of reduction potential, partial disulfide reduction is often stochastic, with the subset of reduced and unreduced disulfides 4201 following a random distribution.

Following optional disulfide cleavage 4210, antibody 4200 free thiols 4211 can be coupled 4220 to a conjugating group 4221. Owing to moderately high nucleophilicities and low reduction potentials, thiols are amenable to a range of coupling chemistries with low or negligible cross-reactivities for other natural amino acids. As with disulfide reduction, thiol functionalization can be stoichiometric (e.g., each thiol is functionalized) or sub-stoichiometric (e.g., a subset of thiols are functionalized).

The conjugating group 4221 can contain a chemical handle 4222, which can facilitate a range of functions, including detection, purification, partitioning, binding, and protein agonism and antagonism.

In the example depicted in FIG. 42A, free thiols are alkylated with maleimide conjugating groups 4221 to form thiol-4211 succinimide 4223 adducts. While these adducts are typically stable, maleimide conjugation can exhibit reversibility through reverse Michael addition, in which succinimide reverts to maleimide through thiol elimination. This process can lead to appreciable loss of functionalization over physiological timescales, such as multi-week systemic circulation. Hydrolysis of the succinimide, which is outlined in SCHEME 1 below, can inhibit reverse Michael addition by stabilizing succinimide reaction adducts. In this scheme, A″ denotes a substrate, such as a nanoparticle, microparticle, polymer matrix, or a surface; a linker, such as a polyethylene glycol linker; a biomolecule, such as a protein; a molecule, such as a small molecule activator; or a combination thereof. For example, A″ can be the chemical handle 4222 depicted in FIG. 42A. Accordingly, a functionalized biomolecule can include a hydrolyzed succinimide conjugating group, and a method of the present disclosure can comprise hydrolyzing a biomolecule-bound succinimide.

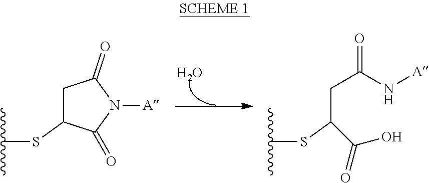

### K. Lysine and N-terminal Functionalization

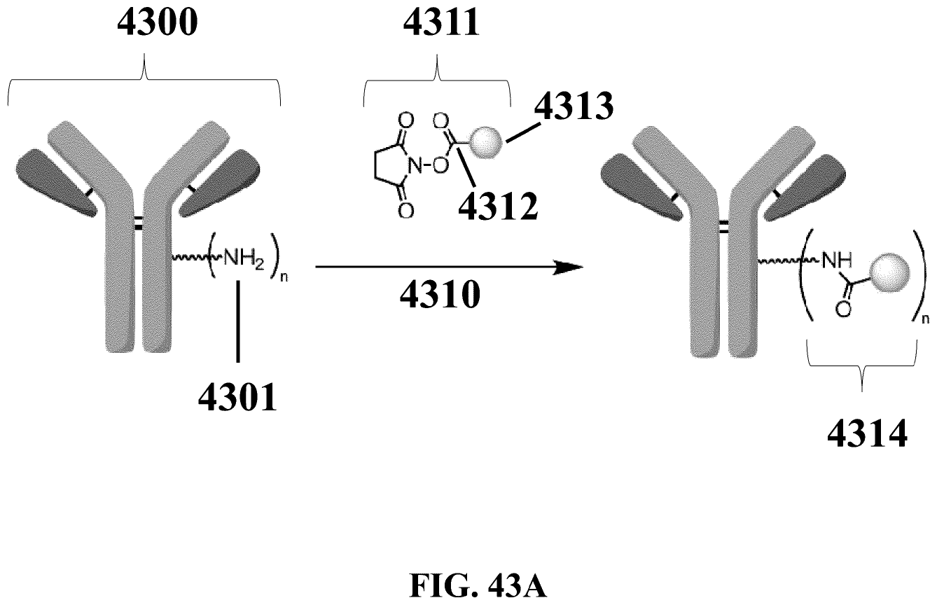

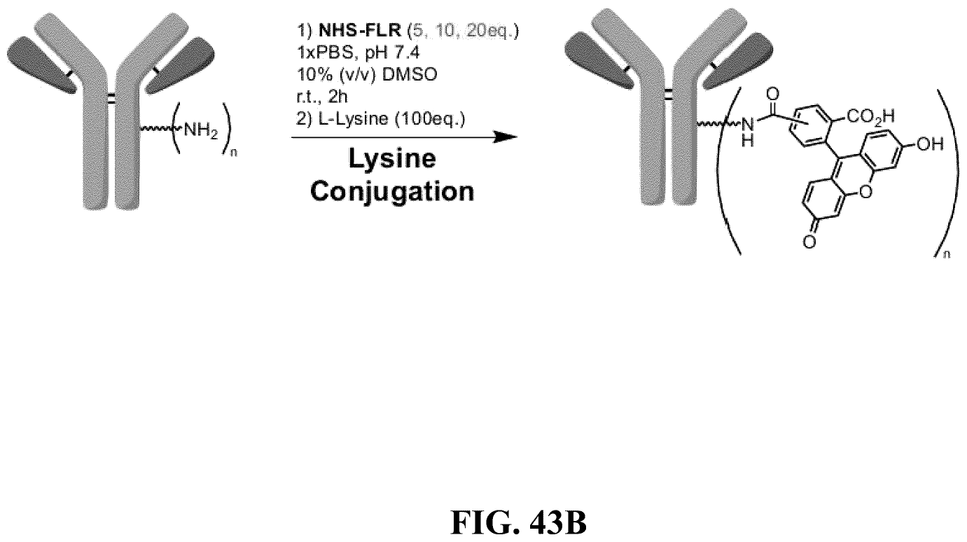

Proteins can also be functionalized at lysine residues and at the N-terminus. While lysine side chains are less nucleophilic than cysteine thiols, their differences in basicity can often be exploited to achieve lysine specific couplings. In many cases, the absence of free thiols (e.g., due to thiol sequestration within disulfide bonds) enables lysine selective reactivity with relatively strong electrophiles. An example of a lysine-selective functionalization method consistent with the present disclosure is provided in FIG. 43A. In this method, a solvent exposed lysine 4301 of an antibody 4300 is coupled 4310 to an NHS ester 4311 to yield a lysyl amide bound to a chemical handle 4313.

Whereas antibodies typically contain relatively low numbers of solvent-exposed cysteine thiols, antibodies often have tens or hundreds of surface-exposed lysines which are capable of functionalization. However, because excessive lysine functionalization can deleteriously alter protein activity and stability, lysine coupling methods typically target stoichiometries of fewer than 10 lysines per IgG1 antibody. As disclosed herein, reagent types and stoichiometries, reaction conditions, and reaction times can be varied to control the degree of lysine functionalization.

As a non-limiting example, a protein can comprise an average of 5 to 20 lysine functionalizations, an average of 2 to 6 lysine functionalizations, an average of 4 to 10 lysine functionalizations, or an average of 6 to 12 lysine functionalizations. Among the protein population, the average number of lysine functionalizations can have a standard deviation of less than about 0.25 lysine functionalizations, less than about 0.5 lysine functionalizations, less than about 0.75 lysine functionalizations, less than about 1 lysine functionalization, less than about 1.5 lysine functionalizations, less than about 2 lysine functionalizations, less than about 3 lysine functionalizations, or less than about 4 lysine functionalizations. Among the protein population, the average number of lysine functionalizations can have a standard deviation of at least about 5 lysine functionalizations, at least about 3 lysine functionalizations, at least about 2 lysine functionalizations, at least about 1.5 lysine functionalizations, at least about 1 lysine functionalization, at least about 0.75 lysine functionalizations, or at least about 0.5 lysine functionalizations. For example, a population of IgG1 antibodies prepared according to a method of the present disclosure can have an average of 5.5 lysine functionalizations with a standard deviation of 1.25 lysine functionalizations per antibody.

### L. Degree of Functionalization

In some embodiments, an immune-activating substrate of the present disclosure includes an activator coupled to a substrate at a desired degree of functionalization. In embodiments where the substrate is an individual molecule, such as a carrier protein (e.g., an antibody, an antibody fragment, a single chain variable fragment (scFv), a nanobody, or a peptide), a carrier polymer (e.g., a synthetic polymer, a biopolymer such as dextran), or a carrier nucleic acid (e.g., an oligonucleotide, a polynucleotide), the degree of functionalization may be expressed as an activator-to-substrate ratio that indicates the average number of activator molecules that are coupled to each substrate molecule. For instance, in embodiments where the substrate is an antibody, the degree of functionalization of the antibody may be expressed as a drug-to-antibody ratio (DAR). In some embodiments, the activator-to-substrate ratio is about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 15, about 20, about 30, about 40, about 50, about 60, about 70, about 80, about 90, or about 100. In some embodiments, the activator-to-substrate ratio is greater than or equal to 1, 1.5, 2, 2.5, 3, 3.5, 4, 4.5, 5, 5.5, 6, 6.5, 7, 7.5, 8, 8.5, 9, 9.5, 10, 15, 20, 30, 40, 50, 60, 70, 80, 90, or 100; and/or the activator-to-substrate ratio is less than or equal to 100, 90, 80, 70, 60, 50, 40, 30, 20, 15, 10, 9.5, 9, 8.5, 8, 7.5, 7, 6.5, 6, 5.5, 5, 4.5, 4, 3.5, 3, 2.5, 2, 1.5, or 1. In some embodiments, the activator-to-substrate ratio is within a range from 1 to 20, 1 to 10, 1 to 5, 1 to 2, 2 to 20, 2 to 10, 2 to 5, 5 to 20, 5 to 10, or 10 to 20. The activator-to-substrate ratio may influence the efficacy with which the immune-activating substrate activates an engineered cytokine switch of the present disclosure. For instance, a higher activator-to-substrate ratio may be advantageous in some instances to facilitate dimerization of the cytokine receptor chains of an engineered cytokine receptor switch. A higher activator-to-substrate ratio may lead to an increased binding avidity that increases activation.

### IX. EXAMPLES

The present technology is further illustrated by the following non-limiting examples.

### Example 1: NHS-Activated Bead Functionalization

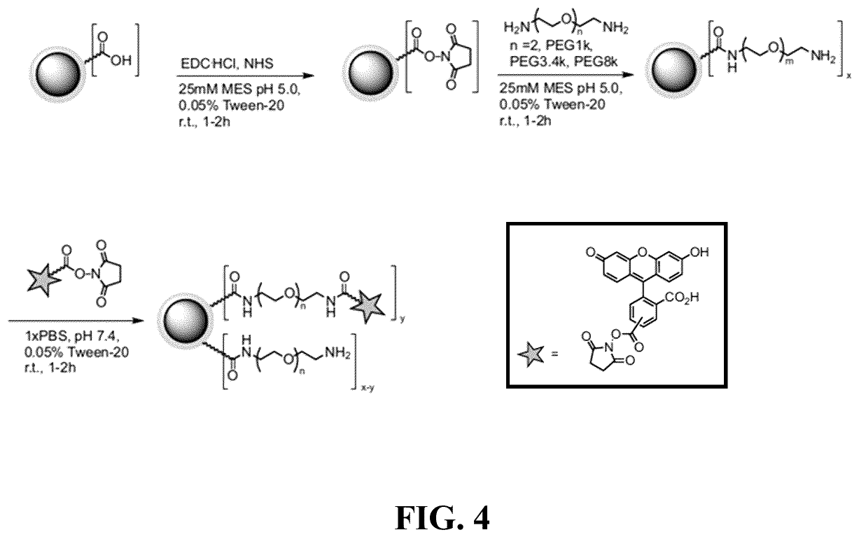

This example covers the effect of linker length and concentration on chemical handle density achieved during bead functionalization. As outlined in FIGS. 4, 0.5 to 1.5 mg of 1 and 2.8 μm carboxylate-functionalized magnetic beads were activated and then coupled to fluorescein through various length flexible PEG linkers. In order to activate the beads for linker coupling, the carboxylate surface groups were converted to acylisourea with excess 130 mM 1-Ethyl-3-(3-dimethylaminopropyl) carbodiimide HCl (EDC-HCl), and were then substituted with 217 mM NHS to form NHS esters. This step was performed at room temperature in 25 mM pH 5.0 MES buffer with 0.05% nonionic detergent for 1 to 2 hours. Using the same conditions as the first step, the resultant NHS ester functionalized beads were then contacted with 0.1-100 mM of PEG bis-amine linkers with length 2 (PEG2), 23 (PEG1k), 77 (PEG3.4k), and 182 (PEG8k) PEG backbones, generating amide bonds coupling the beads to the linkers. Finally, in pH 7.4 phosphate buffered saline with 0.05% nonionic detergent, the terminal primary amines of the linkers were acylated with of 1 mM NHS ester-functionalized fluorescein (chemical handles), yielding beads with fluorescein densities ranging from 11.0-205.0 pmol/mg (molar content to mass of beads). Fluorescein content of the resultant bead preparations were determined with fluorescence, of which some syntheses are summarized in Table 11 below.

TABLE 11

| | | | | | Ratio of |
|---|---|---|---|---|---|
| Bead | Carboxylate | | Mass Of | | Fluorescein |
| Size | Density | | Beads | Number of | to Beads |
| (μm) | (pmol/mg) | Linker | (mg) | Beads | (pmol/mg) |
| 2.8 | 200-250 | PEG2 | 1.0 | 6.6 × 10⁷ | 22.1 |
| 2.8 | 200-250 | PEG2 | 1.0 | 6.6 × 10⁷ | 38.4 |
| 2.8 | 200-250 | PEG2 | 0.5 | 3.3 × 10⁷ | 75.6 |
| 2.8 | 200-250 | PEG1k | 1.5 | 9.9 × 10⁷ | 39.4 |
| 2.8 | 200-250 | PEG3.4k | 1.0 | 6.6 × 10⁷ | 61.9 |
| 2.8 | 200-250 | PEG8k | 1.0 | 6.6 × 10⁷ | 80.6 |
| 1.0 | 400-800 | PEG2 | 0.5 | 4.8 × 10⁸ | 84.4 |
| 1.0 | 400-800 | PEG1k | 0.5 | 4.8 × 10⁸ | 120.6 |
| 1.0 | 400-800 | PEG3.4k | 0.5 | 4.8 × 10⁸ | 189.5 |
| 1.0 | 400-800 | PEG8k | 0.5 | 4.8 × 10⁸ | 205.0 |

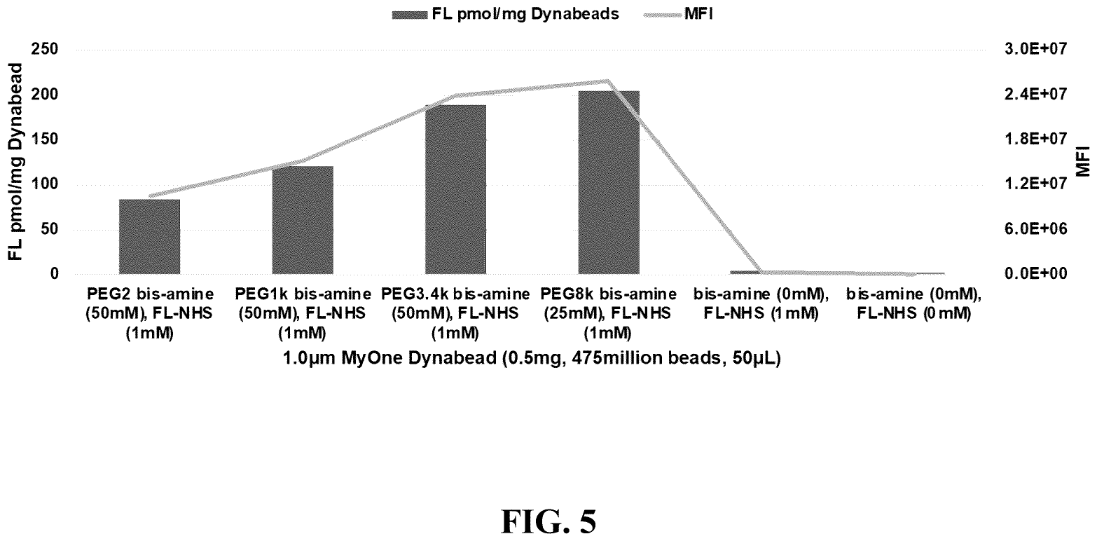

FIG. 5 provides fluorescein activator loading for the functionalized 1.0 μm diameter beads, with the left axis and bars indicating fluorescein to bead ratios (pmol fluorescein to mg beads), and the right axis and line indicating mean fluorescence intensity. As can be seen from the plot, fluorescein functionalization densities correlated with PEG linker length, with PEG2-functionalized beads exhibiting less than half of the fluorescein functionalization density of the PEG3.4k and PEG8k beads. Control beads contacted with fluorescein but unfunctionalized with PEG linkers and not contacted with fluorescein or PEG linkers (5^{th} and 6^{th} entries from the left, respectively), exhibited no fluorescence intensity, indicating that the fluorescein chemical handles did not couple directly to the beads or non-covalently adsorb to the surfaces of the beads.

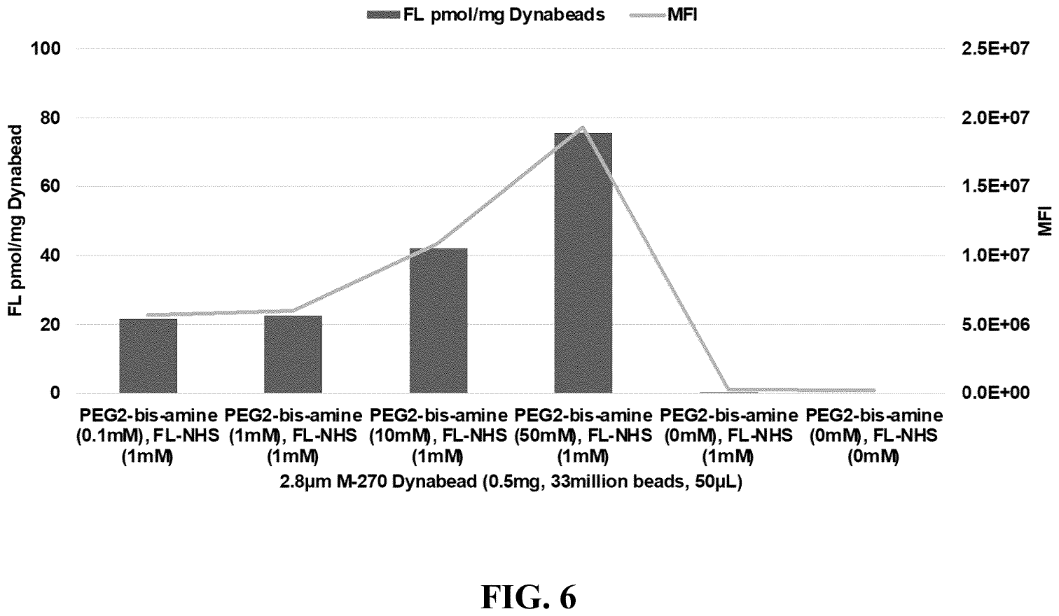

FIG. 6 provides fluorescein activator loading for the 2.8 μm diameter beads functionalized with varying concentrations of PEG2-bis-amine linker prior to fluorescein functionalization. In the plot, left axis and bars provide fluorescein to bead ratios (pmol fluorescein to mg beads), while the right axis and line indicate mean fluorescence intensity measured for each bead. From left to right, the beads correspond to syntheses which utilized 0.1 mM PEG2-bis amine, 1 mM PEG2-bis-amine, 10 mM PEG-2-bis-amine, 50 mM PEG2-bis-amine, and no PEG2-bis-amine during the linker coupling step. While only a small increase in fluorescein density was achieved by increasing PEG linker concentration from 0.1 to 1 mM, approximately 2-fold increases in fluorescein density were achieved by increasing the PEG linker concentration from 1 to 10 mM, and from 10 mM to 50 mM during the linker functionalization step.

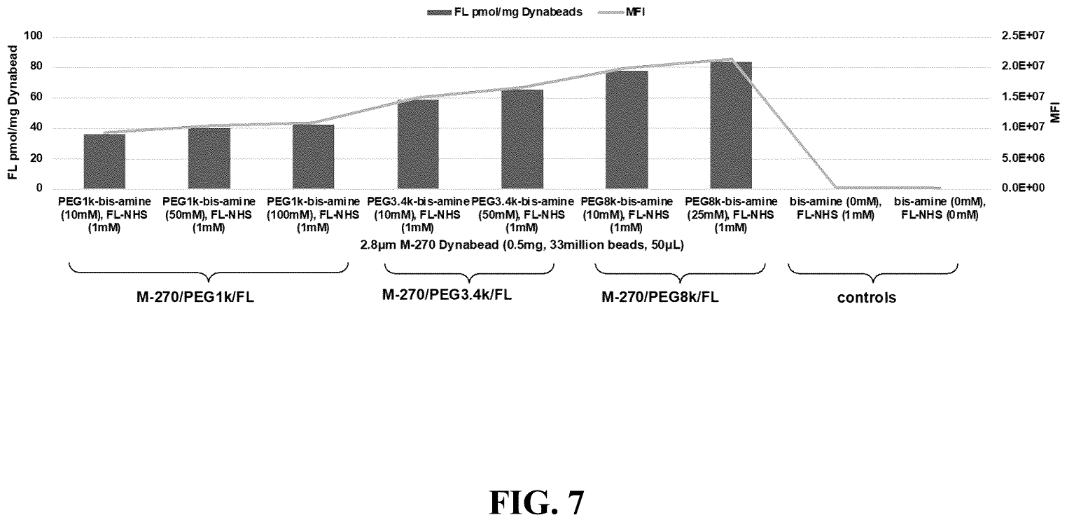

FIG. 7 provides fluorescein activator loading for the 2.8 μm diameter beads functionalized with varying concentrations of PEG1k-bis-amine, PEG3.4k-bis-amine, and PEG8k-bis-amine linkers. In the plot, the left axis and bars provide fluorescein to bead ratios (pmol fluorescein to mg beads), while the right axis and line indicate mean fluorescence intensity measured for each bead. Fluorescein functionalization density increased with linker length and concentration, with PEG8k-bis-amine functionalized beads exhibiting about 30% higher fluorescein densities than PEG4k-bis-amine functionalized beads and about 50% higher fluorescein densities than PEG1k-bis-amine functionalized beads.

### Example 2: Tosyl-Activated Bead Functionalization

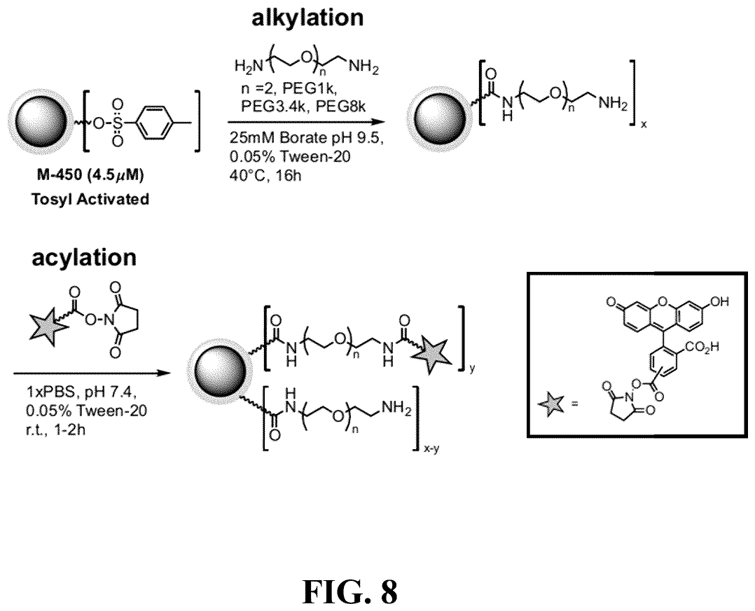

This example overviews a method for functionalizing beads through tosylate surface functionalizations. As outlined in FIG. 8, 4.5 μm diameter beads with tosyl-activated surfaces were contacted with PEG2-, PEG1k-, PEG3.4k-, and PEG8k-bis-amine linkers for 16 hours at 37-40° C. in pH 9.0 50 mM borate buffer with 0.05% nonionic surfactant, yielding resulting in coupling between the beads and PEG-bis-amine linkers. The free amine-ends of the linkers were then acylated with 1 mM NHS ester-activated fluorescein for 1-2 hours in either pH 7.4 phosphate buffered saline (PBS) with 0.05% nonionic surfactant and optionally with 50% DMSO (volume/volume). The resultant beads contained a mixture of fluorescein functionalized and unfunctionalized PEG-bis-amine linkers coupled to the beads through amide bonds.

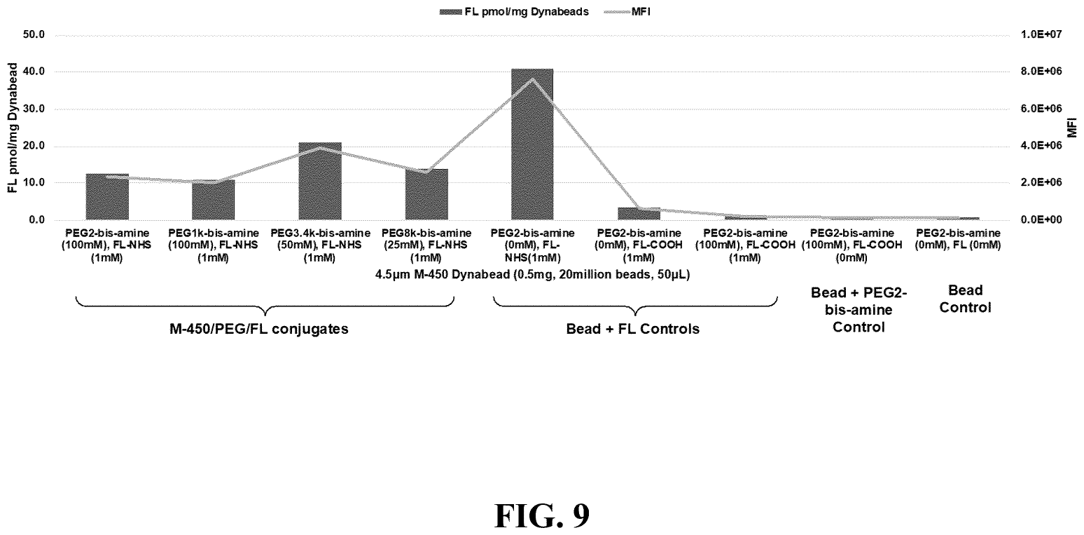

Fluorescein functionalization densities were determined by fluorescence, the results of which analyses are summarized in Table 12 and FIG. 9. In FIG. 9, the left axis and bars provide fluorescein to bead ratios (pmol fluorescein to mg beads), while the right axis and line indicate mean fluorescence intensity measured for each bead. From left to right, FIG. 9 covers (1) beads prepared with 100 mM PEG2-bis-amine and 1 mM NHS ester-activated fluorescein, (2) beads prepared with 100 mM PEG1k-bis-amine and 1 mM NHS ester-activated fluorescein, (3) beads prepared with 50 mM PEG3.4k-bis-amine and 1 mM NHS ester-activated fluorescein, (4) beads prepared with 25 mM PEG8k-bis-amine and 1 mM NHS ester-activated fluorescein, (5) beads prepared without PEG-bis-amine linkers and 1 mM NHS ester-activated fluorescein, (6) beads prepared without PEG-bis-amine linkers and 1 mM carboxylate-functionalized (non-activated) fluorescein, (7) beads prepared with 100 mM PEG2-bis-amine and 1 mM carboxylate-functionalized fluorescein, (8) beads prepared with 100 mM PEG-2-bis-amine and no fluorescein, and (9) beads prepared without PEG-bis-amine or fluorescein. While fluorescein functionalization density did not appear to correlate with PEG-bis-amine linker length, the highest fluorescein functionalization was observed for direct NHS ester-activated fluorescein coupling to the bead surfaces (beads prepared without PEG-bis-amine linkers and 1 mM NHS ester-activated fluorescein). For the beads prepared with non-activated fluoresecein, PEG linker functionalization decreased fluorescein functionalization, indicating that the non-activated fluorescein may react more readily with bead tosyl groups than with free amine ends of the linkers.

TABLE 12

| | | | | Ratio of |
|---|---|---|---|---|
| Bead | | Mass of | | Fluorescein |
| Size | | Beads | Number of | to Beads |
| (μm) | Linker | (mg) | Beads | (pmol/mg) |
| 4.5 | PEG2 | 1.5 | 6.0 × 10⁷ | 12.7 |
| 4.5 | PEG1k | 1.5 | 6.0 × 10⁷ | 11.0 |
| 4.5 | PEG3.4k | 1.5 | 6.0 × 10⁷ | 21.1 |
| 4.5 | PEG8k | 1.5 | 6.0 × 10⁷ | 14.0 |

### Example 3: Substrate Functionalization with Variable Length Carrier Complexes

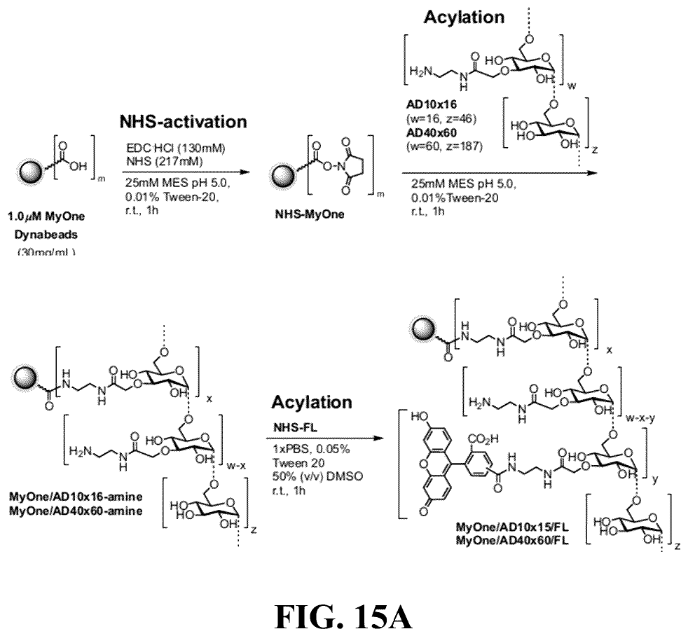

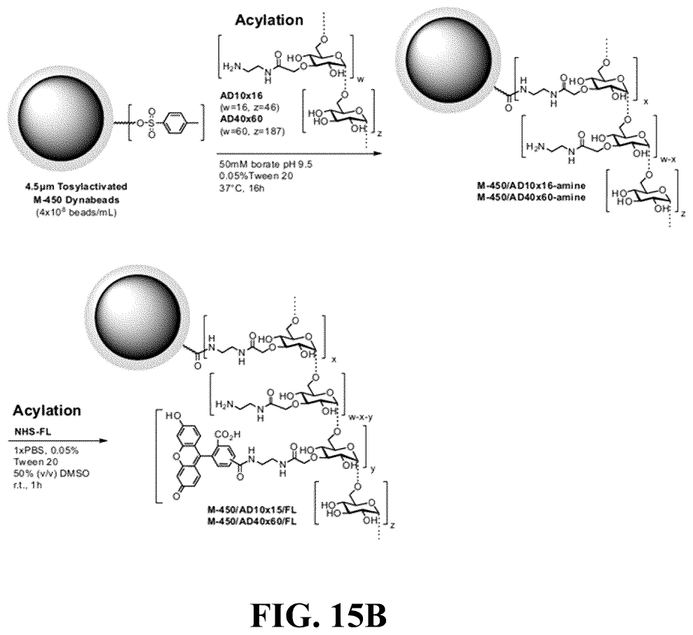

This example covers substrate functionalization with an aminosaccharide carrier complex. Carboxylate- and tosyl-functionalized beads were functionalized as outlined in FIG. 15A and FIG. 15B, respectively. The 1.0, 2.8, and 4.5 μm diameter carboxylate- or tosyl-functionalized beads were coupled to various dextran carrier complexes outlined in Table 13 according to Example 1 and Example 2, respectively, generating amide (or alkyl amine) linkages between the beads and aminodextrans of the dextran carrier complexes. The carrier complex-functionalized beads were then acylated with NHS ester-activated fluorescein, generating fluorescein functionalizations on subsets of the remaining, non-bead coupled aminodextran amines. This coupling step was performed for 1 hour at room temperature with excess 1-15 mM NHS ester-activated fluorescein in PBS buffer with 0.05% nonionic surfactant and 50% (volume/volume) DMSO. The density of fluorescein functionalizations on each bead were determined by fluorescence.

TABLE 13

| Compound | Structure |
|---|---|
| Com- pound 1 | 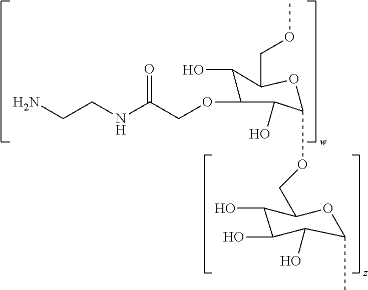 |
| | w = 16; z = 46; w and z distributed within copolymer |
| Com- pound 2 | 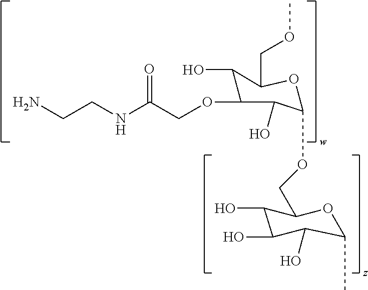 |
| | w = 60; z = 187; w and z distributed within copolymer |

Exemplary bead preparations are summarized in Table 14. While the 2.8 μm beads exhibited higher fluorescein densities with the larger compound 2 aminodextran functionalizations, the 1.0 μm and 4.5 μm beads developed nearly identical fluorescein densities with the smaller (compound 1) and larger (compound 2) carrier complexes. While carboxylate functionalized beads exhibited higher fluorescein densities than the tosyl functionalized beads, this may have been due to differences in surface functionality densities on the beads.

TABLE 14

| Bead | | Mass | | Fluorescein |
|---|---|---|---|---|
| Functionalization, | Carrier | Beads | Number Of | Density |
| Size | Complex | (mg) | Beads | (pmol/mg) |
| Carboxylate, 1.0 μm | Compound | 1.5 | 1.4 × 10⁹ | 48.8 |
| | 1 | | | |
| Carboxylate, 1.0 μm | Compound | 1.5 | 1.4 × 10⁹ | 49.7 |
| | 2 | | | |
| Carboxylate, 2.8 μm | Compound | 1.0 | 6.7 × 10⁷ | 22.6 |
| | 1 | | | |
| Carboxylate, 2.8 μm | Compound | 1.5 | 6.6 × 10⁷ | 55.7 |
| | 2 | | | |
| Tosyl, 4.5 μm | Compound | 1.0 | 6.6 × 10⁷ | 9.1 |
| | 1 | | | |
| Tosyl, 4.5 μm | Compound | 1.5 | 6.0 × 10⁷ | 12.8 |
| | 2 | | | |

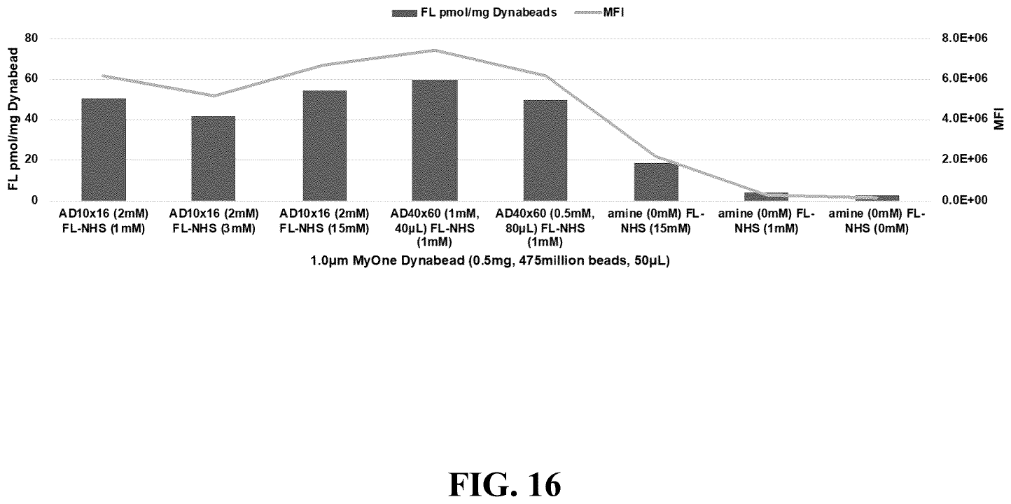

FIG. 16 provides fluorescein activator loading in terms of fluorescence intensities (right axis) and fluorescein functionalization densities (pmol/mg bead, left axis) for multiple bead preparations generated with 1.0 μm carboxylate surface-functionalized beads. From left to right, the samples correspond to beads prepared with (1) 2 mM compound 1 and 1 mM NHS ester activated fluorescein, (2) 2 mM compound 1 and 3 mM NHS ester activated fluorescein, (3) 2 mM compound 1 and 15 mM NHS ester activated fluorescein, (4) 1 mM compound 2 and 1 mM NHS ester activated fluorescein, (5) 0.5 mM compound 1 and 1 mM NHS ester activated fluorescein, (6) no aminodextran carrier complex (no compound 1 or compound 2) and 15 mM NHS ester activated fluorescein, (7) no aminodextran carrier complex and 1 mM NHS ester activated fluorescein, and (8) no aminodextran carrier complex or NHS ester activated fluorescein. For these bead preparations, the concentration of NHS ester activated fluorescein used during functionalization did not appear to affect the final fluorescein densities on the beads. However, lowering the concentration of carrier complex (entries 4 and 5 in FIG. 16) did lead to diminished functionalization density. Notably, the NHS ester activated fluorescein appeared to complex directly with the carboxylate-functionalized beads, with bead preparations generated with 0 mM aminodextran carrier complex and either 1 or 15 mM NHS ester activated fluorescein exhibiting non-zero fluorescence intensities.

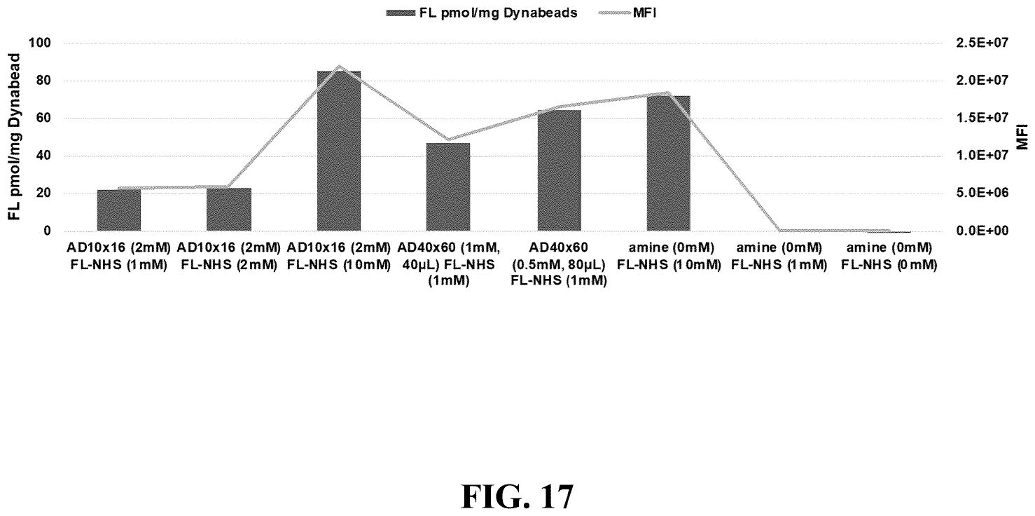

FIG. 17 provides fluorescein activator loading in terms of fluorescence intensities (right axis) and fluorescein functionalization densities (pmol/mg bead, left axis) for multiple bead preparations generated with 2.8 μm hydrophilic, carboxylate functionalized beads. From left to right, the samples correspond to beads prepared with (1) 2 mM compound 1 and 1 mM NHS ester activated fluorescein, (2) 2 mM compound 1 and 3 mM NHS ester activated fluorescein, (3) 2 mM compound 1 and 10 mM NHS ester activated fluorescein, (4) 1 mM compound 2 and 1 mM NHS ester activated fluorescein, (5) 0.5 mM compound 1 and 1 mM NHS ester activated fluorescein, (6) no aminodextran carrier complex (no compound 1 or compound 2) and 10 mM NHS ester activated fluorescein, (7) no aminodextran carrier complex and 1 mM NHS ester activated fluorescein, and (8) no aminodextran carrier complex or NHS ester activated fluorescein.

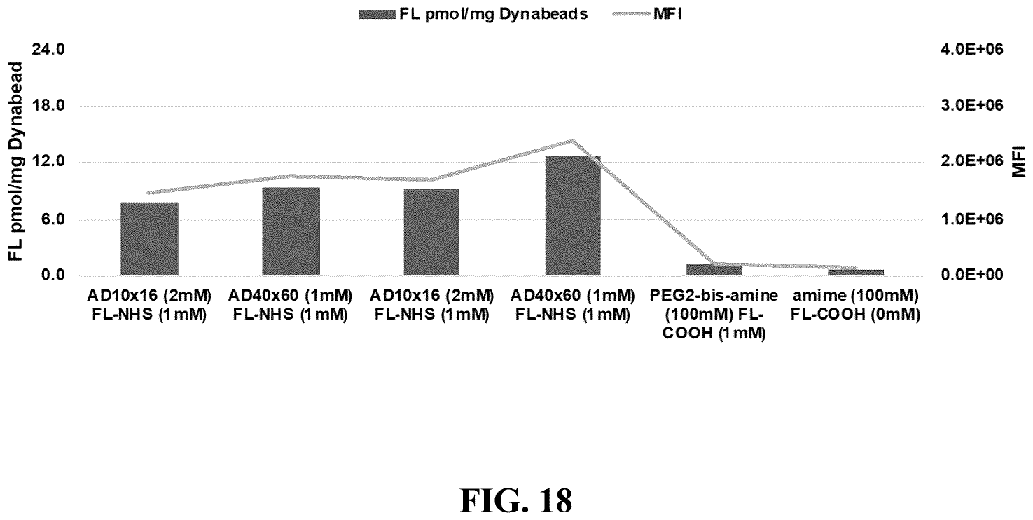

FIG. 18 provides fluorescein activator loading in terms of fluorescence intensities (right axis) and fluorescein functionalization densities (pmol/mg bead, left axis) for multiple bead preparations generated with 4.5 μm hydrophobic, tosyl-activated beads. From left to right, the samples correspond to beads prepared with (1) 2 mM compound 1 and 1 mM NHS ester activated fluorescein, (2) 1 mM compound 2 and 1 mM NHS ester activated fluorescein, (3) 2 mM compound 1 and 1 mM NHS ester activated fluorescein, (4) 1 mM compound 2 and 1 mM NHS ester activated fluorescein, (5) 100 mM PEG2-bis-amine and 1 mM fluorescein carboxylate (prepared as outlined in Example 1), and (6) 100 mM PEG2-bis-amine and no fluorescein. As can be seen in the figure, the final fluorescein densities were higher for the aminodextran-functionalized beads than for the PEG2-bis-amine functionalized beads. However, the concentration of aminodextran used during functionalization did not appear to affect final fluorescein density on the bead surfaces.

### Example 4: Streptavidin-Coated Beads

This example covers functionalization and characterization of streptavidin-coated beads. As outlined in FIG. 19, 1.0 μm hydrophobic streptavidin-coated beads, 1.0 μm hydrophilic streptavidin-coated beads, 2.8 μm hydrophobic streptavidin-coated beads, and 2.8 μm hydrophilic streptavidin-coated beads were functionalized with biotin-based carrier complexes. Following an initial wash step, the beads were contacted to 10-500 μM the carrier complexes in pH 7.4 PBS at room temperature, and again washed to remove unbound carrier complex. The carrier complexes, which are detailed in Table 15A, contained biotin and fluorescein connected through linear PEG linkers of varying lengths.

TABLE 15A

| Compound | Structure |
|---|---|
| Compound 3 | 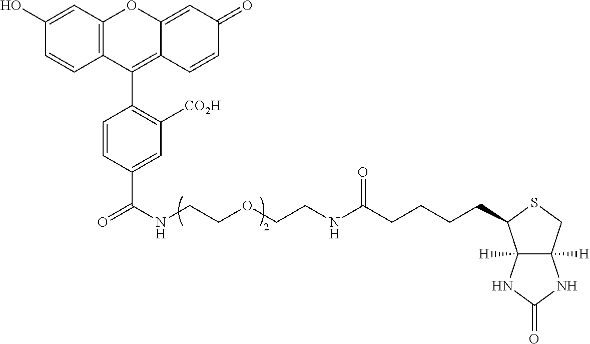 |
| Compound 4 | 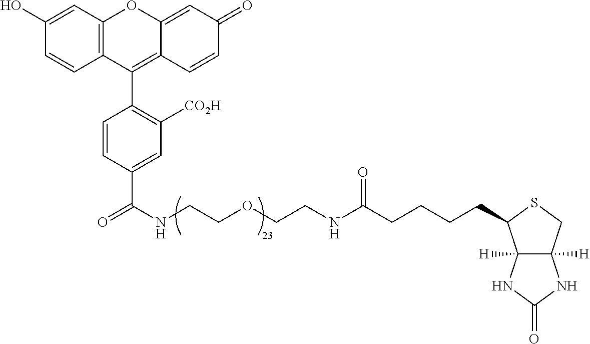 |
| Compound 5 | 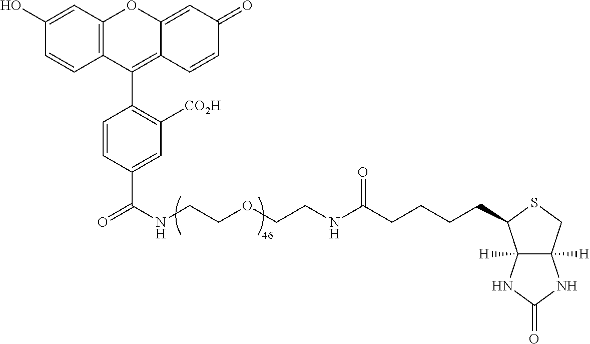 |
| Compound 6 | 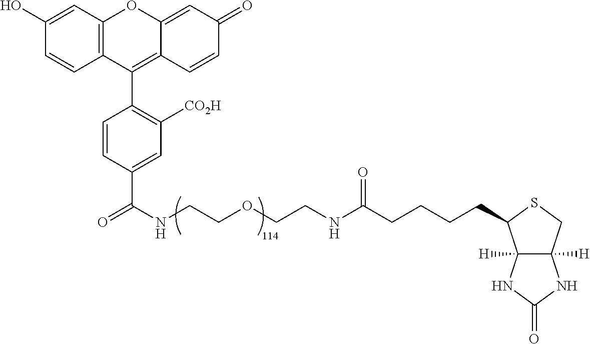 |

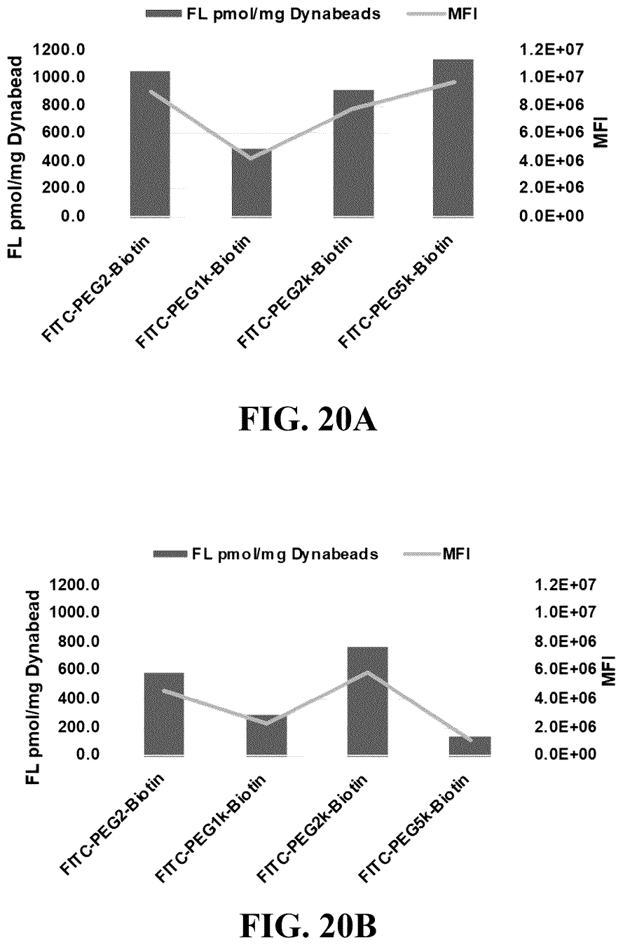

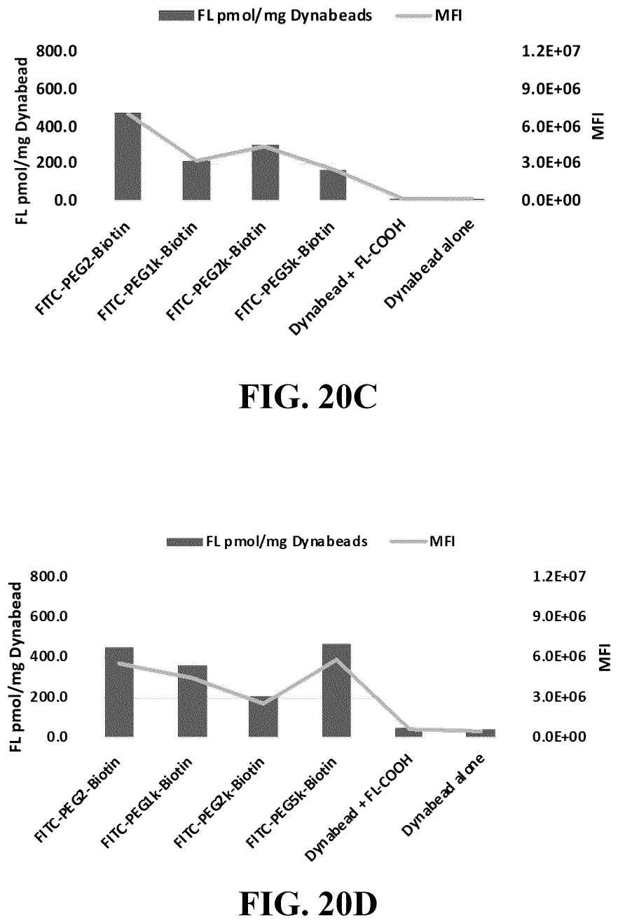

Fluorescein functionalization densities of the resultant beads were determined with fluorimetry and are summarized in Table 15B and FIGS. 20A-20D, with FIG. 20A covering 1.0 μm hydrophilic beads, FIG. 20B covering 1.0 μm hydrophobic beads, FIG. 20C covering 2.8 μm hydrophilic beads, and FIG. 20D covering 2.8 μm hydrophobic beads. FIG. 20C and FIG. 20D further provide fluorescence intensities of the 2.8 μm beads functionalized with fluorescein carboxylate. In FIGS. 20A-20D, the left axes and bars provide fluorescein functionalization densities (pmol fluorescein per mg bead), while the right axes and lines provide uncorrected mean fluorescence intensities. The fluorescein functionalization densities varied over an order of magnitude, or from approximately 143.8 pmol per mg beads to 1136.5 pmol per mg beads. In the batches tested, the 1.0 μm beads generated higher fluorescein surface densities than the 2.8 μm beads, with the 1.0 μm hydrophilic beads generating the highest surface functionalization densities. These differences may have been due, in part, to higher streptavidin surface densities on the 1.0 μm beads (which contained about 400 pmol streptavidin per mg beads) than on the 2.8 μm beads (which contained about 200 pmol streptavidin per mg beads).

TABLE 15B

| Bead Type/Streptavidin | | Mass | | Fluorescein Density |
|---|---|---|---|---|
| Functionalization Density | Carrier | Beads | Number of | (pmol/mg), |
| (pmol/mg beads) | Complex | (mg) | Beads | (fluorescein/μm²) |
| 1.0 μm hydrophilic/400 | Compound 3 | 0.4 | 3.8 × 10⁸ | 1056.4, 213263 |
| 1.0 μm hydrophilic/400 | Compound 4 | 0.6 | 5.7 × 10⁸ | 482.5, 97405 |
| 1.0 μm hydrophilic/400 | Compound 5 | 0.2 | 1.9 × 10⁸ | 906.7, 183042 |
| 1.0 μm hydrophilic/400 | Compound 6 | 0.3 | 2.85 × 10⁸ | 1136.5, 229433 |
| 1.0 μm hydrophobic/400 | Compound 3 | 0.3 | 2.85 × 10⁸ | 585.1, 118118 |
| 1.0 μm hydrophobic/400 | Compound 4 | 0.3 | 2.85 × 10⁸ | 292.4, 59028 |
| 1.0 μm hydrophobic/400 | Compound 5 | 0.3 | 2.85 × 10⁸ | 765.2, 154476 |
| 1.0 μm hydrophobic/400 | Compound 6 | 0.3 | 2.85 × 10⁸ | 143.8, 29029 |
| 2.8 μm hydrophilic/200 | Compound 3 | 0.2 | 6.5 × 10⁷ | 476.2, 35839 |
| 2.8 μm hydrophilic/200 | Compound 4 | 0.2 | 6.5 × 10⁷ | 217.1, 16339 |
| 2.8 μm hydrophilic/200 | Compound 5 | 0.2 | 6.5 × 10⁷ | 297.6, 22397 |
| 2.8 μm hydrophilic/200 | Compound 6 | 0.2 | 6.5 × 10⁷ | 164.0, 12342 |
| 2.8 μm hydrophobic/200 | Compound 3 | 0.3 | 6.5 × 10⁷ | 449.2, 50710 |
| 2.8 μm hydrophobic/200 | Compound 4 | 0.3 | 6.5 × 10⁷ | 357.1, 40313 |
| 2.8 μm hydrophobic/200 | Compound 5 | 0.3 | 6.5 × 10⁷ | 203.8, 23007 |
| 2.8 μm hydrophobic/200 | Compound 6 | 0.3 | 6.5 × 10⁷ | 472.9, 53386 |

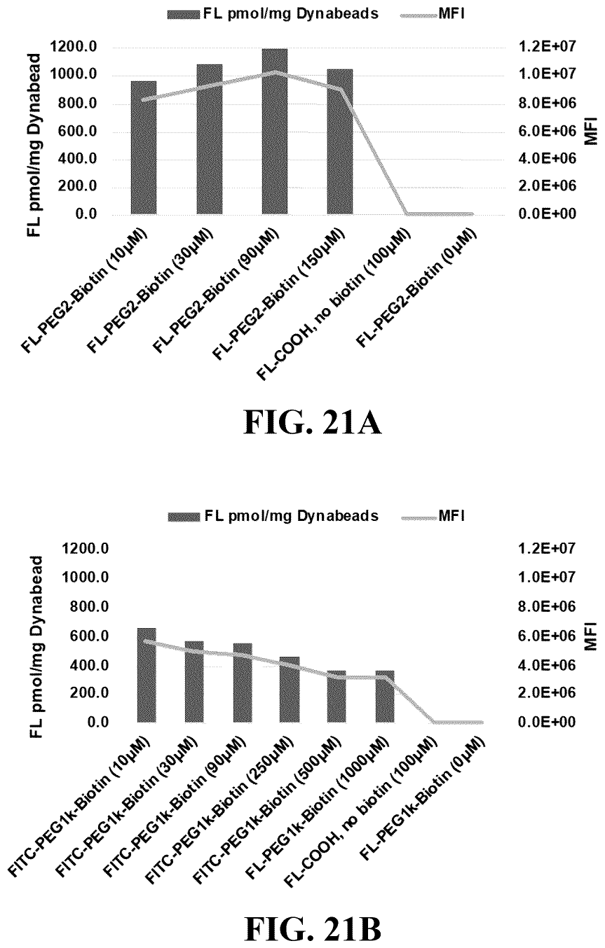

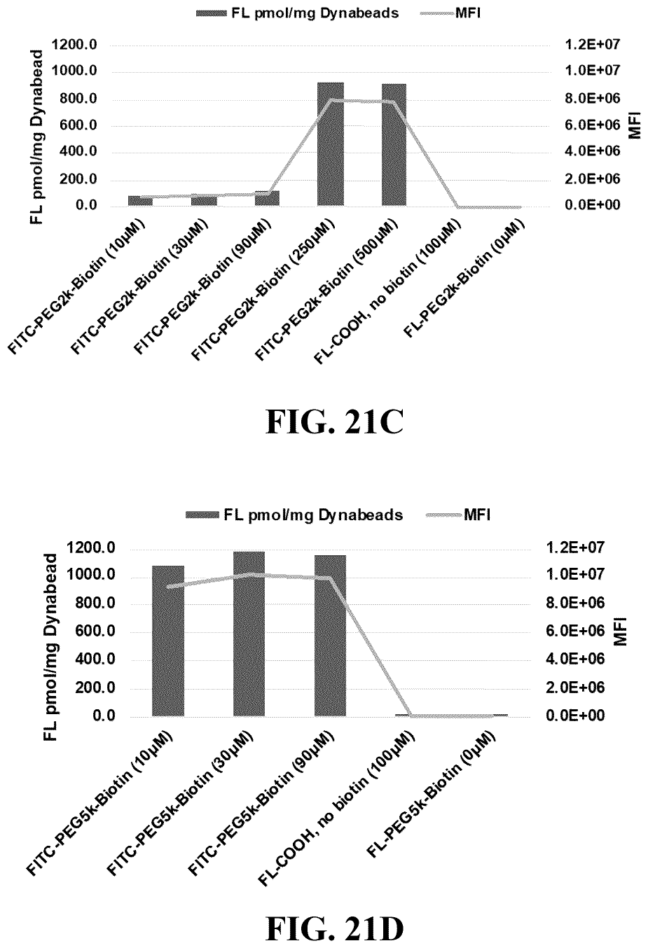

FIGS. 21A-21D provide fluorescein activator loading in terms of fluorescence intensities (right axis) and fluorescein functionalization densities (pmol fluorescein per mg bead, left axis) for 1.0 μm hydrophilic streptavidin beads functionalized with multiple concentrations of fluorescein-PEG-biotin carrier complexes, with FIG. 21A providing data for beads functionalized with compound 3, FIG. 21B providing data for beads functionalized with compound 4, FIG. 21C providing data for beads functionalized with compound 5, and FIG. 21D providing data for beads functionalized with compound 6. As controls, the figures also include data for beads contacted with non-biotinylated fluorescein, and for beads in the absence of fluorescein.

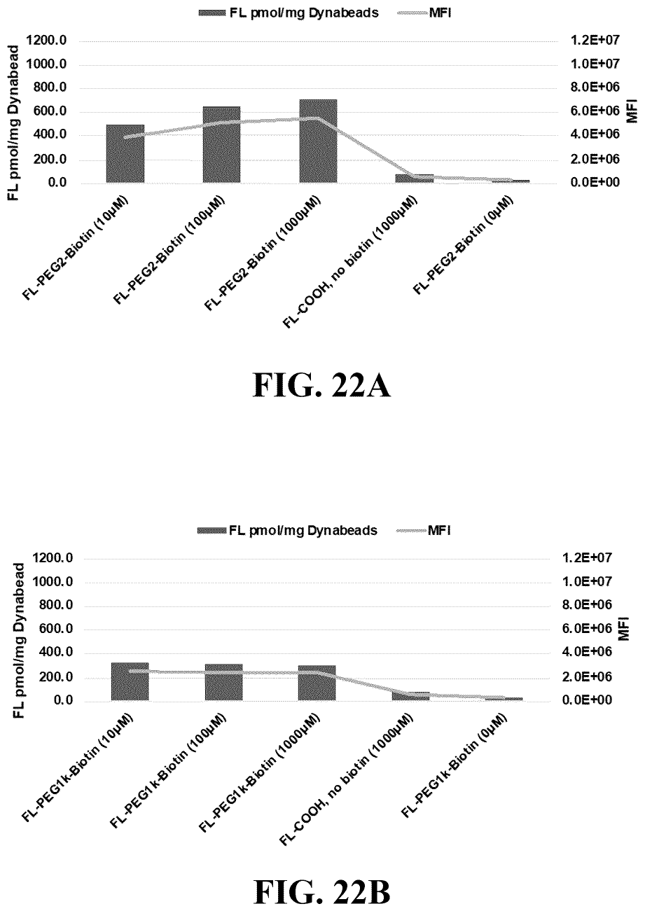

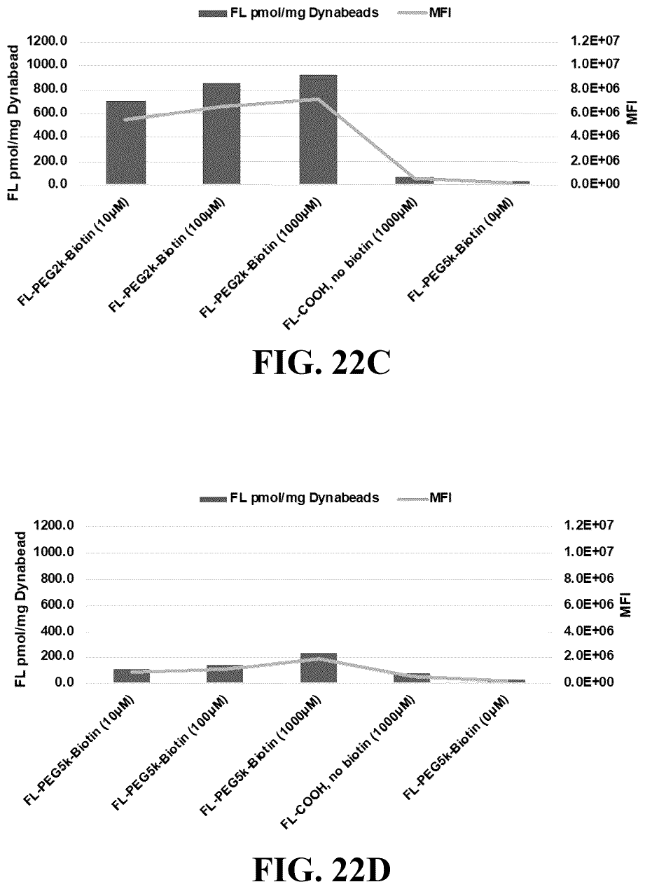

FIGS. 22A-22D provide fluorescein activator loading in terms of fluorescence intensities (right axis) and fluorescein functionalization densities (pmol fluorescein per mg bead, left axis) for 1.0 μm hydrophobic streptavidin beads functionalized with multiple concentrations of fluorescein-PEG-biotin carrier complexes, with FIG. 22A providing data for beads functionalized with compound 3, FIG. 22B providing data for beads functionalized with compound 4, FIG. 22C providing data for beads functionalized with compound 5, and FIG. 22D providing data for beads functionalized with compound 6. Each figure also includes control data for beads contacted with non-biotinylated fluorescein, and for beads in the absence of fluorescein.

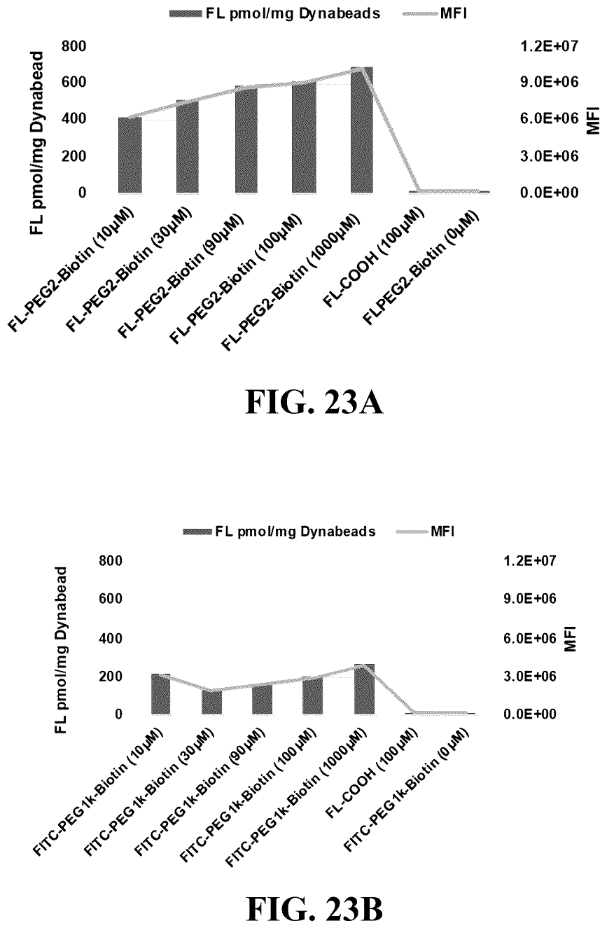

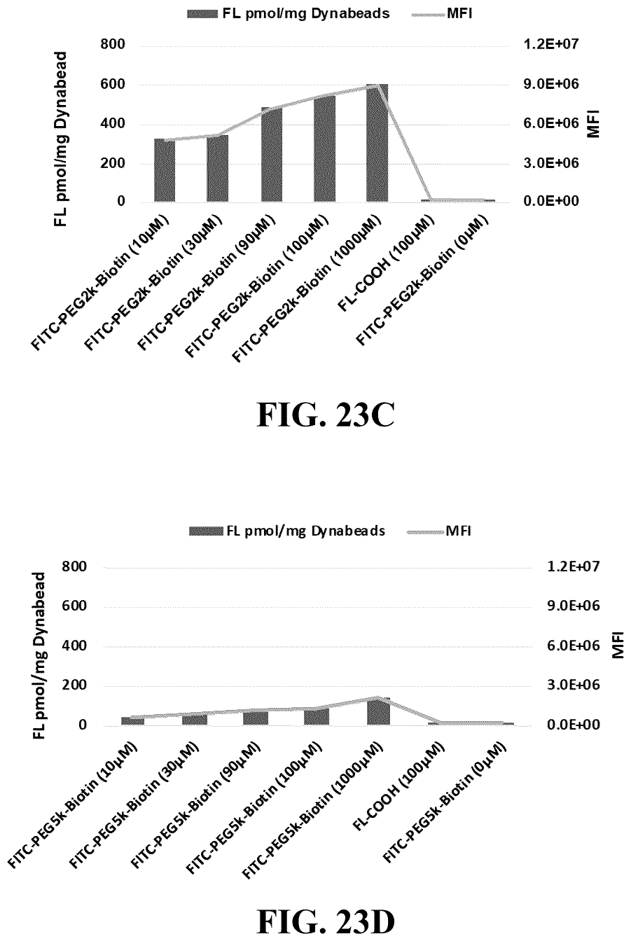

FIGS. 23A-23D provide fluorescein activator loading in terms of fluorescence intensities (right axis) and fluorescein functionalization densities (pmol fluorescein per mg bead, left axis) for 2.8 μm hydrophilic streptavidin beads functionalized with multiple concentrations of fluorescein-PEG-biotin carrier complexes, with FIG. 23A providing data for beads functionalized with compound 3, FIG. 23B providing data for beads functionalized with compound 4, FIG. 23C providing data for beads functionalized with compound 5, and FIG. 23D providing data for beads functionalized with compound 6, as well as for beads contacted with non-biotinylated fluorescein, and for beads in the absence of fluorescein.

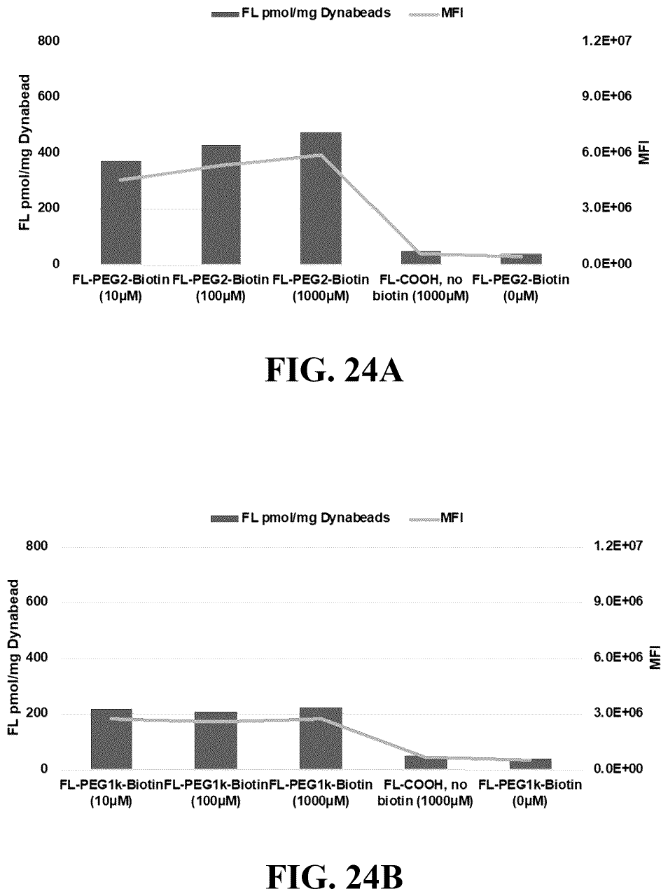

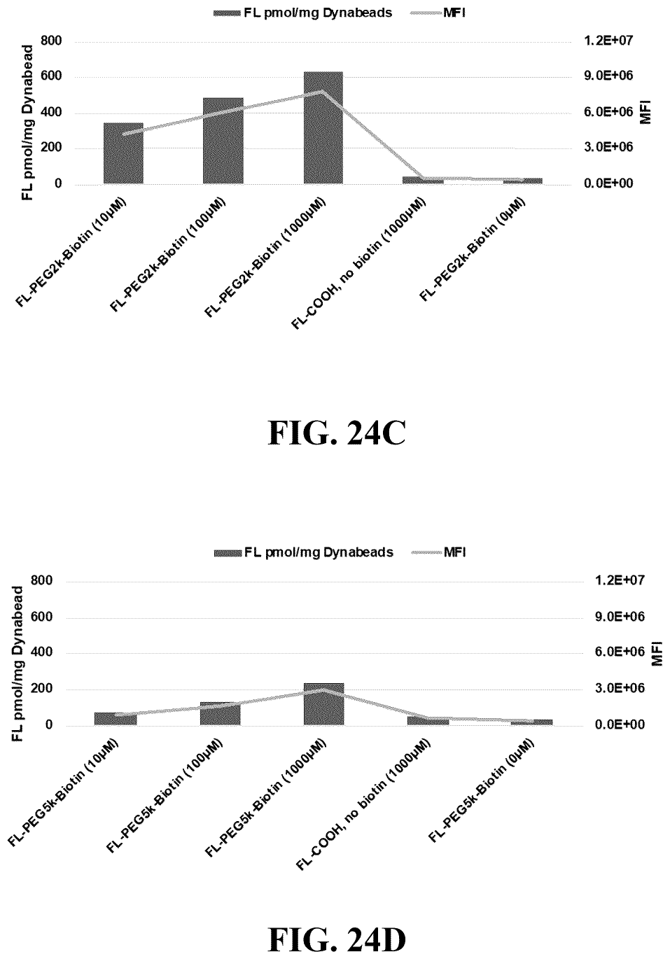

FIGS. 24A-24D provide fluorescein activator loading in terms of fluorescence intensities (right axis) and fluorescein functionalization densities (pmol fluorescein per mg bead, left axis) for 2.8 μm hydrophobic streptavidin beads functionalized with multiple concentrations of fluorescein-PEG-biotin carrier complexes, with FIG. 24A providing data for beads functionalized with compound 3, FIG. 24B providing data for beads functionalized with compound 4, FIG. 24C providing data for beads functionalized with compound 5, and FIG. 24D providing data for beads functionalized with compound 6. Each figure also includes fluorescein functionalization data for beads contacted with non-biotinylated fluorescein, and for beads in the absence of fluorescein.

### Example 5: Streptavidin-Functionalized Polymer Matrices

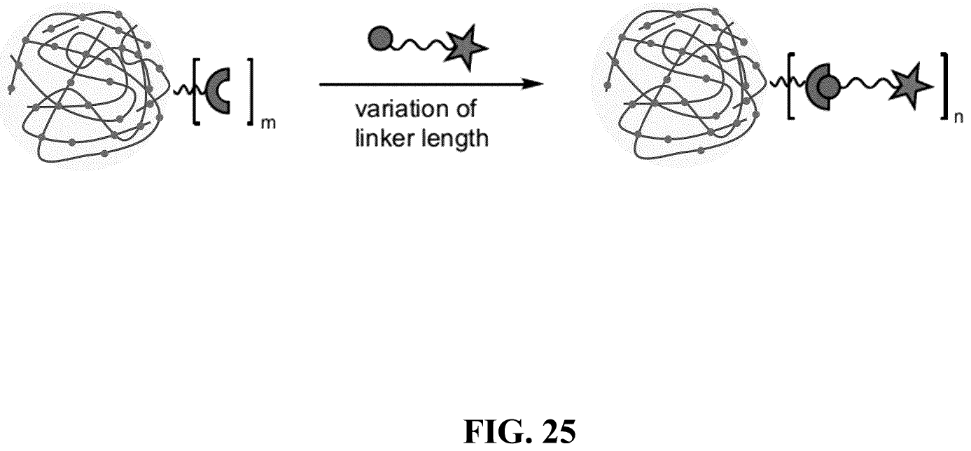

This example covers functionalization of a polymer matrix substrate with a fluorescent carrier complex. Calcium alginate hydrogel microspheres were functionalized with streptavidin and then coupled to biotinylated fluorescein carrier complexes with various length PEG linkers (as depicted in FIG. 25 and as outlined in Table 15A). The coupling steps utilized between 10 μM and 10 mM of the carrier complexes and resulted in different degrees of fluorescein labeling as measured by fluorescence.

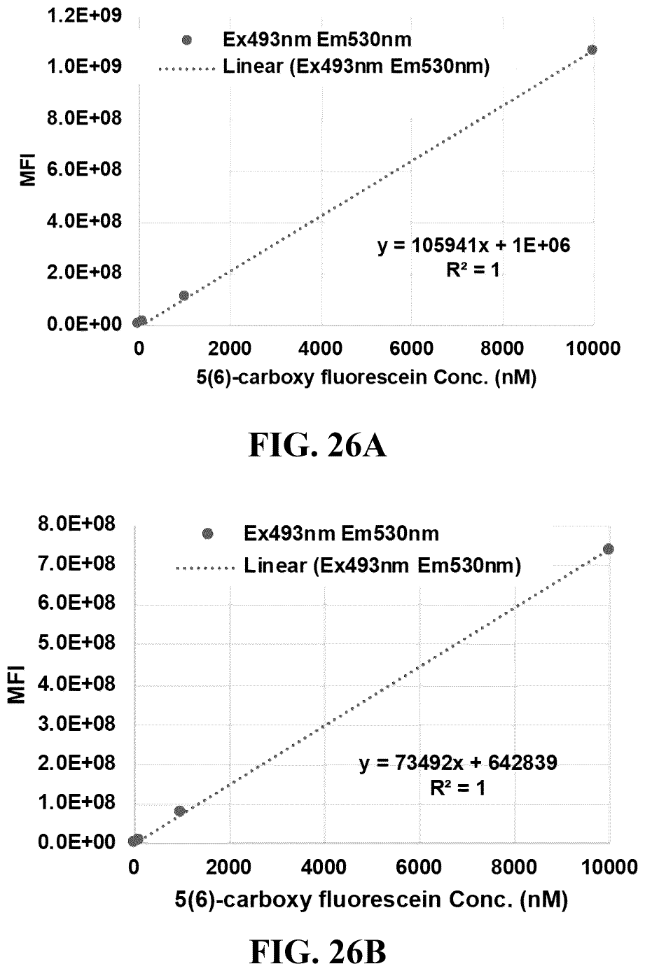

As controls for the fluorescence measurements, free 5 (6)-carboxy fluorescein fluorescence was measured in the absence (FIG. 26A) and presence (FIG. 26B) of the calcium alginate hydrogel microspheres. While the fluorescence intensities were linear with respect to the fluorescein activator concentration for both sample types, the microspheres diminished the fluorescence intensities of the samples. Accordingly, the fluorescence curve of 5 (6)-carboxy fluorescein in the presence of the calcium alginate hydrogel microspheres were used as a calibration curve for approximating the fluorescein activator loading per mL of the functionalized microspheres.

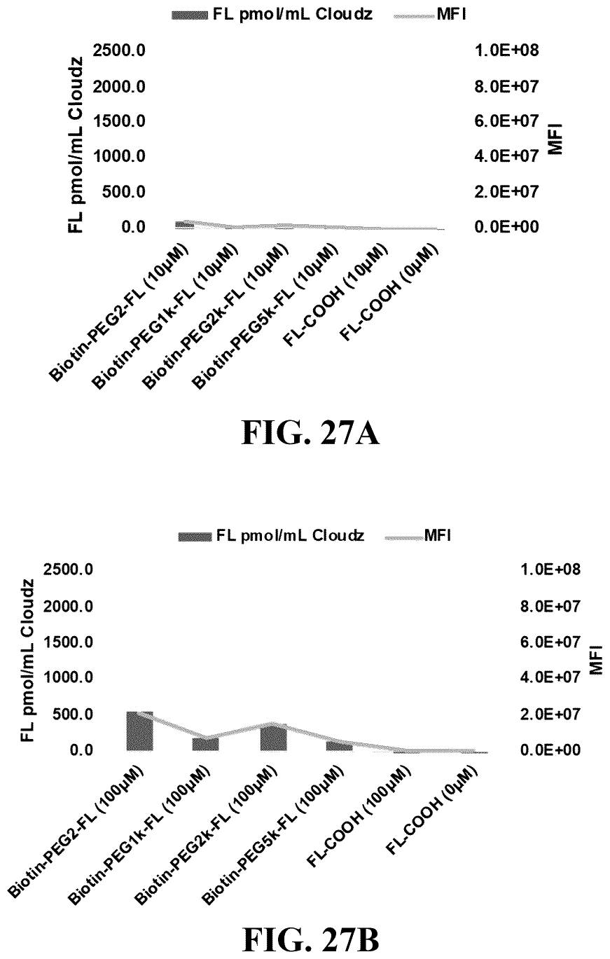

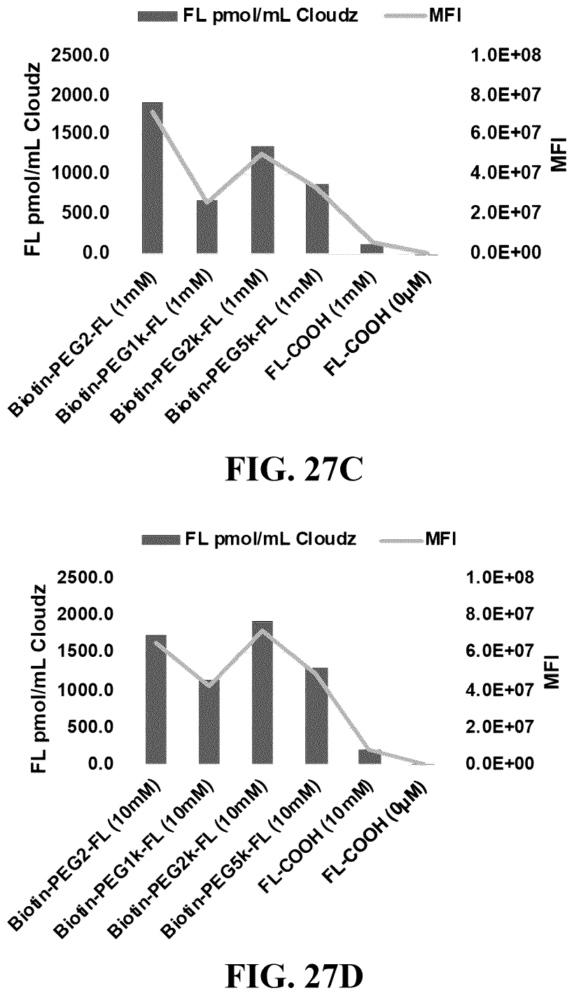

The fluorescein activator loading of the microspheres in terms of fluorescence intensity (left axis) and fluorescein functionalization densities (pmol fluorescein per mL microspheres) are summarized in FIGS. 27A-27D, which provide data for streptavidin-functionalized calcium alginate hydrogel microspheres contacted with 10 μM, 100 μM, 1 mM, and 10 mM of carrier complexes (compound 3-compound 6). Increased carrier complex concentration correlated with increased fluorescein functionalization density, reaching an apparent asymptotic maximum around 1 mM carrier complex. The highest fluorescein functionalization densities were generated with compound 3 and compound 5, corresponding to PEG linker lengths of 2 and 46, respectively.

### Example 6: Expansion and Activation of Lentivirus Transduced Lymphocytes

This example covers naïve T-cell transfection with a lentiviral vector system. PBMCs are isolated from bone marrow samples collected from donors. Monocytes are sedimented from the cell preparations to prevent inhibition of T-cell activation and transfection. The cells are suspended in culture media containing CD3 and CD28 antibody-coated beads for activation, and then contacted with 6×10⁷ transducing units/mL of lentivirus carrying an expression plasmid encoding a cytokine receptor switch and a chimeric antigen receptor. The transfected cells are maintained in the log-phase for at least five days through addition of fresh media. During the expansion period, the resultant engineered immune cells are contacted with a substrate carrying cytokine receptor switch agonist surface functionalizations. The cells are analyzed by flow cytometry and are determined to be comprised of at least 25% memory T-cells expressing the cytokine receptor switch and chimeric antigen receptor.

### Example 7: T-Cell Activation, Transduction, Expansion, and Differentiation

This example covers activation, differentiation, and expansion of immune cells with engineered cytokine receptor switches. The engineered cytokine receptor switches included fluorescein-binding receptor domains and IL-7 receptor-a intracellular signaling domains. The cells were activated with CD3 and CD28 antibody-coated beads, followed by small molecule activators for the engineered cytokine switches to promote differentiation to memory phenotypes. The proportion of stem-cell memory, central memory, and effector memory cells were measured following differentiation.

Activation, transfection, expansion, and differentiation were performed according to the timeline in FIG. 28A. The cells were maintained in media with 20 units/ml IL2 throughout the incubation. The cells were first contacted with CD3/CD28 activating beads at a ratio of 1:1 (cells to beads). Following three days of activation and expansion, the cells were transfected with a gamma retroviral vector encoding the engineered cytokine receptor switch with fluorescein binding affinity and mCherry for fluorescence detection. The cells were then transferred to a multi-well plate. The wells were either coated with fluorescein-labeled dextran or contained streptavidin-coated hydrophobic beads coupled to fluorescein carrier complexes (as outlined in FIG. 28B), or no fluorescein to serve as a control group. Seven days after activation, the cells were restimulated with additional CD3/CD28 activating beads to achieve a ratio of 10 cells per bead.

FIG. 28B provides a depiction of immune cell activation by the streptavidin-coated beads. The beads 2810 contained streptavidin 2811 coatings, which coupled to carrier complexes through biotin chemical handles 2817, which were coupled to fluorescein 2813 by linkers 2812. Contact between the fluorescein 2813 and the receptor domain 2814 of an engineered cytokine receptor switch activated its intracellular signaling domain 2815, thereby promoting differentiation in the immune cell 2816.

Following 5 total days of incubation in the multi-well plates, the cells were analyzed for (1) stem-cell memory, (2) central memory, (3) effector memory, and (4) terminally differentiated effector memory re-expressing CD45RA phenotype. Each cell population was then quantified fluorometrically, the results of which are summarized in FIG. 28C. In this figure, the y-axis denotes the percentage of cells belonging to each phenotype. The leftmost group corresponds to cells not contacted with fluorescein. The middle group corresponds to cells contacted with fluorescein-labeled dextran coated wells. The rightmost group corresponds to cells contacted with streptavidin-coated beads coupled to fluorescein carrier complexes. Within each group, the indicated cell phenotypes are stem-cell memory (T_{scm}), central memory (T_{cm}), effector memory (Tₑₘ), and terminally differentiated effector memory re-expressing CD45RA (Tₑₘᵣₐ), from left-to-right, respectively. As can be seen from this figure, the cells contacted with fluorescein contained a higher proportion of central memory cells (about 50% vs about 30%) and a lower proportion of non-memory cells than the non-fluorescein contacted cells.

### Example 8: Effect of Beads on Chemical Handle Activity

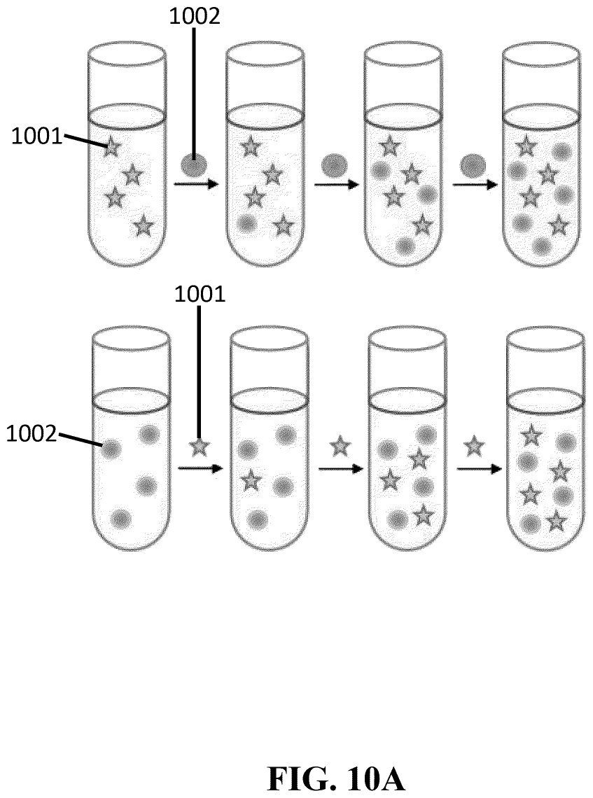

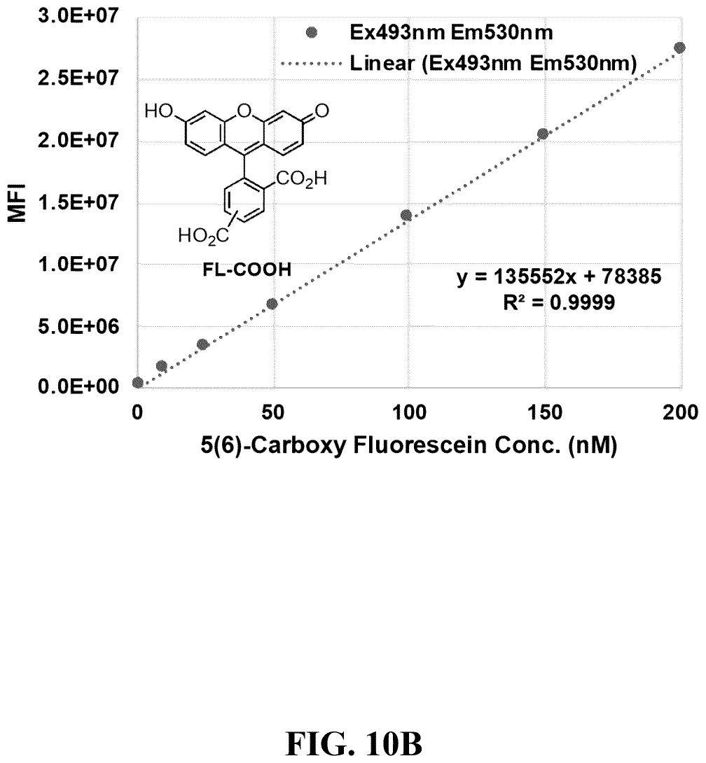

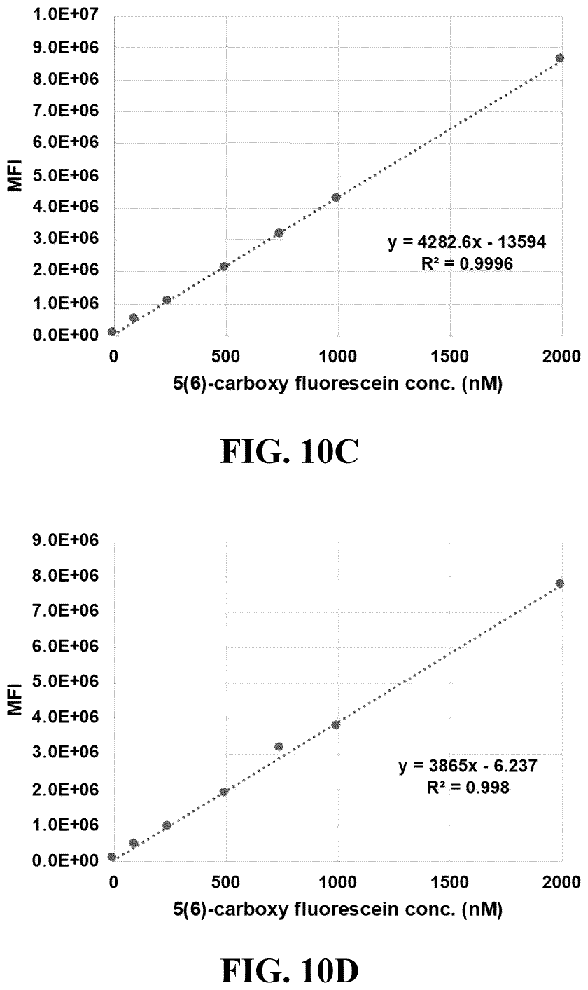

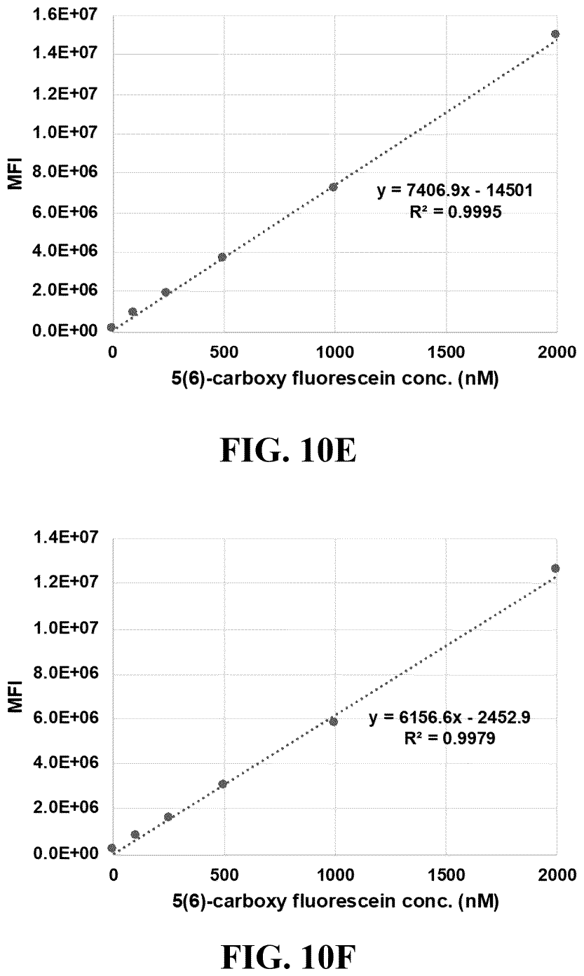

This example covers the effect of substrates on chemical handle fluorescence. Titrations were performed with multiple beads to determine whether the beads altered fluorescence outputs. As depicted in FIG. 10A, the titrations either utilized constant fluorescein 1001 concentrations with stepwise bead 1002 addition (top), or constant bead concentrations with stepwise fluorescein addition (bottom). As a control, the fluorescence profile of 5 (6)-carboxy fluorescein was characterized in the absence of bead substrates, producing a linear fluorescence response over a 0-200 nM concentration range (FIG. 10B). 5 (6)-Carboxy fluorescein was then titrated into 50 μL solutions containing 0.1 mg of 1.0 μm diameter hydrophilic beads (FIG. 10C), 1.0 μm diameter hydrophobic beads (FIG. 10D), 2.8 μm diameter hydrophilic beads (FIG. 10E), and 2.8 μm diameter hydrophobic beads (FIG. 10F). While 5 (6)-carboxy fluorescein maintained linear fluorescence profiles in the presence of all four bead types, the presence of the beads diminished overall fluorescence intensities of the fluorescein samples.

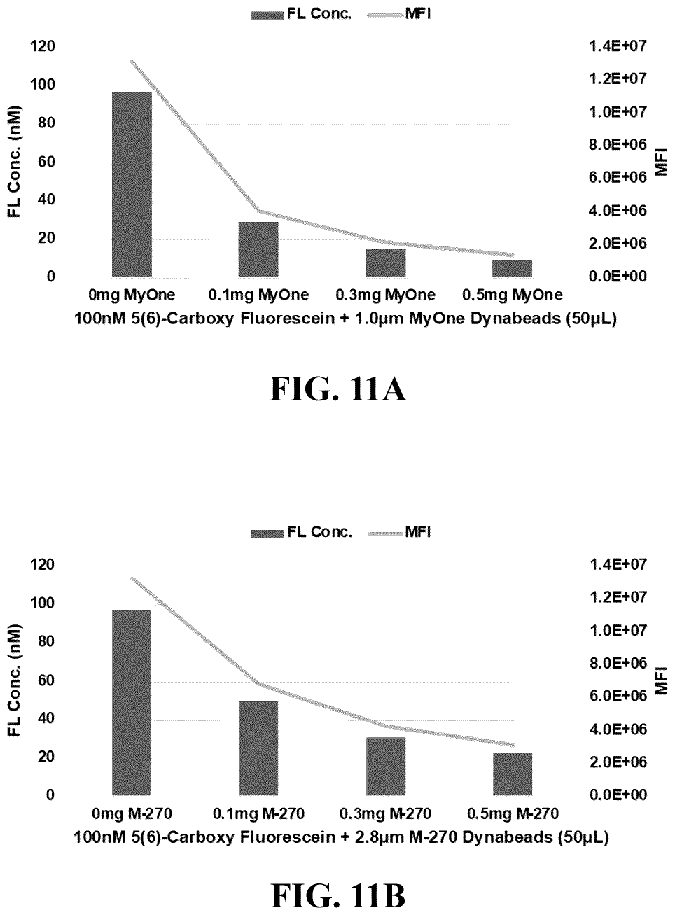

To further explore the effect of beads on fluorescein fluorescence intensity, titrations were performed using constant fluorescein and varying bead concentrations. FIG. 11A and FIG. 11B summarize fluorescence intensities of 100 nM 5 (6)-carboxy fluorescein in the presence of 0, 0.1, 0.3, and 0.5 mg 1.0 μm diameter and 2.8 μm diameter hydrophilic beads. For each bead, fluorescence intensity diminished by about 13-fold in going from 0 to 0.5 mg beads.

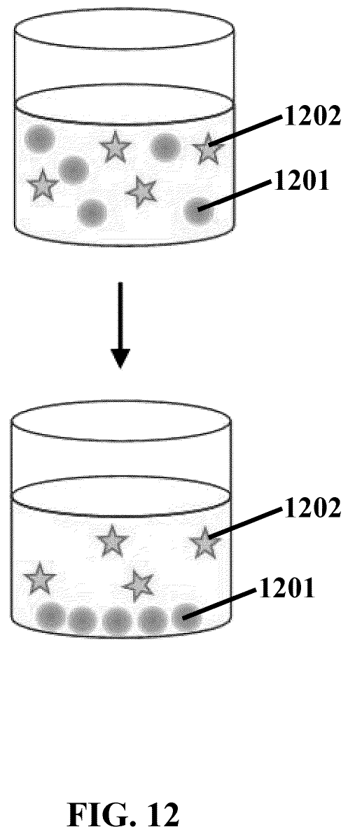

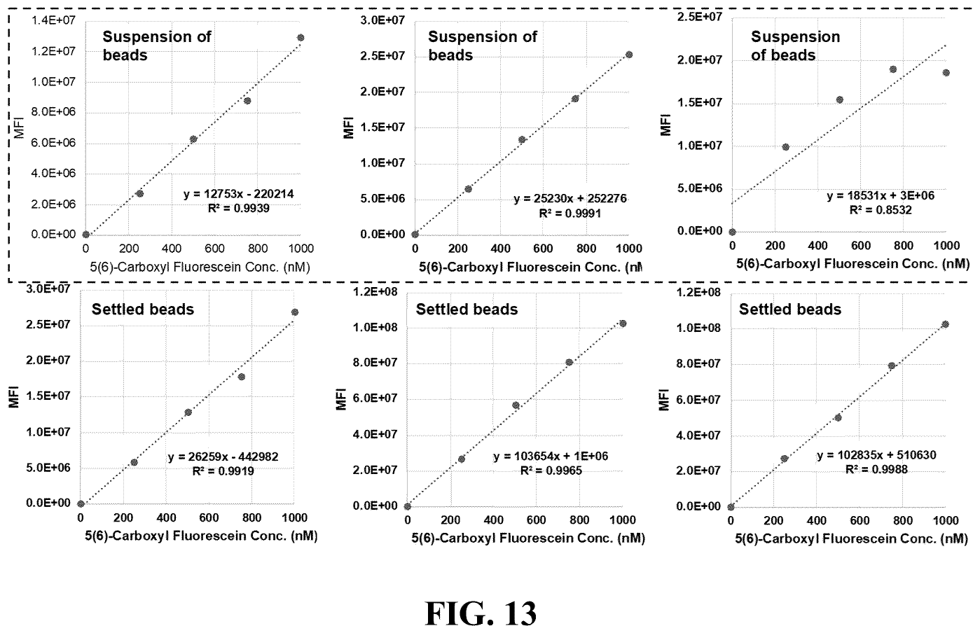

To further assess the effect of beads on fluorescein fluorescence intensity, fluorescein titration assays were performed in samples with suspended and settled beads. As depicted in FIG. 12, samples were analyzed with beads 1201 homogenously suspended throughout a fluorescein 1202 solution (top), and with the beads 1201 settled at the bottom of the fluorescein 1202 solutions (bottom). Each assay was performed in a 50 μL volume with 0.5 mg of 1.0 μm (left), 2.8 μm (middle), or 4.5 μm (right) beads. FIG. 13 provides the results of the bead settling titrations, with the top, boxed plots corresponding to suspended beads and the bottom plots corresponding to settled beads. Each of the three bead types exhibited greater fluorescence quenching while suspended than while settled, indicating that interactions between the beads and fluorescein inhibit fluorescence intensity.

### Example 9: Effects of Bead and Linker Parameters on Ex Vivo Activation of Engineered T-cells

This example covers the effects of varying small molecule activator functionalized beads on engineered immune cell activation. Immune cells were transfected, activated, and expanded as outlined in Example 7 with beads outlined in Table 16. Two bead types (2.8 μm hydrophobic and 2.8 μm hydrophilic) four PEG linkers (PEG2, 1 kD PEG, 2 kD PEG, and 5 kD PEG), and three cell-to-bead ratios (1:2, 1:5, and 1:10 during 2805 of FIG. 28A) were used for these assays. Control assays with no fluorescein-functionalized substrate, fluorescein-coated dextran wells, and fluorescein-labeled CD3 antibodies were performed in parallel.

TABLE 16

| | | PEG Linker | Cell:bead |
|---|---|---|---|
| Diameter | Bead Surface | length | ratio |
| 2.8 um | Hydrophobic | PEG2 (n = 2) | 1:2, 1:5, and 1:10 |
| 2.8 um | Hydrophobic | 1 kD (n = 24) | 1:2, 1:5, and 1:10 |
| 2.8 um | Hydrophobic | 2 kD (n = 45) | 1:2, 1:5, and 1:10 |
| 2.8 um | Hydrophobic | 5 kD (n = 113) | 1:2, 1:5, and 1:10 |
| 2.8 um | Hydrophilic | PEG2 (n = 2) | 1:2, 1:5, and 1:10 |
| 2.8 um | Hydrophilic | 1 kD (n = 24) | 1:2, 1:5, and 1:10 |
| 2.8 um | Hydrophilic | 2 kD (n = 45) | 1:2, 1:5, and 1:10 |
| 2.8 um | Hydrophilic | 5 kD (n = 113) | 1:2, 1:5, and 1:10 |

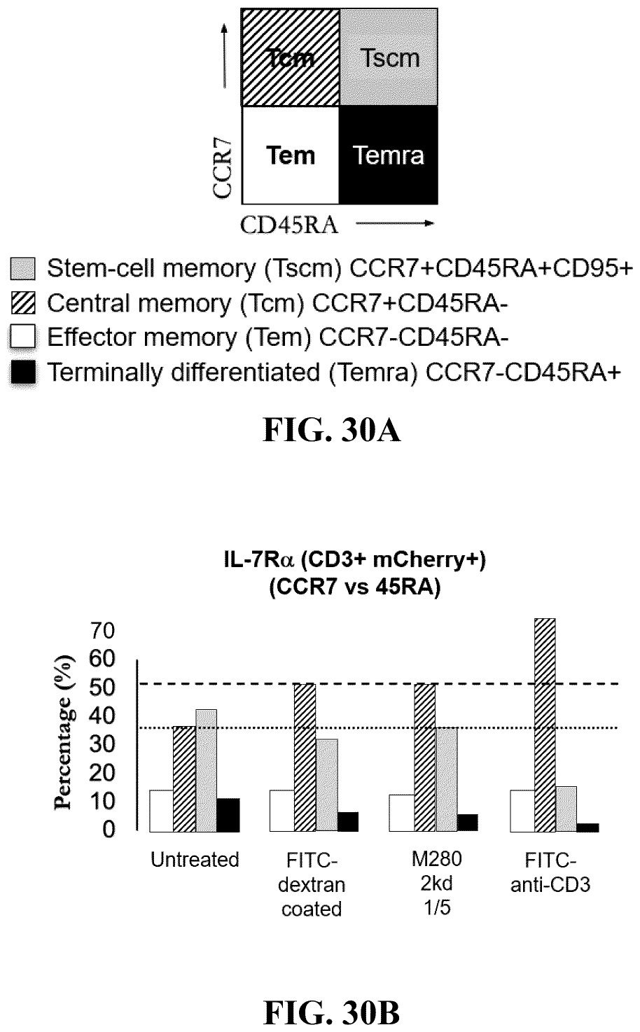

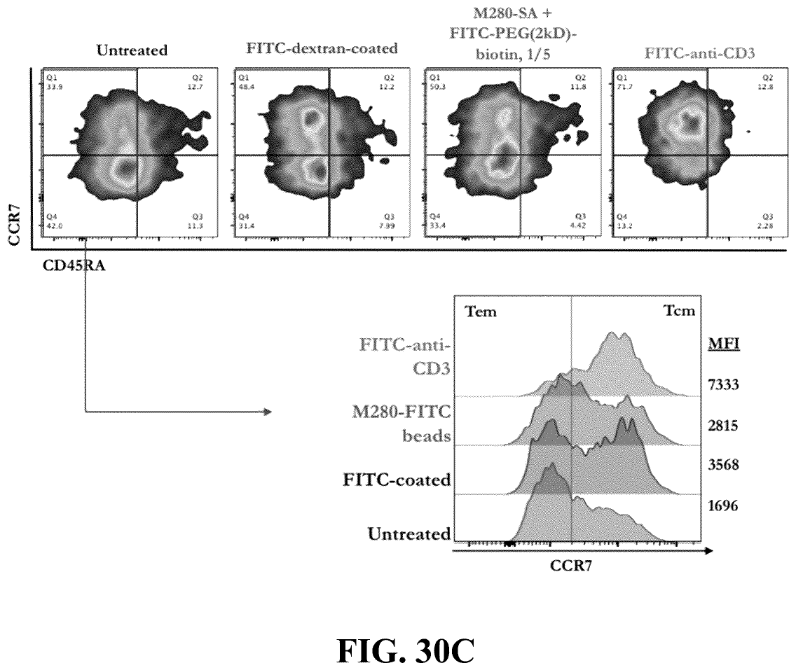

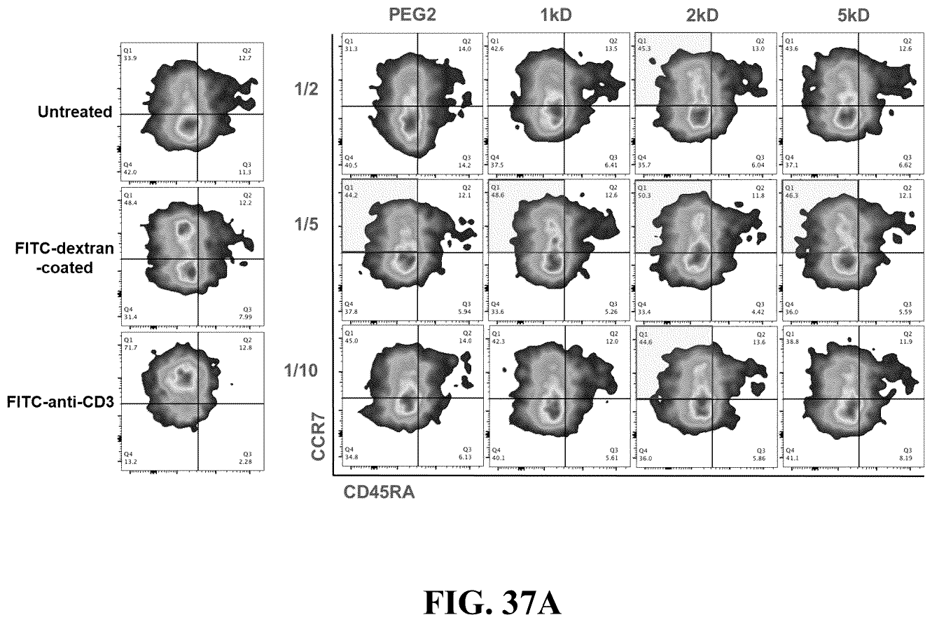

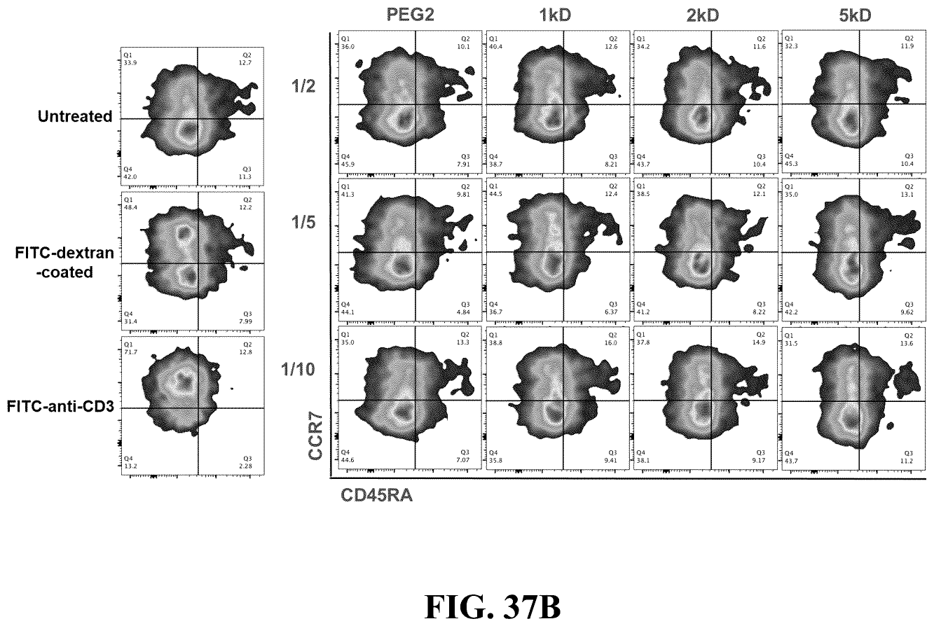

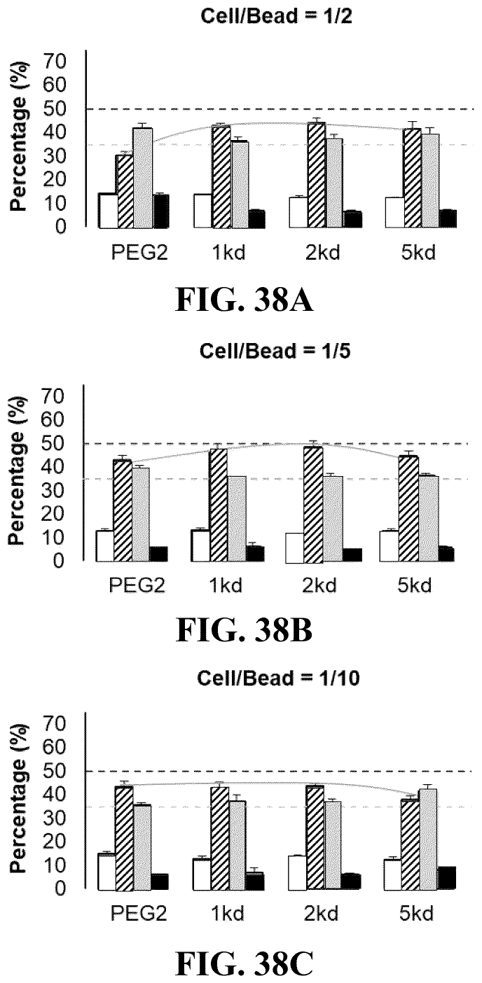

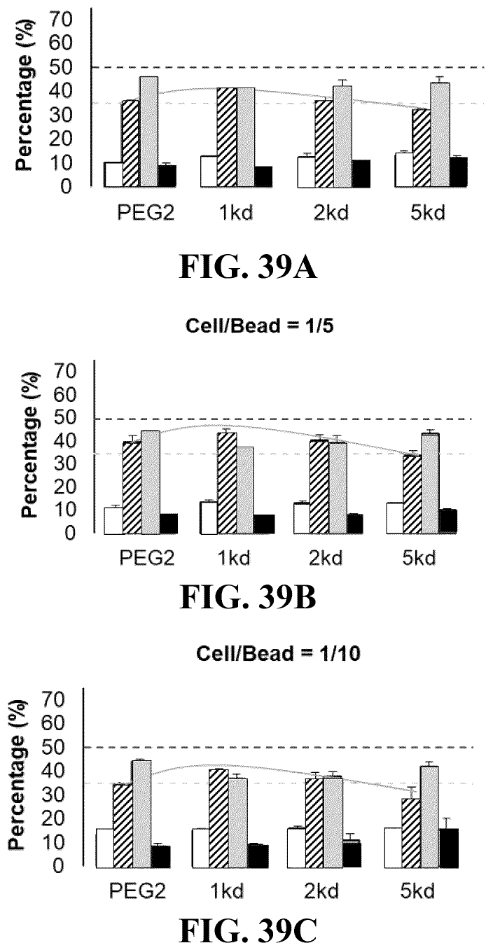

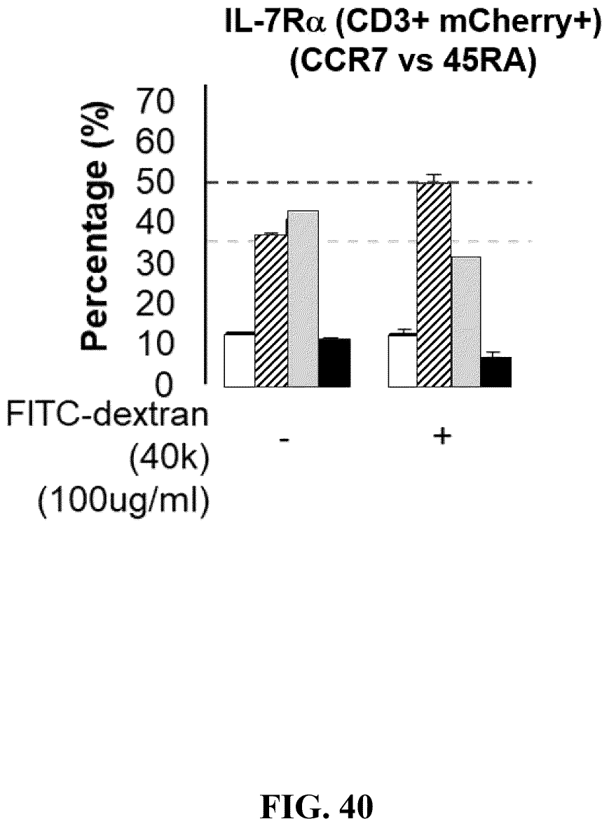

Following transfection, activation, and expansion, the cells were profiled for CCR7 and CD45RA expression. Results of the hydrophobic and hydrophilic bead activations are provided in FIG. 37A and FIG. 37B, respectively. In each figure, results from the control assays with fluorescein-functionalized substrate, fluorescein-coated dextran wells, and fluorescein-labeled CD3 antibodies are shown on the top, middle, and bottom of the leftmost columns, columns 2-5 correspond to beads with PEG2, 1 kD PEG, 2 kD PEG, and 5 kD PEG linkers, respectively, while the top, middle, and bottom rows of columns 2-5 correspond to 1:2, 1:5, and 1:10 cell: bead ratios, respectively. The percentage of effector memory, central memory, stem-cell memory, and terminally differentiated effector memory cells (left to right among each data cluster) from each assay are summarized in FIGS. 38A-38C for the hydrophobic bead assays and FIGS. 39A-39C for the hydrophilic bead assays, respectively (the legend for FIGS. 38A-38C and FIGS. 39A-39C is shown in FIG. 30A). These plots demonstrate that both beads activated the engineered cytokine receptors and promoted central memory phenotypes, with the hydrophobic beads generating more pronounced effects than the hydrophilic beads. T-cell phenotypes from control assays utilizing fluorescein-labeled CD3 antibodies and either including or lacking fluorescein-coated dextran wells are provided in FIG. 40.

### Example 10: Ex Vivo Activation of Engineered T-cells

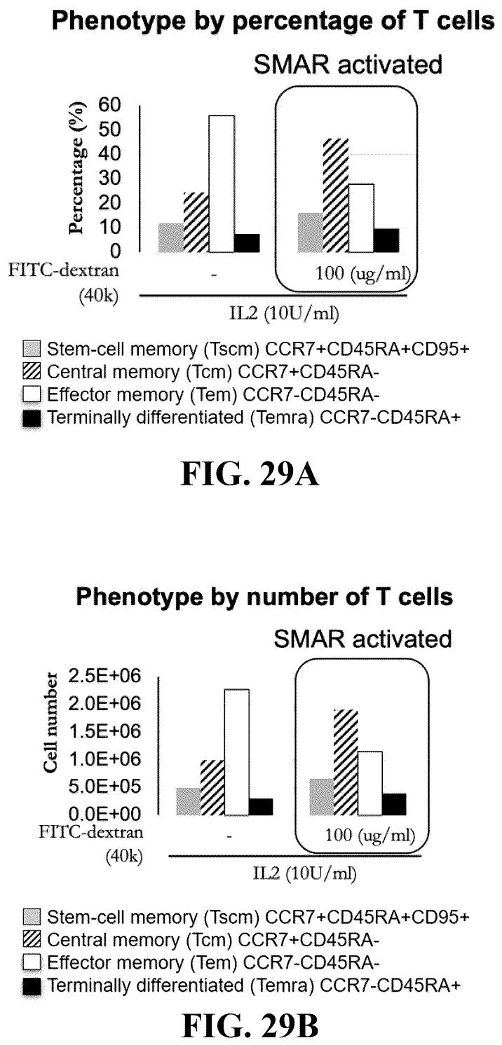

This example covers lymphocyte differentiation into various forms of memory T-cells. The lymphocytes were transformed to express fluorescein-activatable engineered cytokine receptor switches. The lymphocytes were contacted with 10 units/ml IL2, and either no fluorescein or 100 μg/ml fluorescein in the form of fluorescein-labeled dextran coating to the incubation wells or fluorescein-coated hydrophobic beads. Effector memory T-cell, central memory T-cell, stem-cell memory T-cells, and terminally differentiated effector memory T-cell populations were then quantified in each sample. The results of these analysis are provided in FIG. 29A and FIG. 29B, with FIG. 29A providing the memory phenotypes by percentage of T-cells, and FIG. 29B providing raw cell numbers for each memory phenotype. As can be seen from these plots, fluorescein increased the proportion of stem-cell memory T-cells and central memory T-cells and diminished the proportion of effector memory T-cells in the samples.

### Example 11: T-Cell Expression Profiles Following Engineered Cytokine Receptor Switch Activation

This example covers changes in T-cell expression profiles following engineered cytokine receptor switch-driven differentiation. As depicted in FIG. 30A, terminally differentiated effector memory T-cells (Tₑₘᵣₐ) are typically CCR7-/CD45RA+, effector memory T-cells (Tₑₘ) are typically CCR7-/CD45RA-, central memory T-cells (T_{cm}) are typically CCR7+/CD45RA-, and stem cell memory T-cells (T_{scm}) are typically CCR7+/CD45RA+/CD95+. T-cells with engineered cytokine receptor switches were contacted with fluorescein-labeled dextran surfaces, fluorescein coated hydrophobic beads, fluorescein labeled CD3-targeting antibodies, or were left untreated. The cells were then profiled for CCR7, CD45RA, and CD95 expression to quantitate memory T-cell populations.

The percentage of T_{scm}, T_{cm}, Tₑₘ, and Tₑₘᵣₐ in the various treatment groups (from left to right, untreated, fluorescein coated dextran wells, fluorescein coated hydrophobic bead activated, and fluorescein labeled anti-CD3 activated T-cells, respectively) are summarized in FIG. 30B. As can be seen from this plot, all three treatments decreased the proportion of Tₑₘ and increased the proportion of Tₑₘ in the samples. While the two fluorescein treatments generated similar differentiation profiles, the fluorescein labeled anti-CD3 affected approximately 20% greater conversion to Tₑₘ and 20% lower Tₑₘ than either of the beads. FIG. 30C provides plots of CCR7 and CD45RA expression levels in the various treatment groups (from left to right, untreated, fluorescein coated dextran surface activated, fluorescein coated hydrophobic bead activated, and fluorescein labeled anti-CD3 activated T-cells, respectively). All three treatments increased CCR7 expression, with the fluorescein labeled anti-CD3 treatment affecting the greatest increase CCR7 expression relative to the untreated sample.

### Example 12: Engineered Immune Cell Collection Through Engineered Cytokine Receptor Switch Adhesion

This example covers engineered immune cell collection through engineered cytokine receptor switch binding. In addition to engineered immune cell activation, this example demonstrates that engineered cytokine receptor switches can adhere engineered immune cells to substrates by binding to substrate-coupled small molecule activators. Immune cells were collected from a subject and engineered to express a cytokine receptor switch with fluorescein binding affinity. The immune cells were then incubated in uncoated containers (FIG. 33A) and in containers coated with fluorescein (FIG. 33B). The immune cells adhered to the surfaces of the functionalized container only, thus indicating that engineered cytokine receptor switches can have sufficient small molecule activator binding activity to facilitate collection and separation via surface-functionalized containers.

### Example 13: Recovery Rate and Purity of Engineered Immune Cells

This example tests the recovery rate and purity of engineered immune cells collected from a small molecule activator coated surface and from bulk solution. A fluorescein-coated and an uncoated well plate were seeded to 30% confluence with Jurkat cells engineered to express mCherry fluorescent protein and an engineered cytokine receptor switch with binding affinity for fluorescein and an IL7Rα intracellular signaling domain. The cells were expanded for three days. Suspended and adherent cells were separately collected from each plate and analyzed for viability, diameter, percentage of cells which expressed the engineered cytokine receptor switch, total cell count (as determined by side scatter area (SSC-A) in flow cytometry), percent of total cells collected, and mCherry expression.

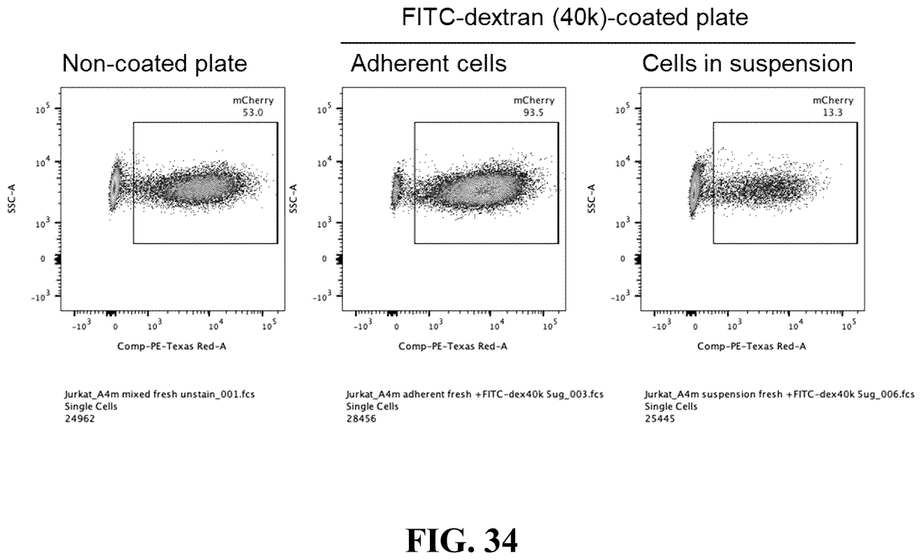

The results of the mCherry expression profiling and SSC-A cell counts are summarized in FIG. 34, with the leftmost plot providing expression levels in cells cultured in the noncoated well plate, the middle plot providing expression levels in cells which adhered to the fluorescein-coated well plate, the rightmost plot providing expression levels in cells collected from suspension in the fluorescein-coated well plate, each y-axis denoting SSC-A cell counts, and each x-axis denoting mCherry expression. As can be seen from these figures, cells collected from the noncoated well plate and from suspension in the fluorescein-coated well plate exhibited lower mCherry expression than cells collected from the surface of the fluorescein-coated well plate, indicating that cells expressing the engineered cytokine receptor switch could be recovered from the surface adherent population with greater purity. Cell viability, diameter, percentage of cells which expressed the engineered cytokine receptor switch, total cell count, and percent of total cells collected for the cells cultured in the fluorescein-coated well plate are summarized in Table 17.

TABLE 17

| | | Viability | Diameter | Purity | Total cell | Yield (% |
|---|---|---|---|---|---|---|
| | Population | (%) | (um) | SMAR+ (%) | # per well | of total) |
| Before enrichment | whole | 97% | 11.0 | 53% | 7.86 × 10⁶ | 100% |
| After enrichment | Adherent cells | 99.3 | 11.5 | 93.5% | 3.74 × 10⁶ | 47.5% |
| | Cells in suspension | 96.2 | 11.0 | 13.3% | 4.12 × 10⁶ | 52.5% |

### Example 14: Methods for Detecting Expression of Engineered Cytokine Receptor Switches

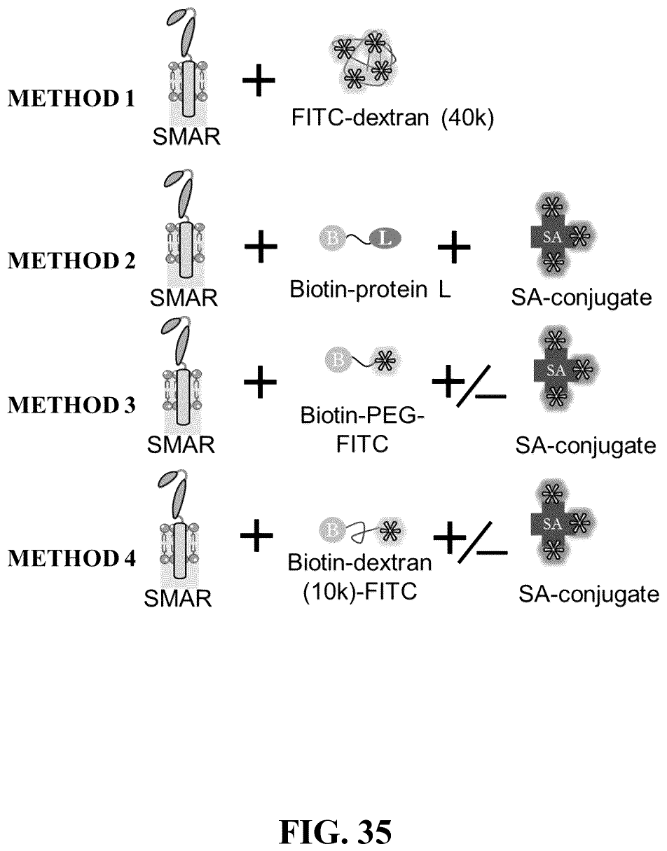

This example compares four methods for detecting engineered cytokine receptor switch expression. In these methods, which are summarized in FIG. 35, immune cells engineered to express engineered cytokine receptor switches with fluorescein binding affinity were contacted to various fluorescently-labeled substrates.

The first method for quantifying engineered cytokine receptor switch expression (FIG. 35 ‘Method 1’) involved contacting the engineered immune cells to fluorescein-functionalized dextran and detecting the cells through mCherry fluorescence. The signals correlated with mCherry expression level, suggesting that this method is effective for quantifying engineered cytokine receptor switch expression. However, this method was not as effective for identifying low mCherry expression levels as Methods 2-4.

As a control, a second detection method (FIG. 35 ‘Method 2’) employed fluorescently-labeled streptavidin and biotin conjugated to Protein L. As neither of these species included streptavidin, this method did not detect engineered cytokine receptor switches.

A third detection method (FIG. 35 ‘Method 3’) utilized fluorescein-functionalized biotin with a short linker (PEG) and fluorescently-labeled streptavidin for engineered cytokine receptor switch detection. This method was able to detect engineered cytokine receptor switch expression but included high background staining.

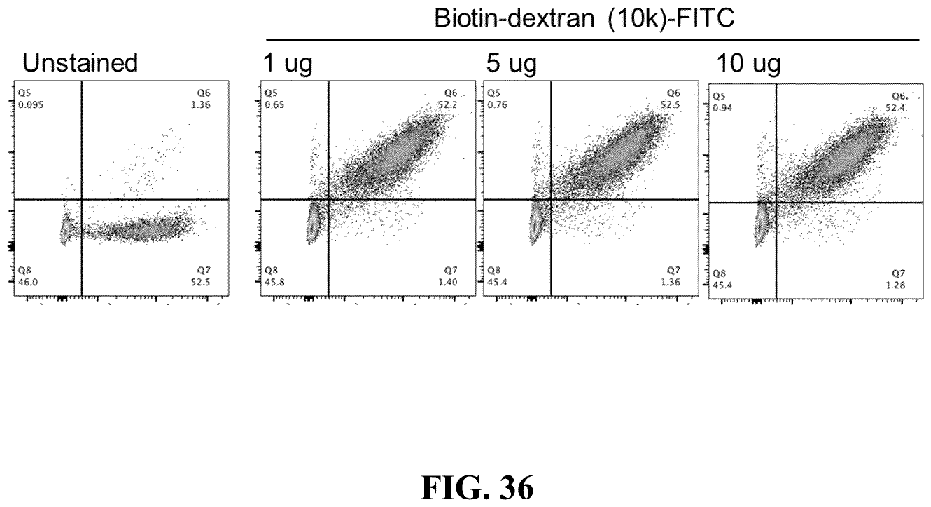

A fourth detection method (FIG. 35 ‘Method 4’) utilized fluorescein-functionalized biotin with a long linker (10k dextran) and fluorescently-labeled streptavidin for engineered cytokine receptor switch detection. This method was able to detect high and low levels of engineered cytokine receptor switch expression. Results from Method 4 analyses are presented in FIG. 36. In this figure, the leftmost plot corresponds to unstained cells, the second plot from the left corresponds to cells stained with 1 μg biotin-dextran-fluorescein conjugates, the second plot from the right corresponds to cells stained with 5 μg biotin-dextran-fluorescein conjugates, and the rightmost plot corresponds to cells stained with 10 μg biotin-dextran-fluorescein conjugates.

### Example 15: Drug-To-Antibody Ratio of Multiple Antibody-Drug Conjugates

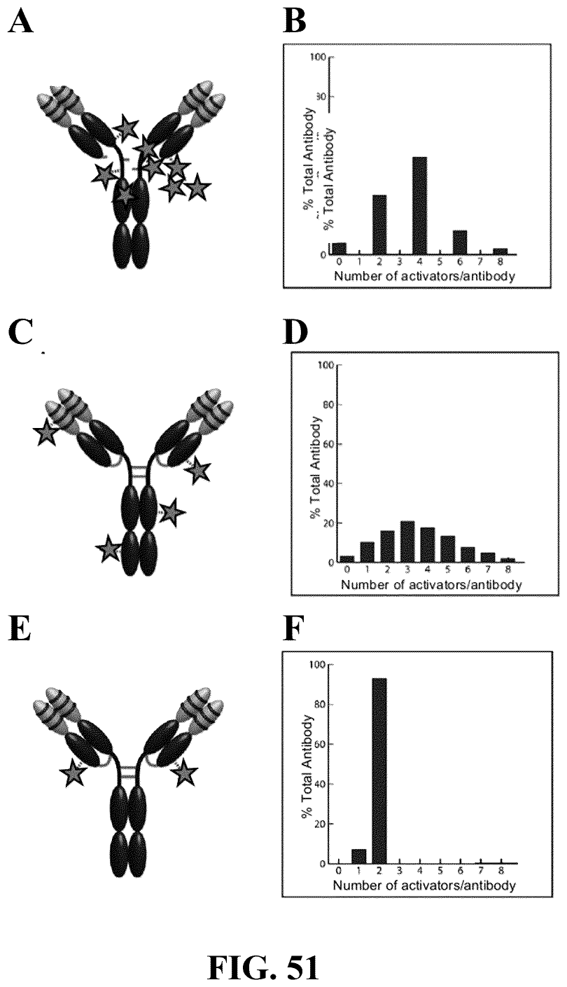

This example covers drug-to-antibody ratio (DAR) measurements for multiple lysine and cysteine-functionalized antibody-drug conjugates (ADCs) consistent with the present disclosure. ADCs are generated by stochastic cysteine conjugation, native lysine conjugation, or site-specific conjugation, and the associated product distribution is dependent on the conjugation technology (FIG. 51). In Examples 15-31, the term “drug” refers to the fluorescein activator. Anti-BCMA antibodies were acylated at lysine using NHS-5 (6)-fluorescein, or alkylated at cysteine with a 5-maleimide-fluorescein or maleimido-mono-amide DOTA compound at target DARs of about 3 to 8 in terms of fluorescein molecules per antibody. Ultraviolet-visible (UV-Vis) spectroscopy was used to measure the DAR for each compound based on the differential absorbance of the antibody at 280 nm and fluorescein at 493 nm. The DAR of DOTA conjugates was determined based on absorbance of the antibody at 280 nm at the time-resolved fluorescence assay described in Example 30. Unfunctionalized anti-BCMA antibody (DAR=0) and free maleimide-fluorescein and 5 (6)-carboxy fluorescein controls were included in the spectral overlay to show their absorbance profile.

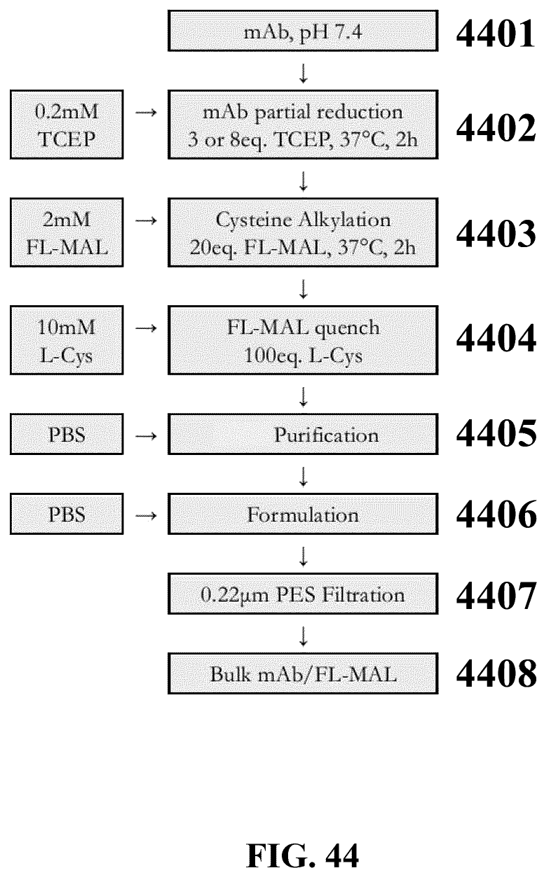

Monoclonal antibodies were thiol functionalized with compound 7 at multiple target DARs as outlined in FIG. 44. pH 7.4 preparations of the antibodies (FIG. 44, 4401) were incubated with either 3 or 8 equivalents of tris(2-carboxyethyl) phosphine (TCEP) at 37° C. for 2 hours to reduce interchain disulfide bonds (FIG. 44, 4402). Following this reduction step, the antibody was incubated with variable amounts of compound 7 at 37° C. (FIG. 44, 4403), resulting in compound 7 conjugation of free thiols generated during reduction. Following 2 hours of incubation, 100 equivalents of L-cysteine were added to the mixture to quench residual unreacted compound 7 (FIG. 44, 4404). The functionalized antibodies were purified by centrifugal filtration against phosphate-buffered saline (PBS) (FIG. 44, 4405), diluted to the target concentration with PBS (FIG. 44, 4406), and filtered through a 0.22 micron polyethersulfone (PES) filter (FIG. 44, 4407) to yield compound 7-functionalized antibodies (FIG. 44, 4408).

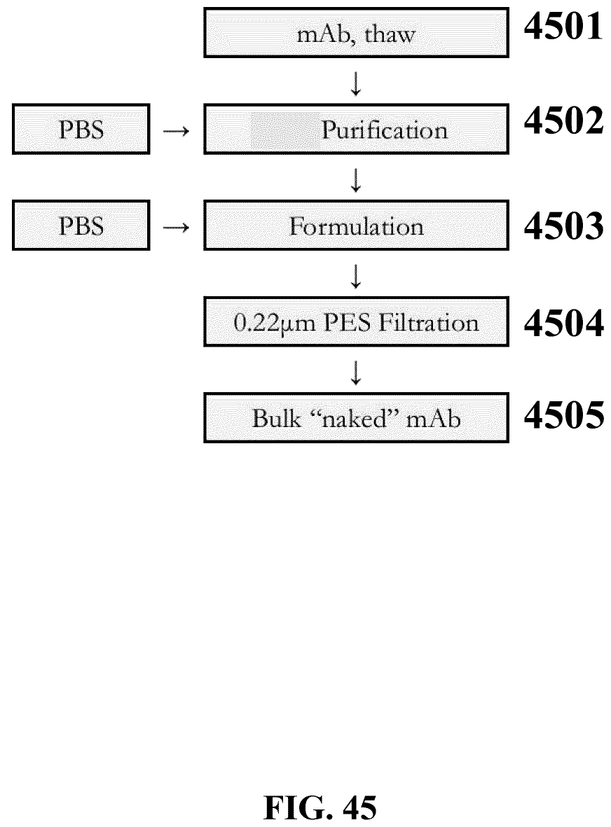

As outlined in FIG. 45, unfunctionalized “naked” antibody controls were formulated with similar purification steps as the functionalized antibodies. Preparations of the antibodies (FIG. 45, 4501) were thawed, and then subjected to buffer exchange purification (FIG. 45, 4502), PBS formulation (FIGS. 45, 4503), and 0.22 micron PES filtration (FIG. 45, 4504), resulting in bulk “naked” antibodies (FIG. 45, 4505) for use as controls in the DAR assays.

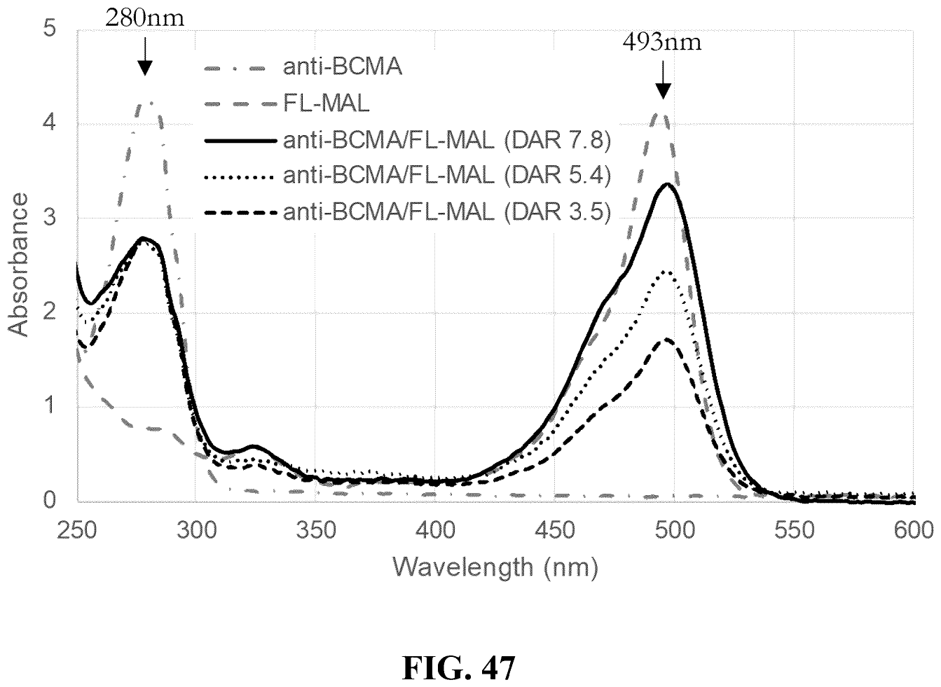

UV-vis absorption profiles were obtained for compound 7-functionalized antibodies, unfunctionalized antibodies, and free compound 7. FIG. 47 provides the results of these analyses. Molar absorptivities (8) were determined for the anti-BCMA antibody at 280 nm (primarily corresponding tyrosine and tryptophan absorbances from the anti-BCMA antibody) and at 493 nm for maleimide fluorescein and 5 (6)-carboxy fluorescein (primarily corresponding to the HOMO to LUMO transition within the fluorescein x-system), and are summarized in Table 18. As the unfunctionalized antibody minimally absorbed around 493 nm, absorbance in this region is quantitative for fluorescein concentration.

TABLE 18

| | ε_{280 nm} | ε493ₙₘ |
|---|---|---|
| Component | (M⁻¹cm⁻¹) | (M⁻¹cm⁻¹) |
| Anti-BCMA Antibody | 229360 | 0 |
| Fluorescein-Maleimide | 7968 | 43908 |
| 5(6)-Carboxy Fluorescein | 14752 | 79828 |

The 280 nm and 493 nm bands (indicated with arrows in FIG. 47) were then used to quantitate conjugated antibody and conjugated fluorescein concentrations, respectively, for the ADCs. As fluorescein comprises an absorption at 280 nm, concentrations of the antibodies and fluorescein maleimide conjugating groups were calculated according to Equations 1-3:

$$Antibody Concentration ( {C}_{mAb} ) = \frac{{A}_{280} {ε}_{LP}^{{λ}_{max}} - {A}_{{λ}_{max}} {ε}_{LP}^{280}}{[ ( {ε}_{mAb}^{280} {ε}_{LP}^{{λ}_{max}} - {ε}_{mAb}^{{λ}_{max}} {ε}_{LP}^{280} ) l ]} \qquad Equation 1$$

$$Conjugating Group Concentration ( {C}_{LP} ) = \frac{{A}_{280} {ε}_{mAb}^{{λ}_{max}} - {A}_{{λ}_{max}} {ε}_{mAb}^{280}}{[ ( {ε}_{mAb}^{280} {ε}_{mAb}^{{λ}_{max}} - {ε}_{LP}^{{λ}_{max}} {ε}_{mAb}^{280} ) l ]} \qquad Equation 2$$

$$Drug - to - Antibody Ratio ( DAR ) = \frac{{C}_{LP}}{{C}_{mAb}} \qquad Equation 3$$

wherein A₂₈₀ denotes absorbance intensity at 280 nm, A_{λmax} denotes absorbance intensity maximum for the 493 nm band, ε_{LP}^{λmax} and ε_{mAb}^{λmax} denote fluorescein and antibody molar absorptivities at 493 nm, respectively, ε_{LP}²⁸⁰ and ε_{mAb}²⁸⁰ denote fluorescein and antibody molar absorptivities at 280 nm, respectively, and I corresponds to path length (in cm).

DARs were calculated as 3.5, 5.4, and 7.8 for the three functionalized antibodies.

### Example 16: Multiple ADCs at Variable DARs

This example covers DAR measurements for multiple lysine-functionalized ADCs with different degrees of functionalization. Monoclonal antibodies were stochastically functionalized with NHS-fluorescein lysine-conjugating groups, and then characterized with UV-vis spectroscopy to determine DARs (conjugating groups per antibody). Unfunctionalized antibodies (DAR=0) and free NHS-fluorescein were characterized as controls as outlined in Example 15.

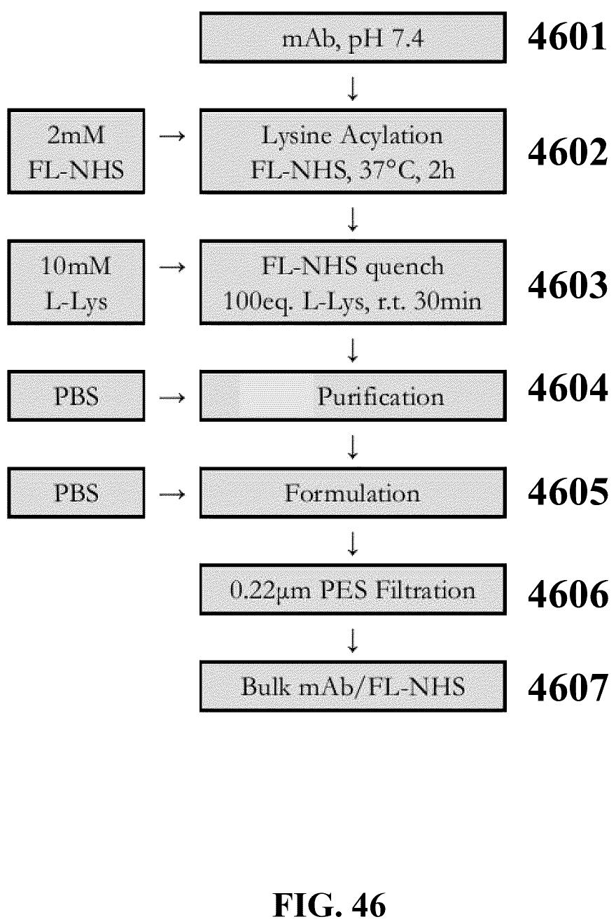

NHS-fluorescein-functionalized antibodies were prepared at multiple DARs. As summarized in FIG. 46, antibodies were prepared at pH 7.4 (FIG. 46, 4601). The antibodies were then functionalized using fluorescein-NHS conjugating groups (FIG. 46, 4602). The antibodies were incubated at 37° C. with compound 8, forming fluorescein-amide constructs from antibody-derived lysyl amines. The reaction was then quenched with 100 equivalents of L-lysine for 30 minutes at room temperature, sequestering remaining unreacted NHS-fluorescein conjugating groups (FIG. 46, 4603). The functionalized antibodies were subjected to TFF purification in PBS (FIG. 46, 4604), and then were further formulated in PBS (FIG. 46, 4605) and purified with a 0.22 micron PES filter (FIG. 46, 4606) to yield bulk compound 8-functionalized antibodies (FIG. 46, 4607).

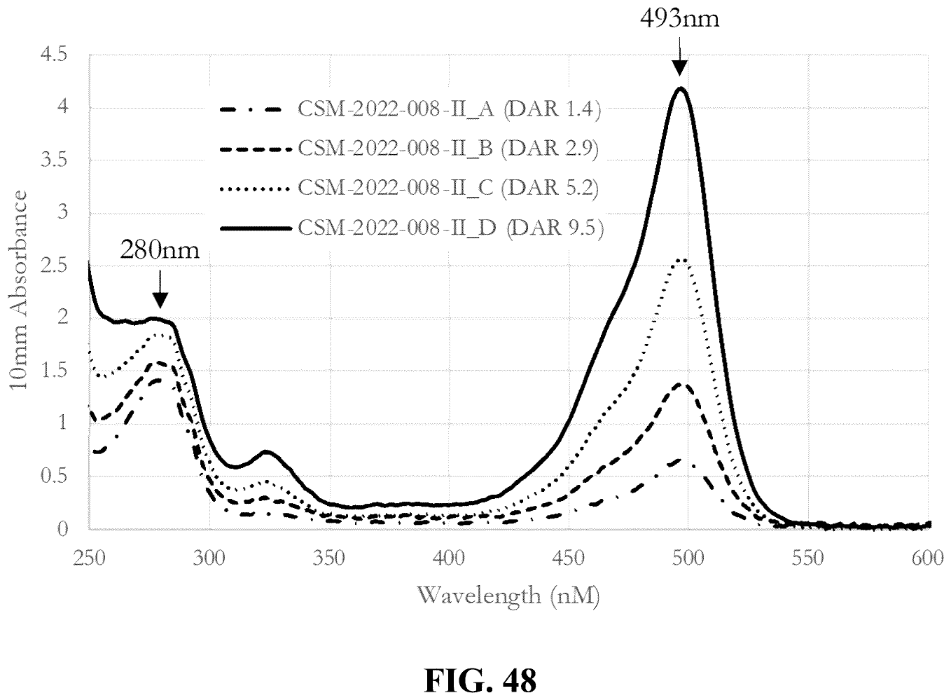

UV-vis absorbance spectra of the resultant ADCs are overlaid in FIG. 48. DARs were assessed based on ratios between the 280 nm (a characteristic protein tyrosine and tryptophan absorbance band) and 493 nm (corresponding to a fluorescein absorbance band) bands as outlined in Example 15. DARs for the four antibody-activator preparations were 1.4, 2.9, 5.2, and 9.5, respectively.

### Example 17: DAR Determination for Metal Chelator-Functionalized Antibodies

This example covers antibody functionalization with the metal chelator 1,4,7,10-tetraazacyclododecane-1,4,7,10-tetraacetate (DOTA), a well-characterized metal chelator capable of binding heavy transition metal ions. Stochastic lysine functionalization was performed with compound 10 according to the procedure outlined in Example 16, resulting in lysyl amide DOTA modified antibodies.

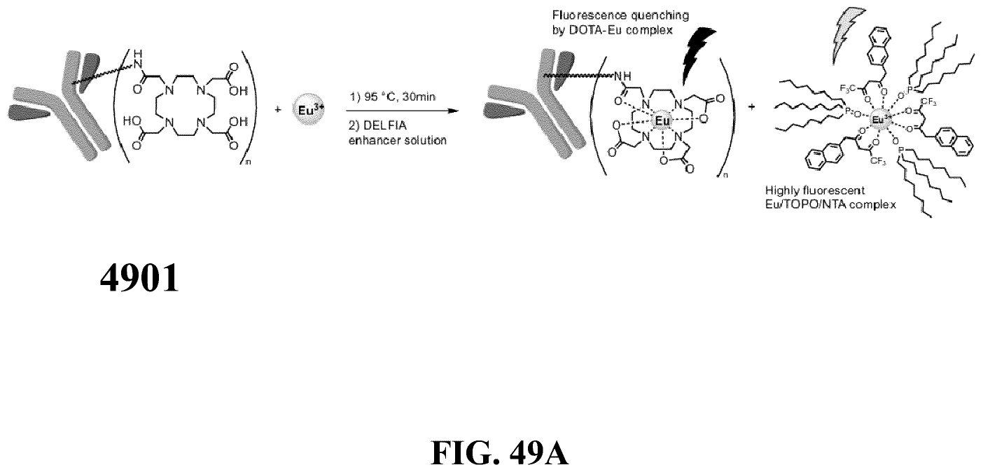

DARs for the resultant antibody-activator complexes were characterized with a europium/DELFIA time-resolved fluorescence (TRF) assay. As outlined in FIG. 49A, the DOTA-functionalized antibodies were incubated with europium (III) ions for 30 minutes at 95° C., allowing portions of the europium (III) ions to bind to the DOTA groups. The samples were then contacted with trioctyl phosphine oxide and 4,4,4-Trifluoro-1-(2-naphthyl)-1,3-butanedione, europium (III) sensitizers and weak metal chelators which complex free europium (III) but do not displace europium (III) from DOTA. As DOTA quenches europium (III) fluorescence, decreases in fluorescence were used to quantify DOTA concentration. The fluorimetry assays were carried out with 340 nm excitation, 615 nm emission detection, 400 microsecond lag times, and 400 microsecond integration times.

### Example 18: Mass Spectrometric DAR Measurements

This example overviews a method for determining antibody DARs by characterizing intact antibody chains with mass spectrometry. In this method, antibodies are fragmented into individual light and heavy chains, and then mass spectrometrically characterized using low intensity, minimally fragmenting ionization. By separately determining the mass of drug-linker conjugates, deviations in light and heavy chain mass can be deconvoluted to determine contributions from drug-linker conjugates, post-translational modifications such as glycosylation, mutations, and other chemical modifications to the antibody, such as cleavage. Whereas other techniques can be limited to determining DARs for whole antibodies (e.g., all four chains of an IgG antibody), intact mass spectrometric characterization can determine separate DARs for each type of chain present in an antibody.

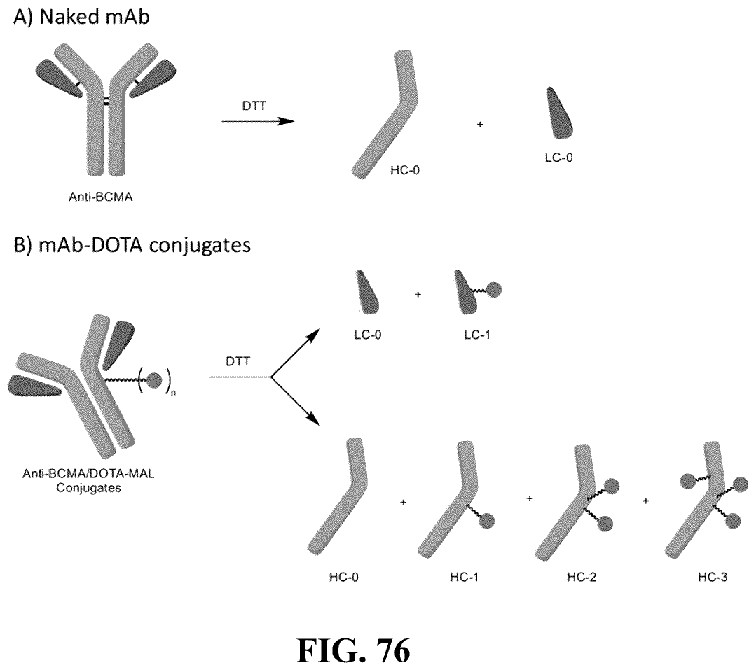

Antibodies were digested prior to mass spectrometric analysis to yield isolated heavy and light chains. As illustrated in FIG. 76, dithiothreitol (DTT) was used to cleave interchain disulfide bonds and separate heavy and light chains without generating intrachain cleavages. While IgG DTT treatment generates 2 identical light and 2 identical heavy chains (panel A), DTT treatment of conjugating group-functionalized antibodies can generate distinct fragments with varying numbers of conjugating groups.

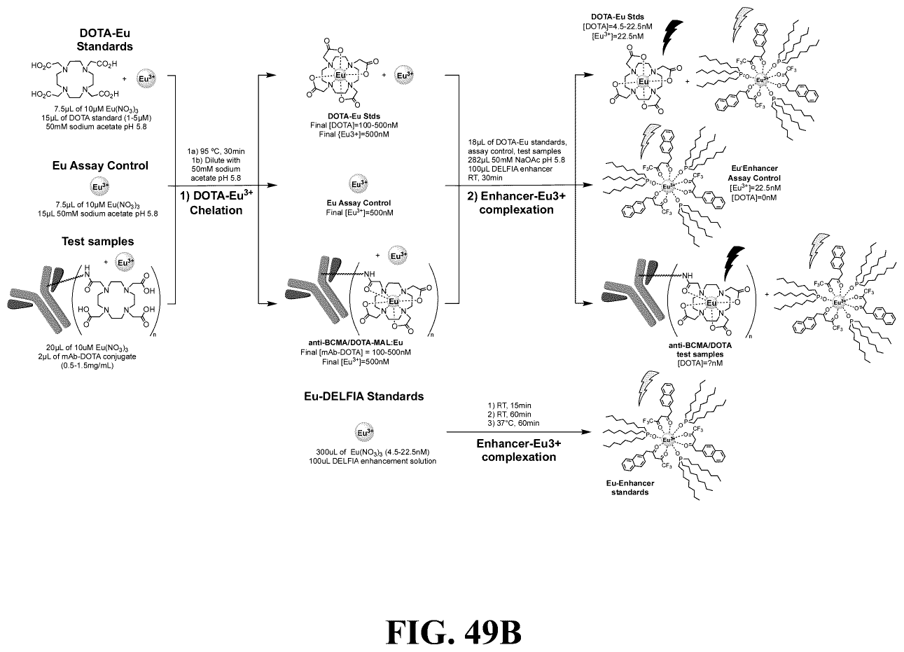

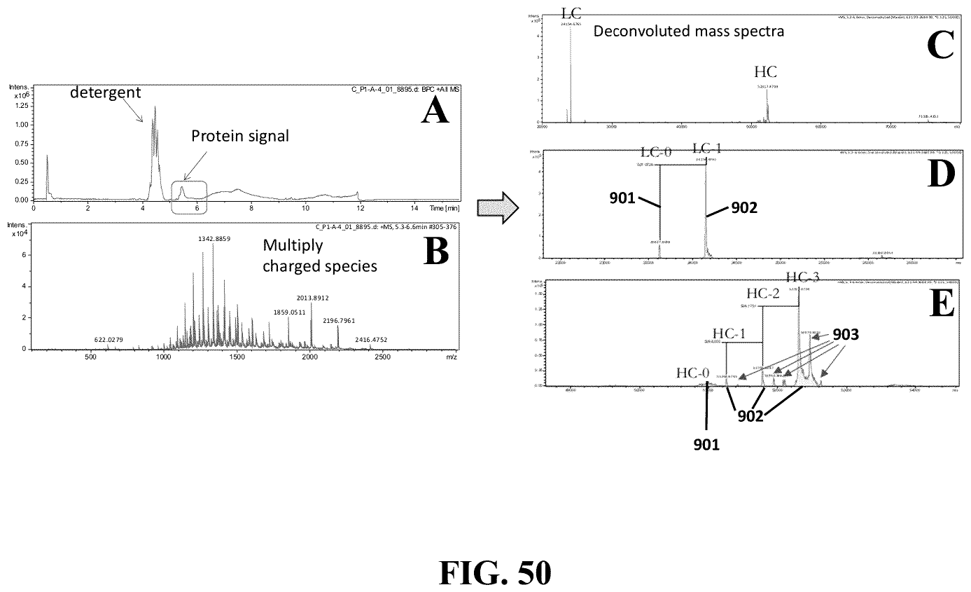

FIG. 50 provides exemplary data from a mass spectrometric DAR measurement. FIG. 50 Panel A provides a liquid chromatogram from liquid chromatography-mass spectrometry analysis of an ADC. The protein signal containing functionalized and unfunctionalized antibody chains is boxed within the figure. FIG. 50 Panel B provides the apparent mass of multiply charged ADC species. This data can then be used to deconvolute spectra of light and heavy chains for DAR determination. FIG. 50 Panel C provides a deconvoluted mass spectrum of the ADC, with light chain (LC) and heavy chain (HC) signals around 25000 and 52000 m/z, respectively. FIG. 50 Panels D & E provide enlarged views of the mass spectra of the light and heavy chains, respectively, of a representative ADC. In these spectra, the parent peaks 901 correspond to light and heavy chains containing no drug-linker conjugates. Peaks with mass-to-charge ratios equal to the sum of the parent peak and integer multiples of the drug-linker mass 902 can be attributed to antibody chains coupled to drug-linker conjugates. In the spectra shown, only light chains coupled to zero and one drug-linker conjugates were observed. Conversely, only a small portion of the heavy chains contained zero drug-linker conjugates, and instead DARs of one, two, and three were primarily observed. Additionally, the heavy chain spectrum includes peaks corresponding to glycosylation 903, allowing further determination of the glycosylation patterns present within the drug-antibody conjugate consortium.

$$Light Chain Drug - to - Antibody Ratio DAR ( LC ) = \frac{\sum n {A}_{{LC}_{N}}}{\sum {A}_{{LC}_{n}}} \qquad Equation 4$$

$$Heavy Chain Drug - to - Antibody Ratio DAR ( HC ) = \frac{\sum n {A}_{{HC}_{N}}}{\sum {A}_{{HC}_{n}}} \qquad Equation 5$$

Light and heavy chain DARs were determined as the number-weighted average for drug-linker conjugates observed on each chain, as outlined in equations 4 and 5. The analyzed ADC was determined to have a light chain DAR of 0.9 and a heavy chain DAR of 2.8, summing to a DAR of 7.4 for the ADC (accounting for two heavy chains and two light chains per antibody).

### Example 19: Optimization of Stochastic Cysteine Conjugations Using TCEP Titration

This example covers a method for controlling and measuring DARs in cysteine-conjugated ADCs. The ADCs were prepared according to FIG. 42B, using variable concentrations of reductant to achieve different degrees of disulfide reduction to liberate maleimide-conjugatable thiols. Specifically, BCMA-targeting antibodies were incubated with 2, 4, 6, 8, or 10 equivalents of Tris(2-carboxyethyl) phosphine (TCEP) reductant for 2.5 hours in pH 7.4 phosphate buffered saline (PBS) at 37° C., producing populations of antibodies with varying degrees of disulfide reduction. ADCs were then generated by coupling the reduced antibodies to an excess of compound 7. In this step, the reduced antibodies were reacted with 20 equivalents of compound 7 for 1 hour in pH 7.4 PBS with 10% (volume/volume) DMSO at 37° C. Following this step, remaining compound 7 was sequestered with 100 equivalents of L-cysteine at room temperature for 30 minutes. The resultant ADCs were then purified with TFF and 0.22 μm PES according to FIG. 44.

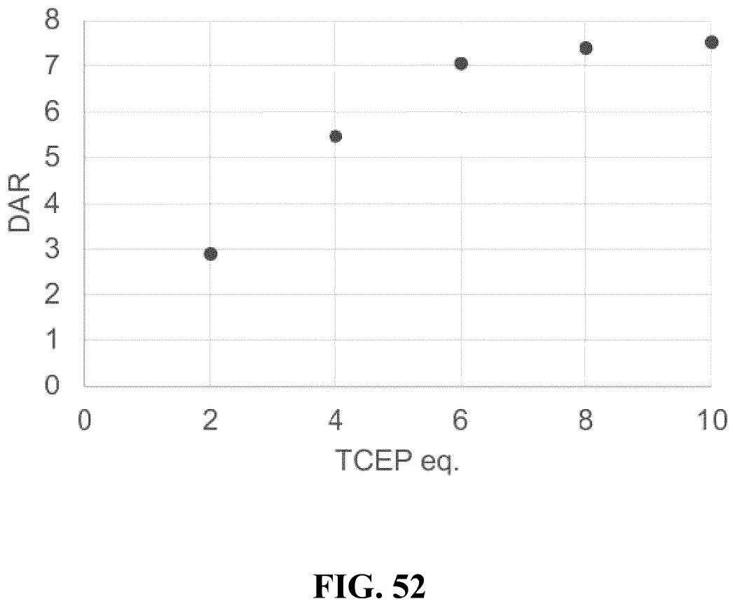

DARs were fluorometrically and mass spectrometrically determined for ADC preparations according to Example 17 and Example 18. FIG. 52 summarizes the DARs achieved with each reductant concentration. DARs ranged from just under 3 for ADCs prepared with 2 equivalents of reductant to about 7.5 for ADCs prepared with 10 equivalents of reductant. While the relationship between reductant concentration and DAR was fairly linear between 2 and 6 equivalents of reductant, yielding a slope of about 1 DAR unit per equivalent of TCEP, the relationship between reductant concentration and DAR was fairly asymptotic past 6 equivalents of reductant, reaching an apparent maximum of about 7.5 equivalents of compound 7 per antibody.

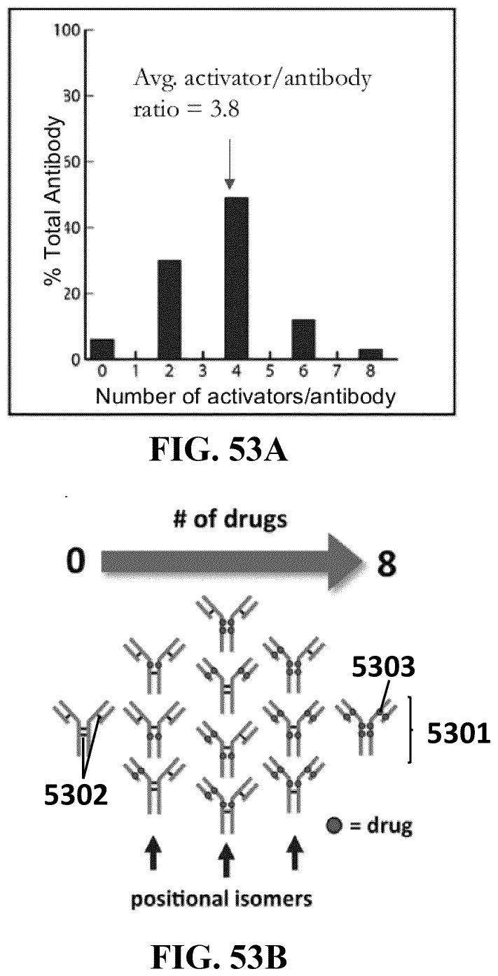

DAR distributions were also determined for the resultant ADCs. As shown in FIG. 53A, the ADCs contained 0, 2, 4, 6, or 8 instances of compound 7, consistent with the model that maleimides predominantly couple to thiols from reduced interchain-disulfides. FIG. 53B depicts positional isomers of stochastic cysteine ADCs. As thiols are liberated in pairs from interchain disulfide bonds 5302, ADCs 5301 primarily contain even numbers of conjugating groups 5303. Increasing the distributional stochasticity of maleimide functionalization, ADCs with 2, 4, and 6 conjugating groups include multiple positional isomers, reflecting the distribution of arrangements reduced and unreduced interchain disulfides.

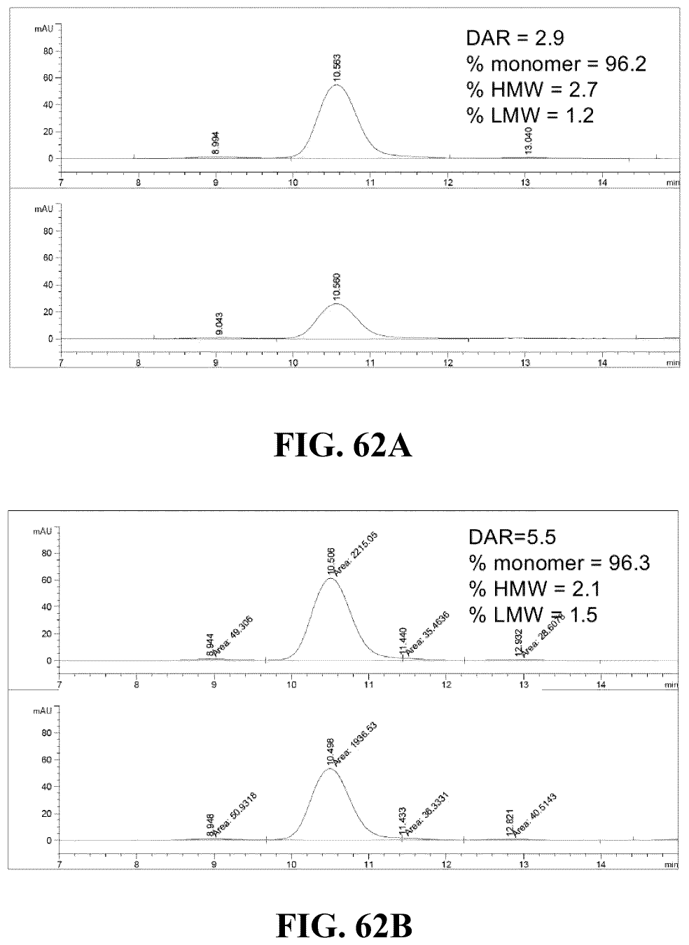

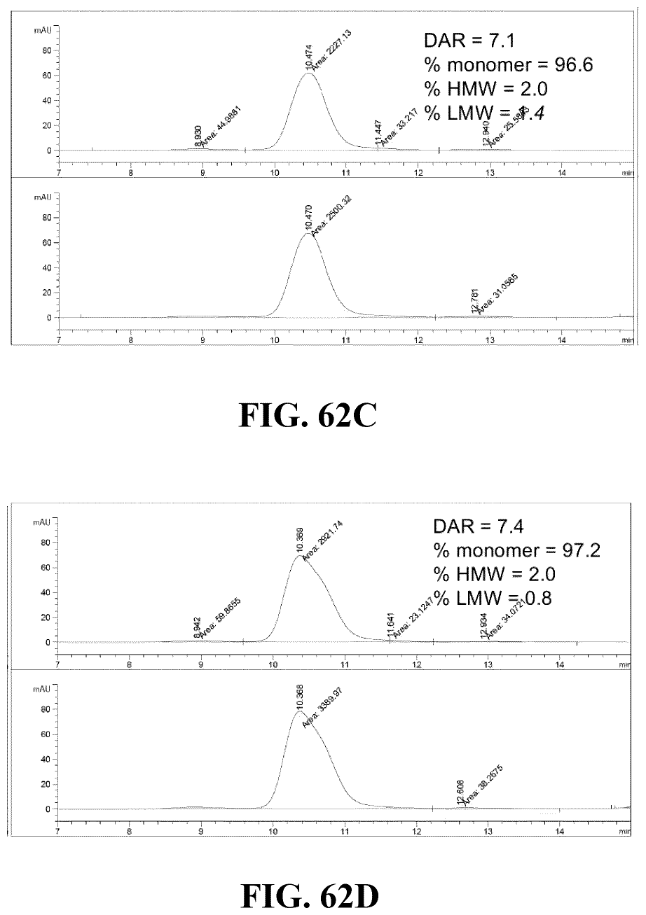

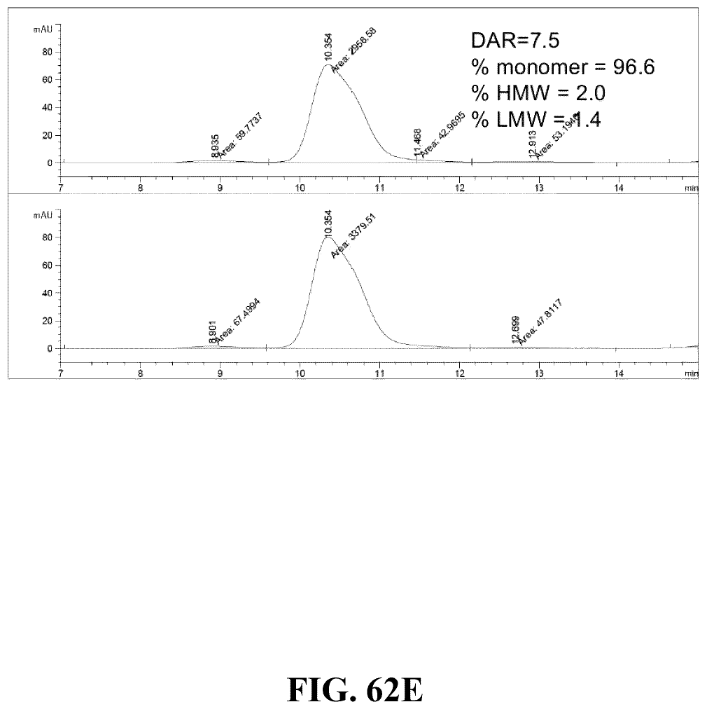

The purity of each ADC preparation was determined with size-exclusion chromatography. Representative chromatograms are provided in FIGS. 62A-62E for the 2.9, 5.5, 7.1, 7.4, and 7.5 DAR ADC preparations respectively. In each figure, the top and bottom plots correspond to chromatograms collected at 280 nm (antibody) and 493 nm (fluorescein) absorbance, respectively. The monomer, HMW, and LMW for each ADC preparation is provided in Table 19. The monomeric content of the ADC preparations ranged from 96.2% to 97.2%, while the HMW and LMW ranged from 2.0% to 2.7% and 0.8% to 1.5%, respectively.

TABLE 19

| | | | | | |
|---|---|---|---|---|---|
| DAR | 2.9 | 5.5 | 7.1 | 7.4 | 7.5 |
| % Monomer | 96.2 | 96.3 | 96.6 | 97.2 | 96.6 |
| % HMW | 2.7 | 2.1 | 2.0 | 2.0 | 2.0 |
| % LMW | 1.2 | 1.5 | 1.4 | 0.8 | 1.4 |

### Example 20: Optimization of Stochastic Cysteine Conjugations Using TCEP Titration

This example covers cysteine conjugation stochasticity in ADCs prepared with varying concentrations of reductant. The ADCs were prepared according to Example 19 and FIG. 42B, using 2.8, 4, and 8 equivalents of TCEP reductant and higher antibody titers. The resultant antibodies were characterized with UV-visible spectroscopy according to Example 16 and separately with size-exclusion chromatography (SEC). As with the UV-visible spectroscopic characterization, the SEC utilized two channels for antibody analysis, 280 nm and 493 nm for antibody and compound 7 quantitation, respectively.

Characteristics of the resultant ADCs are summarized in Table 20. While 90 μg antibodies were used for each preparation, between 57 (63.5%) and 66 μg (73.4%) were recovered. SEC and UV-visible spectroscopy determined different DARs for the ADC preparations. Whereas SEC determined DARs of 2.3, 3.5, and 4.7 for the 2.8, 4.0, and 8.0 TCEP equivalent-preparations, respectively, UV-visible spectroscopy determined DARs of 3.5, 5.4, and 7.8 for the three preparations.

TABLE 20

| | 2.8 Equivalents | 4.0 Equivalents | 8.0 Equivalents |
|---|---|---|---|
| | TCEP | TCEP | TCEP |
| Scale (μg) | 90 | 90 | 90 |
| Recovery (μg) | 66 | 57 | 63 |
| % Recovery | 73.4 | 63.5 | 70.0 |
| DAR (SEC) | 2.3 | 3.5 | 4.7 |
| DAR (UV) | 3.5 | 5.4 | 7.8 |
| % monomer | 90.8 | 98.2 | 92.1 |
| % HMW | 0.6 | 0.5 | 0.0 |
| % LMW | 8.7 | 0.5 | 7.9 |
| Concentration | 1.3 | 1.0 | 1.4 |
| (mg/mL) | | | |

In addition to identifying DARs, the size-exclusion chromatography also determined the proportions of each antibody composition which were intact and monomeric. During antibody and ADC production, antibodies can aggregate to form high molecular weight adducts (HMW), and can cleave to form low molecular weight fragments (LMW), which often diminish antibody and ADC efficacies and can affect adverse immunological responses (see Tan et al. MAbs, 2020; 12 (1): 1829333). Accordingly, therapeutic compositions often require low amounts of HMW and LMW species. The monomer contents ranged from 90.8% to 98.2%. The 2.8- and 8.0-TCEP equivalent preparations had monomer contents of 90.8% and 92.1% primarily reflected LMW contents (8.7% and 7.9%, respectively). The preparation which utilized 4.0-equivalents of TCEP had the highest monomer content (98.2%), with only 0.5% LMW and HMW.

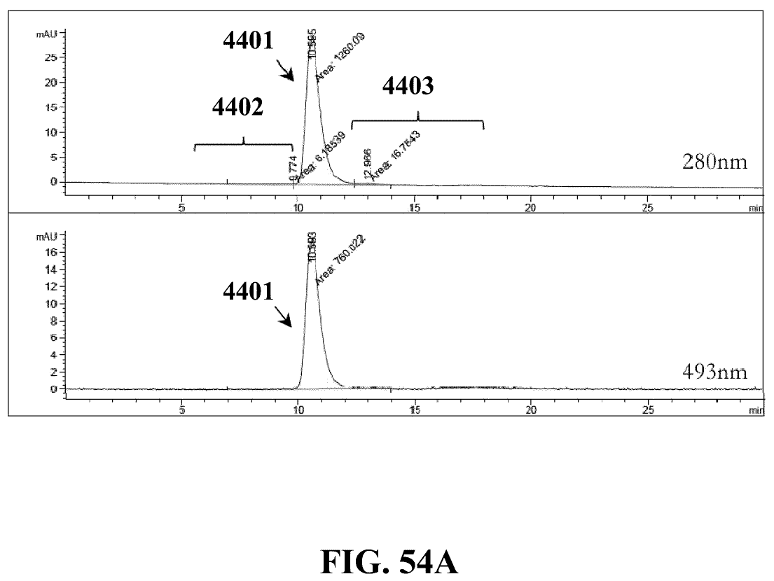

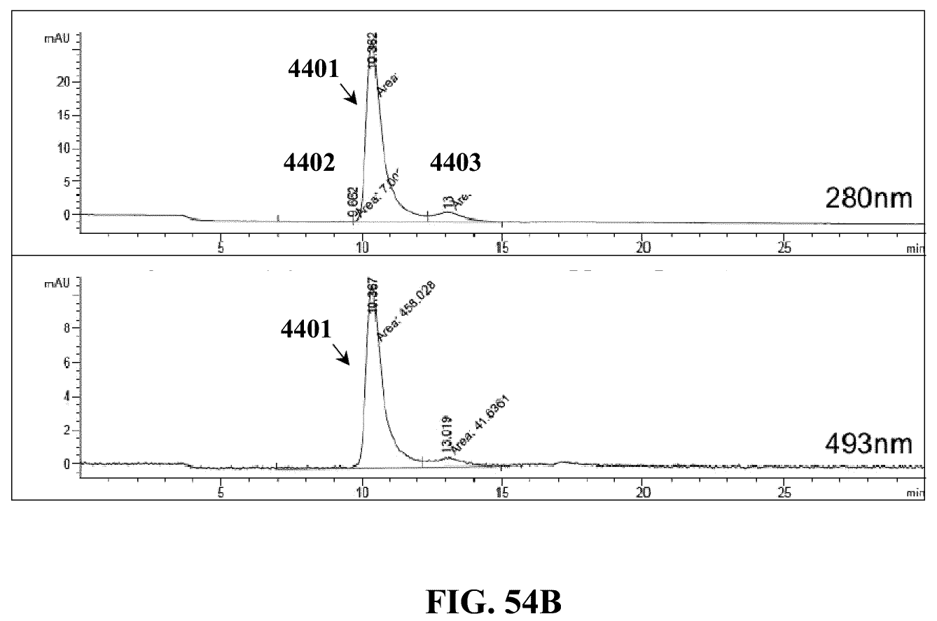

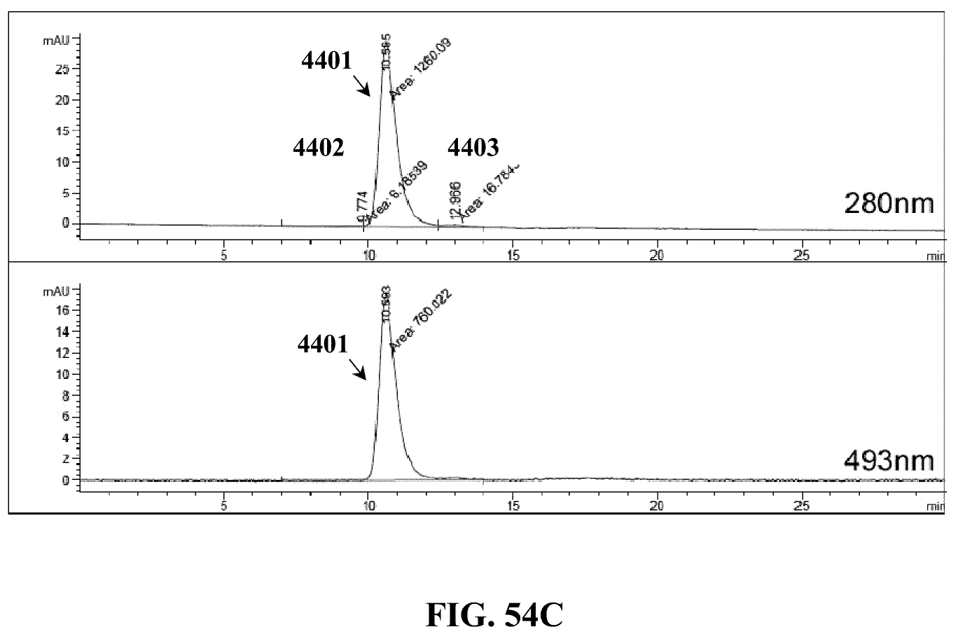

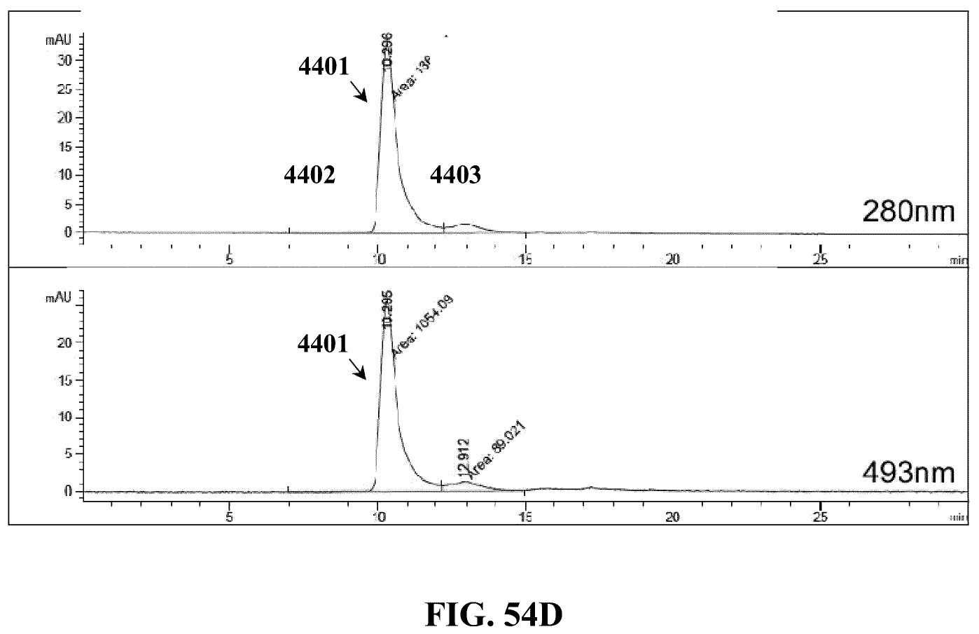

Exemplary SEC results are displayed in FIGS. 54A-54D. In each figure, the upper and lower panels provide the absorbances from the 280 nm and 493 nm channels, respectively. In these plots, the x-axes denote size-exclusion column retention time, while the y-axes provide absorbance intensity. The main peak in the chromatograms 4401 corresponds to the antibody and ADCs. The ratio of this peak between the 280 nm (protein) and 493 nm (fluorescein) bands in the chromatograms can be used to determine ADC DAR according to Equations 1-3. As SEC separates species based on size, region 4402 below the retention time of the main peak was used to determine the relative concentration of HWM, while the region 4403 at higher retention times than the main peak was used to determine the relative concentration of LWM. FIG. 54B, FIG. 54C, and FIG. 54D correspond to exemplary DAR 3.5, 5.4, and 7.8 samples (as determined by UV-visible absorbance spectroscopy), respectively.

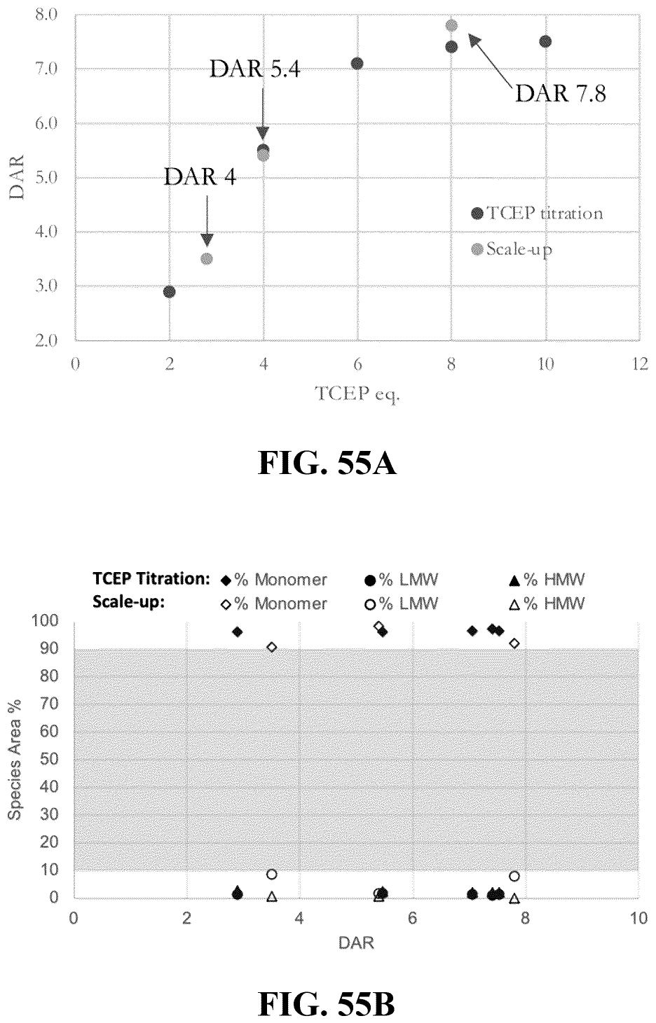

FIG. 55A overlays DAR data for the ADCs with DAR data of smaller-scale syntheses. TCEP titration was used for determining the number of TCEP equivalents required to generate an ADC with a target DAR. While the DAR-reductant ratios were similar within the linear region below 6 reductant equivalents, the DAR at 8 reductant equivalents were slightly greater for the scaled-up synthesis of the present example. FIG. 55B compares monomer, LMW, and HMW prevalence between the Example 19 syntheses. In general, the ADC purity target was >90% monomer and <10% HMW and LMW impurities. While all syntheses yielded at least 90% monomer antibodies, the two lowest monomer percentages were obtained with the scaled-up reaction of the present example.

### Example 21: Scalable Stochastic Cysteine Conjugations with Multiple Antibodies

This example covers ADC preparation with cysteine thiol-conjugating groups. ADCs were prepared with compound 7 or compound 8 according to the methods outlined in Example 19. The antibodies targeted multiple myeloma (MM) antigens BCMA, CS1, or GPRC5D, acute myeloid leukemia antigens CD33, CD123, or CD56, or T cell markers CD3, CD4, or CD38. ADCs were prepared at 80 μg, 90 μg, 100 μg, 500 μg, 800 μg, 2000 μg, or 3800 μg scales (input antibody mass) and DARs spanning 3.4 to 8.1. Synthesis recoveries ranged from 62.4% to 97.0%, with the three highest recoveries of 88.8%, 95.5%, and 97.0% corresponding to 2000 μg and 3800 μg scales.

A parallel preparation of a control antibody (Ab, Table 21A) was subjected to TFF and PES filtration, as outlined in FIG. 45. Comparatively, the control antibody contained similar monomeric content as the ADCs, suggesting that LMW and HMW impurities may be generated during purification or prior to functionalization. Selected MM and T cell marker ADCs, as well as a control antibody, are summarized in Table 21A, Table 21B, and Table 22, respectively.

TABLE 21A

| Target | Ab | BCMA | BCMA | BCMA | BCMA | CS1 | GPRC5D |
|---|---|---|---|---|---|---|---|
| Small | None | Compound 7 | Compound 7 | Compound 8 | Compound 8 | Compound 7 | Compound 7 |
| Molecule | | | | | | | |
| Activator | | | | | | | |
| Scale | 4000 | 2000 | 2000 | 90 | 90 | 3800 | 800 |
| (μg) | | | | | | | |
| Recovery | 2383 | 1775 | 1940 | 65 | 71 | 3723 | 526 |
| (μg) | | | | | | | |
| Recovery | 59.6 | 88.8 | 97.0 | 71.7 | 78.8 | 95.5 | 65.7 |
| (%) | | | | | | | |
| DAR | N/A | 3.4 | 7.9 | 4.5 | 7.6 | 8.1 | 7.4 |
| Monomer | 93.2 | 93.3 | 91.8 | 95.6 | 91.9 | 95.3 | 94.0 |
| (%) | | | | | | | |
| HMW | 4.5 | 1.8 | 1.7 | 1.0 | 5.6 | 2.0 | 1.4 |
| (%) | | | | | | | |
| LMW | 2.3 | 4.9 | 6.4 | 3.3 | 2.5 | 2.7 | 4.6 |
| (%) | | | | | | | |

TABLE 21B

| Target | CD3 | CD3 |
|---|---|---|
| Small Molecule | Compound 7 | Compound 7 |
| Activator | | |
| Scale (μg) | 500 | 500 |
| Recovery (μg) | 387 | 242 |
| Recovery (%) | 77.3 | 48.4 |
| DAR | 4.3 | 8.1 |
| Monomer (%) | 98.2 | 89.8 |
| HMW (%) | 0.5 | 3.0 |
| LMW (%) | 1.3 | .2 |

TABLE 22

| Target | BCMA | BCMA | BCMA |
|---|---|---|---|
| Small Molecule | Compound 7 | Compound 7 | Compound 7 |
| Activator | | | |
| Scale (μg) | 80 | 80 | 100 |
| Recovery (μg) | 56 | 54 | 62 |
| Recovery (%) | 70.3 | 67.9 | 62.4 |
| DAR | 5.0 | 5.0 | 5.8 |
| Monomer (%) | 88.6 | 94.9 | 96.3 |
| HMW (%) | 10.2 | 3.9 | 1.4 |
| LMW (%) | 1.2 | 1.2 | 2.3 |

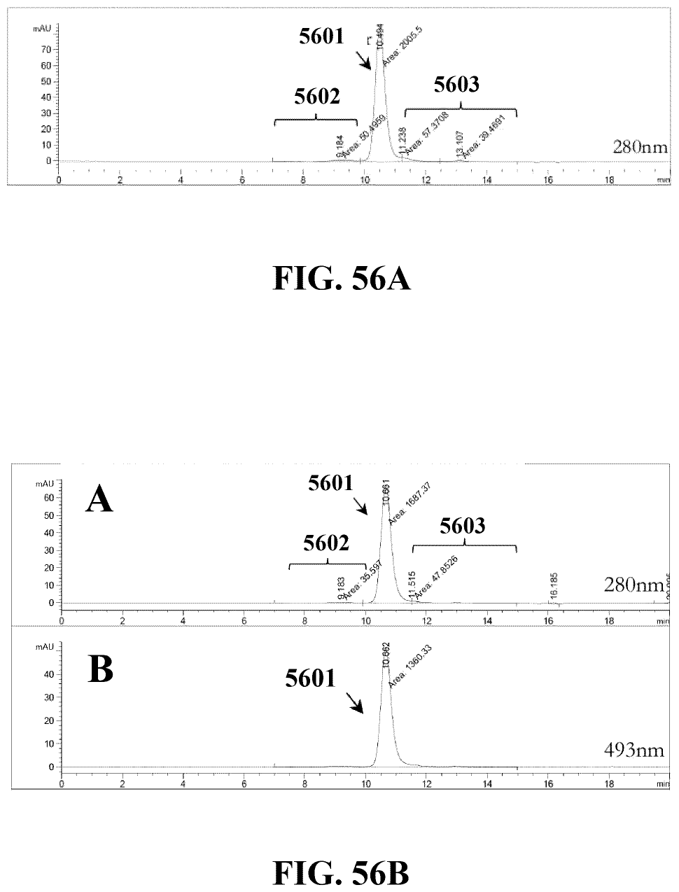

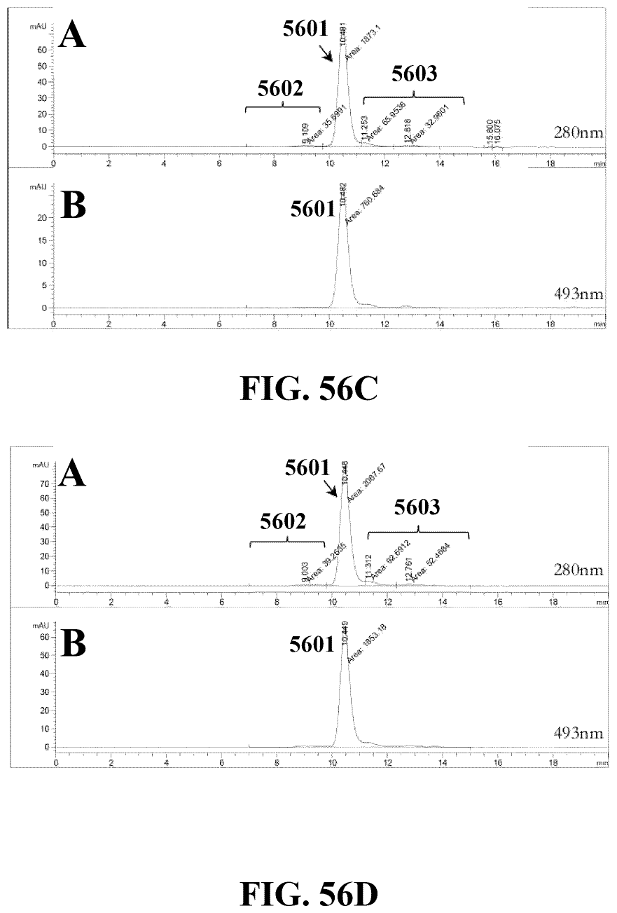

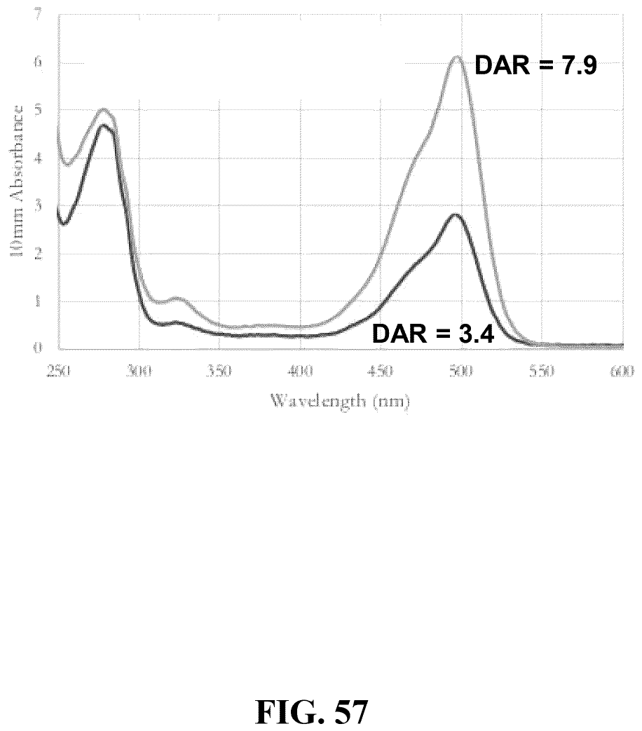

Purity and DAR analyses were performed with SEC and UV-visible spectroscopy for compound 7 functionalized ADCs (as outlined in Example 15 and Example 20) and with fluorimetry and SEC for compound 8 functionalized ADCs (as outlined in Example 17 and Example 20). Representative SEC and UV-visible spectroscopy data are shown in FIGS. 56A-56D and FIG. 57, respectively. FIGS. 56A-56D provide exemplary chromatograms captured at 280 nm and 493 nm absorbance, corresponding to antibody and fluorescein, respectively. In FIGS. 56A-56D, the main peak 5601 corresponds to intact, monomeric antibodies and ADCs, the regions at lower 5602 and higher 5603 retention times correspond to HMW and LMW, respectively. FIG. 56A provides data for the unfunctionalized antibody, while FIG. 56B, FIG. 56C, and FIG. 56D correspond to the ADCs.

FIG. 57 provides exemplary UV-visible absorbance spectroscopy data for anti-BCMA ADCs with compound 7 DARs of 3.4 and 7.9. The ratios of the 280 nm and 493 nm peaks, corresponding to antibody and fluorescein, respectively, were used to determine DARs as outlined in Equations 1-3 and Example 15. As can be seen in FIG. 57, the ratio of these peaks is about 2.3-times higher for the 7.9 DAR ADC than for the 3.4 DAR ADC.

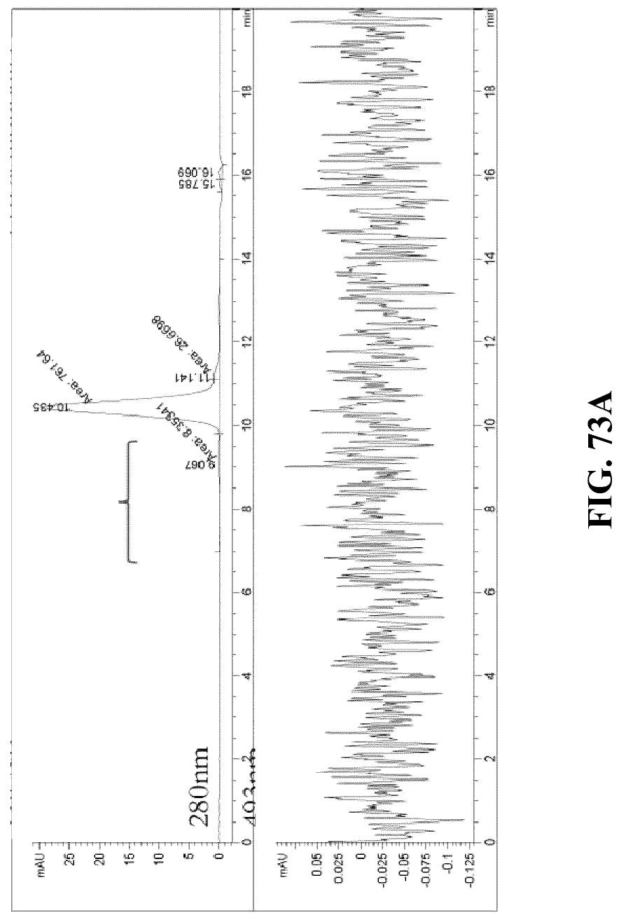

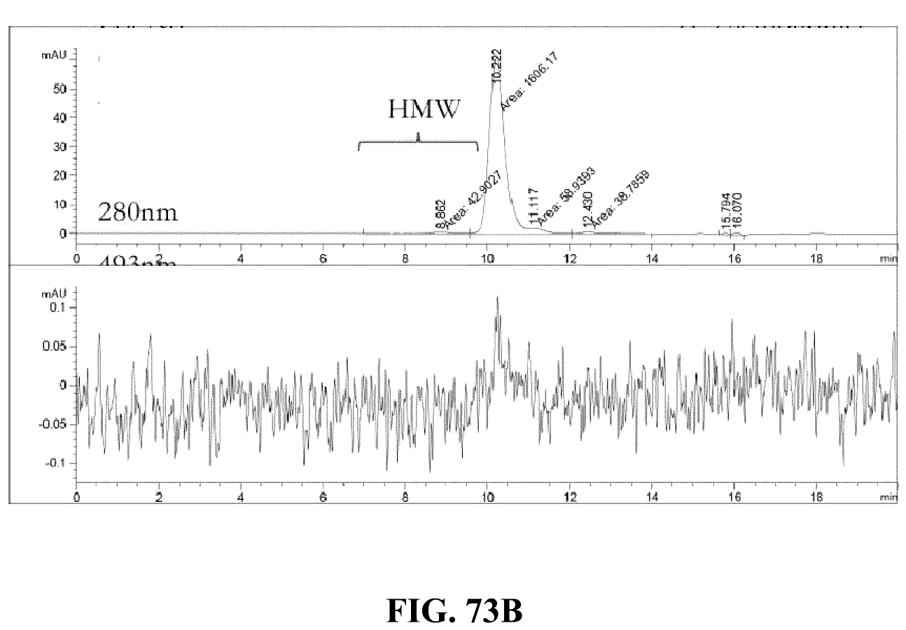

FIGS. 73A and 73B provide size-exclusion chromatograms of the compound 8-functionalized ADCs (outlined in Table 21A). In each figure, the top plot corresponds to 280 nm and the bottom plot corresponds to 493 nm chromatograms. While protein peaks are present in each 280 nm chromatogram, negligible signal is seen in the 493 nm chromatograms.

### Example 22: Optimization of Antibody Lysine Conjugation

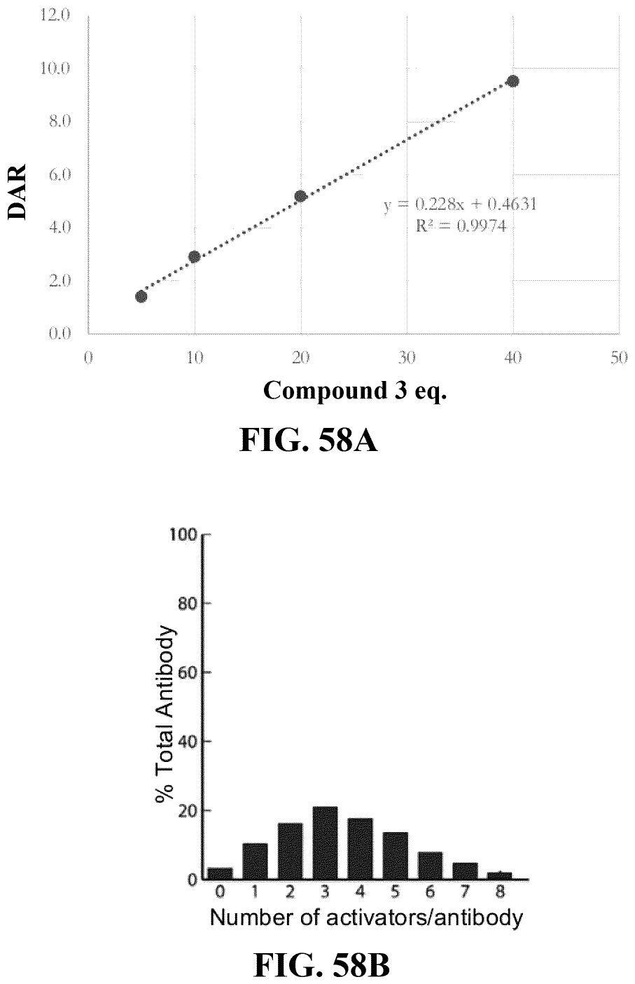

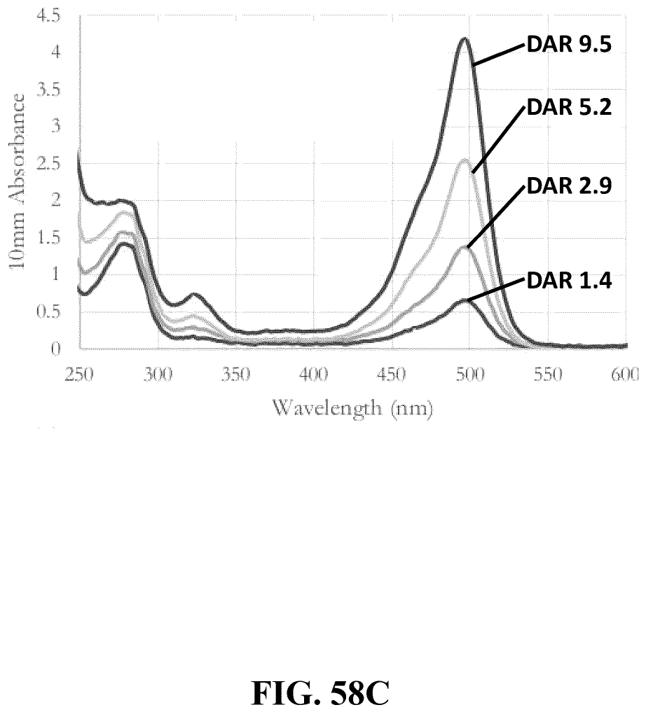

This example covers the effect of varying conjugating group concentration during antibody lysine functionalization. ADCs were prepared as outlined in FIG. 43B. 45 μg antibodies were incubated with either 5, 10, 20, or 40 equivalents of compound 9 for 2 hours at room temperature in pH 7.4 PBS with 10% (volume/volume) DMSO. To sequester unreacted compound 9, the reactions were then quenched with 100 equivalents of L-lysine. The resultant ADCs were then purified by centrifugal filtration and filtered through 0.22 μm PES according to FIG. 46, and then characterized with size-exclusion chromatography and UV-visible absorbance spectroscopy. Characteristics of the resultant ADCs are summarized in Table 23 and FIG. 58A and FIG. 58B.

TABLE 23

| Equivalents of | | | | |
|---|---|---|---|---|
| compound 9 | 5 | 10 | 20 | 40 |
| Scale (μg) | 45 | 45 | 45 | 45 |
| Recovery (μg) | 43 | 40 | 37 | 43 |
| Recovery (%) | 80.9 | 75.5 | 75.7 | 75.9 |
| Concentration | 0.8 | 0.8 | 0.9 | 0.8 |
| (mg/mL) | | | | |
| DAR (SEC) | 1.0 | 2.0 | 3.3 | 5.4 |
| DAR (UV) | 1.4 | 2.9 | 5.2 | 9.5 |
| Monomer (%) | 94.0 | 91.4 | 90.3 | 88.8 |
| HMW (%) | 3.4 | 5.4 | 7.1 | 9.2 |
| LMW (%) | 2.6 | 3.2 | 2.6 | 2.0 |

As displayed in FIG. 58A, the relationship between compound 9 concentration and resultant ADC DAR was fairly linear over the 8-fold range tested, with the 5, 10, 20, and 40 compound 9 equivalent preparations yielding DARs of about 1.4, 2.9, 5.2, and 9.5, respectively. FIG. 58B provides the DAR distribution for the 20-equivalent compound 9 (DAR=5.2) preparation. While the majority of antibodies were coupled to between 2 and 4 conjugating groups, antibodies with 1-, 5-, 6-, and 7-, conjugating groups were also prevalent in this preparation, indicating that the lysine functionalization method was a highly heterogeneous process which produced ADCs with a range of antibody-conjugating group ratios. FIG. 58C provides representative UV-visible absorbance data used for DAR determination. As can be seen from the lots, the ratios of 280 nm to 493 nm peaks increased with compound 9 concentration used during ADC synthesis.

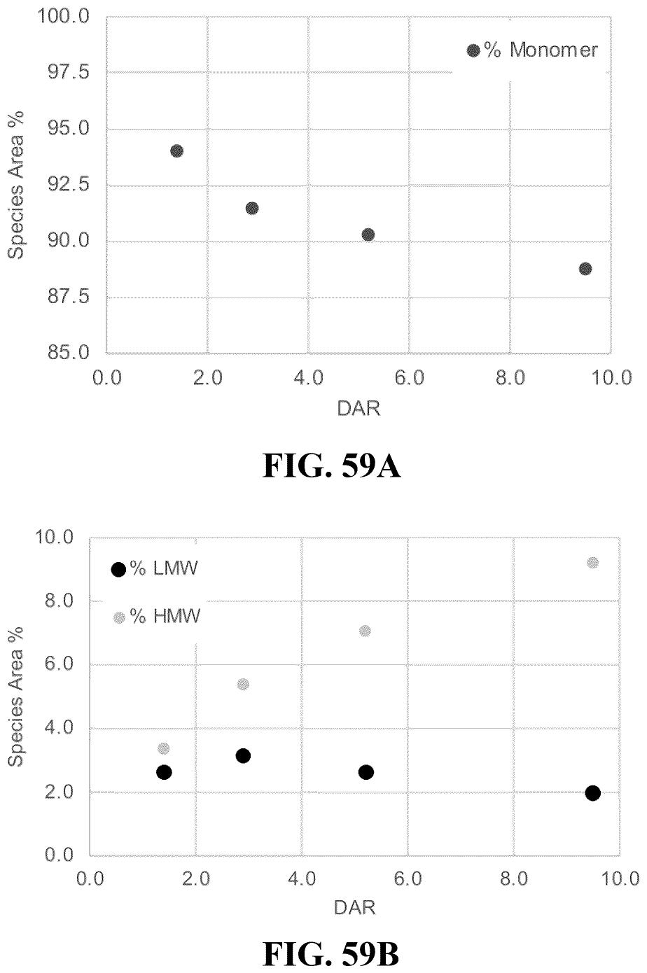

Size-exclusion chromatography was used to determine the purity of each preparation. FIG. 59A summarizes the percent monomer at each obtained DAR. While all preparations included greater than 87.5% monomeric antibodies, monomer concentration decreased with increasing DAR, ranging from about 94% monomer for a DAR of about 1.4 (corresponding to 5 equivalents of compound 9 during preparation) to about 88% for a DAR of about 9.6 (corresponding to 40 equivalents of compound 9 during preparation). FIG. 59B summarizes the prevalence of LMW and HMW species in each ADC preparation. While LMW was fairly consistent across the four preparations-ranging from about 2% to about 3%-HMW increased with DAR, indicating that either greater lysine functionalization or compound 9 exposure caused antibody aggregation.

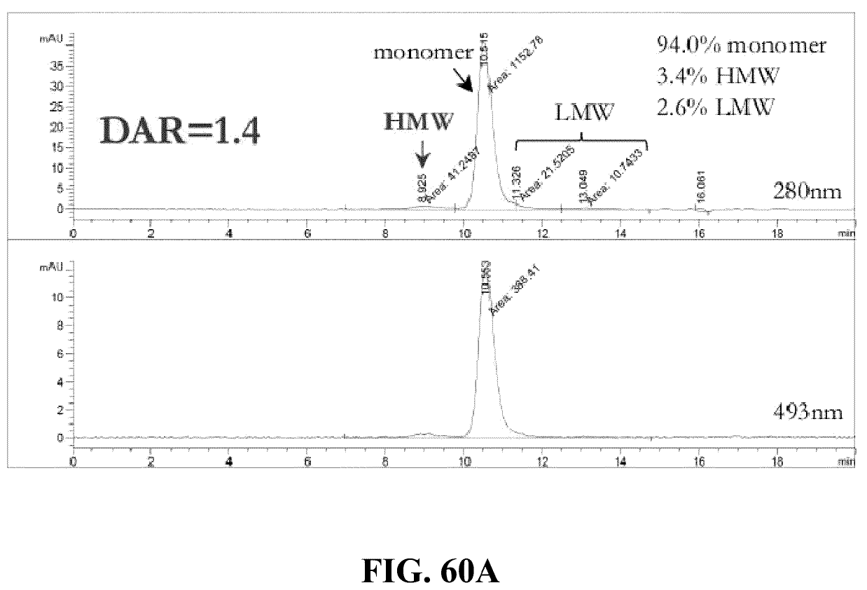

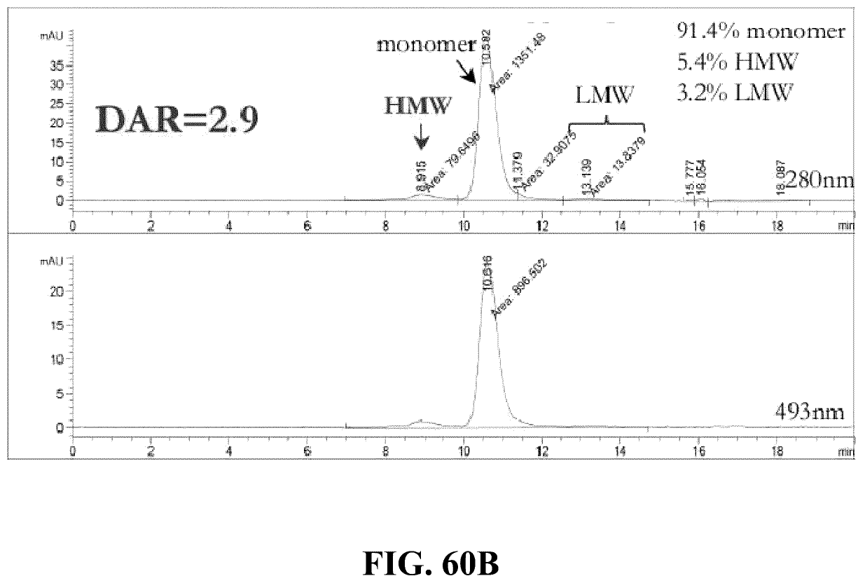

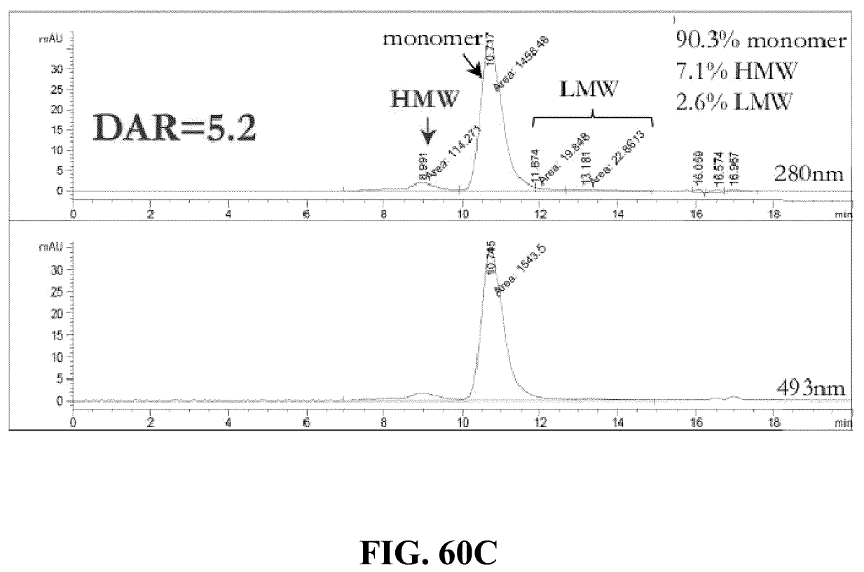

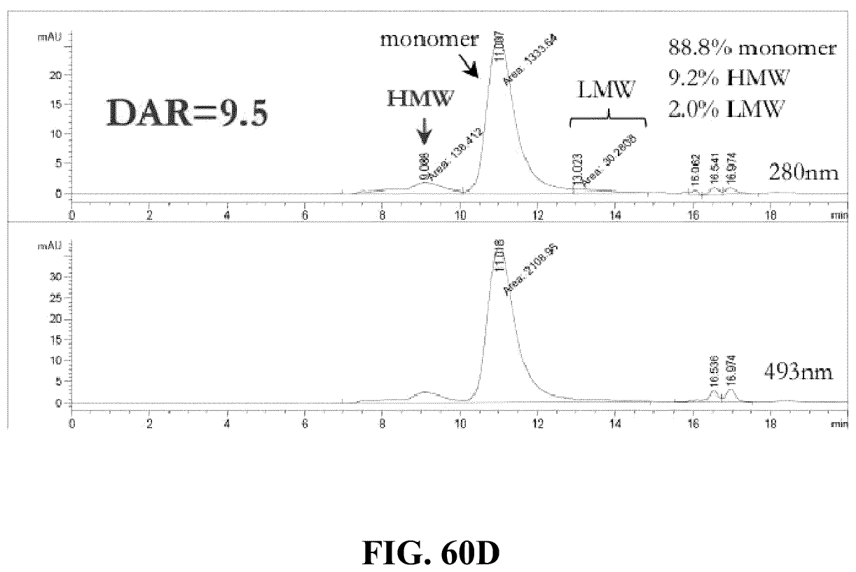

FIGS. 60A-60D provide representative SEC data for the four ADC preparations. In each figure, the top plot corresponds to chromatograms collected on the 280 nm protein channel, while the bottom plots correspond to chromatograms captured on the 493 nm fluorescein channel.

### Example 23: Scalable Lysine Conjugations

This example covers the scale-up of a lysine-functionalization method of the present disclosure. ADC syntheses were performed at two scales, utilizing either 45 or 2000 μg antibody. The antibodies were functionalized with about 13 or 33 equivalents of compound 9 according to Example 22. The ADCs were purified with TFF and 0.22 μm PES according to FIG. 46, and then characterized for purity and DAR with SEC and UV-visible absorbance spectroscopy.

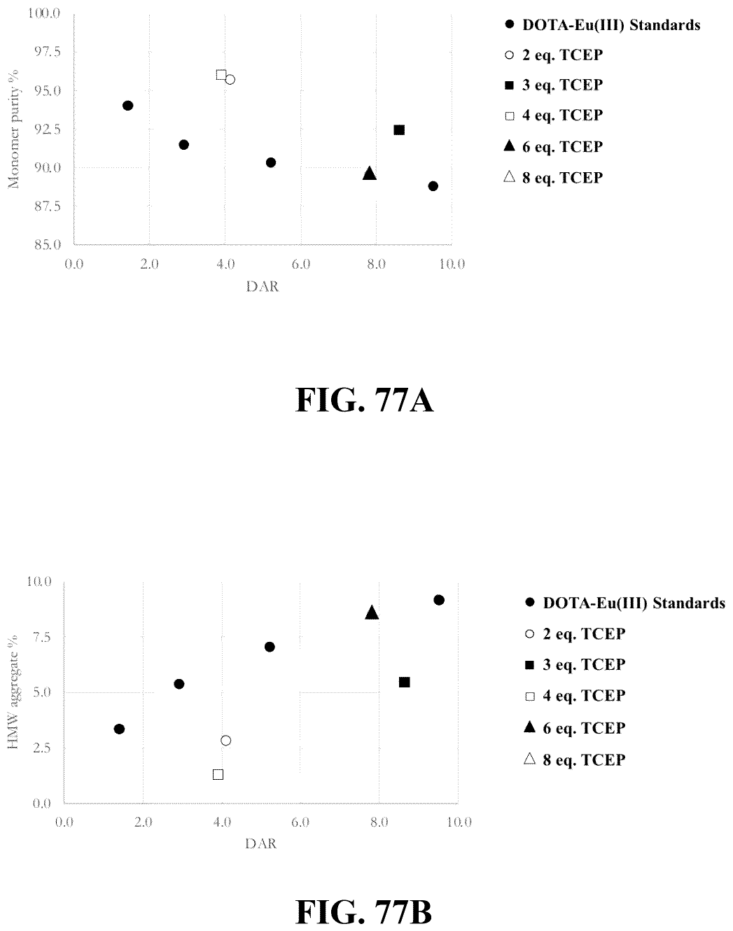

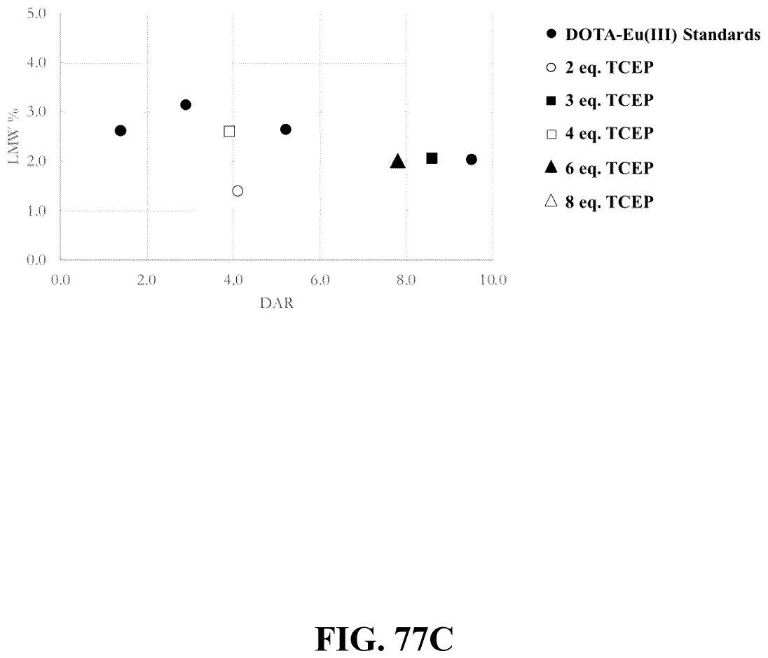

Characteristics of the ADC preparations are summarized in Table 24. FIG. 77A-FIG. 77C compare the monomer (FIG. 77A), HMW (FIG. 77B), and LMW (FIG. 77C) content of four ADC preparations relative to the preparations of Example 22. The 2000 μg DAR 3.9 and 45 μg DAR 4.1 and 8.6 preparations had higher monomer content than the ADCs generated from the Example 22 titrations. As shown in FIG. 77B and FIG. 77C, the higher monomeric content primarily stemmed from lower HMW, as the four preparations had roughly similar LMW as the Example 22 ADCs. Notably, the 2000 μg preparations had nearly 1.5-fold higher recoveries than the 45 μg preparations, at about 95% rather than about 65%, demonstrating that the Example 22 methods are amenable to efficient scale-up.

TABLE 24

| | | | | |
|---|---|---|---|---|
| Scale (μg) | 45 | 45 | 2000 | 2000 |
| Approximate | 13 | 33 | 13 | 33 |
| Equivalents of | | | | |
| Compound 9 | | | | |
| DAR (UV) | 4.1 | 8.6 | 3.9 | 7.8 |
| Recovery (μg) | 30 | 29 | 1912 | 1915 |
| % Recovery | 65.9 | 63.6 | 95.6 | 95.7 |
| % Monomer | 95.7 | 92.7 | 96.1 | 89.5 |
| % HMW | 2.9 | 5.5 | 1.3 | 8.5 |
| % LMW | 1.4 | 2.1 | 2.6 | 2.0 |

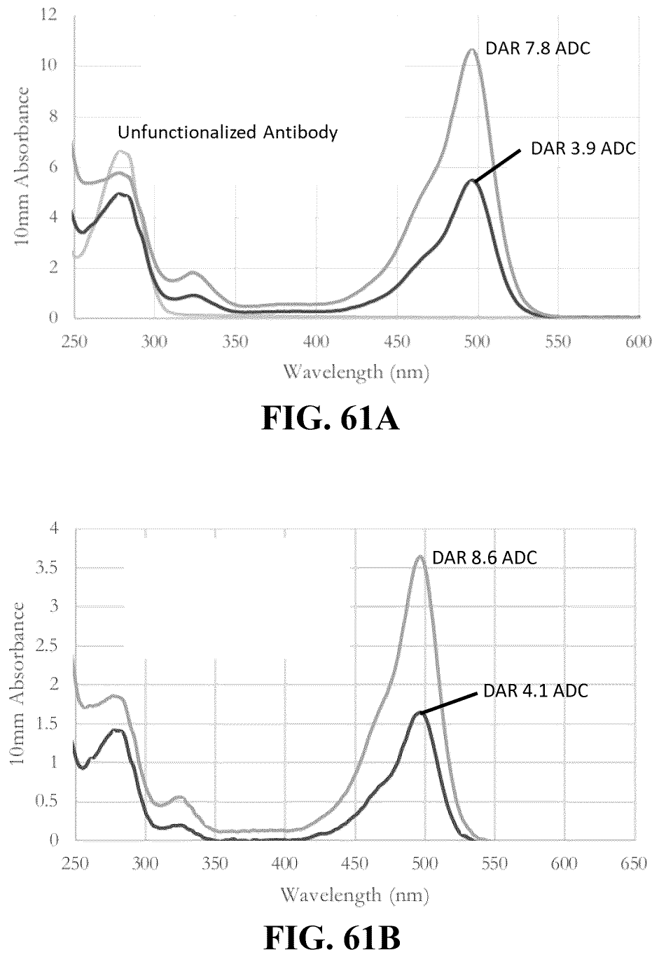

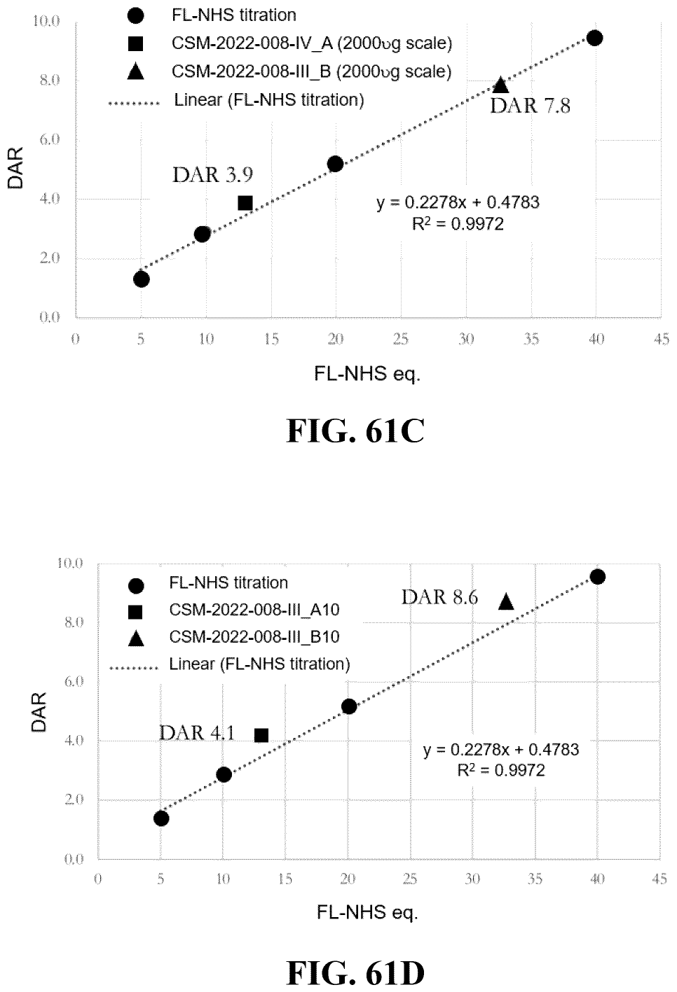

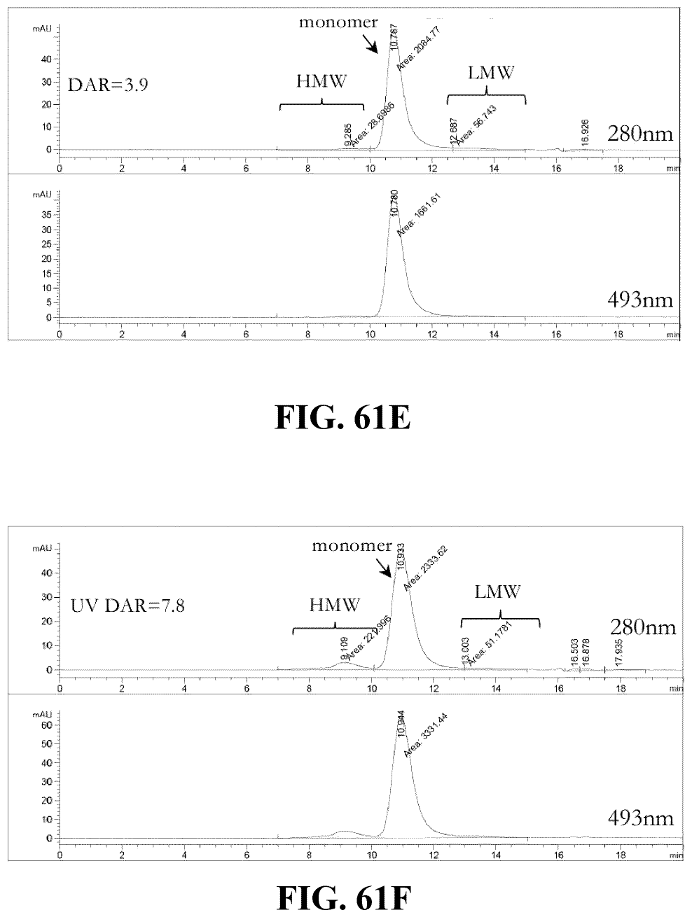

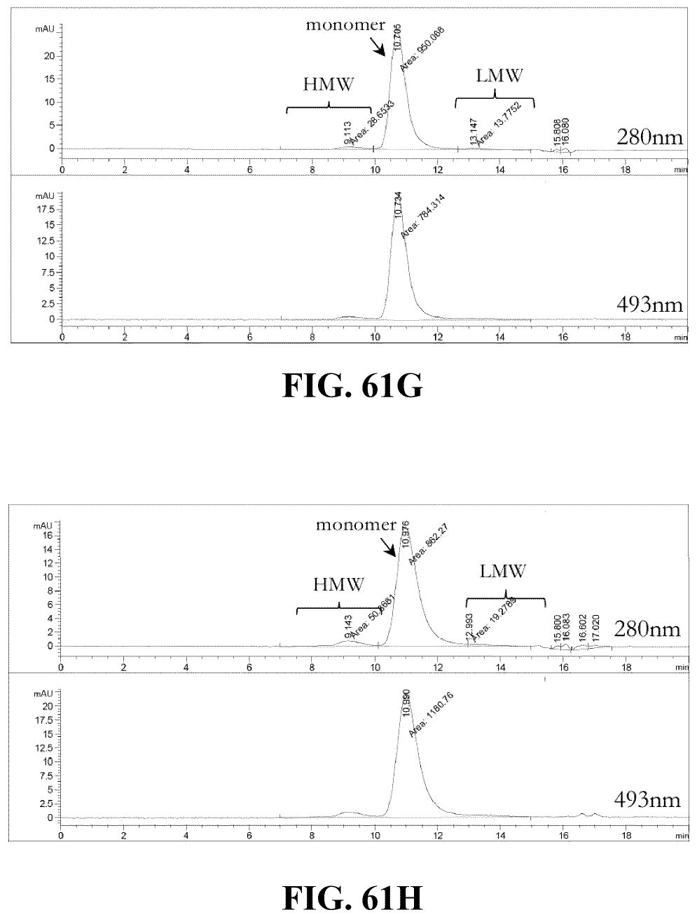

FIG. 61A and FIG. 61B provide exemplary UV-visible absorbance spectroscopy data for the starting, unfunctionalized antibody and for the ADCs generated from the 2000 μg (FIG. 61A) and 45 μg (FIG. 61B) preparations, respectively. FIG. 61C and FIG. 61D provide the DARs of the ADCs relative to the equivalents of compound 9 used in their syntheses for the 2000 μg (FIG. 61C) and 45 μg (FIG. 61D) ADC preparations. For the 2000 μg preparations, the 33-compound 9 equivalent synthesis generated ADCs with the DAR predicted by the Example 22 compound 9 titration, while the compound 9 equivalent syntheses generated a slightly higher DAR than expected. The DARs of both 45 μg preparations were higher than predicted by the Example 22 compound 9 titration. FIG. 61E and FIG. 61F provide representative SEC chromatograms for the 2000 μg, DAR 3.9 and DAR 7.8 ADC preparations, respectively. FIG. 61G and FIG. 61H provide representative SEC chromatograms for the 45 μg, DAR 4.1 and DAR 8.6 ADC preparations, respectively In FIGS. 61E-61H, the top chromatogram corresponds to the 280 nm absorbance channel, and the bottom chromatogram corresponds to the 493 nm absorbance channel.

### Example 24: Anti-CS1-Fluorescein Conjugate Preparation

This example covers ADC syntheses with CS1-targeting antibodies. Elotuzumab thiols were functionalized according to Example 15, utilizing 100 or 3800 μg antibody as starting material, 10 equivalents TCEP for disulfide reduction, and 20 equivalents compound 7 for thiol functionalization. The resultant ADCs were analyzed with UV-visible absorbance spectroscopy and size-exclusion chromatography to determine DAR and purity. Characteristics of the resultant ADC populations are summarized in Table 25.

TABLE 25

| | | | |
|---|---|---|---|
| DAR (UV-Vis) | N/A | 8.8 | 8.1 |
| DAR (SEC) | N/A | 4.4 | 4.7 |
| Scale (μg) | 7000 | 100 | 3800 |
| Recovery (μg) | 3889 | 99 | 3723 |
| Recovery (%) | 55.6 | 98.7 | 95.5 |
| Concentration | 4.47 | 1.5 | 3.4 |
| (mg/mL) | | | |
| % Monomer | 96.2 | 94.6 | 95.3 |
| % HMW | 2.1 | 2.1 | 2.0 |
| % LMW | 1.7 | 3.3 | 2.7 |

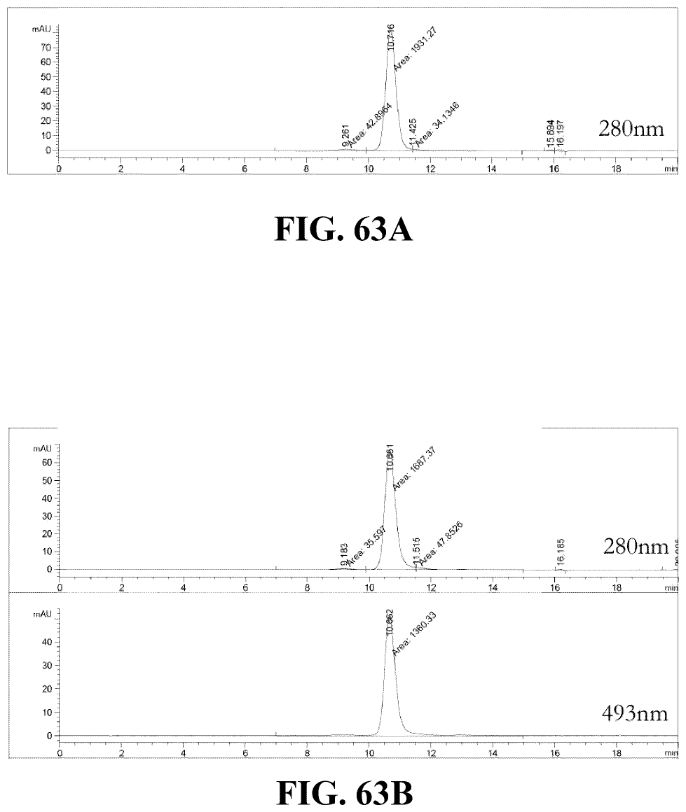

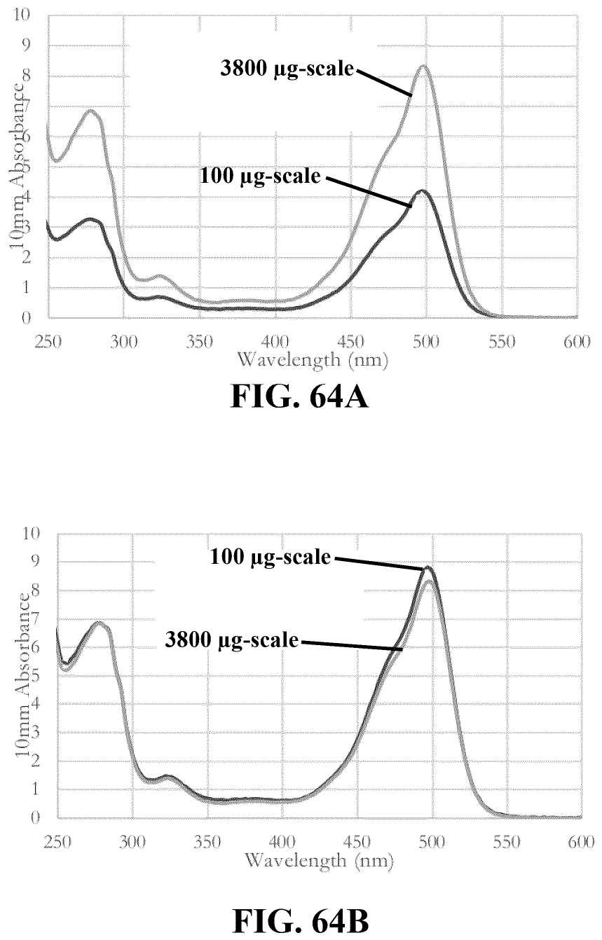

FIGS. 63A and 63B provide size-exclusion chromatography data for the unfunctionalized antibody (FIG. 63A) and the 3800 μg ADC preparation (FIG. 63B; DAR 8.1 as determined by UV-visible absorbance spectroscopy). FIGS. 64A and 64B provide raw (FIG. 64A) and normalized (FIG. 64B) UV-visible absorbance data for the 100 μg and 3800 μg-scale ADC preparations. While the measured DARs from the SEC and UV-vis analyses differ by nearly a factor of two, the DARs measured with SEC were likely not accurate due to low ADC injection quantities below 75 μg.

### Example 25: ADC Preparation with Antibody Stabilizing Agents

This example covers ADC syntheses which use an antibody stabilizer during conjugation. 5-8% trehalose was present in the starting antibodies as a stabilizer to prevent HMW and LMW generation. Antibody thiols were functionalized with compound 7 according to Example 15, utilizing 60 or 90 μg antibody as starting material, 5.2 equivalents TCEP for disulfide reduction, and 20 equivalents compound 7 for thiol functionalization. The 60 and 90 μg preparations yielded relatively high purities with monomeric compositions of 99.1% and 96.1%, respectively. Characteristics of the resultant ADC populations as determined by SEC and UV-vis are summarized in Table 26.

TABLE 26

| | | |
|---|---|---|
| DAR (UV-Vis) | 5.2 | 4.6 |
| DAR (SEC) | 4.8 | 4.1 |
| Scale (μg) | 60 | 90 |
| Recovery (μg) | 27 | 72 |
| Recovery (%) | 45 | 80 |
| Concentration | 0.5 | 0.9 |
| (mg/mL) | | |
| % Monomer | 99.1 | 96.1 |
| % HMW | 1.5 | 3.2 |
| % LMW | 0 | 0.7 |

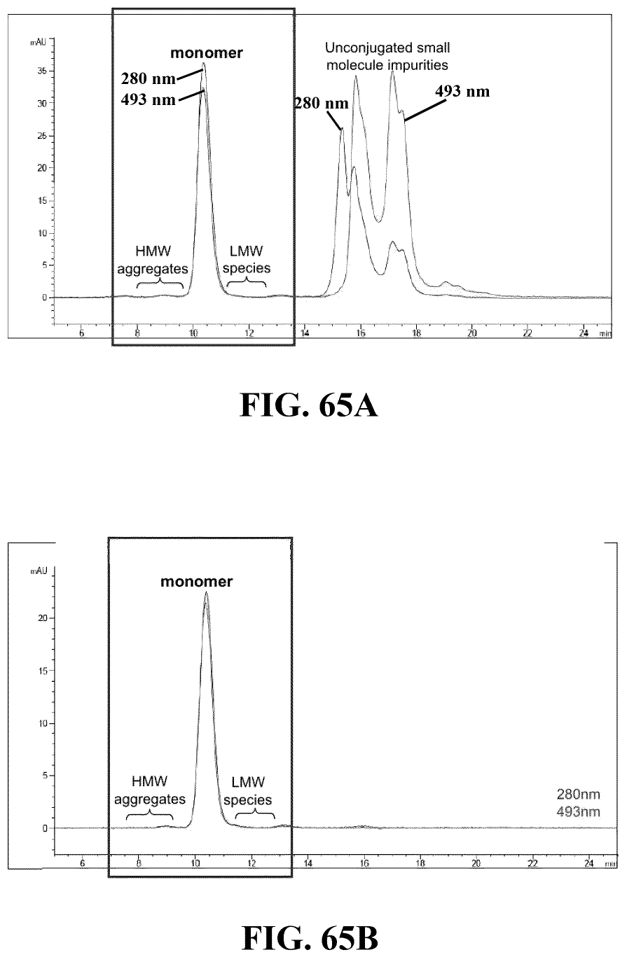

FIGS. 65A and 65B provide overlaid 280 nm and 493 nm SEC chromatograms of a crude reaction mixture collected during compound 7 antibody functionalization (FIG. 65A) and of the purified ADC generated from the 90 μg preparation (FIG. 65B). HMW, monomeric, and LMW antibody species are boxed in FIG. 65A and FIG. 65B. In FIG. 65A unconjugated small molecule impurities, including unreacted compound 7, are visible at high retention times in the 280 nm and 493 nm chromatograms. These peaks are not present in FIG. 65B, indicating that purification removed the small molecule impurities from the ADC preparation.

### Example 26: Scaled-Up ADC Synthesis with Antibody Reduction in Absence of Trehalose

This example covers 100-800 μg-scale ADC syntheses. Antibodies were buffer exchanged against PBS and functionalized with compound 7 according to Example 15, utilizing 100 or 800 μg antibody as starting material, 10 equivalents TCEP for disulfide reduction, and 20 equivalents compound 7 for thiol functionalization. The ADC preparations were analyzed by SEC and UV-vis, the results of which are summarized in Table 27. As SEC-based DAR measurements can be inaccurate for antibody injection quantities below 75 μg, SEC was run on pooled lots of the two ADC preparations. The combined preparations had a monomeric content of 94.0%, with 1.4% HMW and 4.6% LMW.

TABLE 27

| | | |
|---|---|---|
| DAR (UV-Vis) | 7.7 | 7.4 |
| DAR (SEC) | N/A | 4.0 |
| Scale (μg) | 100 | 800 |
| Recovery (μg) | 76 | 526 |
| Recovery (%) | 76.4 | 65.7 |
| Concentration | 1.8 | 1.2 |
| (mg/mL) | | |
| % Monomer | N/A | 94.0 |
| % HMW | N/A | 1.4 |
| % LMW | N/A | 4.6 |

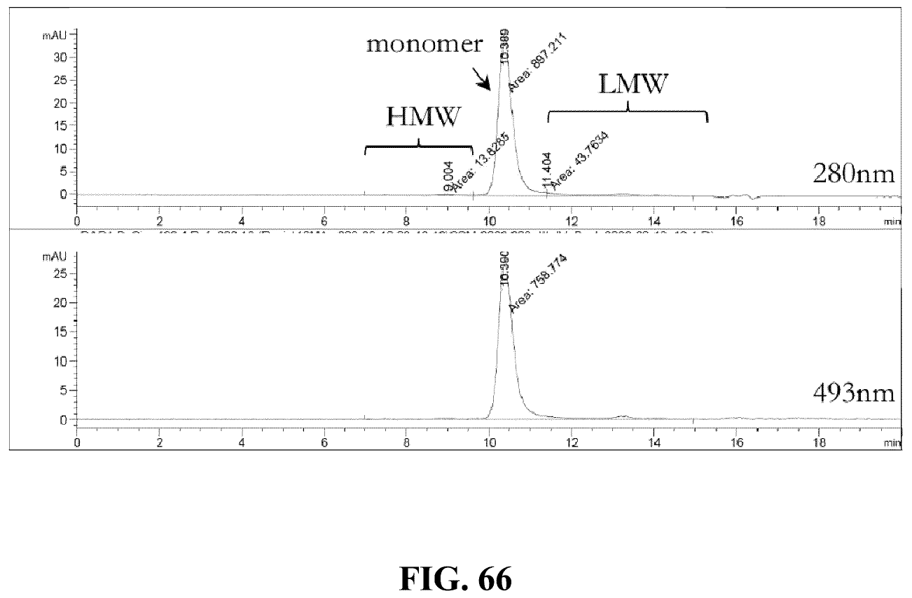

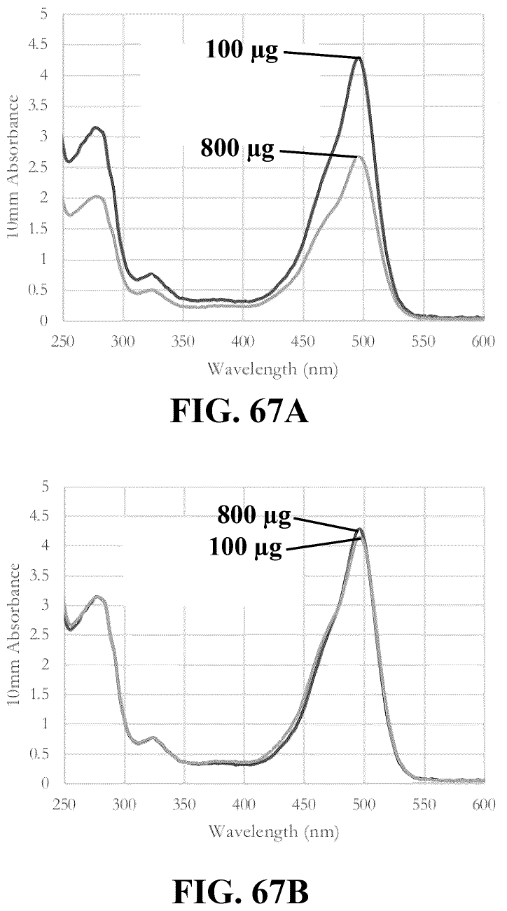

FIG. 66 provides exemplary chromatograms of the ADC preparations, with the top plot corresponding to 280 nm absorbance and the bottom plot corresponding to 493 nm absorbance. FIG. 67A and FIG. 67B provide raw (FIG. 67A) and normalized (FIG. 67B) UV-visible absorbance data for the 100 μg and 800 μg-scale ADC preparations.

### Example 27: Trehalose-Stabilized ADC Syntheses

This example covers an antibody thiol-functionalization method utilizing trehalose during disulfide reduction. 80 μg antibody was functionalized with compound 7 according to Example 15, utilizing either 5.8 or 6.4 equivalents TCEP in the presence of 5-8% trehalose for disulfide reduction and 20 equivalents compound 7 for thiol functionalization. The resulting ADC preparations are summarized in Table 28. While both ADC preparations had DARs of 5.0 (as determined by UV-visible absorbance), the 5.8 TCEP-equivalent preparation had higher combined HMW and LMW content of 11.4%, as opposed to 5.1% for the 6.4 TCEP equivalent preparation.

TABLE 28

| | | |
|---|---|---|
| TCEP (equivalents) | 5.8 | 6.4 |
| DAR (UV-Vis) | 5.0 | 5.0 |
| DAR (SEC) | 2.8 | 2.8 |
| Recovery (μg) | 56 | 54 |
| Recovery (%) | 70.3 | 67.9 |
| Concentration | 1.6 | 0.9 |
| (mg/mL) | | |
| % Monomer | 88.6 | 94.9 |
| % HMW | 10.2 | 3.9 |
| % LMW | 1.2 | 1.2 |

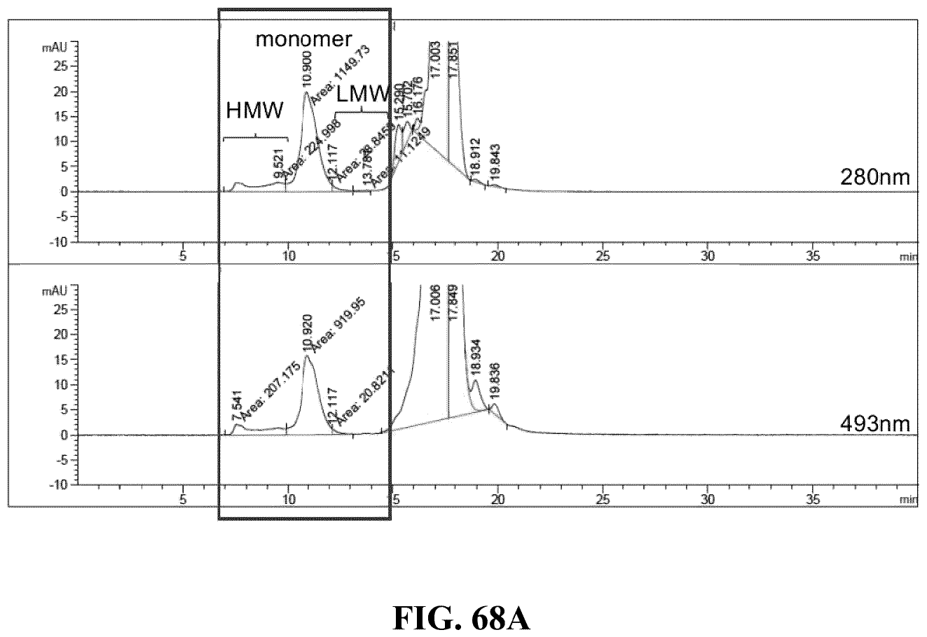

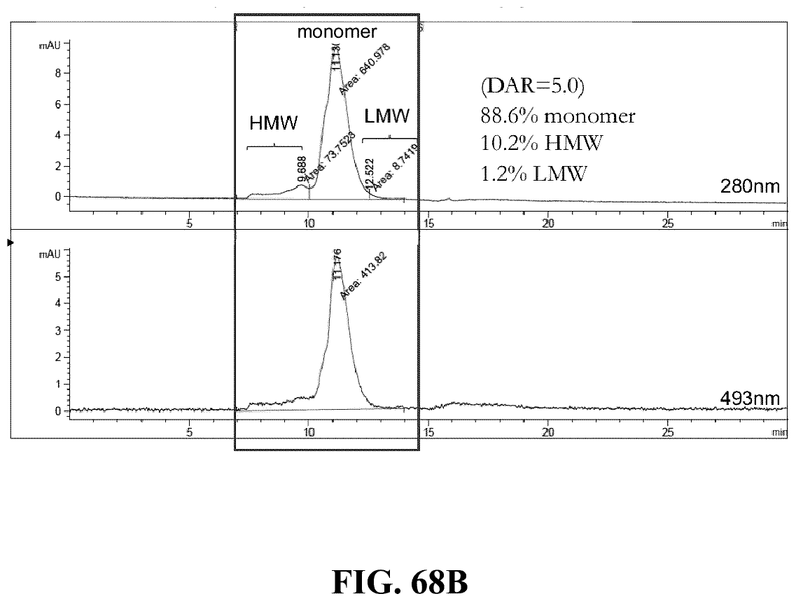

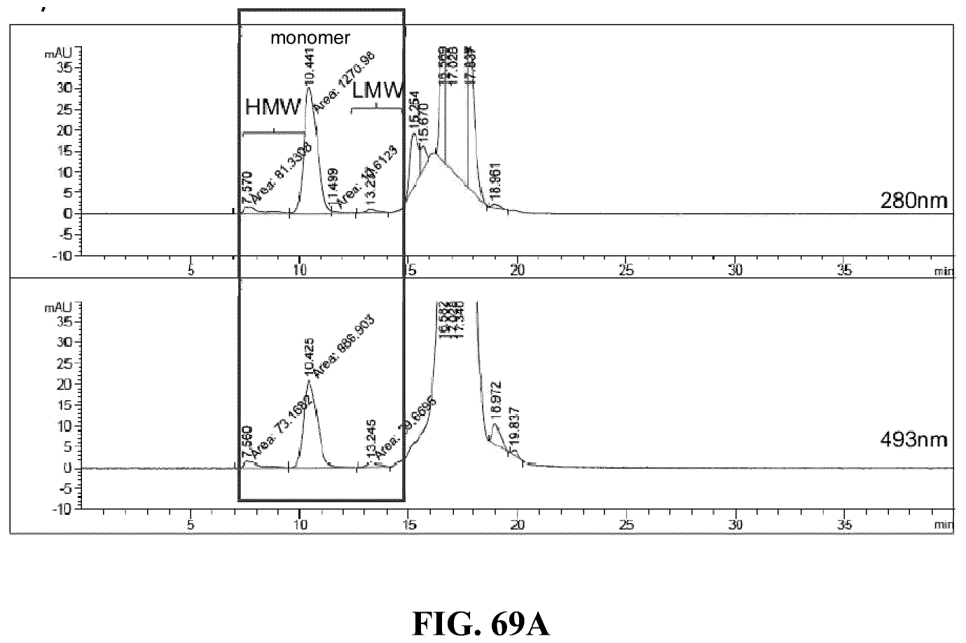

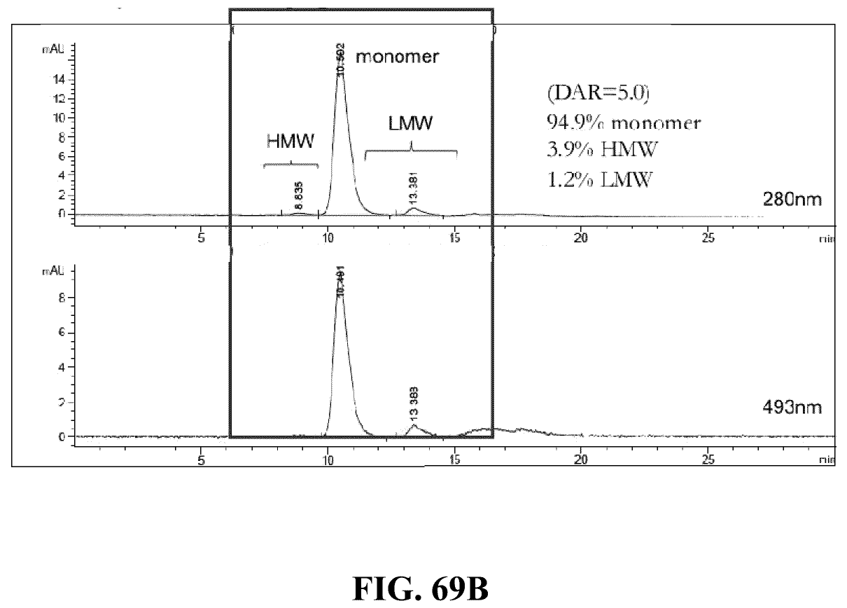

FIG. 68A and FIG. 68B provide SEC chromatograms of the crude conjugation mixture (FIG. 68A) and the purified ADC product (FIG. 68B) from the 5.8-equivalent TCEP synthesis. FIG. 69A and FIG. 69B provide SEC chromatograms of the crude conjugation mixture (FIG. 69A) and the purified ADC product (FIG. 69B) from the 6.4-equivalent TCEP synthesis. In each figure, the top plot corresponds to 280 nm (protein) absorbance, while the bottom plot corresponds to 493 nm (fluorescein) absorbance. As indicated by the loss of 15-20 minute peaks in the chromatograms, TFF and PES purification cleared unbound small molecule impurities and unreacted compound 7 from ADC preparations.

### Example 28: Thiol-Functionalized ADC Syntheses with Low Equivalents of Reducing Agents

This example covers an antibody thiol-functionalization method utilizing relatively low equivalents of reducing agents during disulfide reduction. 30 and 100 μg antibody was reduced in the presence of 4 equivalents TCEP, and functionalized with 20 equivalents of compound 7 and purified according to Example 15, and then characterized with UV-visible absorbance and size-exclusion chromatography. The resulting ADC preparations are summarized in Table 29. Both ADC preparations had DARs of about 6 (as determined by UV-visible absorbance spectroscopy) and monomeric content of about 96%. 33 μg unfunctionalized antibody was purified in parallel according to FIG. 45 as a control.

TABLE 29

| | | | |
|---|---|---|---|
| Scale (μg) | 33 | 30 | 100 |
| TCEP (equivalents) | 0 | 4 | 4 |
| DAR (SEC) | N/A | 3.4 | 3.5 |
| DAR (UV-Vis) | N/A | 6.0 | 5.8 |
| Recovery (%) | 94.2 | 76.0 | 62.4 |
| Concentration | 1.11 | 0.71 | 1.16 |
| (mg/mL) | | | |
| % Monomer | 98.6 | 95.9 | 96.3 |
| % HMW | 0.7 | 1.0 | 1.4 |
| % LMW | 0.7 | 3.1 | 2.3 |

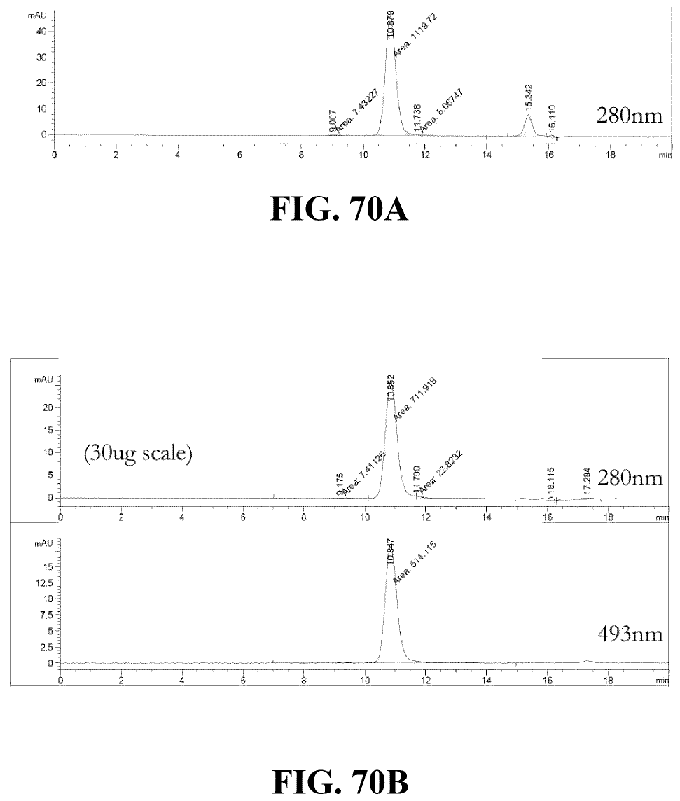

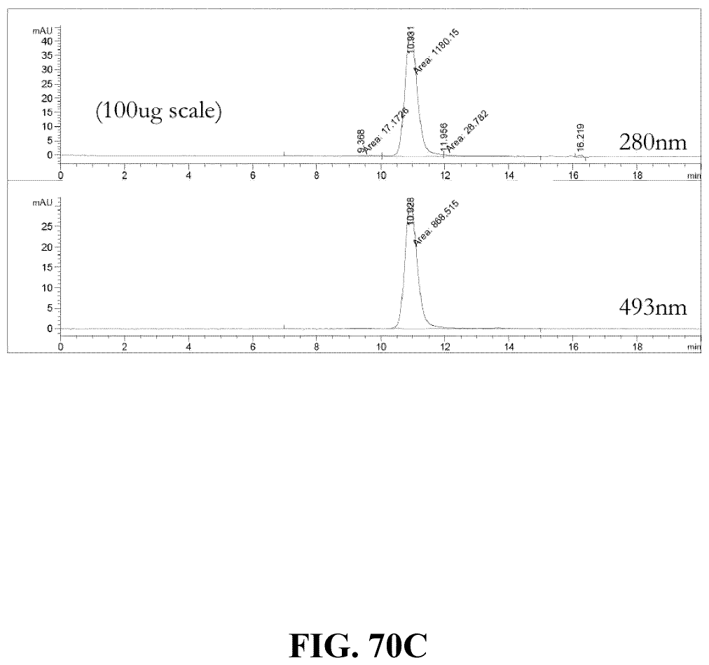

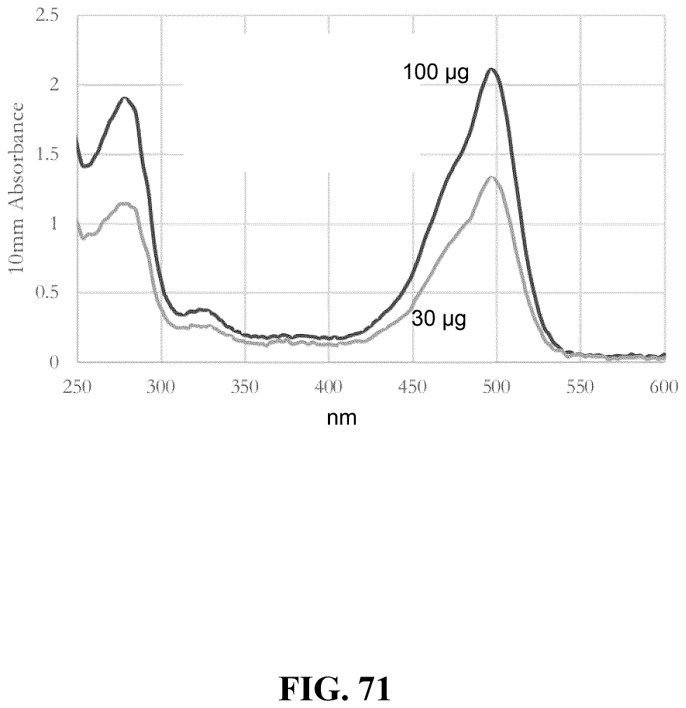

FIGS. 70A-70C provide SEC chromatograms of the unfunctionalized antibody (FIG. 70A), ADCs from the 30 μg-preparation (FIG. 70B), and ADCs from the 100 μg-preparation (FIG. 70C). In each of FIG. 70B and FIG. 70C, the top chromatogram corresponds to 280 nm collection, while the bottom chromatogram corresponds to 493 nm collection. The low signal directly above and below the central peaks are reflective of the high purities of the antibody and ADC preparations. FIG. 71 provides representative UV-visible absorbance spectra of ADCs generated from the 30 μg and 100 μg preparations.

### Example 29: Crude Reaction Analysis for Antibody Amine Functionalizations

This example details crude reaction products following antibody lysine functionalization with NHS-ester conjugating groups. ADCs were prepared as outlined in FIG. 43B. 20 μg antibodies were incubated with either 5, 10, or 20 equivalents of compound 9 for 2 hours at room temperature in pH 7.4 PBS with 10% (volume/volume) DMSO. Following sequestration of unreacted compound 9 with 100 equivalents of L-lysine, the resultant ADCs were purified with TFF and 0.22 μm PES according to FIG. 46, and then characterized with size-exclusion chromatography. Characteristics of the resultant ADCs are summarized in Table 30. Across the three preparations, DAR correlated with HMW, suggesting that antibody functionalization may increase antibody aggregation.

TABLE 30

| | | | |
|---|---|---|---|
| Compound 9 | 5 | 10 | 20 |
| (equivalents) | | | |
| DAR (SEC) | 1.6 | 2.7 | 4.2 |
| % Monomer | 89.9 | 83.6 | 83.3 |
| % HMW | 3.0 | 8.5 | 10.8 |
| % LMW | 7.2 | 7.9 | 5.9 |

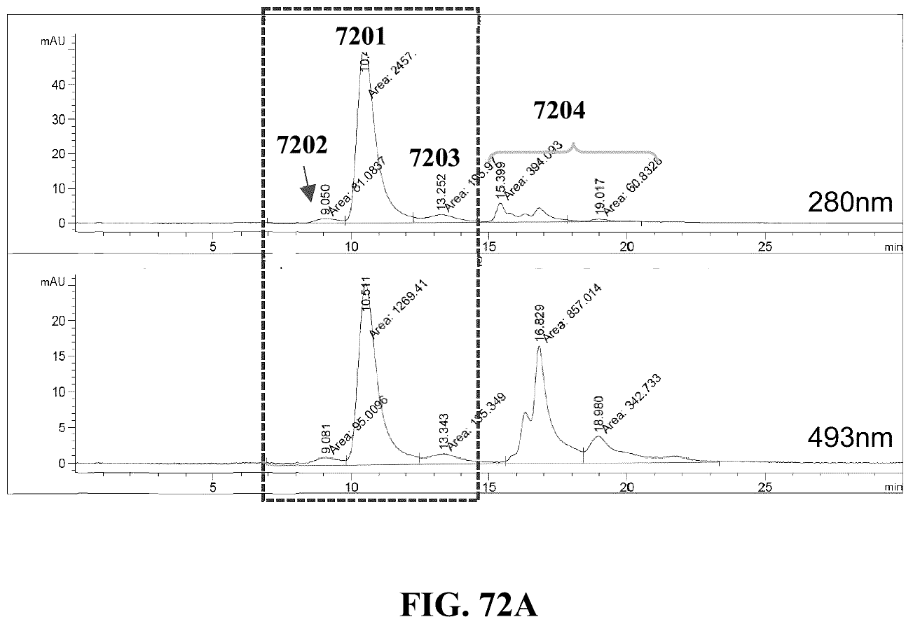

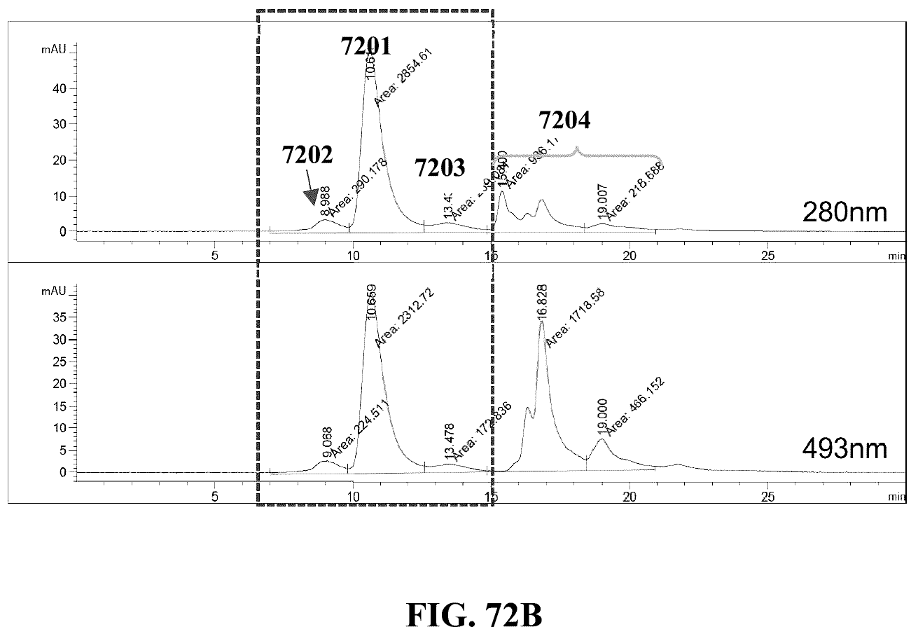

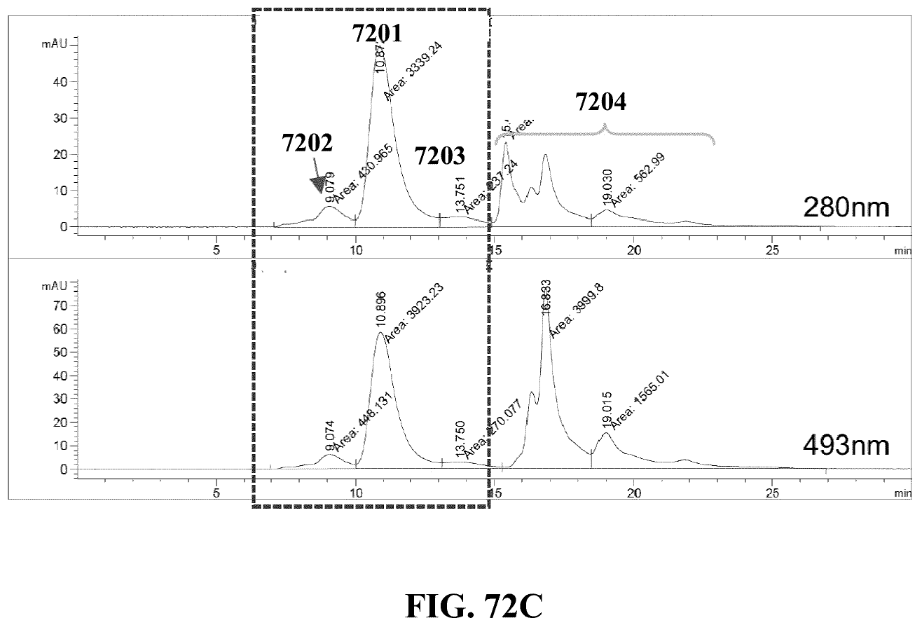

FIGS. 72A-72C provide size-exclusion chromatograms of crude (pre-purification) reaction mixtures prepared with 5 equivalents of compound 9 (FIG. 72A), 10 equivalents of compound 9 (FIG. 72B), and 20 equivalents of compound 9 (FIG. 72C). In each figure, the top plot corresponds to 280 nm absorbance (primarily reflecting protein-content) and 493 nm absorbance (primarily reflecting compound 9 content). Monomer 7201, HMW 7202, LMW 7203, and small molecule impurities 7204 are indicated in each figure.

### Example 30: Time-Resolved Fluorescence Assay for Fluorometric DAR-Characterization

This example covers a time-resolved fluorescence method for determining concentration of metal-chelating conjugating groups conjugated to antibodies, which can be used for determining DAR. To determine the concentration of conjugated metal-chelating DOTA groups on ADCs, changes in fluorescence of Eu(III)/enhancer complex were used to indirectly quantify the concentration of antibody conjugated DOTA. Preferential binding of DOTA to Eu(III) quenches fluorescence of the Eu(III)/DELFIA ligand complex. Decreases in the Eu(III)/enhancer complex signal of DOTA-EU (III) standards were used to determine concentration of conjugated DOTA for multiple ADC preparations. The DAR could be determined by taking the ratio of concentrations of conjugated DOTA to conjugated antibody (determined by total protein assay). ADCs were prepared with 2-8 equivalents of TCEP reductant and excess compound 8. As outlined in FIG. 49B, to determine the concentration of conjugated DOTA groups on the resultant ADCs, three separate assays were performed in parallel for Eu(III)-DOTA standards, Eu assay control, and test samples. Eu(III)/DOTA standards were used to determine linear dependence of fluorescence of Eu(III)/enhancer complex on concentrations of unbound Eu(III). The Eu(III)/DOTA standards were prepared using 7.5 μL 10 μM Eu(NO₃)₃, and 15 μL of 1 to 5 μM DOTA in 50 mM sodium acetate pH 5.8. An Eu(III) assay control was used to verify the linearity of the Eu-DOTA standards. The Eu(III) assay control was prepared using 7.5 μL of 10 μM Eu(NO₃)₃ in 50 mM sodium acetate. ADC samples were prepared with 20 μL of 10 μM Eu(NO₃)₃ and 2 μL of variable dilutions of ADC in 50 mM sodium acetate. DOTA-Eu(III) complexation was conducted at 95° C. for 30 minutes.

Eu(III)/enhancer complexation was accomplished with 18 μL of each DOTA-Eu(III) standard, assay control, and test ADC sample, mixed with 282 μL pH 5.8 sodium acetate, and 100 μL of enhancer solution containing phosphine oxide and 2,4-dione ligands configured to complex with aqueous Eu(III) and enhance its fluorescence. The samples were incubated at room temperature for 30 minutes, and then analyzed with fluorescence using 340 nm excitation, 615 nm emission detection, 400 microsecond lag times, and 400 microsecond integration times. The DOTA-Eu(III) standards contained 22.5 nM Eu(III) and between 4.5 and 22.5 nM DOTA. As Eu(III)-DOTA complexes have diminished fluorescence signal relative to Eu(III)-phosphine and dione complexes, decreases in fluorescence correlated with increased DOTA concentration. A standard fluorescence curve generated from the DOTA-Eu(III) standards was used to determine the concentration of DOTA in the ADC test samples. Eu(III) nitrate standards with 4.5 to 22.5 nM Eu(III) and 100 μL enhancer solution were analyzed to determine Eu(III) fluorescence concentration dependence.

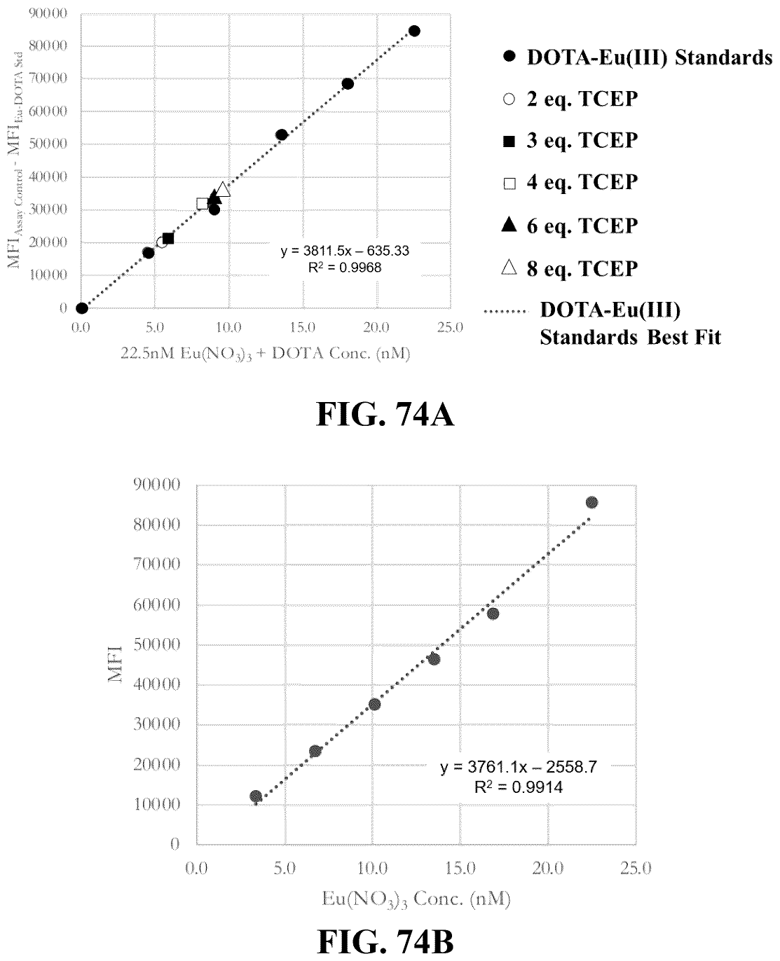

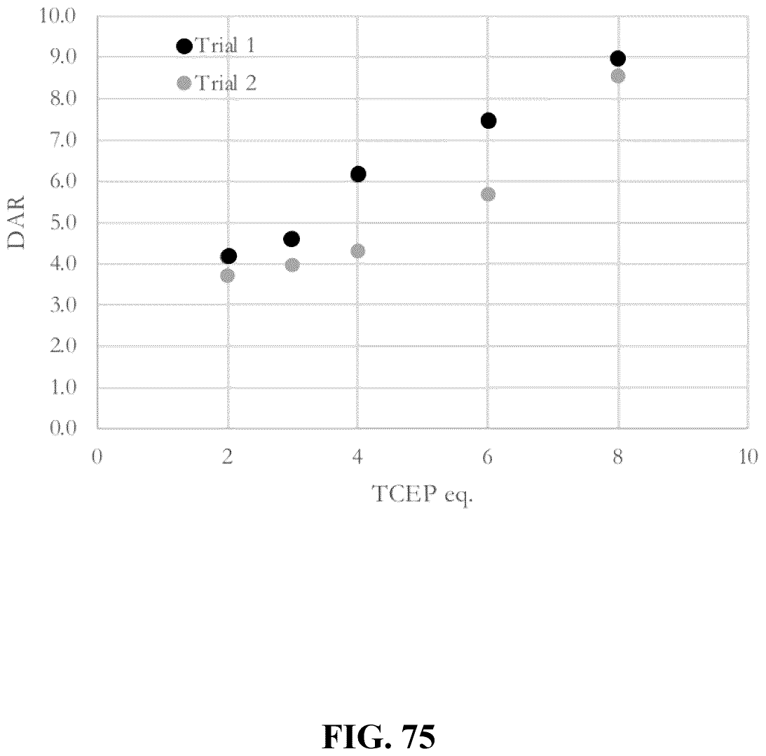

Fluorescence signals for the DOTA-Eu(III) standards and ADC test solutions are provided in FIG. 74A, while fluorescence signals from the free-Eu(III) standards (assay control) are provided in FIG. 74B. FIG. 74A provides fluorescence signal as a function of DOTA concentration, while FIG. 74B provides fluorescence signal as a function of Eu(III) concentration in the absence of DOTA. As can be seen from the figures, the Eu(III) fluorescence signals were highly linear with respect to Eu(III) and DOTA concentrations, with R² fits of 0.9914 and 0.9968, respectively. FIG. 75 summarizes the DARs measured for each ADC preparation. In total, 2 replicates of ADCs prepared with 2-, 3-, 4-, 6-, and 8-equivalents of TCEP, respectively.

### Example 31: Fluorometric DAR-Characterization

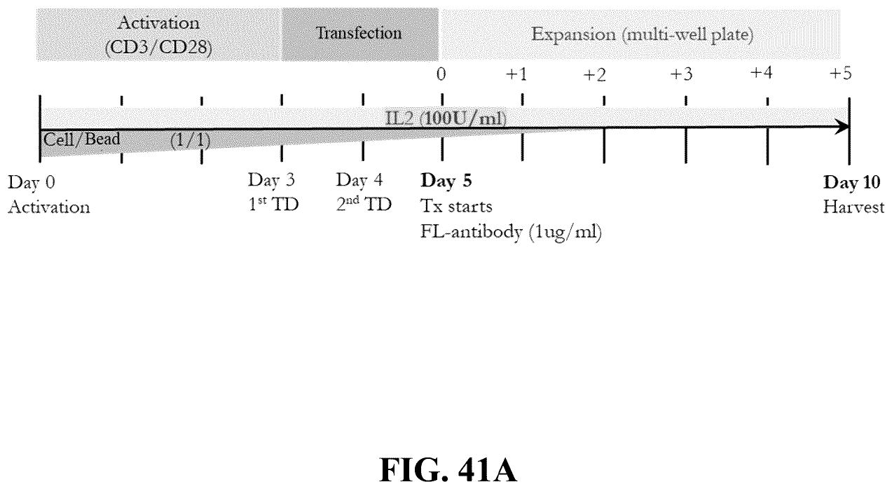

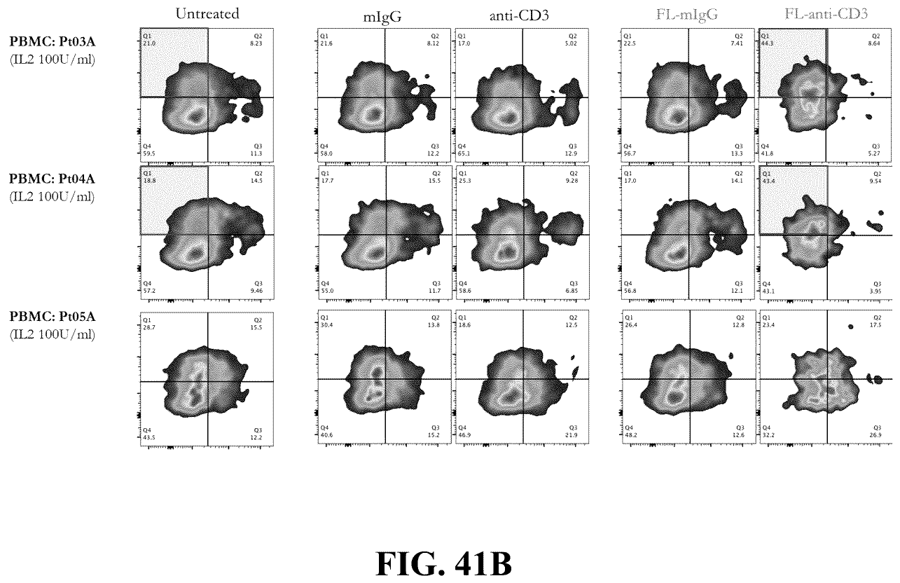

This example covers transfection, activation, differentiation, and expansion of engineered immune cells. As outlined in FIG. 41A, immune cells were activated with CD3/CD28 beads, transfected with a vector encoding an engineered cytokine receptor switch and mCherry fluorescent protein, and contacted to an activator of the cytokine receptor switch. This method differed from the method outlined in Example 7 and FIG. 28A in that it utilized a single dosage of CD3/CD28 beads and 100 (rather than 20) units/ml of IL2. As in Example 7, following CD3/CD28 activation and transfection with a vector encoding an engineered cytokine receptor switch, the cells were transferred to a multi-well plate containing an activator of the engineered cytokine receptor switches. The cells differentiated towards memory phenotypes similarly to those detailed in Example 7. FIG. 41B details the CCR7 and CD45RA expression profiles of the activated engineered immune cells following contact to various activator-functionalized substrates, demonstrating that the single dose of CD3/CD28 and higher dose of IL2 can achieve similar memory phenotype profiles as multiple doses of CD3/CD28 with lower doses of IL2.

### Example 32: Synthesis of Non-Cleavable and Cleavable Carrier-Small Molecule Activator Conjugates

This example describes the preparation of non-cleavable and cleavable carrier-small molecule activator conjugates for CAR T-cell release during ex vivo manufacturing.

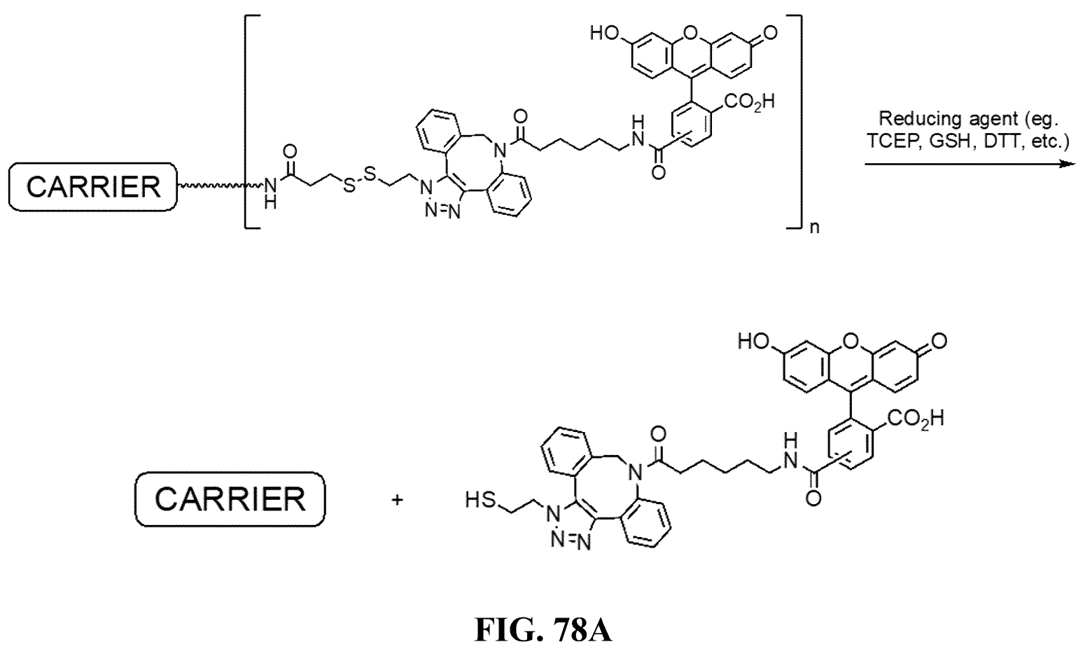

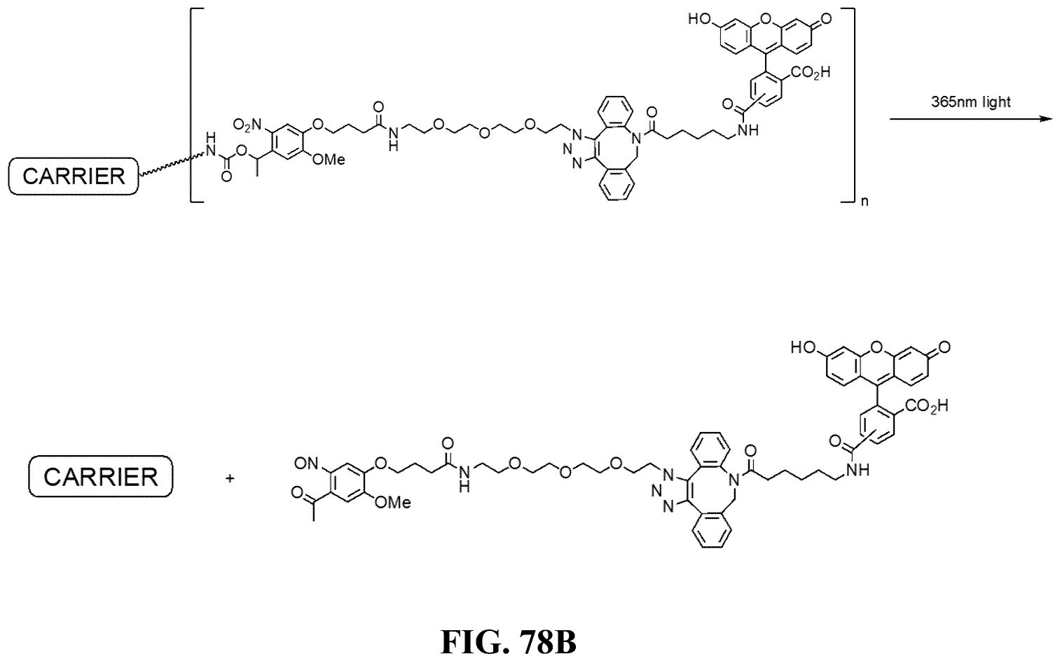

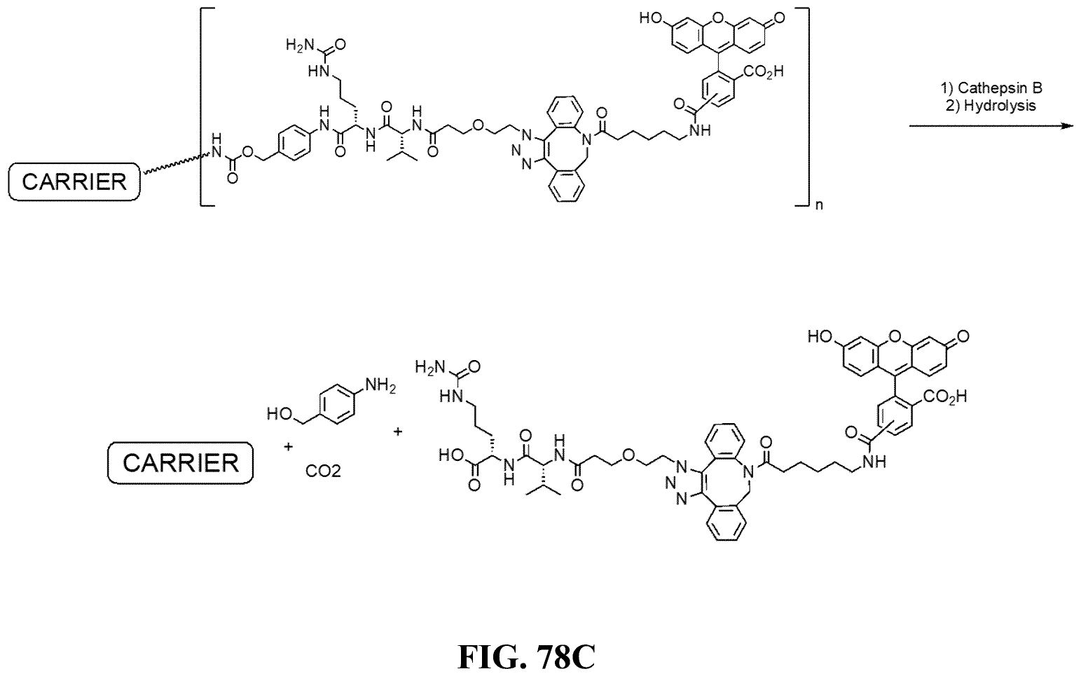

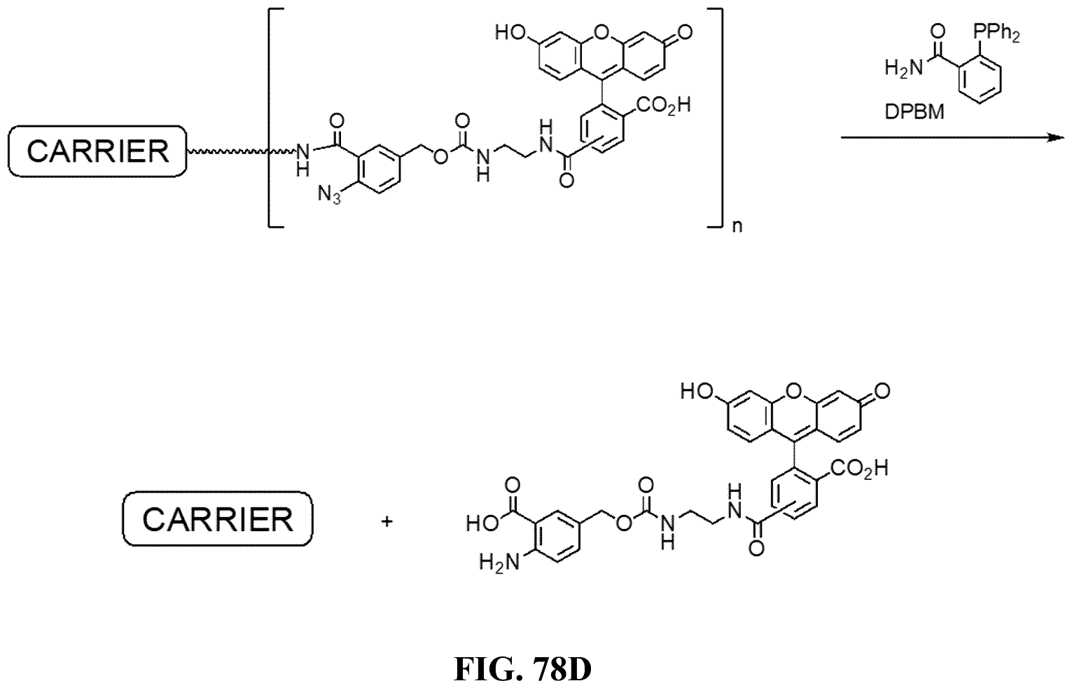

FIGS. 78A-78D provide schematics of various cleavable carrier-small molecule activator conjugates of the present disclosure for CAR-T cell release during ex vivo manufacturing. FIG. 78A provides a schematic representation of disulfide cleavage of a carrier from a small molecule under reducing conditions. FIG. 78B provides a schematic representation of photocleavage of a carrier from a small molecule after exposure to 365 nm light. FIG. 78C provides a schematic representation of protease-mediated cleavage of a carrier from a small molecule. FIG. 78D provides a schematic representation of a Staudinger reduction of an azide to an amine with concomitant release of a carrier from a small molecule. Conjugates prepared with the linkers shown in FIGS. 78A and 78B were prepared and tested for their effects on cell phenotype and signaling.

Dynabeads M-450 tosylactivated (Catalog #14013) were purchased from ThermoFisher Scientific. 3-azido-propanoic acid NHS ester and PC Azido-PEG3-NHS carbonate ester were purchased from BroadPharm. 2,2′-(ethylenedioxy)bis(ethylamine) (PEG2 bis-amine) was purchased from Sigma Aldrich. Polyoxyethylene bis(amine) MW=1,000 (PEG1 kDa bis-amine) and olyoxyethylene bis(amine) MW=3,400 (PEG3.4 kDa bis-amine) were purchased from Alfa Aesar. Fluorescein dibenzocyclooctyne (FAM-DBCO, 6-isomer) was purchased from Lumiprobe. Amino Dextran 40 kDa, containing an average of 10 amines (AD40×10) and 60 amines (AD40×60), was purchased from FinaBioSolutions.

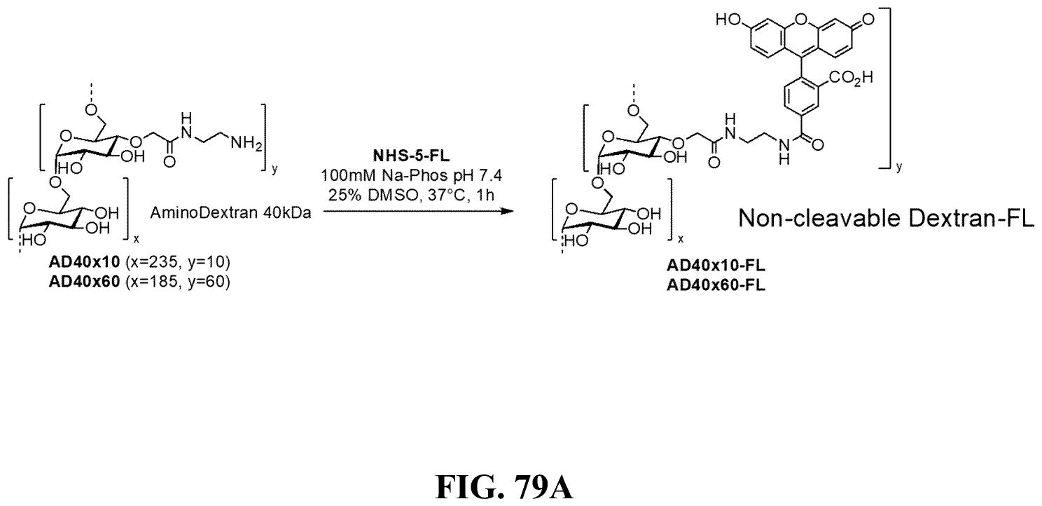

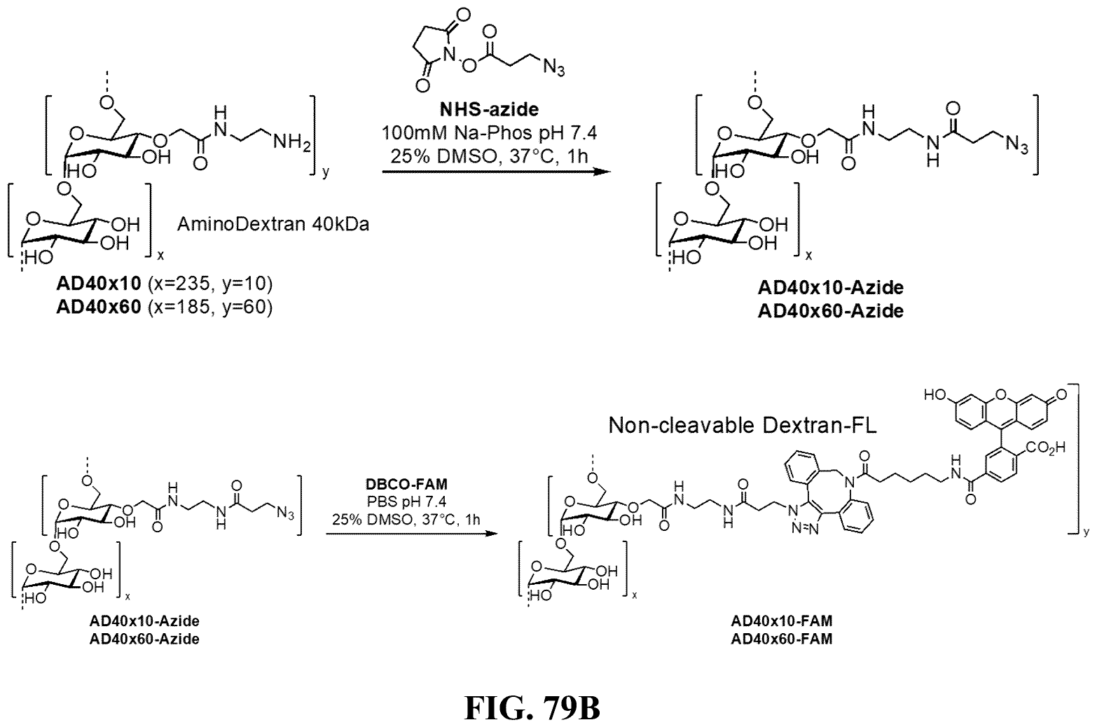

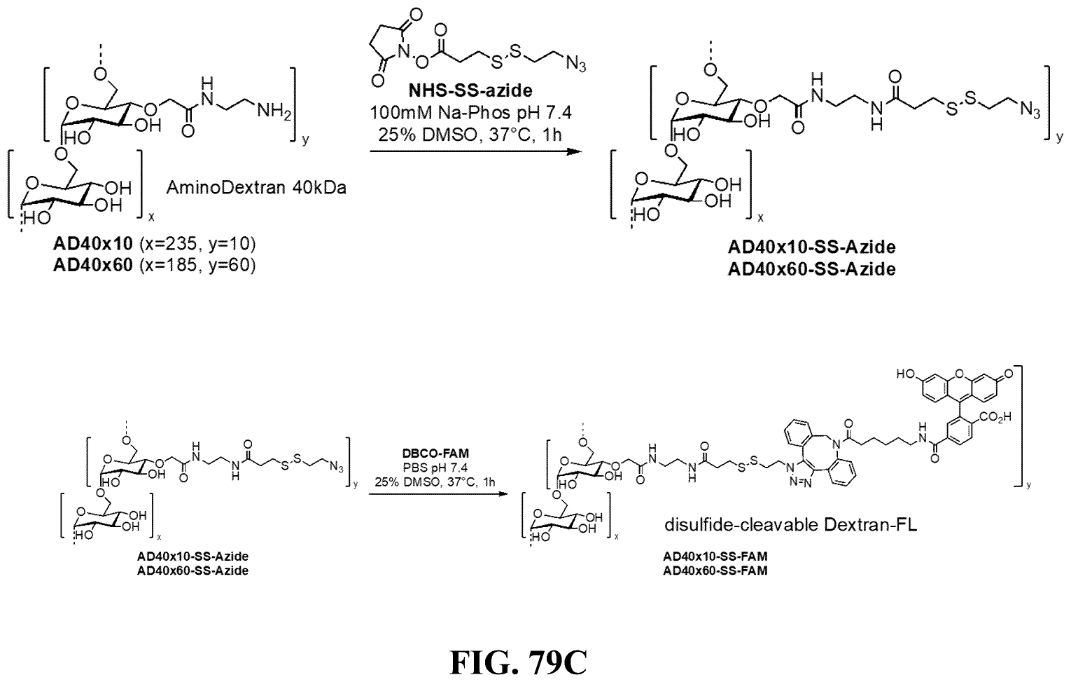

Synthesis of dextran-fluorescein conjugates was performed according to the reaction schemes illustrated in FIGS. 79A-79C. Specifically, FIG. 79A provides a schematic representation of a non-cleavable dextran-fluorescein conjugate prepared by direct acylation, FIG. 79B provides a schematic representation of a non-cleavable dextran-fluorescein conjugate prepared by sequential acylation and strain-promoted azide-alkyne cycloaddition (SPAAC), and FIG. 79C provides a schematic representation of a disulfide-cleavable dextran-fluorescein conjugate prepared by sequential acylation and SPAAC.

Non-cleavable AD40×10-fluorescein (AD40×10-FL) conjugates were prepared as follows (FIG. 79A): AD40×10 (47 μL from 80 mg/mL solution in 100 mM sodium phosphate pH 7.0, 3.81 mg, 95.2 nmol), 5-carboxyfluorescein N-hydroxysuccinimidyl ester (FL NHS ester, 150 μL from 10 mM DMSO stock), and 100 mM sodium phosphate pH 7.0 (802 μL) were combined. The reaction was incubated at 37° C. for 1 h and the AD40×10-FL conjugate was purified by Sephadex G15 eluting against PBS. Absorbance of the final conjugate was measured and concentration was approximated based on 70% recovery.

Non-cleavable AD40×60-fluorescein (AD40×60-FL) conjugates were prepared as follows (FIG. 79A): AD40×60 (153 μL from 20 mg/mL solution in 100 mM sodium phosphate pH 7.0, 3.07 mg, 75.8 nmol), 5-carboxyfluorescein N-hydroxysuccinimidyl ester (600 μL from 10 mM DMSO stock, 6 μmol), and 100 mM sodium phosphate pH 7.0 (1647 μL) were combined. The reaction was incubated at 37° C. for 1 h and the AD40×60-FL conjugate was purified by Sephadex G15 eluting against PBS. Absorbance of the final conjugate was measured and concentration was approximated based on 70% recovery.

Non-cleavable AD40×10 FAM conjugates were prepared as follows (FIG. 79B): AD40×10 (60 μL from 80 mg/mL stock in 100 mM sodium phosphate pH 7.0, 4.8 mg, 120 nmol), 3-azidopropanoic acid NHS ester (120 μL from 20 mM DMSO stock, 2400 nmol), DMSO (120 μL) and 100 mM sodium phosphate pH 7.0 (220 μL) were combined. The reaction was incubated at 37° C. for 1 h and the AD40×10-azide conjugate was purified by Sephadex G25 eluting against PBS. AD40×10-azide (approximately 2 mL after purification) was then combined with DBCO-FAM (6-isomer) (200 μL from 10 mM stock in DMSO, 2000 nmol). The reaction was incubated at 37° C. for 1 h and the AD40×10-FAM conjugate was purified by PD10 Sephadex G25 eluting against PBS.

Non-cleavable AD40×60 FAM conjugates were prepared as follows (FIG. 79B): AD40×60 (215.6 μL from 20 mg/mL stock in 100 mM sodium phosphate pH 7.0, 4.3 mg, 108 nmol), 3-azidopropanoic acid NHS ester (400 μL from 20 mM DMSO stock, 8000 nmol) and 100 mM sodium phosphate pH 7.0 (584 μL) were combined. The reaction was incubated at 37° C. for 1 h and the AD40×60-azide conjugate was purified by Sephadex G25 eluting against PBS. AD40×60-azide (approximately 4 mL after purification) was then combined with DBCO-FAM (6-isomer) (700 μL from 10 mM stock in DMSO, 2000 nmol). The reaction was incubated at 37° C. for 1 h and the AD40×60-FAM conjugate was purified by Sephadex G25 eluting against PBS.

Disulfide-cleavable AD40×10 FAM conjugates (AD40×10-SS-FAM) were prepared as follows (FIG. 79C): AD40×10 (60 μL from 80 mg/mL stock in 100 mM sodium phosphate pH 7.0, 4.8 mg, 120 nmol), 3-azidopropanoic acid NHS ester (120 μL from 20 mM DMSO stock, 2400 nmol), DMSO (120 μL), and 100 mM sodium phosphate pH 7.0 (220 μL) were combined. The reaction was incubated at 37° C. for 1 h and the AD40×10-SS-azide conjugate was purified by Sephadex G25 eluting against PBS. AD40×10-azide (approximately 1.8 mL after purification) was then combined with DBCO-FAM (6-isomer) (200 μL from 10 mM stock in DMSO, 2000 nmol) and DMSO (200 μL). The reaction was incubated at 37° C. for 1 h and the AD40×10-SS-FAM conjugate was purified by PD10 Sephadex G25 eluting against PBS. Absorbance of the final conjugate was measured and concentration was approximated based on 70% recovery.

Disulfide-cleavable AD40×60 FAM conjugates (AD40×60-SS-FAM) were prepared as follows (FIG. 79C): AD40×60 (215.6 μL from 20 mg/mL stock in 100 mM sodium phosphate pH 7.0, 4.3 mg, 108 nmol), 3-azidopropanoic acid NHS ester (400 μL from 20 mM DMSO stock, 8000 nmol) and 100 mM sodium phosphate pH 7.0 (584 μL) were combined. The reaction was incubated at 37° C. for 1 h and the AD40×60-azide conjugate was purified by Sephadex G25 eluting against PBS. AD40×60-azide (approximately 4 mL after purification) was then combined with DBCO-FAM (6-isomer) (700 μL from 10 mM stock in DMSO, 2000 nmol). The reaction was incubated at 37° C. for 1 h and the AD40×60-SS-FAM conjugate was purified by Sephadex G25 eluting against PBS. Absorbance of the final conjugate was measured and concentration was approximated based on 70% recovery.

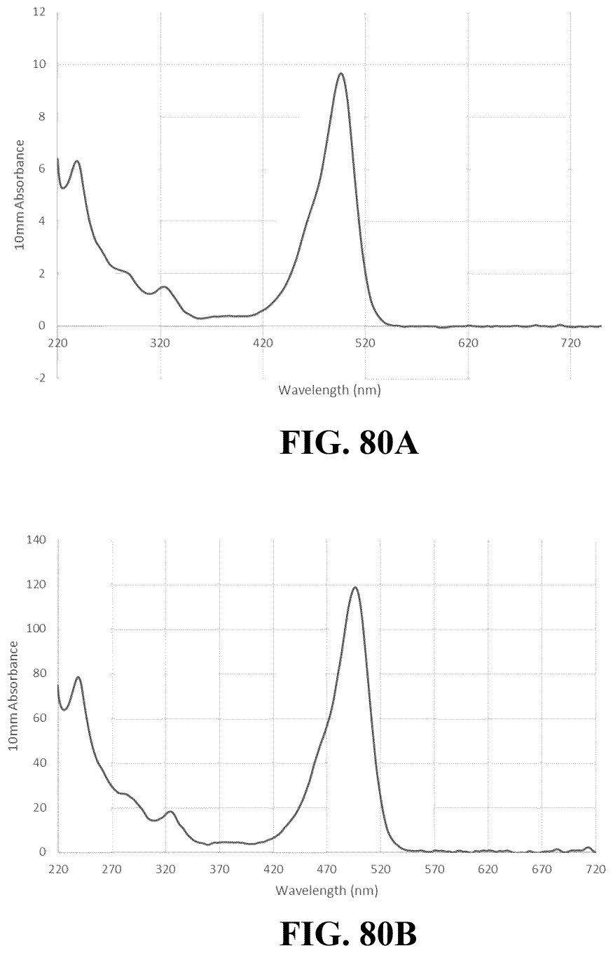

FIGS. 80A and 80B show the UV absorbance spectra of the AD40×10-FL non-cleavable dextran-fluorescein conjugates at 0.75 mg/mL (FIG. 80A) and 10 mg/mL (FIG. 80B) in PBS.

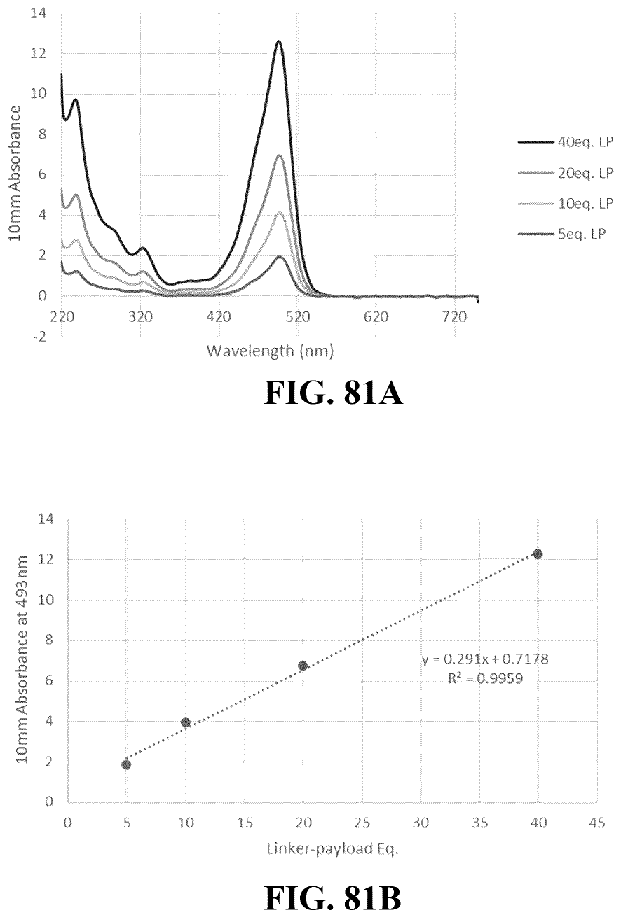

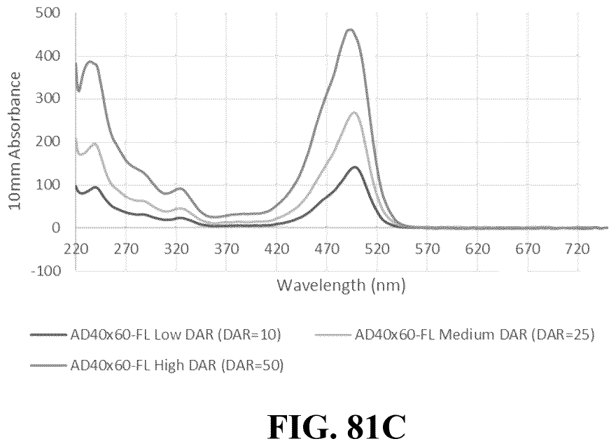

FIGS. 81A-81C show the UV absorbance data for the AD40×60-FL non-cleavable dextran-fluorescein conjugates obtained at different molar equivalents of FL NHS ester. Specifically, FIG. 81A illustrates the UV absorbance spectra of various AD40×60-FL conjugates containing different fluorescein (FL) loadings at 0.375 mg/mL in PBS (40 molar equivalents of linker-payload (eq. LP), 20 eq. LP, 10 eq. LP, and 5 eq. LP). FIG. 81B illustrates the absorbance at 493 nm of the various AD40×60-FL conjugates, showing the linear relationship between fluorescein loading and molar excess of FL NHS ester. FIG. 81C illustrates the UV absorbance for 10 mg/mL solutions of AD40×60-FL containing low (DAR=10), medium (DAR=25), and high (DAR=50) FL loading per dextran (in this and subsequent Examples, “DAR” refers to the activator-to-carrier (FL-to-dextran) ratio). These results indicate that varying the molar excess of FL NHS ester enabled changing of the relative loading of fluorescein per dextran.

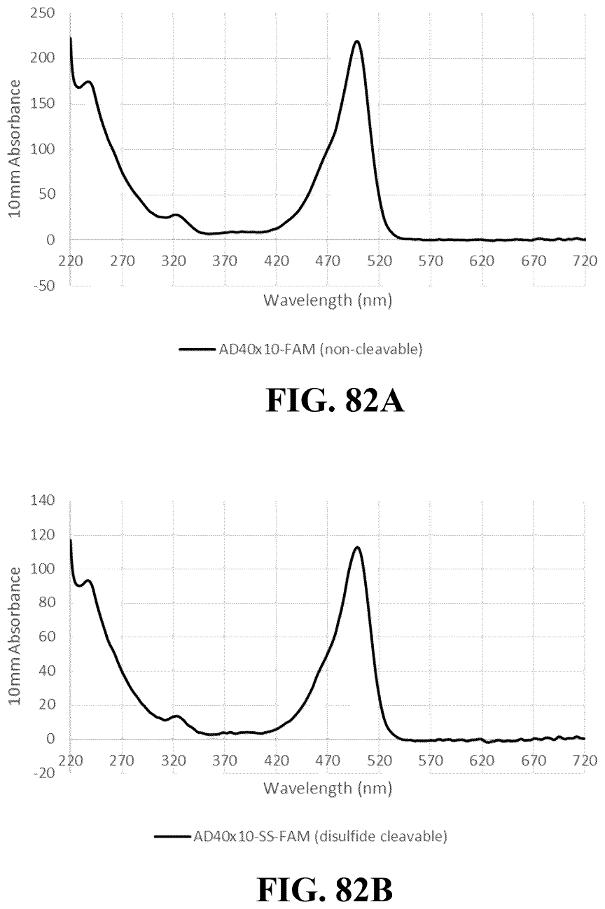

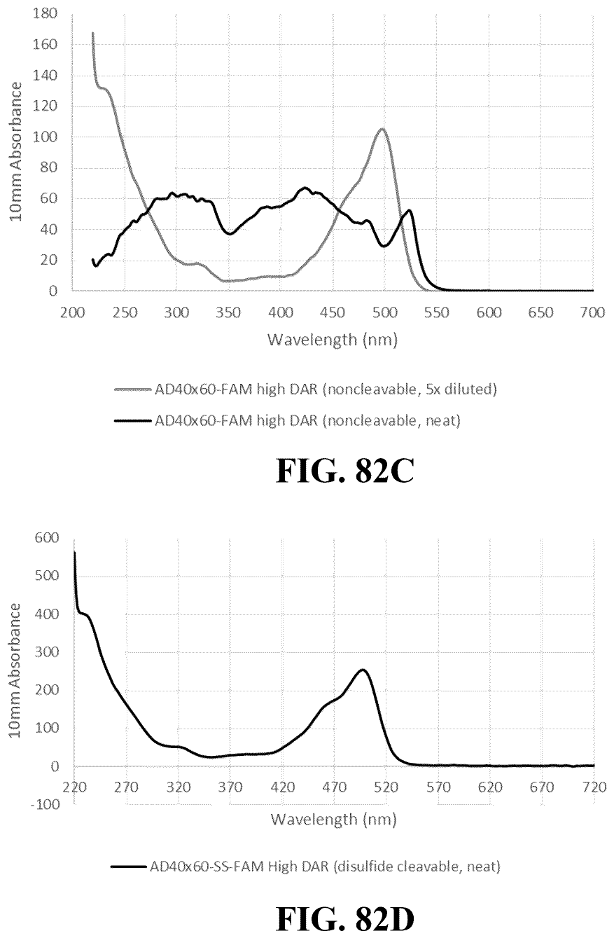

FIGS. 82A-82D show the UV absorbance spectra of non-cleavable and disulfide-cleavable 40 kDa dextran-fluorescein conjugates containing different fluorescein loading per dextran. The absorbance spectra were obtained prior to immobilization on M-450 amine modified Dynabeads. Specifically, FIG. 82A illustrates the UV absorbance of non-cleavable AD40×10-FL conjugates, FIG. 82B illustrates the UV absorbance of disulfide-cleavable AD40×10-SS-FAM conjugates, FIG. 82C illustrates the UV absorbance of non-cleavable AD40×60-FAM conjugates (black trace shows the absorbance spectrum at a higher concentration, which resulted in detector saturation; gray trace shows the absorbance spectrum after 5× dilution in PBS), and FIG. 82D illustrates the UV absorbance of disulfide-cleavable AD40×60-SS-FAM conjugates. The signature absorbance of fluorescein at 493 nm was observed for all conjugates.

These results indicate that dextran-fluorescein conjugates with non-cleavable and disulfide-cleavable linkers were successfully synthesized, and that the degree of fluorescein loading per dextran could be controlled.

### Example 33: Covalent Immobilization of Cleavable and Non-Cleavable Carrier-Small Molecule Activator Conjugates on Beads

This example describes the covalent immobilization of cleavable and non-cleavable carrier-small molecule activator conjugates on beads.

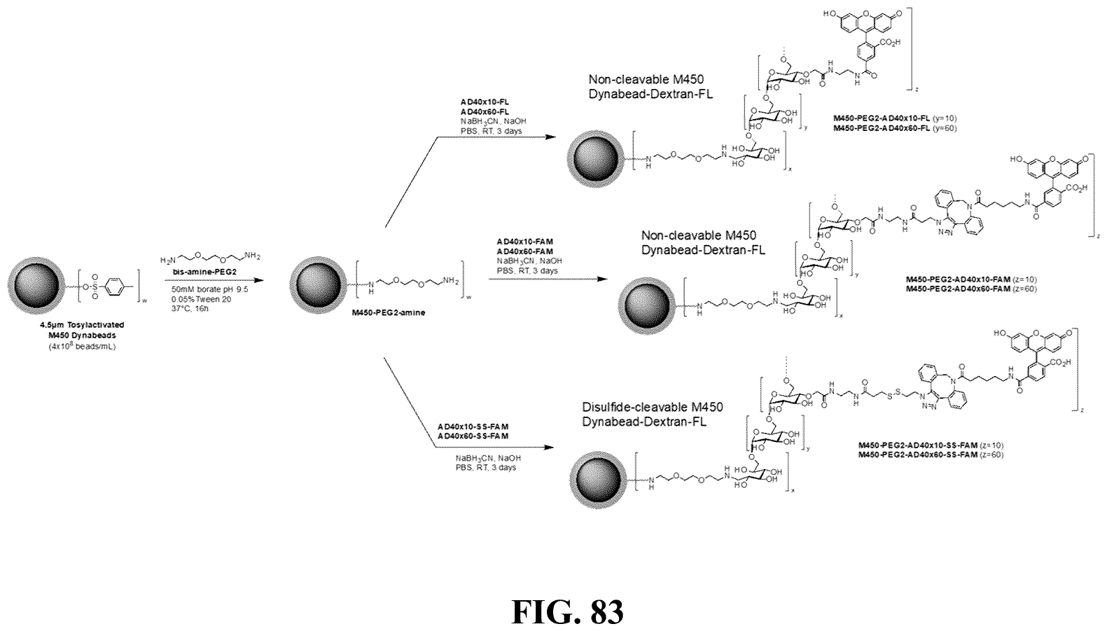

FIG. 83 schematically illustrates the covalent immobilization of non-cleavable and disulfide-cleavable dextran-fluorescein conjugates on M-450 tosylactivated Dynabeads by reductive amination. 4.5 μm tosylactivated M-450 Dynabeads (40 million beads/mL) were reacted with bis-amine-PEG2 in 50 mM borate pH 9.5 and 0.05% polysorbate-20 at 37° C. and 16 h. The resulting aminated beads (M450-PEG2-amine) were used for end group covalent immobilization of the conjugates of Example 32 by reductive amination.

Non-cleavable M-450 Dynabead-dextran-fluorescein conjugates (M450-PEG2-AD40×10-FL and M450-PEG2-AD40×60-FL) were prepared as follows (FIG. 83, top): M450-PEG2-amine Dynabeads (100 μL of 100 mM sodium borate pH 9.5 and 0.05% (w/v) polysorbate-20, 40 million beads) were resuspended. 250 μM AD40×10-FL or AD40×60-FL in PBS (40 μL, 10 mmol) was added. 5 M sodium cyanoborohydride in 1 M sodium hydroxide (20 μL, 100 μmol) was added and the solution was incubated at room temperature for 3 days. Upon completion, beads were placed on a magnet and the supernatant was discarded. Beads were washed three times with 200 μL of 100 mM sodium phosphate pH 7, 0.05% (w/v) polysorbate-20, and 0.09% sodium azide, incubated with shaking at 1300 rpm for 1-2 min during each wash step, and the supernatant was discarded. Beads were resuspended in 100 μL of 100 mM sodium phosphate pH 7.0, 0.05% (w/v) polysorbate-20, and 0.09% sodium azide and stored at 2-8° C.

Non-cleavable M-450 Dynabead-dextran-fluorescein conjugates (M450-PEG2-AD40×10-FAM and M450-PEG2-AD40×60-FAM) were prepared as follows (FIG. 83, middle): M450-PEG2-amine Dynabeads (200 μL of 100 mM sodium borate pH 9.5 and 0.05% (w/v) polysorbate-20, 80 million beads) were resuspended. 250 μM AD40×10-FAM or AD40×60-FAM) in PBS (80 μL, 20 mmol) was added. 5 M sodium cyanoborohydride in 1 M sodium hydroxide (40 μL, 200 μmol) was added and the solution was incubated at room temperature for 3 days. Upon completion, beads were placed on a magnet and the supernatant was discarded. Beads were washed three times with 400 μL of 100 mM sodium phosphate pH 7.0, 0.05% (w/v) polysorbate-20, and 0.09% sodium azide, incubated with shaking at 1300 rpm for 1-2 min during each wash step, and the supernatant was discarded. Beads were resuspended in 200 μL of 100 mM sodium phosphate pH 7.0, 0.05% (w/v) polysorbate-20, and 0.09% sodium azide and stored at 2-8° C.

Disulfide-cleavable M-450 Dynabead-dextran-fluorescein conjugates (M450-PEG2-AD40×10-SS-FAM and M450-PEG2-AD40×60-SS-FAM) were prepared as follows (FIG. 83, bottom): M450-PEG2-amine Dynabeads (200 μL of 100 mM sodium borate pH 9.5 and 0.05% (w/v) polysorbate-20, 80 million beads) were resuspended. 250 μM AD40×10-SS-FAM or AD40×60-SS-FAM in PBS (80 μL, 20 mmol) was added. 5 M sodium cyanoborohydride in 1 M sodium hydroxide (40 μL, 200 μmol) was added and the solution was incubated at room temperature for 3 days. Upon completion, beads were placed on a magnet and the supernatant was discarded. Beads were washed three times with 400 μL of 100 mM sodium phosphate pH 7.0, 0.05% (w/v) polysorbate-20, and 0.09% sodium azide, incubated with shaking at 1300 rpm for 1-2 min during each wash step, and the supernatant was discarded. Beads were resuspended in 200 μL of 100 mM sodium phosphate pH 7.0, 0.05% (w/v) polysorbate-20, and 0.09% sodium azide and stored at 2-8° C.

Fluorescence measurements of non-cleavable and disulfide-cleavable M-450-PEG-FL Dynabeads were performed as follows. Dynabeads were resuspended at 400 million beads/mL in 100 mM sodium phosphate pH 7.0, 0.05% (w/v) polysorbate-20, and 0.09% sodium azide. 50 μL of each solution was aliquoted to a 96-well half area opaque microplate and fluorescein fluorescence (excitation at 493 nm, emission at 530 nm) was measured. The conjugated fluorescein concentration was approximated based on the linear relationship of fluorescence vs. unbound fluorescein concentration in the presence of suspended M-450 tosylactivated Dynabeads.

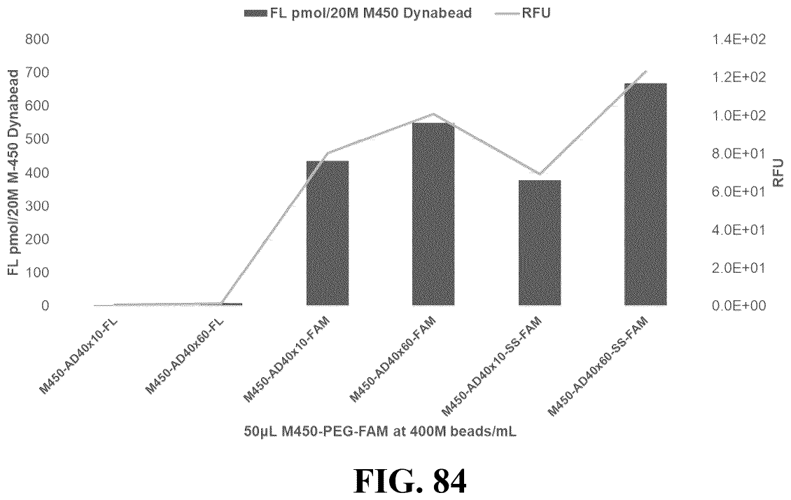

FIG. 84 shows the relative fluorescence of non-cleavable and disulfide cleavable 4.5 μm M-450 Dynabead-dextran-fluorescein conjugates. Dynabead-dextran-fluorescein conjugates prepared by SPAAC (M450-AD40×10-FAM, M450-AD40×60-FAM, M450-AD40×10-SS-FAM, M450-AD40×60-SS-FAM) displayed higher fluorescein fluorescence per bead. The higher fluorescein loaded dextran (M450-AD40×60-FAM and M450-AD40×60-SS-FAM) showed higher fluorescence than the lighter loaded Dynabead-dextran-fluorescein conjugates (M450-AD40×10-FAM and M450-AD40×10-SS-FAM). These results indicate that dextran-fluorescein conjugates with non-cleavable and disulfide-cleavable linkers were successfully immobilized on beads.

### Example 34: Preparation of Non-Cleavable and Photocleavable Bead-Activator Conjugates

This example describes the preparation of non-cleavable and photocleavable bead-small molecule activator conjugates.

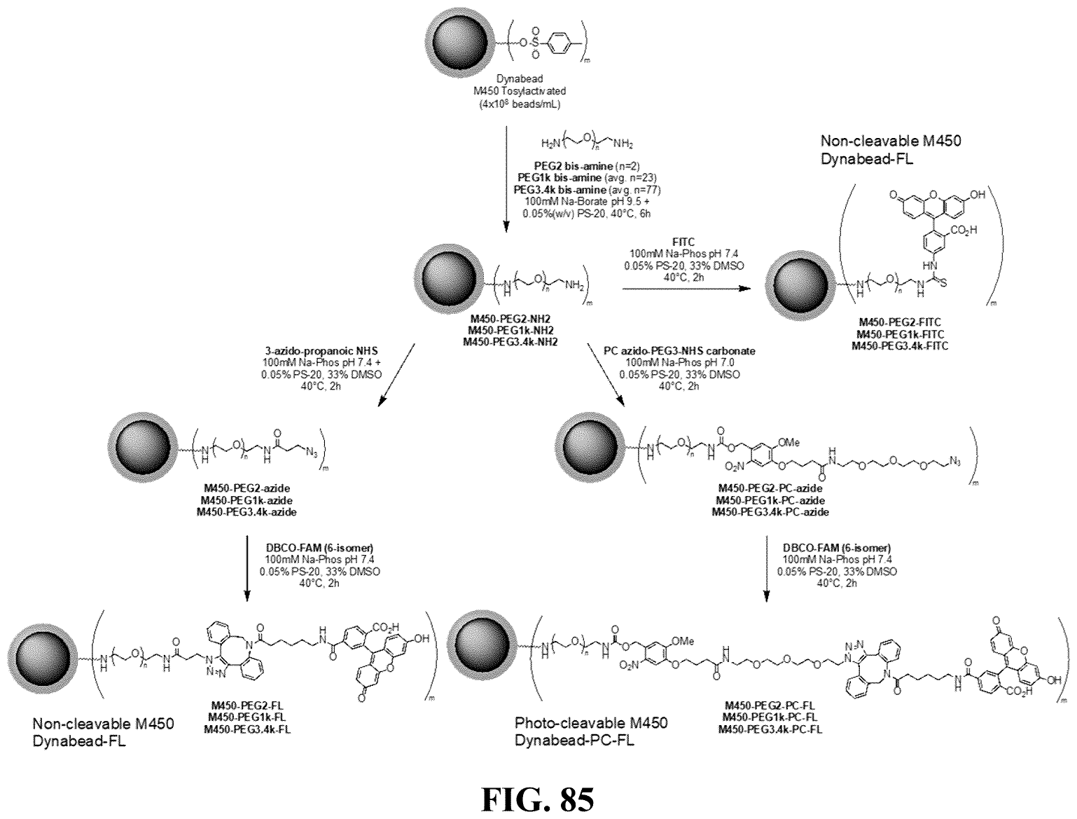

FIG. 85 schematically illustrates the preparation of non-cleavable and photocleavable 4.5 μm M-450 Dynabead-fluorescein conjugates by direct isothiocyanate/amine coupling or copper (I)-catalyzed azide-alkyne cycloaddition (CuAAC) (click chemistry). Each conjugate contained a flexible polyethylene glycol (PEG) linker having a different average number of PEG units. The relative fluorescence of each conjugate was compared to systematically determine the impact of linker length on SMAR activation by the fluorescein-conjugated Dynabeads.

Aminated M-450 Dynabeads with varying PEG linker lengths (M450-PEG2-amine, M450-PEG1k-amine, M450-PEG3.4k-amine) were prepared as follows: M-450 tosylactivated Dynabead stock (400 million beads/mL) was vortexed to form a suspension. 40 million beads (100 μL from 400 million beads/mL stock,) were aliquoted to a 1.5 mL microfuge tube. Beads were washed three times with 200 μL of 100 mM sodium borate pH 9.5 and 0.05% (w/v) polysorbate-20, incubated with shaking at 1300 rpm for 1-2 min for each wash step, placed on a magnet, and the supernatant was discarded. Beads were resuspended in 100 μL of 100 mM sodium borate pH 9.5 and 0.05% (w/v) polysorbate-20, PEG bis-amine linker (PEG2 bis-amine (n=2), PEG1k bis-amine (average n=23), or PEG3.4k bis-amine (average n=77); 40 μL from 50 mM stock solution in 100 mM sodium borate pH 9.5, 2 μmol) was added, and the solution was incubated with shaking at 1300 rpm, 40° C. for 6 h. Upon completion, beads were placed on a magnet and the supernatant was discarded. Beads were washed three times with 200 μL of 100 mM sodium phosphate pH 7.0 and 0.05% (w/v) polysorbate-20, incubated with shaking at 1300 rpm for 1-2 min during each wash step, placed on a magnet, and the supernatant was discarded.

Preparation of non-cleavable Dynabead-fluorescein conjugates (M450-PEG2-FITC, M450-PEG1k-FITC, M450-PEG3.4k-FITC) by direct acylation was performed as follows: M450-PEG2-amine, M450-PEG1 kDa-amine, or M450-PEG3.4 kDa-amine Dynabeads were suspended in 100 μL of 100 mM sodium borate pH 9.5 and 0.05% (w/v) polysorbate-20. 4 mM FITC (5-isomer) in DMSO (50 μL, 0.2 μmol) was added and the solution was incubated with shaking at 1300 rpm, 40° C. for 2 h. Upon completion, beads were placed on a magnet and the supernatant was discarded. Beads were washed three times with 200 μL of 100 mM sodium phosphate pH 7.0, 0.05% (w/v) polysorbate-20, and 0.09% sodium azide, incubated with shaking at 1300 rpm for 1-2 min during each wash step, and the supernatant was discarded. The beads were resuspended in 100 μL of 100 mM sodium phosphate pH 7.0, 0.05% (w/v) polysorbate-20, and 0.09% sodium azide and stored at 2-8° C.

Preparation of non-cleavable azide-functionalized Dynabeads for click chemistry (M450-PEG2-azide, M450-PEG1k-azide, M450-PEG3.4k-azide) was performed as follows: M450-PEG2-amine, M450-PEG1 kDa-amine, or M450-PEG3.4 kDa-amine Dynabeads were resuspended in 100 μL of 100 mM sodium phosphate pH 7.0 and 0.05% (w/v) polysorbate-20. 4 mM 3-azido-propanoic acid NHS ester in DMSO (50 μL, 0.2 μmol) was added and the solution was incubated with shaking at 1300 rpm, 40° C. for 2 h. Upon completion, beads were placed on a magnet and the supernatant was discarded. Beads were washed three times with 200 μL of 100 mM sodium phosphate pH 7.0 and 0.05% polysorbate-20, incubated with shaking at 1300 rpm for 1-2 min during each wash step, and the supernatant was discarded.

Preparation of photocleavable azide-functionalized Dynabeads for click chemistry (M450-PEG2-PC-azide, M450-PEG1k-PC-azide, M450-PEG3.4k-PC-azide) was performed as follows: M450-PEG2-amine, M450-PEG1 kDa-amine, or M450-PEG3.4 kDa-amine Dynabeads were resuspended in 100 μL of 100 mM sodium phosphate pH 7.0 and 0.05% (w/v) polysorbate-20. 4 mM PC Azido-PEG3-NHS carbonate ester in DMSO (50 μL, 0.2 μmol) was added and the solution was incubated with shaking at 1300 rpm, 40° C. for 2 h. Upon completion, beads were placed on a magnet and the supernatant was discarded. Beads were washed three times with 200 μL of 100 mM sodium phosphate pH 7.0 and 0.05% (w/v) polysorbate-20, incubated with shaking at 1300 rpm for 1-2 min during each wash step, and the supernatant was discarded.

Preparation of non-cleavable Dynabead-fluorescein conjugates (M450-PEG2-FL or M450-PEG2-FAM, M450-PEG1k-FL or M450-PEG1k-FAM, M450-PEG3.4k-FL or M450-PEG3.4k-FAM) by click chemistry was performed as follows: M450-PEG2-azide, M450-PEG1k-azide, or M450-PEG3.4k-azide Dynabeads were resuspended in 100 μL of 100 mM sodium phosphate pH 7.0 and 0.05% (w/v) polysorbate-20. 2 mM DBCO-FAM, 6 isomer in DMSO (50 μL, 0.2 μmol) was added and the solution was incubated with shaking at 1300 rpm, 40° C. for 2 h. Upon completion, beads were placed on a magnet and the supernatant was discarded. Beads were washed three times with 200 μL of 100 mM sodium phosphate pH 7.0, 0.05% (w/v) polysorbate-20, and 0.09% sodium azide, incubated with shaking at 1300 rpm for 1-2 min during each wash step, and the supernatant was discarded. Beads were resuspended in 100 μL of 100 mM sodium phosphate pH 7.0, 0.05% (w/v) polysorbate-20, and 0.09% sodium azide and stored at 2-8° C.

Preparation of photocleavable Dynabead-fluorescein conjugates (M450-PEG2-PC-FL or M450-PEG2-PC-FAM, M450-PEG1k-PC-FL or M450-PEG1k-PC-FAM, M450-PEG3.4k-PC-FL or M450-PEG3.4k-PC-FAM) by click chemistry was performed as follows: M450-PEG2-PC-azide, M450-PEG1k-PC-azide, or M450-PEG3.4k-PC-azide Dynabeads were resuspended in 100 μL of 100 mM sodium phosphate pH 7.0 and 0.05% (w/v) polysorbate-20. 2 mM DBCO-FAM, 6 isomer in DMSO (50 μL, 0.2 μmol) was added and the solution was incubated with shaking at 1300 rpm, 40° C. for 2 h. Upon completion, beads were placed on a magnet and the supernatant was discarded. Beads were washed three times with 200 μL of 100 mM sodium phosphate pH 7.0, 0.05% (w/v) polysorbate-20, and 0.09% sodium azide, incubated with shaking at 1300 rpm for 1-2 min during each wash step, and the supernatant was discarded. Beads were resuspended in 100 μL of 100 mM sodium phosphate pH 7.0, 0.05% (w/v) polysorbate-20, and 0.09% sodium azide and stored at 2-8° C.

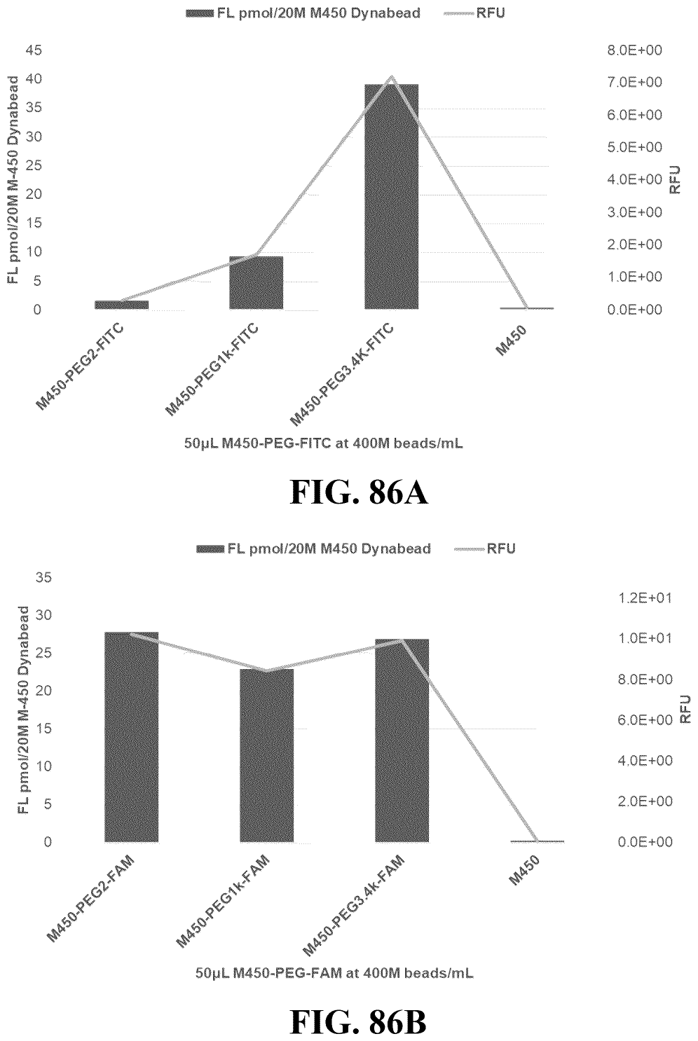

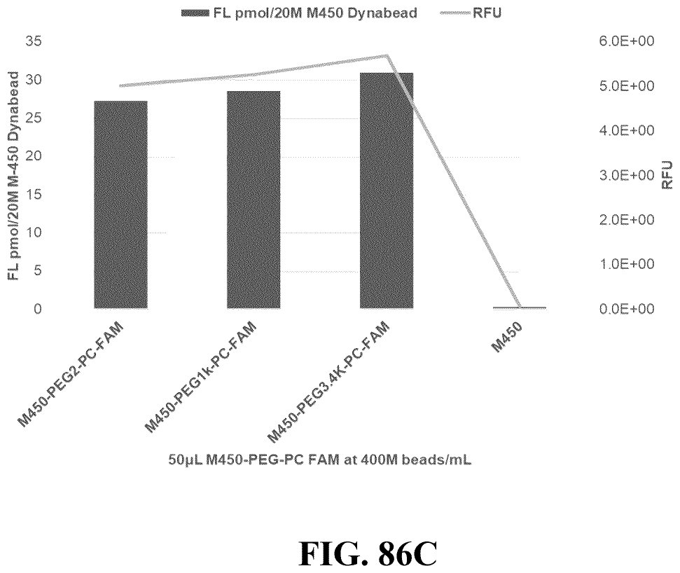

FIGS. 86A-86C show the relative fluorescence and approximate concentrations of conjugated fluorescein for non-cleavable M450-PEG-FITC Dynabeads (FIG. 86A), non-cleavable M450-PEG-FAM Dynabeads (FIG. 86B), and photocleavable M450-PEG-PC-FAM Dynabeads (FIG. 9C). The non-cleavable conjugate prepared by direct amine-isothiocyanate coupling showed a marked increase in fluorescence with longer linker length (FIG. 86A). The non-cleavable and photocleavable conjugates prepared by click chemistry showed similar fluorescence for all three linker lengths (FIGS. 86B and 86C). These results demonstrate that non-cleavable and photocleavable fluorescein-Dynabead conjugates could be successfully prepared, and that fluorescein could be incorporated at ligand loadings comparable to what is typically observed for M-450 4.5 μm tosylactivated Dynabeads coupled to other ligands.

### Example 35: Fluorescence Measurements of Non-Cleavable and Photocleavable Bead-Activator Conjugates

This example describes studies to investigate the fluorescence of non-cleavable and photocleavable bead-small molecule activator conjugates. The objective was to determine whether Dynabeads contribute to quenching of the fluorescence signal, since it may be desirable to use fluorescein fluorescence to approximate the amount of fluorescein conjugated to beads. A suspension of unbound fluorescein of known concentration was used in the presence of M-450 Dynabeads, and the fluorescence versus concentration was measured to determine a linear relationship. This was intended to mimic the fluorescence of fluorescein when covalently bound to the Dynabead surface.

M-450 tosylactivated Dynabeads were resuspended at 400 million beads/mL in 100 mM sodium phosphate pH 7.0, 0.05% (w/v) polysorbate-20, and 0.09% sodium azide. 50 μL of each solution was aliquoted to a 96-well half area opaque microplate and fluorescein fluorescence (excitation at 493 nm, emission at 530 nm) was measured. The conjugated fluorescein concentration was approximated based on the linear relationship of fluorescence versus unbound fluorescein concentration in the presence of suspended M-450 tosylactivated Dynabeads.

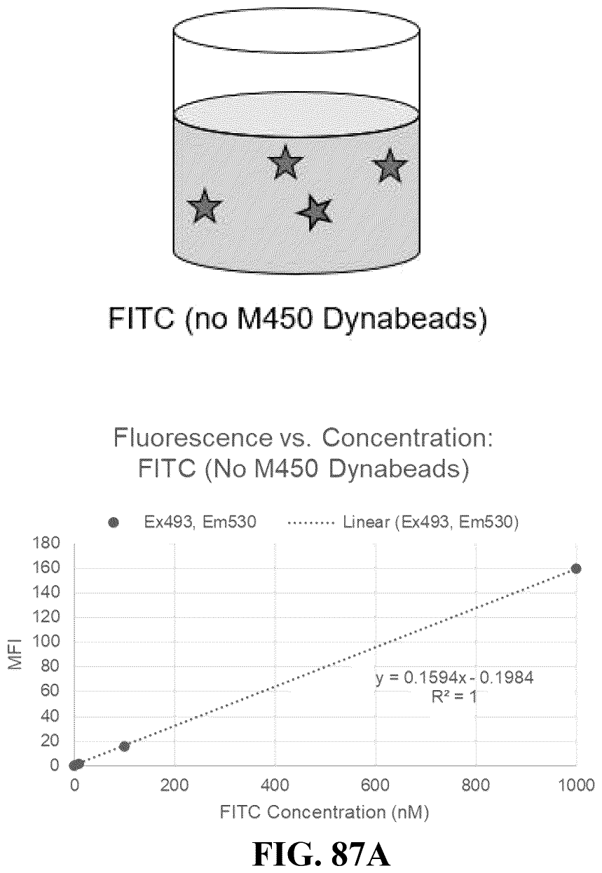

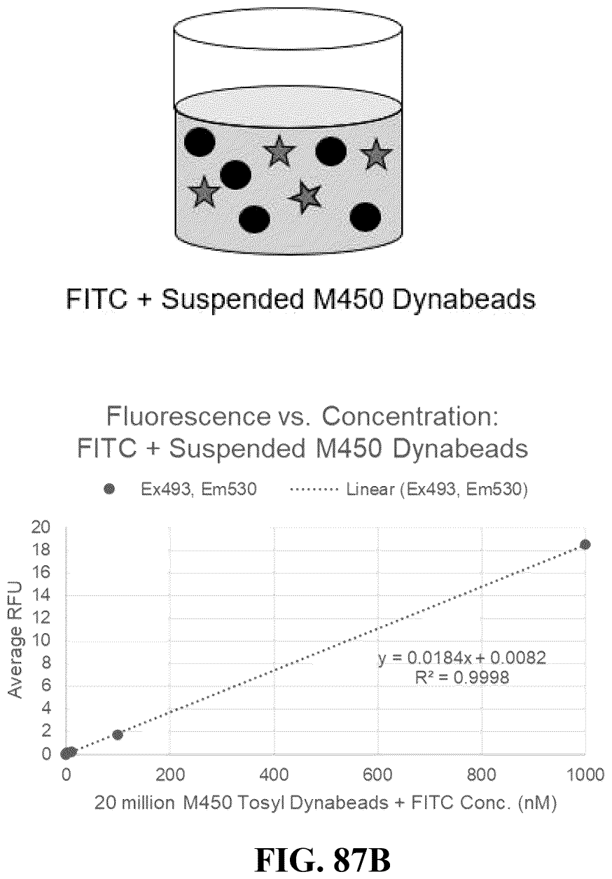

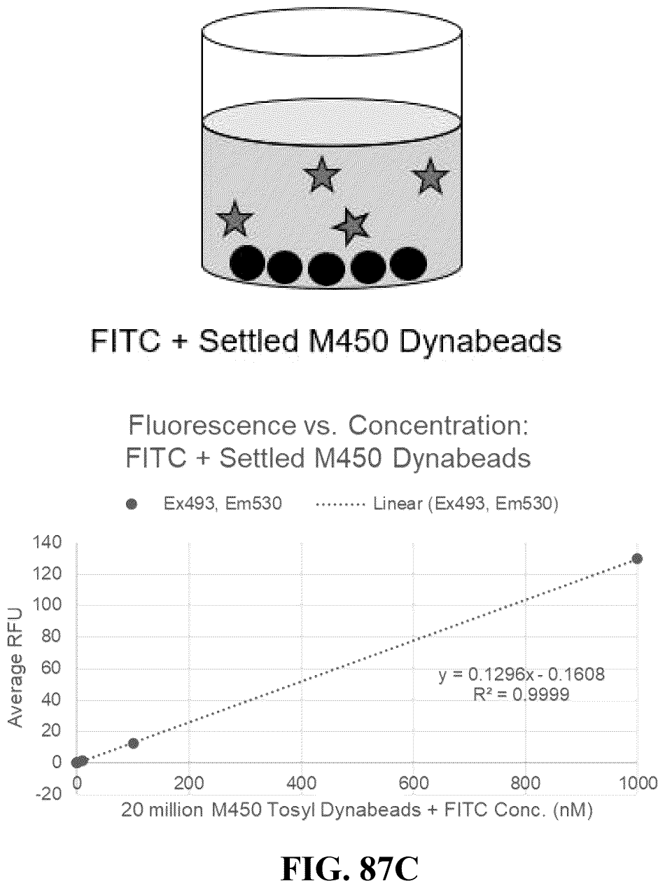

FIGS. 87A-87C illustrate calibration curves of fluorescence vs unbound fluorescein concentration for FITC in the absence of Dynabeads (FIG. 87A), FITC in the presence of suspended M-450 tosylactivated Dynabeads (FIG. 87B), and FITC in the presence of settled M540 Dynabeads (FIG. 87C). The linear relationship obtained for unbound fluorescein in the presence of suspended M-450 Dynabeads was used to approximate the concentration of fluorescein when conjugated to M-450 Dynabeads. Briefly, the concentration of FITC was varied in the presence of 50 μL of M-450 tosylactivated Dynabeads (50 μL from 400 million beads/mL) and fluorescence (excitation 493 nm, emission 530 nm) was measured.

As shown by FIGS. 87A and 87B, unbound fluorescein showed excellent fluorescence relative to the same concentration of fluorescein in the presence of a suspension of M-450 Dynabeads, which suggests that the Dynabeads produce some quenching of the fluorescence signal. When beads were settled by centrifugation (FIG. 87C), the fluorescein fluorescence was similar to the fluorescein fluorescence in the absence of Dynabeads. The relationship shown in the graph of FIG. 87B was used to approximate the concentration of fluorescein per Dynabead in FIGS. 86A-86C.

### Example 36: Activation of Cytokine Receptor Switches by Bead-Activator Conjugates

This example describes studies to investigate activation of an engineered cytokine receptor switch by bead-activator conjugates. Other studies have used a FITC-dextran-coated plate to activate an IL7Rα SMAR. The objective of this study was to determine whether soluble mediators such as an anti-CD3-FL conjugated antibody or FITC-conjugated Dynabeads can also activate the IL7Rα SMAR.

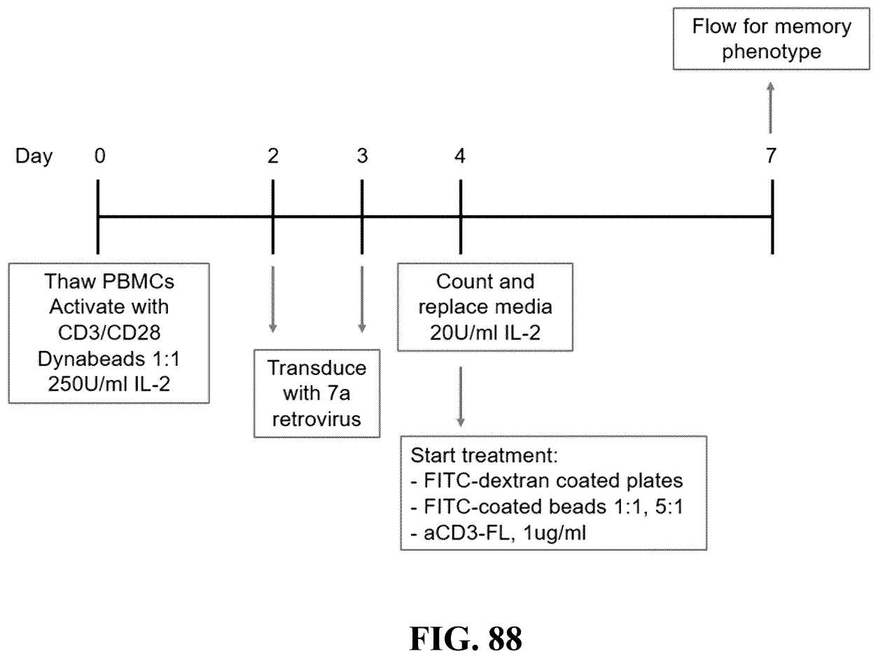

FIG. 88 illustrates the experimental design. At Day 0, peripheral blood mononuclear cells (PBMCs) were thawed and activated with CD3/CD28 Dynabeads at a 1:1 bead-to-cell ratio in the presence of 250 U/mL IL2. At Days 2 and 3, the cells were transduced with a retrovirus to express the IL7Rα SMAR. At Day 4, cells were counted, the media was replaced, and 20 U/mL IL2 was added. On Day 4, cells were treated with FITC-dextran coated plates, FITC-coated beads at bead-to-cell ratios of 1:1 and 5:1, or an anti-CD3-FL antibody (aCD3-FL, 1 μg/mL). The FITC-coated beads that were tested were: FITC-dextran coated beads (AD40×60-FL High DAR and AD40×60-FL Low DAR of Examples 32 and 33), non-cleavable FITC-PEG conjugated beads (M450-PEG2-FL, M450-PEG1k-FL, and M450-PEG3.4k-FL of Example 34), and photocleavable FITC-PEG conjugated beads (M450-PEG2-PC-FL, M450-PEG1k-PC-FL, and M450-PEG3.4k-PC-FL of Example 34). At Day 7, cell samples were collected, and phenotypes were assessed by flow cytometry.

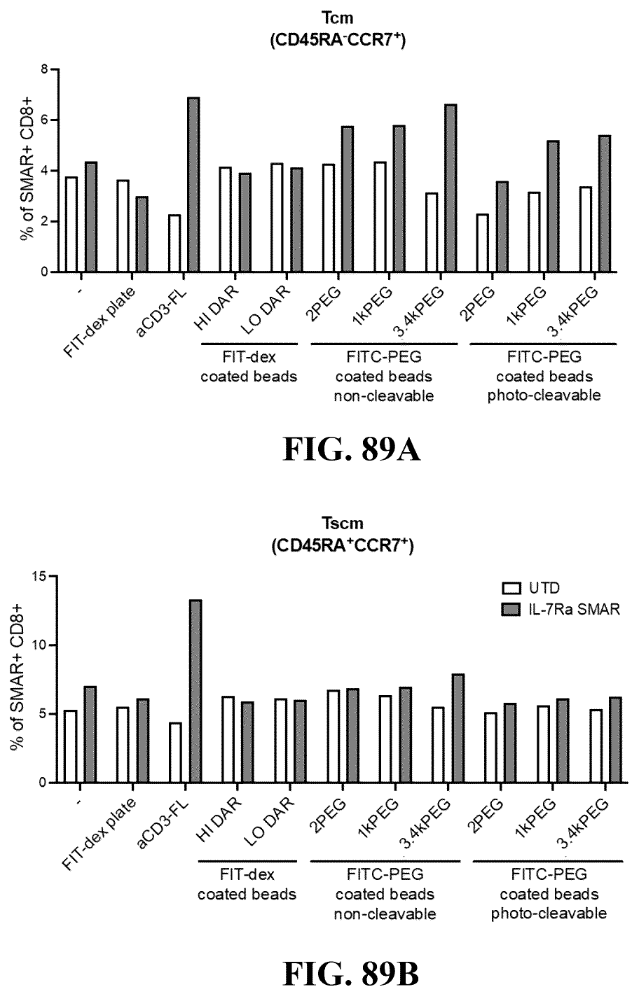

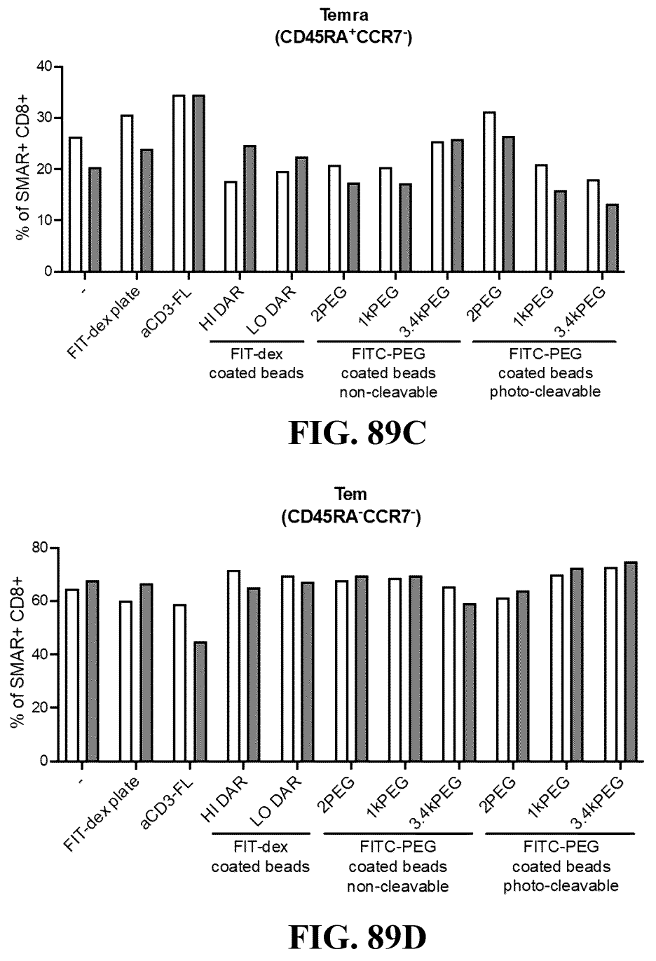

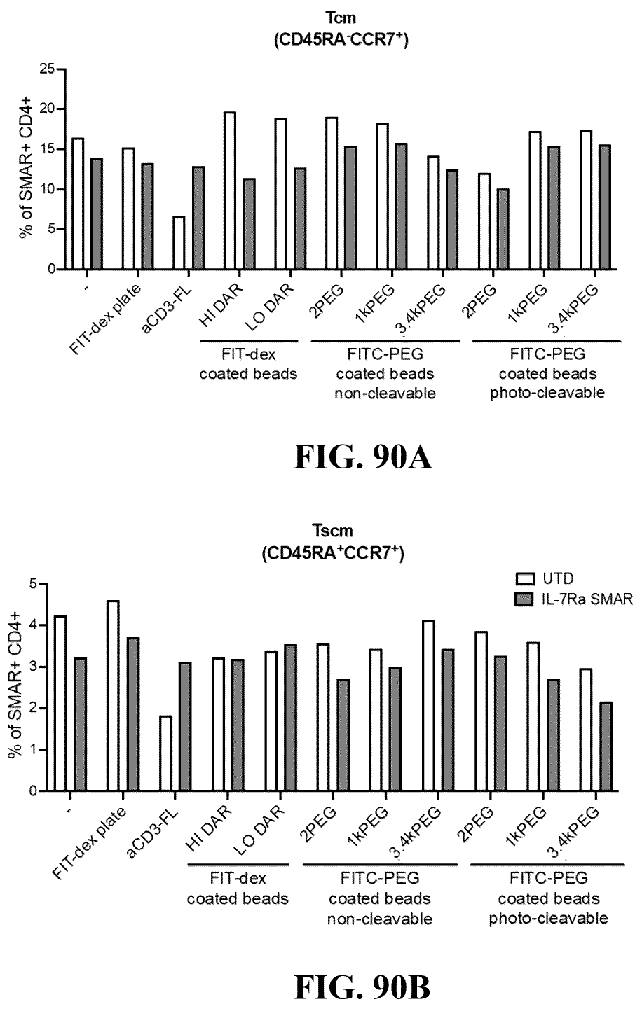

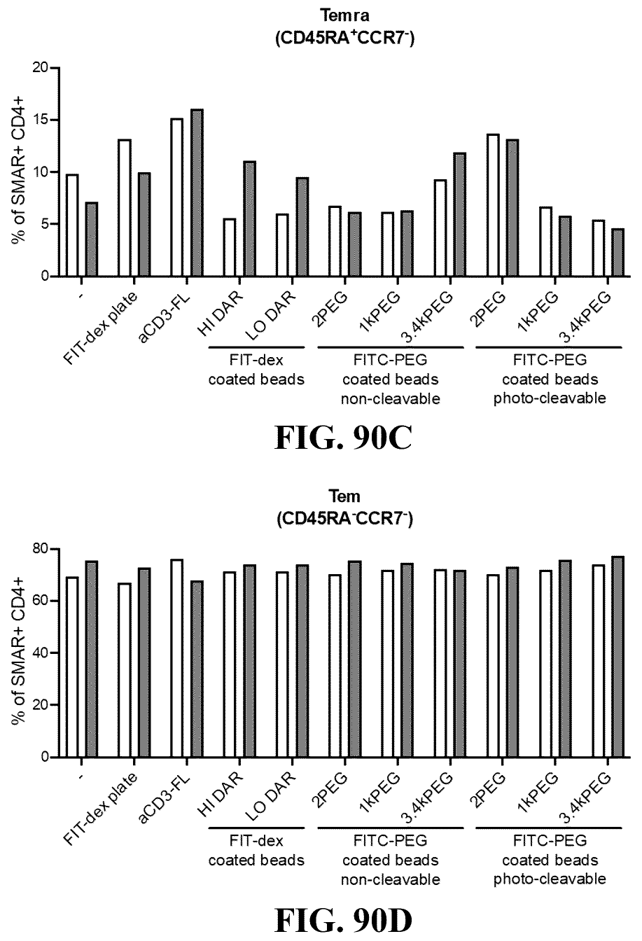

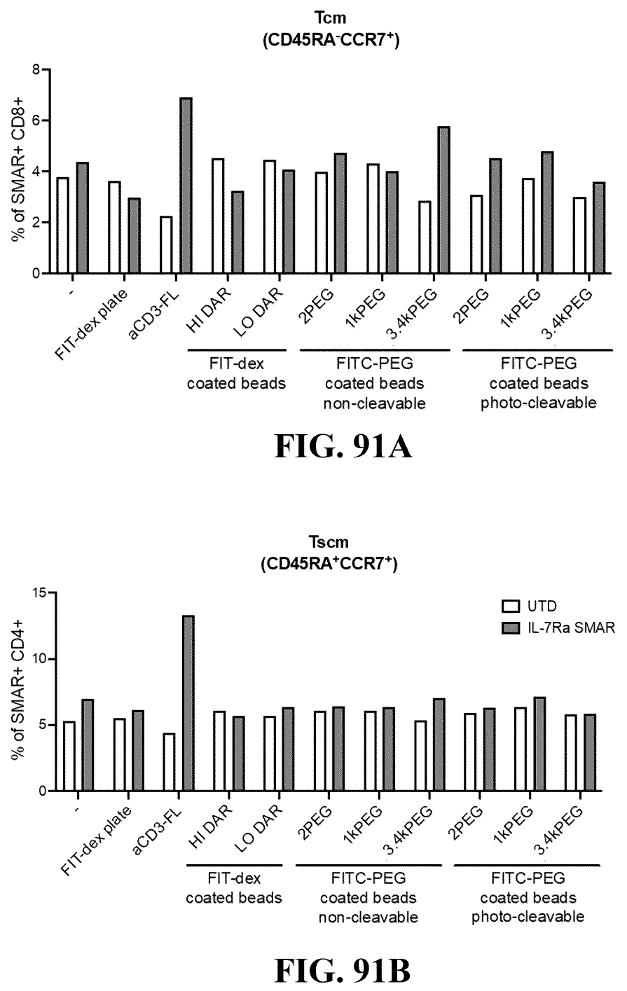

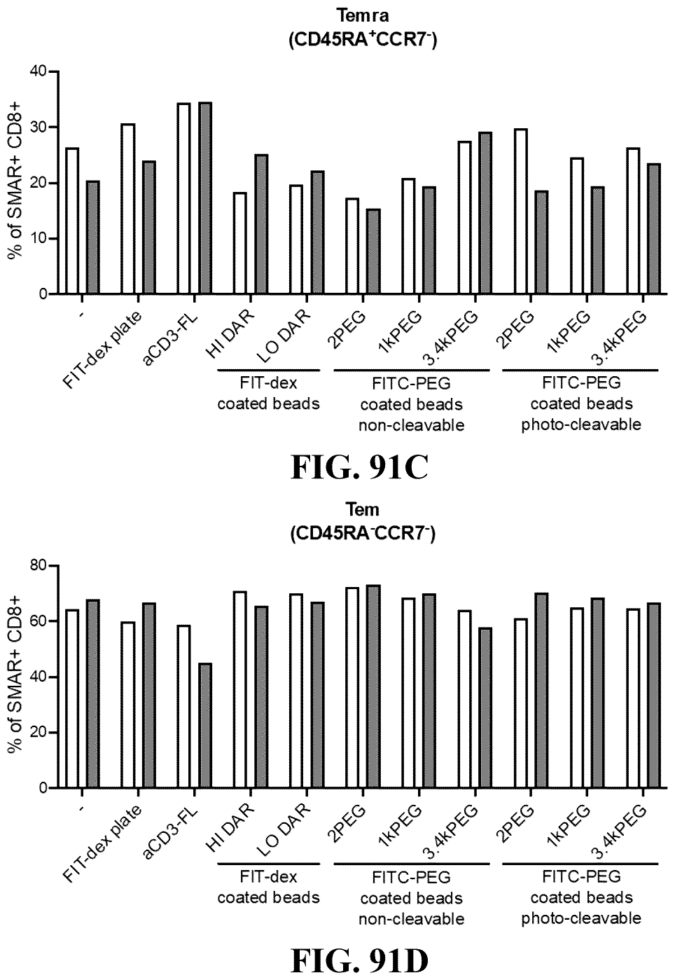

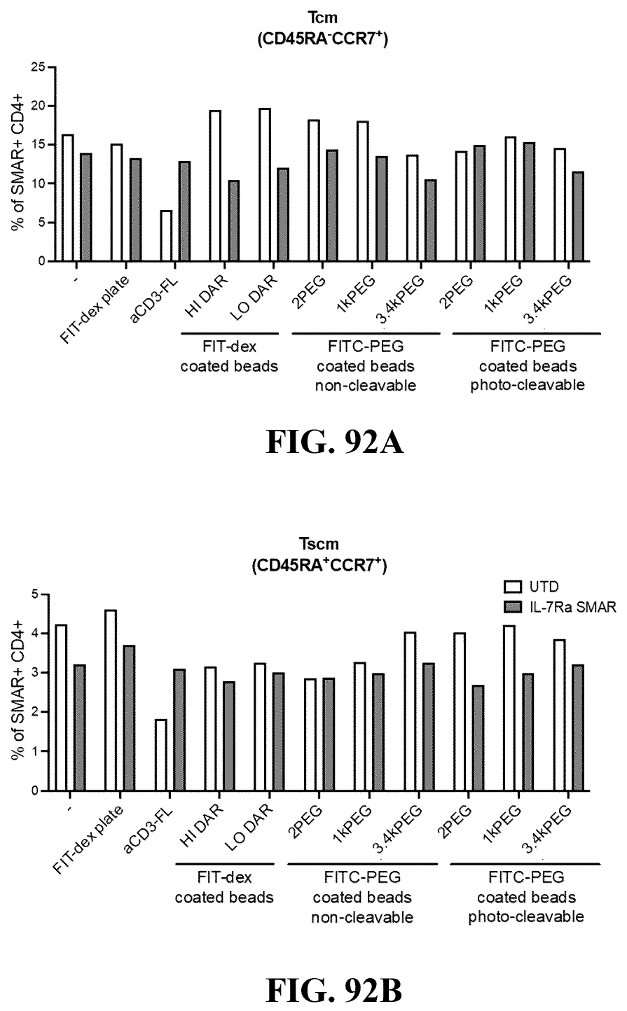

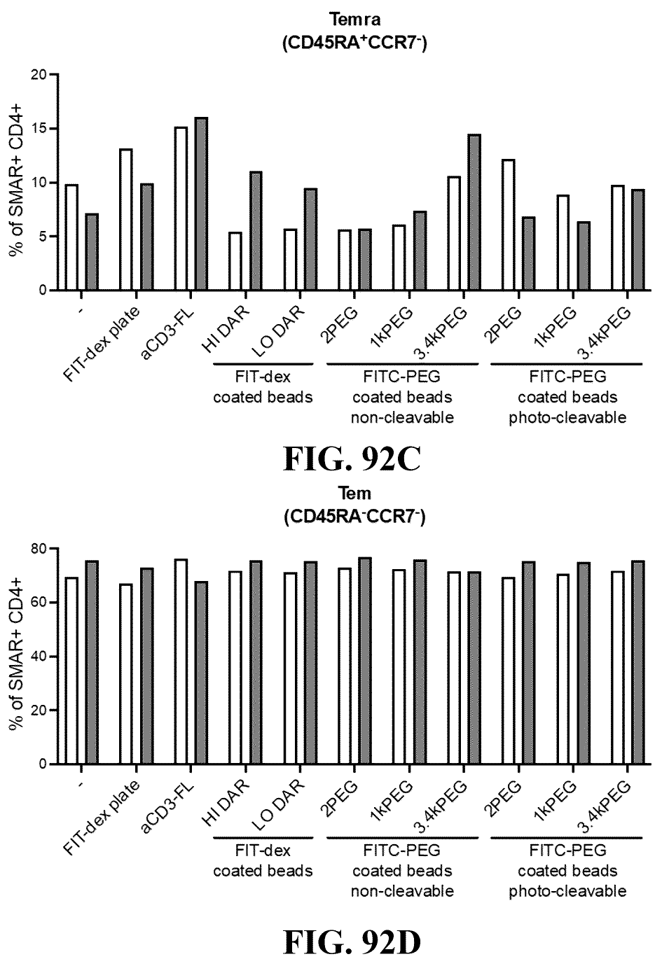

FIGS. 89A-89D illustrate cell phenotypes in SMAR+CD8+ cells (FITC-coated beads were used at bead-to-cell ratio of 5:1), FIGS. 90A-90D illustrate cell phenotypes in SMAR+CD4+ cells (FITC-coated beads were used at bead-to-cell ratio of 5:1), FIGS. 91A-91D illustrate cell phenotypes in SMAR+CD8+ cells (FITC-coated beads were used at bead-to-cell ratio of 1:1), and FIGS. 92A-92D illustrate cell phenotypes in SMAR+CD4+ cells (FITC-coated beads were used at bead-to-cell ratio of 1:1). These results show that FITC-PEG-conjugated Dynabeads activated the IL7Rα SMAR, resulting in an increase in Tₑₘ and T_{scm} memory populations, and a reduction of Tₑₘᵣₐ terminally differentiated effector cells within the CD8+ SMAR+ T-cell population. The FITC-PEG-conjugated Dynabeads and anti-CD3-FL antibody produced improved SMAR activation and memory phenotype shifts towards Tom and Tₛₑₘ compared to the FITC-dextran-coated plates. SMAR activation also improved memory phenotype of CD8+ T-cells more significantly than CD4+ T-cells. An improved effect was seen when using a 5:1 bead-to-cell ratio compared to a 1:1 bead-to-cell ratio.

### IX. Additional Examples

Additional examples of aspects of the present technology are described below as numbered clauses (1, 2, 3, etc.) for convenience. These are provided as examples and do not limit the subject technology.
1. A method of activating an immune cell population, the method comprising:
   providing an immune cell population expressing an engineered cytokine receptor switch comprising an activator binding domain configured to bind to an activator, a transmembrane domain, and an intracellular domain;
   contacting the immune cell population to a substrate comprising the activator, thereby activating the engineered cytokine receptor switch; and
   converting at least a portion of the immune cell population to an effector phenotype, a memory phenotype, or a combination thereof, thereby activating the immune cell population.
2. The method of Clause 1, wherein the activator binding domain comprises a single-chain variable fragment (scFv), a peptide, or a nanobody.
3. The method of Clause 2, wherein the peptide comprises a molecular weight from 1 kDa to 10 kDa.
4. The method of any one of Clauses 1-3, wherein the activator binding domain comprises an scFv.
5. The method of any one of Clauses 1-4, wherein the activator binding domain is humanized.
6. The method of any one of Clauses 1-5, wherein the activator binding domain comprises a binding affinity of between 10 μM and 1 fM for the activator.
7. The method of any one of Clauses 1-6, wherein the activator binding domain comprises a sequence having at least 80%, at least 90%, at least 95%, at least 97%, or 100% sequence identity to SEQ ID NO: 21.
8. The method of any one of Clauses 1-7, wherein the activator binding domain comprises a sequence of SEQ ID NO: 21.
9. The method of any one of Clauses 1-8, wherein the intracellular domain comprises a cytokine receptor intracellular domain.
10. The method of any one of Clauses 1-9, wherein the intracellular domain is derived from an endogenous cytokine receptor intracellular domain.
11. The method of Clause 10, wherein the endogenous cytokine receptor intracellular domain is an IL2Rα intracellular domain, an IL2Rß intracellular domain, an IL2Rγ intracellular domain, an IL4Rα intracellular domain, an IL7Rα intracellular domain, an IL9Rα intracellular domain, an IL15Rα intracellular domain, or an IL21Rα intracellular domain.
12. The method of Clause 10 or 11, wherein the intracellular domain comprises a sequence having at least 80%, at least 90%, at least 95%, at least 97%, or 100% sequence identity to the endogenous cytokine receptor intracellular domain.
13. The method of any one of Clauses 1-12, wherein the intracellular domain comprises a sequence having at least 80%, at least 90%, at least 95%, at least 97%, or 100% sequence identity to any one of SEQ ID NO: 29-SEQ ID NO: 34.
14. The method of any one of Clauses 1-13, wherein the intracellular domain comprises a sequence of any one of SEQ ID NO: 29-SEQ ID NO: 34.
15. The method of any one of Clauses 1-14, wherein the intracellular domain comprises or is derived from an intracellular domain of any of the following: IL2Rα, IL2Rß, IL2Rγ, IL4Rα, IL7Rα, IL15Rα, IL21Rα, ILIR, CD123, CD124, IL5Rα, IL5Rß, CD126, CD132, CD129, IL11Rα, IL12Rß1, IL12Rß2, IL13Rα1, CD122, IL18R, IL23R, IL27Rα, CD130, or GM-CSF.
16. The method of any one of Clauses 1-15, wherein the intracellular domain comprises a single intracellular domain.
17. The method of any one of Clauses 1-15, wherein the intracellular domain comprises a plurality of intracellular domains in tandem.
18. The method of any one of Clauses 1-17, wherein the activator binding domain, the intracellular domain, and the transmembrane domain are a single expression construct.
19. The method of any one of Clauses 1-18, wherein the transmembrane domain connects the activator binding domain and the intracellular domain.
20 The method of any one of Clauses 1-19, wherein the transmembrane domain is a cytokine receptor transmembrane domain.
21. The method of any one of Clauses 1-20, wherein the transmembrane domain is derived from an endogenous cytokine receptor transmembrane domain.
22. The method of Clause 21, wherein the endogenous cytokine receptor transmembrane domain is an IL2Rα transmembrane domain, an IL2Rß transmembrane domain, an IL2Rγ transmembrane domain, an IL4Rα transmembrane domain, an IL7Rα transmembrane domain, an IL9Rα transmembrane domain, an IL15Rα transmembrane domain, or an IL21Rα transmembrane domain.
23. The method of Clause 21 or 22, wherein the transmembrane domain comprises a sequence having at least 80%, at least 90%, at least 95%, at least 97%, or 100% sequence identity to the endogenous cytokine receptor transmembrane domain.
24. The method of any one of Clauses 1-23, wherein the transmembrane domain comprises a sequence having at least 80%, at least 90%, at least 95%, at least 97%, or 100% sequence identity to any one of SEQ ID NO: 23-SEQ ID NO: 28.
25. The method of any one of Clauses 1-24, wherein the transmembrane domain comprises a sequence of any one of SEQ ID NO: 23-SEQ ID NO: 28.
26. The method of any one of Clauses 1-25, wherein the transmembrane domain comprises or is derived from a transmembrane domain of any of the following: IL2Rα, IL2Rß, IL2Rγ, IL4Rα, IL7Rα, IL15Rα, IL21Rα, ILIR, CD123, CD124, IL5Rα, IL5Rß, CD126, CD132, CD129, IL11Rα, IL12Rß1, IL12Rß2, IL13Rα1, CD122, IL18R, IL23R, IL27Rα, CD130, an immunoglobulin, CD8, CD28, GM-CSF, or EpoR.
27 The method of any one of Clauses 1-26, wherein the engineered cytokine receptor switch further comprises a hinge domain.
28. The method of Clause 27, wherein the hinge domain is derived from an endogenous hinge domain.
29. The method of Clause 28, wherein the endogenous hinge domain is a CD8 hinge, a CD3 hinge, a CD4 hinge, or a CD28 hinge.
30. The method of Clause 28 or 29, wherein the hinge domain comprises a sequence having at least 80%, at least 90%, at least 95%, at least 97%, or 100% sequence identity to the endogenous hinge domain.
31. The method of any one of Clauses 27-30, wherein the hinge domain comprises a sequence having at least 80%, at least 90%, at least 95%, at least 97%, or 100% sequence identity to SEQ ID NO: 22.
32. The method of any one of Clauses 27-31, wherein the hinge domain comprises a sequence of SEQ ID NO: 22.
33 The method of any one of Clauses 27-32, wherein the hinge domain comprises or is derived from a hinge domain of any of the following: CD8, CD3, CD4, CD28, 4-1BB, CD28, OX40, ICOS, CD27, an immunoglobulin, or EpoR.
34. The method of any one of Clauses 27-33, wherein the hinge domain has a length greater than or equal to 1 amino acid, 2 amino acids, 3 amino acids, 4 amino acids, 5 amino acids, 6 amino acids, 7 amino acids, 8 amino acids, 9 amino acids, 10 amino acids, 15 amino acids, 20 amino acids, 25 amino acids, 30 amino acids, 35 amino acids, 40 amino acids, 45 amino acids, or 50 amino acids.
35. The method of any one of Clauses 27-34, wherein the hinge domain has a length less than or equal to 50 amino acids, 45 amino acids, 40 amino acids, 30 amino acids, 25 amino acids, 20 amino acids, 15 amino acids, 10 amino acids, 9 amino acids, 8 amino acids, 7 amino acids, 6 amino acids, 5 amino acids, 4 amino acids, 3 amino acids, 2 amino acids, or 1 amino acid.
36. The method of any one of Clauses 1-35, wherein the engineered cytokine receptor switch further comprises a signal peptide.
37. The method of Clause 36, wherein the signal peptide is a cytokine receptor signal peptide.
38. The method of Clause 36 or 37, wherein the signal peptide is derived from an endogenous cytokine signal peptide.
39. The method of Clause 38, wherein the endogenous cytokine receptor signal peptide is an IL2Rα signal peptide, an IL2Rß signal peptide, an IL2Rγ signal peptide, an IL4Rα signal peptide, an IL7Rα signal peptide, an IL9Rα signal peptide, an IL15Rα signal peptide, or an IL21Rα signal peptide.
40. The method of Clause 38 or 39, wherein the signal peptide comprises a sequence having at least 80%, at least 90%, at least 95%, at least 97%, or 100% sequence identity to the endogenous cytokine receptor signal peptide.
41. The method of any one of Clauses 36-40, wherein the signal peptide is an endoplasmic reticulum localization tag.
42. The method of any one of Clauses 36-41, wherein the signal peptide comprises a sequence having at least 80%, at least 90%, at least 95%, at least 97%, or 100% sequence identity to any one of SEQ ID NO: 15-SEQ ID NO: 20.
43. The method of any one of Clauses 36-42, wherein the signal peptide comprises a sequence of any one of SEQ ID NO: 15-SEQ ID NO: 20.
44. The method of any one of Clauses 36-43, wherein the signal peptide comprises or is derived from a signal peptide of any of the following: IL2Rα, IL2Rß, IL2Rγ, IL4Rα, IL7Rα, IL15Rα, IL21Rα, ILIR, CD123, CD124, IL5Rα, IL5Rß, CD126, CD132, CD129, IL11Rα, IL12Rß1, IL12Rß2, IL13Rα1, CD122, IL18R, IL23R, IL27Rα, CD130, an immunoglobulin, CD8, CD28, or GM-CSF.
45. The method of any one of Clauses 1-44, wherein providing the immune cell population comprises obtaining the immune cell population through leukapheresis, bone marrow biopsy, or a combination thereof.
46. The method of any one of Clauses 1-45, wherein prior to contacting the immune cell population to the substrate, the immune cell population comprises at least 20% naïve T-cells and naïve B-cells as a percentage of total cell population.
47. The method of any one of Clauses 1-46, further comprising transfecting the immune cell population with a vector encoding the engineered cytokine receptor switch.
48. The method of Clause 47, wherein the vector encoding the engineered cytokine receptor switch is a viral vector.
49. The method of Clause 47 or 48, wherein the vector encoding the engineered cytokine receptor switch comprises a sequence having at least 80%, at least 90%, at least 95%, at least 97%, or 100% sequence identity to any one of SEQ ID NO: 8-SEQ ID NO: 14.
50 The method of any one of Clauses 47-49, wherein the vector encoding the engineered cytokine receptor switch comprises a sequence of any one of SEQ ID NO: 8-SEQ ID NO: 14.
51. The method of any one of Clauses 1-50, further comprising transfecting the immune cell population with a vector encoding a chimeric antigen receptor.
52 The method of Clause 51, wherein the vector encoding the chimeric antigen receptor is a viral vector.
53. The method of Clause 51 or 52, wherein the vector encoding the chimeric antigen receptor comprises a sequence having at least 80%, at least 90%, at least 95%, at least 97%, or 100% sequence identity to any one of SEQ ID NO: 40-SEQ ID NO: 45.
54 The method of any one of Clauses 51-53, wherein the vector encoding the chimeric antigen receptor comprises a sequence of any one of SEQ ID NO: 40-SEQ ID NO: 45.
55. The method of any one of Clauses 51-54, wherein a single vector encodes the chimeric antigen receptor and the engineered cytokine receptor switch.
56. The method of Clause 55, wherein the single vector comprises a sequence having at least 80%, at least 90%, at least 95%, at least 97%, or 100% sequence identity to any one of SEQ ID NO: 46-SEQ ID NO: 52.
57. The method of Clause 55 or 56, wherein the single vector comprises a sequence of any one of SEQ ID NO: 46-SEQ ID NO: 52.
58. The method of any one of Clauses 47-57, wherein the viral vector encoding the engineered cytokine receptor switch, the viral vector encoding the chimeric antigen receptor, or a combination thereof is a lentiviral vector, an adeno-associated viral vector, a vaccinia viral vector, a poxvirus viral vector, a herpes viral vector, an alphavirus viral vector, gamma retrovirus, a polyoma viral vector, or a combination thereof.
59. The method of any one of Clauses 47-58, wherein the viral vector encoding the engineered cytokine receptor switch, the viral vector encoding the chimeric antigen receptor, or a combination thereof is a gamma retrovirus, an adeno-associated viral vector, a lentiviral vector, or a combination thereof.
60. The method of Clause 58 or 59, wherein the viral vector encoding the engineered cytokine receptor switch, the viral vector encoding the chimeric antigen receptor, or a combination thereof has a titer of between about 10⁶ and about 109 per ml.
61. The method of any one of Clauses 47-60, wherein the immune cell population has a density of between about 10⁴ and about 10⁷ cells/ml during the transfecting.
62. The method of any one of Clauses 1-61, wherein the activator is non-immunogenic.
63. The method of any one of Clauses 1-62, wherein the activator is an exogenous activator.
64. The method of any one of Clauses 1-63, wherein the activator has low toxicity.
65. The method of any one of Clauses 1-64, wherein the activator has low cross-reactivity.
66. The method of any one of Clauses 1-65, wherein the activator is a small molecule, a peptide, or an oligonucleotide.
67. The method of any one of Clauses 1-66 wherein the activator is selected from the group consisting of fluorescein, fluorescein isothiocyanate (FITC), fluorescein 5-maleimide, fluorescein-5-carboxamide, fluorescein-6-carboxamide, 6-FAM phosphoramidite, topiramate hemisuccinate, creatine, acetaminophen, ketamine, propofol, lidocaine, ractopamine, salicylate, salicylic acid, sulfasalazine, dapsone, albendazole, ivermectin, levamisole, permethrin, pyrantel, thiabendazole, procainamide, sulfamethazine, amikacin, amoxicillin, ampicillin, cefazolin, cefuroxime, cephalexin, chloramphenicol, chloramphenicol, ciprofloxacin, clenbuterol, cloxacillin, colistin A, dicloxacillin, enrofloxacin, furaltadone, gentamicin, gentamicin, kanamycin, kanamycin, kincomycin, lincomycin, metronidazole, nafcillin, nalidixic acid, neomycin, neomycin, nitrofurazone, norfloxacin, ofloxacin, oxacillin, spectinomycin, streptomycin, streptomycin, sulfabenzamide, sulfacetamide, sulfadiazine, sulfadimidine, sulfametoxydiazine, sulfanilamide, trimethoprim, carbamazepine, ethosuximide, lamotrigine, primidone, cetirizine, chlorpheniramine, diphenhydramine, doxylamine, promethazine, sulfadimethoxine, benzothiazinone, butylated hydroxytoluene, tripelennamine, chlorpromazine, clozapine, haloperidol, olanzapine, paliperidone, quetiapine, ribavirin, meprobamate, acebutolol, atenolol, penbutolol, warfarin, salmeterol, aflatoxin B1, tetraxetan (DOTA), 4-[(6-methylpyrazin-2-yl)oxy]benzoate (MPOB), biotin, melamine, methotrexate, amphetamine, diethylpropion, dextromethorphan, pseudoephedrine, dihydrochlorothiazide, hydrochlorothiazide, clonazepam, diazepam, nitrazepam, rhodamine B, fluorescent brightener Ksn, zearalenone, Sudan Red1, acetominophen, acrylamide, benzoic acid, benzophenone, benzothiazine, mercaptobenzothiazole, erythrosine, Sudan, tartrazine, erythromycin, sirolimus, atropine, ethyl glucuronide, aflatoxin M1, methocarbamol, fentanyl, hydromorphone, morphine, remifentanil, tapentadol, tramadol, pregabalin, gabapentin, amitriptyline, desipramine, imipramine, nortriptyline, venlafaxine, dinitrophenyl, His-tag, PEG methoxy group, etodolac, ibuprofen, ketoprofen, meclofenamic acid, phenylbutazone, acetyl salicylic acid, acetamiprid, acetochlor, carbadazim, carbaryl, chlorothalonil, chlorpyrifos, fenpropathrin, imazalil, imidacloprid, parathion, abscisic acid, dibutyl phthalate, clonazepam, lorazepam, oxazepam, phenobarbital, secobarbital, zaleplon, zolpidem, trazodone, fluoxetine, fluvoxamine, cortisone, dexamethasone, dihydrotestosterone, fluocinolone, methylprednisolone, prednisolone, stanozolol, triamcinolone, mazindol, methamphetamine, methylphenidate, modafinil, chrysoidine, deoxynivalenol, fumonisin, microcystin Lr, ochratoxin, sterigmatocystin, T-2 toxin, sildenafil, tadalafil, scopolamine, florfenicol, pirlimycin, and sulfaquinoxaline.
68. The method of any one of Clauses 1-67, wherein the activator is a small molecule.
69. The method of any one of Clauses 66-68, wherein the small molecule comprises fluorescein, tetraxetan, butylated hydroxytoluene, Rhodamine B, benzoic acid, erythrosine, tartrazine, PEG methoxy group, dinitrophenyl (DNP), and polyhistidine tags (e.g., His6), a derivative thereof, or a combination thereof.
70. The method of any one of Clauses 66-69, wherein the small molecule comprises fluorescein, tetraxetan, a derivative thereof, or a combination thereof.
71. The method of any one of Clauses 1-70, wherein the substrate comprises a nanoparticle, a microparticle, a polymer matrix, a surface, a carbon nanomaterial, a quantum dot, or a combination thereof.
72. The method of Clause 71, wherein a ratio of the nanoparticle, the microparticle, the polymer matrix, the quantum dot, or a combination thereof to cells of the immune cell population is between about 10:1 and about 1:10.
73. The method of any one of Clauses 1-72, wherein the activator has a density of between about 10³ and about 2×10⁶ molecules per μm² of a surface of the substrate.
74. The method of any one of Clauses 1-73, wherein the activator is coupled to the substrate at an activator-to-substrate ratio greater than or equal to 1, 1.5, 2, 2.5, 3, 3.5, 4, 4.5, 5, 5.5, 6, 6.5, 7, 7.5, 8, 8.5, 9, 9.5, 10, 15, 20, 30, 40, 50, 60, 70, 80, 90, or 100.
75. The method of any one of Clauses 1-74, wherein the activator is coupled to the substrate at an activator-to-substrate ratio less than or equal to 100, 90, 80, 70, 60, 50, 40, 30, 20, 15, 10, 9.5, 9, 8.5, 8, 7.5, 7, 6.5, 6, 5.5, 5, 4.5, 4, 3.5, 3, 2.5, 2, 1.5, or 1.
76. The method of any one of Clauses 1-75, wherein the activator is coupled to the substrate by a linker.
77. The method of Clause 76, wherein the linker comprises a length greater than its persistence length.
78. The method of Clause 76 or 77, wherein the linker has a length of between about 5 and about 100 nm.
79. The method of any one of Clauses 76-78, wherein the linker comprises at least 2 repeating units, 5 repeating units, 10 repeating units, 15 repeating units, 20 repeating units, 25 repeating units, 30 repeating units, 35 repeating units, 40 repeating units, 45 repeating units, 50 repeating units, 55 repeating units, 60 repeating units, 65 repeating units, 70 repeating units, 75 repeating units, 80 repeating units, 85 repeating units, 90 repeating units, 95 repeating units, 100 repeating units, 110 repeating units, 120 repeating units, 130 repeating units, 140 repeating units, 150 repeating units, 160 repeating units, 170 repeating units, 180 repeating units, 190 repeating units, 200 repeating units, 500 repeating units, 1000 repeating units, or 2000 repeating units.
80. The method of any one of Clauses 76-79, wherein the linker comprises no more than 2000 repeating units, 1000 repeating units, 500 repeating units, 200 repeating units, 190 repeating units, 180 repeating units, 170 repeating units, 160 repeating units, 150 repeating units, 140 repeating units, 130 repeating units, 120 repeating units, 110 repeating units, 100 repeating units, 95 repeating units, 90 repeating units, 85 repeating units, 80 repeating units, 75 repeating units, 70 repeating units, 65 repeating units, 60 repeating units, 55 repeating units, 50 repeating units, 45 repeating units, 40 repeating units, 35 repeating units, 30 repeating units, 25 repeating units, 20 repeating units, 15 repeating units, 10 repeating units, 5 repeating units or 2 repeating units.
81. The method of any one of Clauses 76-80, wherein the linker is a cleavable linker.
82. The method of Clause 81, wherein the cleavable linker is a photocleavable linker, a chemically cleavable linker, or an enzymatically cleavable linker.
83 The method of Clause 81 or 82, further comprising cleaving the cleavable linker to release the immune cell population from the substrate.
84 The method of any one of Clauses 76-80, wherein the linker is a non-cleavable linker.
85. The method of any one of Clauses 76-84, wherein the linker comprises polyethylene glycol.
86. The method of any one of Clauses 1-85, further comprising expanding the immune cell population by between about 5 and about 1000-fold during the contacting.
87. The method of any one of Clauses 1-86, wherein a ratio of the effector phenotype and the memory phenotype is between 4:1 and 1:4.
88. The method of any one of Clauses 1-87, wherein at least 80% of the immune cell population is converted to the memory phenotype.
89. The method of any one of Clauses 1-88, wherein following the converting, between about 40% and about 85% of T-cells of the immune cell population are central memory
cells.
90 The method of any one of Clauses 1-89, wherein following the converting, between about 3% and about 30% of T-cells of the immune cell population are stem memory T-cells.
91. The method of any one of Clauses 1-90, wherein following the converting, between about 5% and about 35% of T-cells of the immune cell population are effector memory T-cells.
92. The method of any one of Clauses 1-91, wherein following the converting, between about 0.5% and about 20% of T-cells of the immune cell population are terminally differentiated effector memory T-cells.
93. The method of any one of Clauses 1-92, wherein at least 80% of lymphocytes of the immune cell population are converted to the effector phenotype.
94. The method of any one of Clauses 1-93, wherein the memory phenotype is a stem-cell memory phenotype, a central memory phenotype, a transitional memory phenotype, an effector memory phenotype, a terminal effector memory phenotype, or a combination thereof.
95. The method of any one of Clauses 1-94, further comprising contacting the immune cell population with a stimulating agonist comprising a granulocyte macrophage-colony stimulating factor (GM-CSF), stem cell factor (SCF), interleukin-1 (IL1), interleukin-2 (IL2), interleukin-3 (IL3), a CD3 agonist, a CD4 agonist, a CD8 agonist, a CD16 agonist, a CD23 agonist, a CD28 agonist, a CD47 agonist, a CD80 agonist, a CD113 agonist, a CD131 agonist, a CD137 agonist, an HLA-E agonist, a 41BBL agonist, or a combination of agonists thereof.
96. The method of Clause 95, comprising contacting the immune cell population with the CD3 agonist.
97. The method of Clause 95 or 96, wherein the CD3 agonist comprises an antigen.
98. The method of any one of Clauses 95-97, wherein a ratio of the CD3 agonist and the activator is between about 10:1 and about 1:10.
99. The method of any one of Clauses 95-98, comprising contacting the immune cell population with the CD23 agonist, the CD28 agonist, or a combination thereof.
100. The method of any one of Clauses 95-99, comprising contacting the immune cell population with the IL2.
101. The method of any one of Clauses 95-100, comprising about 500 to about 5000 units of the IL2.
102. The method of any one of Clauses 95-101, wherein the stimulating agonist is coupled to the substrate.
103. The method of any one of Clauses 95-102, wherein the stimulating agonist is coupled to a second substrate.
104. The method of any one of Clauses 95-103, wherein contacting the immune cell population with the stimulating agonist is before contacting the immune cell population to the substrate, concurrent with contacting the immune cell population to the substrate, or a combination thereof.
105. The method of any one of Clauses 1-104, further comprising separating the immune cell population from cells which do not express the engineered cytokine receptor switch.
106. The method of Clause 105, wherein the separating comprises affinity purification.
107. The method of Clause 106, wherein the affinity purification comprises binding the immune cell population to the substrate, and separating unbound cells from the substrate.
108. The method of Clause 106 or 107, wherein the affinity purification comprises binding the immune cell population to a third substrate functionalized with the activator, and separating unbound cells from the substrate.
109. The method of any one of Clauses 1-108, wherein the method is completed within about 5 to about 10 days.
110. The method of any one of Clauses 1-109, wherein the cytokine receptor switch comprises a sequence having at least 80%, at least 90%, at least 95%, at least 97%, or 100% sequence identity to any one of SEQ ID NO: 1-SEQ ID NO: 7.
111. The method of any one of Clauses 1-110, wherein the cytokine receptor switch comprises a sequence of any one of SEQ ID NO: 1-SEQ ID NO: 7.
112. An immune cell population generated according to the method of any one of Clauses 1-111.
113. A composition comprising the immune cell population of Clause 112.
114. A method for treating a subject, the method comprising administering the immune cell population of Clause 112 or the composition of Clause 113 to the subject.
115. The method of Clause 114, wherein the administering treats a cancer in the subject.
116. The method of Clause 114 or 115, wherein between about 10⁵ and about 5×10⁷ immune cells of the immune cell population are administered to the subject.
117. The method of any one of Clauses 114-116, wherein between about 5×10⁴ and about 3×10⁷ memory T-cells and memory B-cells of the immune cell population are administered to the subject.
118. The method of any one of Clauses 114-117, wherein between about 5×10⁴ and about 3×10⁷ effector T-cells and effector B-cells of the immune cell population are administered to the subject.
119. The method of any one of Clauses 114-118, further comprising recruiting an immune cell of the immune cell population to a cancer cell by binding a chimeric antigen receptor expressed by the immune cell to a target antigen on the cancer cell.
120. The method of any one of Clauses 114-118, further comprising recruiting the immune cell to a cancer cell by administering a bispecific agent to the subject, wherein the bispecific agent comprises a targeting moiety that binds to a target antigen on the cancer cell and a synthetic antigen, and wherein a chimeric antigen receptor expressed by the immune cell binds to the synthetic antigen.
121. The method of Clause 119 or 120, further comprising activating an immune response against the cancer cell.
122. The method of any one of Clauses 119-121, further comprising killing the cancer cell.
123. The method of any one of Clauses 115-122, wherein the cancer is acute myeloid leukemia, multiple myeloma, ovarian cancer, mesothelioma, non-Hodgkin lymphoma, acute lymphoblastic leukemia, mantle cell lymphoma, follicular lymphoma, glioma, pancreatic cancer, prostate cancer, or gastric cancer.
124. The method of any one of Clauses 119-123, wherein the target antigen comprises CD19, CD20, CD22, CD123, CD33, CD3, CD4, CD8, CD38, SLAMF7, BCMA, GD2, GPRC5D, MUC16, HER2, EGFR, EGFRVIII, CLL-1, CD44v6, folate receptor-α, B7-H3, EphA2, GRP78, NKG2D, CD70, or mesothelin.
125. The method of any one of Clauses 115-124, wherein treating the cancer comprises preventing recurrence of the cancer.
126. The method of any one of Clauses 114-125, wherein an immune cell of the immune cell population persists in the subject for at least 1 week, at least 1 month, at least 1 year, or at least 10 years.
127. The method of any one of Clauses 114-126, wherein an immune cell of the immune cell population persists in the subject at least 2 times, at least 5 times, or at least 10 times as long as an immune cell that does not express the engineered cytokine receptor switch.
128. The method of any one of Clauses 114-127, wherein 4 weeks after the administering, the immune cell population comprises at least 5%, at least 10%, at least 15%, at least 20%, at least about 25%, at least about 30%, at least about 40%, or at least about 50% as many cells as the immune cell population at a time of administration.
129. A method for treating a subject, the method comprising administering a first dose of immune cells comprising a greater number of effector phenotype immune cells than memory phenotype immune cells, and administering a second dose of engineered immune cells comprising a greater number of memory phenotype immune cells than effector phenotype immune cells.
130. The method of Clause 129, wherein the first dose of engineered immune cells and the second dose of engineered immune cells express an engineered cytokine receptor switch.
131. The method of Clause 129 or 130, wherein the second dose of engineered immune cells is administered at least 1 day after the first dose of engineered immune cells.
132. A composition comprising:
   an immune cell population, wherein the immune cell population comprises immune cells expressing a cytokine receptor switch comprising:
   an activator binding domain configured to bind an activator,
   a signal peptide,
   a hinge domain,
   a transmembrane domain, and
   an intracellular domain, wherein the intracellular domain comprises a cytokine receptor intracellular domain; and
an activator coupled to a substrate.
133. The composition of Clause 132, wherein at least 30%, at least 40%, or at least 50% of the immune cell population has a memory phenotype.
134. The composition of Clause 132 or 133, wherein not less than 20% and not more than 90%, not less than 30% and not more than 80%, not less than 40% and not more than 70% of the immune cell population has a memory phenotype.
135. The composition of any one of Clauses 132-134, wherein at least 10% of the immune cell population has an effector phenotype.
136. The composition of any one of Clauses 132-135, wherein at least 20%, at least 30%, at least 40%, at least 50%, or at least 60% of the immune cell population has an effector phenotype.
137. The composition of Clause 135 or 136, wherein immune cells having the memory phenotype persist for longer than immune cells having the effector phenotype.
138. The composition of any one of Clauses 135-137, wherein immune cells having the effector phenotype have a stronger anti-tumor potency than immune cells having the memory phenotype.
139. The composition of any one of Clauses 132-138, wherein the activator binding domain comprises a single-chain variable fragment (scFv), a peptide, or a nanobody.
140. The composition of Clause 139, wherein the peptide comprises a molecular weight of from 1 kDa to 10 kDa.
141. The composition of any one of Clauses 132-140, wherein the activator binding domain comprises an scFv.
142. The composition of any one of Clauses 132-141, wherein the activator binding domain is humanized.
143. The composition of any one of Clauses 132-142, wherein the activator binding domain comprises a binding affinity of between 10 μM and 1 fM for the activator.
144. The composition of any one of Clauses 132-143, wherein the activator binding domain comprises a sequence having at least 80%, at least 90%, at least 95%, at least 97%, or 100% sequence identity to SEQ ID NO: 21.
145. The composition of any one of Clauses 132-144, wherein the activator binding domain comprises a sequence of SEQ ID NO: 21.
146. The composition of any one of Clauses 132-145, wherein the intracellular domain comprises a cytokine receptor intracellular domain.
147. The composition of any one of Clauses 132-146, wherein the intracellular domain is derived from an endogenous cytokine receptor intracellular domain.
148. The composition of Clause 147, wherein the endogenous cytokine receptor intracellular domain is an IL2Rα intracellular domain, an IL2Rß intracellular domain, an IL2Rγ intracellular domain, an IL4Rα intracellular domain, an IL7Rα intracellular domain, an IL9Rα intracellular domain, an IL15Rα intracellular domain, or an IL21Rα intracellular domain.
149. The composition of Clause 147 or 148, wherein the intracellular domain comprises a sequence having at least 80%, at least 90%, at least 95%, at least 97%, or 100% sequence identity to the endogenous cytokine receptor intracellular domain.
150. The composition of any one of Clauses 132-149, wherein the intracellular domain comprises a sequence having at least 80%, at least 90%, at least 95%, at least 97%, or 100% sequence identity to any one of SEQ ID NO: 29-SEQ ID NO: 34.
151. The composition of any one of Clauses 132-150, wherein the intracellular domain comprises a sequence of any one of SEQ ID NO: 29-SEQ ID NO: 34.
152. The composition of any one of Clauses 132-151, wherein the intracellular domain comprises or is derived from an intracellular domain of any of the following: IL2Rα, IL2Rß, IL2Rγ, IL4Rα, IL7Rα, IL15Rα, IL21Rα, ILIR, CD123, CD124, IL5Rα, IL5Rß, CD126, CD132, CD129, IL11Rα, IL12Rß1, IL12Rß2, IL13Rα1, CD122, IL18R, IL23R, IL27Rα, CD130, or GM-CSF.
153. The composition of any one of Clauses 132-152, wherein the intracellular domain comprises a single intracellular domain.
154. The composition of any one of Clauses 132-152, wherein the intracellular domain comprises a plurality of intracellular domains in tandem.
155. The composition of any one of Clauses 132-154, wherein the activator binding domain, the intracellular domain, and the transmembrane domain are a single expression construct.
156. The composition of any one of Clauses 132-155, wherein the transmembrane domain connects the activator binding domain and the intracellular domain.
157. The composition of any one of Clauses 132-156, wherein the transmembrane domain is a cytokine receptor transmembrane domain.
158. The composition of any one of Clauses 132-157, wherein the transmembrane domain is derived from an endogenous cytokine receptor transmembrane domain.
159. The composition of Clause 158, wherein the endogenous cytokine receptor transmembrane domain is an IL2Rα transmembrane domain, an IL2Rß transmembrane domain, an IL2Rγ transmembrane domain, an IL4Rα transmembrane domain, an IL7Rα transmembrane domain, an IL9Rα transmembrane domain, an IL15Rα transmembrane domain, or an IL21Rα transmembrane domain.
160. The composition of Clause 158 or 159, wherein the transmembrane domain comprises a sequence having at least 80%, at least 90%, at least 95%, at least 97%, or 100% sequence identity to the endogenous cytokine receptor transmembrane domain.
161. The composition of any one of Clauses 132-160, wherein the transmembrane domain comprises a sequence having at least 80%, at least 90%, at least 95%, at least 97%, or 100% sequence identity to any one of SEQ ID NO: 23-SEQ ID NO: 28.
162. The composition of any one of Clauses 132-161, wherein the transmembrane domain comprises a sequence of any one of SEQ ID NO: 23-SEQ ID NO: 28.
163. The composition of any one of Clauses 132-162, wherein the transmembrane domain comprises or is derived from a transmembrane domain of any of the following: IL2Rα, IL2Rß, IL2Rγ, IL4Rα, IL7Rα, IL15Rα, IL21Rα, ILIR, CD123, CD124, IL5Rα, IL5Rß, CD126, CD132, CD129, IL11Rα, IL12Rß1, IL12Rß2, IL13Rα1, CD122, IL18R, IL23R, IL27Rα, CD130, an immunoglobulin, CD8, CD28, GM-CSF, or EpoR.
164. The composition of any one of Clauses 132-163, wherein the engineered cytokine receptor switch further comprises a hinge domain.
165. The composition of Clause 164, wherein the hinge domain is derived from an endogenous hinge domain.
166. The composition of Clause 165, wherein the endogenous hinge domain is a CD8 hinge, a CD3 hinge, a CD4 hinge, or a CD28 hinge.
167. The composition of Clause 165 or 166, wherein the hinge domain comprises a sequence having at least 80%, at least 90%, at least 95%, at least 97%, or 100% sequence identity to the endogenous hinge domain.
168. The composition of any one of Clauses 164-167, wherein the hinge domain comprises a sequence having at least 80%, at least 90%, at least 95%, at least 97%, or 100% sequence identity to SEQ ID NO: 22.
169. The composition of any one of Clauses 164-168, wherein the hinge domain comprises a sequence of SEQ ID NO: 22.
170. The composition of any one of Clauses 164-169, wherein the hinge domain comprises or is derived from a hinge domain of any of the following: CD8, CD3, CD4, CD28, 4-1BB, CD28, OX40, ICOS, CD27, an immunoglobulin, or EpoR.
171. The composition of any one of Clauses 164-170, wherein the hinge domain has a length greater than or equal to 1 amino acid, 2 amino acids, 3 amino acids, 4 amino acids, 5 amino acids, 6 amino acids, 7 amino acids, 8 amino acids, 9 amino acids, 10 amino acids, 15 amino acids, 20 amino acids, 25 amino acids, 30 amino acids, 35 amino acids, 40 amino acids, 45 amino acids, or 50 amino acids.
172. The composition of any one of Clauses 164-171, wherein the hinge domain has a length less than or equal to 50 amino acids, 45 amino acids, 40 amino acids, 30 amino acids, 25 amino acids, 20 amino acids, 15 amino acids, 10 amino acids, 9 amino acids, 8 amino acids, 7 amino acids, 6 amino acids, 5 amino acids, 4 amino acids, 3 amino acids, 2 amino acids, or 1 amino acid.
173. The composition of any one of Clauses 132-172, wherein the engineered cytokine receptor switch further comprises a signal peptide.
174. The composition of Clause 173, wherein the signal peptide is a cytokine receptor signal peptide.
175. The composition of Clause 173 or 174, wherein the signal peptide is derived from an endogenous cytokine signal peptide.
176. The composition of Clause 175, wherein the endogenous cytokine receptor signal peptide is an IL2Rα signal peptide, an IL2Rß signal peptide, an IL2Rγ signal peptide, an IL4Rα signal peptide, an IL7Rα signal peptide, an IL9Rα signal peptide, an IL15Rα signal peptide, or an IL21Rα signal peptide.
177. The composition of Clause 175 or 176, wherein the signal peptide comprises a sequence having at least 80%, at least 90%, at least 95%, at least 97%, or 100% sequence identity to the endogenous cytokine receptor signal peptide.
178. The composition of any one of Clauses 173-177, wherein the signal peptide is an endoplasmic reticulum localization tag.
179. The composition of any one of Clauses 173-178, wherein the signal peptide comprises a sequence having at least 80%, at least 90%, at least 95%, at least 97%, or 100% sequence identity to any one of SEQ ID NO: 15-SEQ ID NO: 20.
180. The composition of any one of Clauses 173-179, wherein the signal peptide comprises a sequence of any one of SEQ ID NO: 15-SEQ ID NO: 20.
181. The composition of any one of Clauses 173-180, wherein the signal peptide comprises or is derived from a signal peptide of any of the following: IL2Rα, IL2Rß, IL2RY, IL4Rα, IL7Rα, IL15Rα, IL21Rα, ILIR, CD123, CD124, IL5Rα, IL5Rß, CD126, CD132, CD129, IL11Rα, IL12Rß1, IL12Rß2, IL13Rα1, CD122, IL18R, IL23R, IL27Rα, CD130, an immunoglobulin, CD8, CD28, or GM-CSF.
182. The composition of any one of Clauses 132-181, wherein the activator is non-immunogenic.
183. The composition of any one of Clauses 132-182, wherein the activator is an exogenous activator.
184. The composition of any one of Clauses 132-183, wherein the activator has low toxicity.
185. The composition of any one of Clauses 132-184, wherein the activator has low cross-reactivity.
186. The composition of any one of Clauses 132-185, wherein the activator is a small molecule, a peptide, or an oligonucleotide.
187. The composition of any one of Clauses 132-186, wherein the activator is selected from the group consisting of fluorescein, fluorescein isothiocyanate (FITC), fluorescein 5-maleimide, fluorescein-5-carboxamide, fluorescein-6-carboxamide, 6-FAM phosphoramidite, topiramate hemisuccinate, creatine, acetaminophen, ketamine, propofol, lidocaine, ractopamine, salicylate, salicylic acid, sulfasalazine, dapsone, albendazole, ivermectin, levamisole, permethrin, pyrantel, thiabendazole, procainamide, sulfamethazine, amikacin, amoxicillin, ampicillin, cefazolin, cefuroxime, cephalexin, chloramphenicol, chloramphenicol, ciprofloxacin, clenbuterol, cloxacillin, colistin A, dicloxacillin, enrofloxacin, furaltadone, gentamicin, gentamicin, kanamycin, kanamycin, kincomycin, lincomycin, metronidazole, nafcillin, nalidixic acid, neomycin, neomycin, nitrofurazone, norfloxacin, ofloxacin, oxacillin, spectinomycin, streptomycin, streptomycin, sulfabenzamide, sulfacetamide, sulfadiazine, sulfadimidine, sulfametoxydiazine, sulfanilamide, trimethoprim, carbamazepine, ethosuximide, lamotrigine, primidone, cetirizine, chlorpheniramine, diphenhydramine, doxylamine, promethazine, sulfadimethoxine, benzothiazinone, butylated hydroxytoluene, tripelennamine, chlorpromazine, clozapine, haloperidol, olanzapine, paliperidone, quetiapine, ribavirin, meprobamate, acebutolol, atenolol, penbutolol, warfarin, salmeterol, aflatoxin B1, tetraxetan (DOTA), 4-[(6-methylpyrazin-2-yl)oxy]benzoate (MPOB), biotin, melamine, methotrexate, amphetamine, diethylpropion, dextromethorphan, pseudoephedrine, dihydrochlorothiazide, hydrochlorothiazide, clonazepam, diazepam, nitrazepam, rhodamine B, fluorescent brightener Ksn, zearalenone, Sudan Red1, acetominophen, acrylamide, benzoic acid, benzophenone, benzothiazine, mercaptobenzothiazole, erythrosine, Sudan, tartrazine, erythromycin, sirolimus, atropine, ethyl glucuronide, aflatoxin M1, methocarbamol, fentanyl, hydromorphone, morphine, remifentanil, tapentadol, tramadol, pregabalin, gabapentin, amitriptyline, desipramine, imipramine, nortriptyline, venlafaxine, dinitrophenyl, His-tag, PEG methoxy group, etodolac, ibuprofen, ketoprofen, meclofenamic acid, phenylbutazone, acetyl salicylic acid, acetamiprid, acetochlor, carbadazim, carbaryl, chlorothalonil, chlorpyrifos, fenpropathrin, imazalil, imidacloprid, parathion, abscisic acid, dibutyl phthalate, clonazepam, lorazepam, oxazepam, phenobarbital, secobarbital, zaleplon, zolpidem, trazodone, fluoxetine, fluvoxamine, cortisone, dexamethasone, dihydrotestosterone, fluocinolone, methylprednisolone, prednisolone, stanozolol, triamcinolone, mazindol, methamphetamine, methylphenidate, modafinil, chrysoidine, deoxynivalenol, fumonisin, microcystin Lr, ochratoxin, sterigmatocystin, T-2 toxin, sildenafil, tadalafil, scopolamine, florfenicol, pirlimycin, and sulfaquinoxaline.
188. The composition of any one of Clauses 132-187, wherein the activator is a small molecule.
189. The composition of any one of Clauses 186-188, wherein the small molecule comprises fluorescein, tetraxetan, butylated hydroxytoluene, Rhodamine B, benzoic acid, erythrosine, tartrazine, PEG methoxy group, dinitrophenyl (DNP), and polyhistidine tags (e.g., His6), a derivative thereof, or a combination thereof.
190. The composition of any one of Clauses 186-189, wherein the small molecule comprises fluorescein, tetraxetan, a derivative thereof, or a combination thereof.
191. The composition of any one of Clauses 132-190, wherein the substrate comprises a nanoparticle, a microparticle, a polymer matrix, a surface, a carbon nanomaterial, a quantum dot, or a combination thereof.
192. The composition of Clause 191, wherein a ratio of the nanoparticle, the microparticle, the polymer matrix, the quantum dot, or a combination thereof to cells of the immune cell population is between about 10:1 and about 1:10.
193. The composition of any one of Clauses 132-192, wherein the activator has a density of between about 10³ and about 2×10⁶ molecules per μm² of a surface of the substrate.
194. The composition of any one of Clauses 132-193, wherein the activator is coupled to the substrate at an activator-to-substrate ratio greater than or equal to 1, 1.5, 2, 2.5, 3, 3.5, 4, 4.5, 5, 5.5, 6, 6.5, 7, 7.5, 8, 8.5, 9, 9.5, 10, 15, 20, 30, 40, 50, 60, 70, 80, 90, or 100.
195. The composition of any one of Clauses 132-194, wherein the activator is coupled to the substrate at an activator-to-substrate ratio less than or equal to 100, 90, 80, 70, 60, 50, 40, 30, 20, 15, 10, 9.5, 9, 8.5, 8, 7.5, 7, 6.5, 6, 5.5, 5, 4.5, 4, 3.5, 3, 2.5, 2, 1.5, or 1.
196. The composition of any one of Clauses 132-195, wherein the activator is coupled to the substrate by a linker.
197. The composition of Clause 196, wherein the linker comprises a length greater than its persistence length.
198. The composition of Clause 196 or 197, wherein the linker has a length of between about 5 and about 100 nm.
199. The composition of any one of Clauses 196-198, wherein the linker comprises at least 2 repeating units, 5 repeating units, 10 repeating units, 15 repeating units, 20 repeating units, 25 repeating units, 30 repeating units, 35 repeating units, 40 repeating units, 45 repeating units, 50 repeating units, 55 repeating units, 60 repeating units, 65 repeating units, 70 repeating units, 75 repeating units, 80 repeating units, 85 repeating units, 90 repeating units, 95 repeating units, 100 repeating units, 110 repeating units, 120 repeating units, 130 repeating units, 140 repeating units, 150 repeating units, 160 repeating units, 170 repeating units, 180 repeating units, 190 repeating units, 200 repeating units, 500 repeating units, 1000 repeating units, or 2000 repeating units.
200. The composition of any one of Clauses 196-199, wherein the linker comprises no more than 2000 repeating units, 1000 repeating units, 500 repeating units, 200 repeating units, 190 repeating units, 180 repeating units, 170 repeating units, 160 repeating units, 150 repeating units, 140 repeating units, 130 repeating units, 120 repeating units, 110 repeating units, 100 repeating units, 95 repeating units, 90 repeating units, 85 repeating units, 80 repeating units, 75 repeating units, 70 repeating units, 65 repeating units, 60 repeating units, 55 repeating units, 50 repeating units, 45 repeating units, 40 repeating units, 35 repeating units, 30 repeating units, 25 repeating units, 20 repeating units, 15 repeating units, 10 repeating units, 5 repeating units, or 2 repeating units.
201. The composition of any one of Clauses 196-200, wherein the linker is a cleavable linker.
202. The composition of Clause 201, wherein the cleavable linker is a photocleavable linker, a chemically cleavable linker, or an enzymatically cleavable linker.
203. The composition of any one of Clauses 196-200, wherein the linker is a non-cleavable linker.
204. The composition of any one of Clauses 196-203, wherein the linker comprises polyethylene glycol.
205. The composition of any one of Clauses 132-204, wherein the cytokine receptor switch comprises a sequence having at least 80%, at least 90%, at least 95%, at least 97%, or 100% sequence identity to any one of SEQ ID NO: 1-SEQ ID NO: 7.
206. The composition of any one of Clauses 132-205, wherein the cytokine receptor switch comprises a sequence of any one of SEQ ID NO: 1-SEQ ID NO: 7.
207. The composition of any one of Clauses 132-205, wherein the immune cells express a chimeric antigen receptor.
208. The composition of Clause 207, wherein the chimeric antigen receptor binds to a target antigen on a cancer cell.
209. The composition of Clause 208, wherein the target antigen is selected from the group consisting of CD19, CD20, CD22, CD123, CD33, CD3, CD4, CD8, CD38, SLAMF7, BCMA, GD2, GPRC5D, MUC16, HER2, EGFR, EGFRVIII, CLL-1, CD44v6, folate receptor-α, B7-H3, EphA2, GRP78, NKG2D, CD70, and mesothelin.
210. The composition of Clause 207, wherein the chimeric antigen receptor binds to a synthetic antigen.
211. The composition of Clause 210, wherein the synthetic antigen is selected from the group consisting of fluorescein, fluorescein isothiocyanate (FITC), fluorescein 5-maleimide, fluorescein-5-carboxamide, fluorescein-6-carboxamide, 6-FAM phosphoramidite, topiramate hemisuccinate, creatine, acetaminophen, ketamine, propofol, lidocaine, ractopamine, salicylate, salicylic acid, sulfasalazine, dapsone, albendazole, ivermectin, levamisole, permethrin, pyrantel, thiabendazole, procainamide, sulfamethazine, amikacin, amoxicillin, ampicillin, cefazolin, cefuroxime, cephalexin, chloramphenicol, chloramphenicol, ciprofloxacin, clenbuterol, cloxacillin, colistin A, dicloxacillin, enrofloxacin, furaltadone, gentamicin, gentamicin, kanamycin, kanamycin, kincomycin, lincomycin, metronidazole, nafcillin, nalidixic acid, neomycin, neomycin, nitrofurazone, norfloxacin, ofloxacin, oxacillin, spectinomycin, streptomycin, streptomycin, sulfabenzamide, sulfacetamide, sulfadiazine, sulfadimidine, sulfametoxydiazine, sulfanilamide, trimethoprim, carbamazepine, ethosuximide, lamotrigine, primidone, cetirizine, chlorpheniramine, diphenhydramine, doxylamine, promethazine, sulfadimethoxine, benzothiazinone, butylated hydroxytoluene, tripelennamine, chlorpromazine, clozapine, haloperidol, olanzapine, paliperidone, quetiapine, ribavirin, meprobamate, acebutolol, atenolol, penbutolol, warfarin, salmeterol, aflatoxin B1, tetraxetan (DOTA), 4-[(6-methylpyrazin-2-yl)oxy]benzoate (MPOB), biotin, melamine, methotrexate, amphetamine, diethylpropion, dextromethorphan, pseudoephedrine, dihydrochlorothiazide, hydrochlorothiazide, clonazepam, diazepam, nitrazepam, rhodamine B, fluorescent brightener Ksn, zearalenone, Sudan Red1, acetominophen, acrylamide, benzoic acid, benzophenone, benzothiazine, mercaptobenzothiazole, erythrosine, Sudan, tartrazine, erythromycin, sirolimus, atropine, ethyl glucuronide, aflatoxin M1, methocarbamol, fentanyl, hydromorphone, morphine, remifentanil, tapentadol, tramadol, pregabalin, gabapentin, amitriptyline, desipramine, imipramine, nortriptyline, venlafaxine, dinitrophenyl, His-tag, PEG methoxy group, etodolac, ibuprofen, ketoprofen, meclofenamic acid, phenylbutazone, acetyl salicylic acid, acetamiprid, acetochlor, carbadazim, carbaryl, chlorothalonil, chlorpyrifos, fenpropathrin, imazalil, imidacloprid, parathion, abscisic acid, dibutyl phthalate, clonazepam, lorazepam, oxazepam, phenobarbital, secobarbital, zaleplon, zolpidem, trazodone, fluoxetine, fluvoxamine, cortisone, dexamethasone, dihydrotestosterone, fluocinolone, methylprednisolone, prednisolone, stanozolol, triamcinolone, mazindol, methamphetamine, methylphenidate, modafinil, chrysoidine, deoxynivalenol, fumonisin, microcystin Lr, ochratoxin, sterigmatocystin, T-2 toxin, sildenafil, tadalafil, scopolamine, florfenicol, pirlimycin, and sulfaquinoxaline.
212. The composition of any one of Clauses 207-211, wherein the chimeric antigen receptor is encoded by a sequence having at least 80%, at least 90%, at least 95%, at least 97%, or 100% sequence identity to any one of SEQ ID NO: 40-SEQ ID NO: 45.
213. The composition of any one of Clauses 207-212, wherein the chimeric antigen receptor is encoded by a sequence of any one of SEQ ID NO: 40-SEQ ID NO: 45.
214. The composition of any one of Clauses 207-213, wherein the cytokine receptor switch and the chimeric antigen receptor are encoded by a sequence having at least 80%, at least 90%, at least 95%, at least 97%, or 100% sequence identity to any one of SEQ ID NO: 46-SEQ ID NO: 52.
215. The composition of any one of Clauses 207-214, wherein the cytokine receptor switch and the chimeric antigen receptor are encoded by a sequence of any one of SEQ ID NO: 46-SEQ ID NO: 52.
216. A method for activating an immune cell population using the composition of any one of Clauses 132-215.
217. A kit for manufacturing an immune cell population comprising the composition of any one of Clauses 132-215.

### CONCLUSION

Although many of the embodiments are described above with respect to systems, devices, and methods for cancer immunotherapy, the technology is applicable to other applications and/or other approaches, such as immunotherapy for other types of diseases and conditions. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-92D.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the term “ex vivo” can denote manipulation, modification, or expansion outside of a living organism for cells derived from a living organism.

As used herein, the term “transfection” refers to any method for introducing a nucleic acid into a cell, including both viral and non-viral methods, and encompasses both transient and stable modifications.

The term “lentivirus” as used herein can refer to any genus of the Retroviridae family, such HIV, SIV, and FIV, as well as natural and synthetic virions and subvirions thereof.

As used herein, the term “peptide” can refer to oligomers (“oligopeptides”) and polymers (“polypeptides”) of amino acids. While many instances of the term “peptide” refer to straight chains of naturally occurring amino acids (e.g., an unmodified or post-translationally modified canonical amino acid) coupled by peptide bonds, a peptide can also include branched chains, peptide bond analogues (e.g., a peptide backbone can include peptide bonds interspersed by a thioether bond), and non-naturally occurring amino acids. In some cases, a peptide is or comprises a protein.

As used herein, the term “antibody” can refer to a peptide with sequence and structural similarity to an IgG, IgM, IgA, IgD, or IgE immunoglobulin, a fragment thereof, an ortholog or homolog thereof (e.g., a camelid antibody), or a combination thereof. An antibody can be derived from a species from which it is naturally produced, or from a cell or species recombinantly modified to produce the antibody. Exemplary IgG, IgD, and IgE antibodies are typically tetramers consisting of two light chain and two heavy chain peptides of about 25 kD and 50-70 kD, respectively.

As used herein, the term percent “identity,” in the context of two or more nucleic acid or polypeptide sequences, may refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity may be performed using the BLAST algorithm, which is described in Altschul et al. (J. Mol. Biol. 215:403-410 (1990)). Software for performing BLAST analyses is publicly available through the National Center for Biotechnology Information.

All terms, chemical names, expressions, and designations have their usual meanings which are well-known to those skilled in the art. When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individually or in any combination. Additionally, unless otherwise specified, all isotopic variants of compounds disclosed herein are intended to be encompassed by the disclosure. Specific names of compounds are intended to be exemplary, as it is known that one of ordinary skill in the art can name the same compounds differently.

As used herein, the term “group” may refer to a reactive functional group of a chemical compound. Groups of the present compounds refer to an atom or a collection of atoms that are a part of the compound. Groups of the present disclosure may be attached to other atoms of the compound via one or more covalent bonds. Groups may also be characterized with respect to their valence state. The present disclosure includes groups characterized as monovalent, divalent, trivalent, etc. valence states.

As used herein, the term “substituted” refers to a compound (e.g., an alkyl chain) wherein a hydrogen is replaced by another reactive functional group or atom, as described herein.

As used herein, a broken line in a chemical structure can be used to indicate a bond to the rest of the molecule. For example, [symbol] in

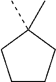

is used to designate the 1-position as the point of attachment of 1-methylcyclopentate to the rest of the molecule. Alternatively, [symbol] in, e.g.,

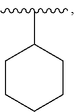 [SMILES: *C1CCCCC1]

can be used to indicate that the given moiety, the cyclohexyl moiety in this example, is attached to a molecule via the bond that is “capped” with the wavy line.

The following definitions are used, unless otherwise described: halo is fluoro, chloro, bromo, or iodo. Alkyl, alkoxy, alkenyl, alkynyl, etc., denote both straight and branched groups; but reference to an individual radical such as propyl embraces only the straight chain radical, a branched chain isomer such as isopropyl being specifically referred to. Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms comprising one to four heteroatoms each selected from the group consisting of non-peroxide oxygen, sulfur, and N(X).

It will be appreciated by those skilled in the art that compounds of the disclosure having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆) cycloalkyl can be cyclopropyl, cyclobutyl, cyclopentyl, or cyclohexyl; (C₃-C₆) cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1, pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The term “alkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) and is derived by the removal of one hydrogen atom from the parent alkane. Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkylene” refers to a bivalent unsubstituted saturated branched or straight chain hydrocarbon of the stated number of carbon atoms (e.g., a C₁-C₆ alkylene has from 1 to 6 carbon atoms) and having two monovalent centers derived by the removal of two hydrogen atoms from the same or two different carbon atoms of the parent alkane. Alkylene groups can be substituted with 1-6 fluoro groups, for example, on the carbon backbone (as —CHF— or —CF₂—) or on terminal carbons of straight chain or branched alkylenes (such as —CHF₂ or —CF₃). Alkylene groups include but are not limited to: methylene (—CH₂—), ethylene (—CH₂CH₂—), n-propylene (—CH₂CH₂CH₂—), n-propylene (—CH₂CH₂CH₂—), n-butylene (—CH₂CH₂CH₂CH₂—), difluoro-methylene (—CF₂—), tetrafluoroethylene (—CF₂CF₂—), and the like.

The term “alkenyl” refers to an unsubstituted straight chain or branched, hydrocarbon having at least one carbon-carbon double bond and the indicated number of carbon atoms (e.g., “C₂-C₅ alkenyl” or “C₂-C₁₀” alkenyl have from 2 to 8 or 2 to 10 carbon atoms, respectively). When the number of carbon atoms is not indicated, the alkenyl group has from 2 to 6 carbon atoms.

The term “heteroalkyl” refers to a stable straight or branched chain saturated hydrocarbon having the stated number of total atoms and at least one (e.g., 1 to 15) heteroatom selected from the group consisting of O, N, Si and S. The carbon and heteroatoms of the heteroalkyl group can be oxidized (e.g., to form ketones, N-oxides, sulfones, and the like) and the nitrogen atoms can be quaternized. The heteroatom(s) can be placed at any interior position of the heteroalkyl group and/or at any terminus of the heteroalkyl group, including termini of branched heteroalkyl groups), and/or at the position at which the heteroalkyl group is attached to the remainder of the molecule. Heteroalkyl groups can be substituted with 1-6 fluoro groups, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂—CH₂—O—CH₃, —CH₂—CH₂—NH—CH₃, —CH₂—CH₂—N(CH₃)₂, —C(═O)—NH—CH₂—CH₂—NH—CH₃, —C(═O)—N(CH₃)—CH₂—CH₂—N(CH₃)₂, —C(═O)—NH—CH₂—CH₂—NH—C(═O)—CH₂—CH₃, —C(═O)—N(CH₃)—CH₂—CH₂—N(CH₃)—C(═O)—CH₂—CH₃, —O—CH₂—CH₂—CH₂—NH(CH₃), —O—CH₂—CH₂—CH₂—N(CH₃)₂, —O—CH₂—CH₂—CH₂—NH—C(═O)—CH₂—CH₃, —O—CH₂—CH₂—CH₂—N(CH₃)—C(═O)—CH₂—CH₃, —CH₂—CH₂—CH₂—NH(CH₃), —O—CH₂—CH₂—CH₂—N(CH₃)₂, —CH₂—CH₂—CH₂—NH—C(═O)—CH₂—CH₃, —CH₂—CH₂—CH₂—N(CH₃)—C(═O)—CH₂—CH₃, —CH₂—S—CH₂—CH₃, —CH₂—CH₂—S(O)—CH₃, —NH—CH₂—CH₂—NH—C(═O)—CH₂—CH₃, —CH₂—CH₂—S(O)₂—CH₃, —CH₂—CH₂—O—CF₃, and —Si(CH₃)₃. Up to two heteroatoms may be consecutive, such as, for example, —CH₂—NH—OCH₃ and —CH₂—O—Si(CH₃)₃. A terminal polyethylene glycol (PEG) moiety is a type of heteroalkyl group.

The term “alkynyl” refers to an unsubstituted straight chain or branched, hydrocarbon having at least one carbon-carbon triple bond and the indicated number of carbon atoms (e.g., “C₂-C₈ alkynyl” or “C₂-C₁₀” alkynyl have from 2 to 8 or 2 to 10 carbon atoms, respectively). When the number of carbon atoms is not indicated, the alkynyl group has from 2 to 6 carbon atoms.

The term “acyl” refers to an alkyl, haloalkyl, alkenyl, alkynyl, aryl cycloalkyl, heteroaryl, or heterocyclyl group, as defined herein, connected to the remainder of the compound by a C═O (carbonyl) group.

The term “carboxamido” refers to a —C(═O) NRR′ group, wherein R and R′ are independently selected from the group consisting of hydrogen, alkyl, alkenyl, alkynyl, aryl cycloalkyl, heteroaryl, and heterocyclyl, as defined herein.

The term “heteroalkylene” refers to a bivalent unsubstituted straight or branched group derived from heteroalkyl (e.g., as defined herein). Examples of heteroalkylene groups include, but are not limited to, —CH₂—CH₂—O—CH₂—, —CH₂—CH₂—O—CF₂—, —CH₂—CH₂—NH—CH₂—, —C(═O)—NH—CH₂—CH₂—NH—CH₂—C(═O)—N(CH₃)—CH₂—CH₂—N(CH₃)—CH₂—, —C(═O)—NH—CH₂—CH₂—NH—C(═O)—CH₂—CH₂—, —C(═O)—N(CH₃)—CH₂—CH₂—N(CH₃)—C(═O)—CH₂—CH₂—, —O—CH₂—CH₂—CH₂—NH—CH₂—, —O—CH₂—CH₂—CH₂—N(CH₃)—CH₂—, —O—CH₂—CH₂—CH₂—NH—C(═O)—CH₂—CH₂—, —O—CH₂—CH₂—CH₂—N(CH₃)—C(═O)—CH₂—CH₂—, —CH₂—CH₂—CH₂—NH—CH₂—, —CH₂—CH₂—CH₂—N(CH₃)—CH₂—, —CH₂—CH₂—CH₂—NH—C(═O)—CH₂—CH₂—, —CH₂—CH₂—CH₂—N(CH₃)—C(═O)—CH₂—CH₂—, —CH₂—CH₂—NH—C(═O)—, —CH₂—CH₂—N(CH₃)—CH₂—, —CH₂—CH₂—N⁺ (CH₃)₂—, —NH—CH₂—CH₂ (NH₂)—CH₂—, and —NH—CH₂—CH₂ (NHCH₃)—CH₂—. A bivalent polyethylene glycol (PEG) moiety is a type of heteroalkylene group.

The term “alkoxy” refers to an alkyl group, as defined herein, which is attached to a molecule via an oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “alkylthio” refers to an alkyl group, as defined herein, which is attached to a molecule via a sulfur atom. For example, alkythio groups include, but are not limited to thiomethyl, thioethyl, thio-n-propyl, thio-iso-propyl, and the like.

The term “haloalkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) wherein at least one hydrogen atom of the alkyl group is replaced by a halogen (e.g., fluoro, chloro, bromo, or iodo). When the number of carbon atoms is not indicated, the haloalkyl group has from 1 to 6 carbon atoms. Representative C₁₋₆ haloalkyl groups include, but are not limited to, trifluoromethyl, 2,2,2-trifluoroethyl, and 1-chloroisopropyl.

The term “cycloalkyl” refers to a cyclic, saturated, or partially unsaturated hydrocarbon having the indicated number of carbon atoms (e.g., “C₃₋₈ cycloalkyl” or “C₃₋₆” cycloalkyl have from 3 to 8 or 3 to 6 carbon atoms, respectively). When the number of carbon atoms is not indicated, the cycloalkyl group has from 3 to 6 carbon atoms. Cycloalkyl groups include bridged, fused, and spiro ring systems, and bridged bicyclic systems where one ring is aromatic and the other is unsaturated. Representative “C₃₋₆ cycloalkyl” groups include, cyclopropyl, cyclobutyl, cyclopentyl, and cyclohexyl.

The term “aryl” refers to an unsubstituted monovalent carbocyclic aromatic hydrocarbon group of 6-10 carbon atoms derived by the removal of one hydrogen atom from a single carbon atom of a parent aromatic ring system. Aryl groups include, but are not limited to, phenyl, naphthyl, anthracenyl, biphenyl, and the like.

The term “heterocycle” refers to a saturated or partially unsaturated ring or a multiple condensed ring system, including bridged, fused, and spiro ring systems. Heterocycles can be described by the total number of atoms in the ring system, for example a 3-10 membered heterocycle has 3 to 10 total ring atoms. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3)oxo groups and the sulfur and nitrogen atoms may also be present in their oxidized forms. Such rings include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more heterocycles (e.g., decahydronapthyridinyl), carbocycles (e.g., decahydroquinolyl) or aryls. The rings of a multiple condensed ring system can be connected to each other via fused, spiro and bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, and 1,4-benzodioxanyl.

The term “heteroaryl” refers to an aromatic hydrocarbon ring system with at least one heteroatom within a single ring or within a fused ring system, selected from the group consisting of O, N and S. The ring or ring system has 4n+2 electrons in a conjugated x system where all atoms contributing to the conjugated x system are in the same plane. In some embodiments, heteroaryl groups have 5-10 total ring atoms and 1, 2, or 3 heteroatoms (referred to as a “5-10 membered heteroaryl”). Heteroaryl groups include, but are not limited to, imidazole, triazole, thiophene, furan, pyrrole, benzimidazole, pyrazole, pyrazine, pyridine, pyrimidine, and indole.

The term “hydroxyl” refers to an —OH group. The term “cyano” refers to α-CN group. The term “carboxy” refers to α—C(═O) OH group. The term “oxo” refers to a ═O group.

The term “alkanoyl” refers to an alkyl group, as defined herein, connected to the remainder of the molecule by α—C(═O) group. Exemplary alkanoyl groups include, but are not limited to acetyl, n-propanoyl, and n-butanoyl.

The term “alkanoyloxy” refers to an alkyl group, as defined herein, connected to the remainder of the molecule by an —OC(═O) group. Exemplary alkanoyloxy groups include, but are not limited to acetoxy, n-propanoyloxy, and n-butanoyloxy.

The term “alkoxycarbonyl” refers to an alkoxy group, as defined herein, connected to a C(═O)-alkyl group via the oxygen atom of the alkoxy (i.e., an alkyl ester group).

The terms “arylalkyl” and “cycloalkylalkyl” refer to an aryl group or a cycloalkyl group (as defined herein) connected to the remainder of the molecule by an alkyl group, as defined herein. Exemplary arylalkyl groups include but are not limited to benzyl and phenethyl. Exemplary cycloalkylalkyl groups include, but are not limited to cyclopropylmethyl, cyclobutylmethyl, cyclopentylethyl, and cyclohexylethyl.

A composition can be administered alone or in combination with other treatments, either simultaneously or sequentially dependent upon the condition to be treated.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo, or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21st Ed., Mack Publ. Co., Easton, Pa. (2005), incorporated herein by reference in its entirety.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-217. (canceled)

218. A method of activating an immune cell population, the method comprising:
providing an immune cell population expressing an engineered cytokine receptor switch comprising an activator binding domain configured to bind to an activator, a transmembrane domain, and an intracellular domain;
contacting the immune cell population to a substrate comprising the activator, thereby activating the engineered cytokine receptor switch; and
converting at least a portion of the immune cell population to an effector phenotype, a memory phenotype, or a combination thereof, thereby activating the immune cell population.

219. The method of claim 218, wherein the activator binding domain comprises a single-chain variable fragment (scFv), a peptide, or a nanobody.

220. The method of claim 218, wherein the intracellular domain comprises a cytokine receptor intracellular domain.

221. The method of claim 218, wherein the intracellular domain comprises a sequence having at least 80% sequence identity to any one of SEQ ID NO: 29-SEQ ID NO: 34.

222. The method of claim 218, wherein the transmembrane domain comprises a sequence having at least 80% sequence identity to any one of SEQ ID NO: 23-SEQ ID NO: 28.

223. The method of claim 218, further comprising transfecting the immune cell population with a vector encoding a chimeric antigen receptor.

224. The method of claim 218, wherein the activator is a small molecule.

225. The method of claim 224, wherein the small molecule comprises fluorescein, tetraxetan, a derivative thereof, or a combination thereof.

226. The method of claim 218, wherein the substrate comprises a nanoparticle, a microparticle, a polymer matrix, a surface, a carbon nanomaterial, a quantum dot, or a combination thereof.

227. The method of claim 218, wherein the activator is coupled to the substrate by a linker.

228. The method of claim 227, wherein the linker has a length of between about 5 and about 100 nm.

229. The method of claim 227, wherein the linker is a cleavable linker.

230. The method of claim 229, wherein the cleavable linker is a photocleavable linker, a chemically cleavable linker, or an enzymatically cleavable linker.

231. The method of claim 229, further comprising cleaving the cleavable linker to release the immune cell population from the substrate.

232. The method of claim 227, wherein the linker is a non-cleavable linker.

233. The method of claim 218, wherein at least 80% of the immune cell population is converted to the memory phenotype.

234. The method of claim 218, wherein the cytokine receptor switch comprises a sequence having at least 80% sequence identity to any one of SEQ ID NO: 1-SEQ ID NO: 7.

235. The method of claim 218, further comprising administering the immune cell population to a subject.

236. The method of claim 218, further comprising recruiting an immune cell of the immune cell population to a cancer cell by binding a chimeric antigen receptor expressed by the immune cell to a target antigen on the cancer cell.

237. The method of claim 236, further comprising recruiting the immune cell to a cancer cell by administering a bispecific agent to the subject, wherein the bispecific agent comprises a targeting moiety that binds to a target antigen on the cancer cell and a synthetic antigen, and wherein a chimeric antigen receptor expressed by the immune cell binds to the synthetic antigen.
